SEQUENCE LISTING (281 sequences)

SEQ ID NO: 1 - protein, 152 aa, Homo sapiens
  UBE2A E2 domain
MSTPARRRLMRDFKRLQEDPPAGVSGAPSENNIMVWNAVIFGPEGTPFEDGTFKLTIEFTEEYPNKPPTVRFVSKMFHPNVYADGSICLDILQNRWSPTYDVSSILTSIQSLLDEPNPNSPANSQAAQLYQENKREYEKRVSAIVEQSWRDC

SEQ ID NO: 2 - protein, 152 aa, Homo sapiens
  UBE2B E2 domain
MSTPARRRLMRDFKRLQEDPPVGVSGAPSENNIMQWNAVIFGPEGTPFEDGTFKLVIEFSEEYPNKPPTVRFLSKMFHPNVYADGSICLDILQNRWSPTYDVSSILTSIQSLLDEPNPNSPANSQAAQLYQENKREYEKRVSAIVEQSWNDS

SEQ ID NO: 3 - protein, 179 aa, Homo sapiens
  UBE2C E2 domain
MASQNRDPAATSVAAARKGAEPSGGAARGPVGKRLQQELMTLMMSGDKGISAFPESDNLFKWVGTIHGAAGTVYEDLRYKLSLEFPSGYPYNAPTVKFLTPCYHPNVDTQGNICLDILKEKWSALYDVRTILLSIQSLLGEPNIDSPLNTHAAELWKNPTAFKKYLQETYSKQVTSQEP

SEQ ID NO: 4 - protein, 147 aa, Homo sapiens
  UBE2D1 E2 domain
MALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAM

SEQ ID NO: 5 - protein, 147 aa, Homo sapiens
  UBE2D2 E2 domain
MALKRIHKELNDLARDPPAQCSAGPVGDDMFHWQATIMGPNDSPYQGGVFFLTIHFPTDYPFKPPKVAFTTRIYHPNINSNGSICLDILRSQWSPALTISKVLLSICSLLCDPNPDDPLVPEIARIYKTDREKYNRIAREWTQKYAM

SEQ ID NO: 6 - protein, 147 aa, Homo sapiens
  UBE2D3 E2 domain
MALKRINKELSDLARDPPAQCSAGPVGDDMFHWQATIMGPNDSPYQGGVFFLTIHFPTDYPFKPPKVAFTTRIYHPNINSNGSICLDILRSQWSPALTISKVLLSICSLLCDPNPDDPLVPEIARIYKTDRDKYNRISREWTQKYAM

SEQ ID NO: 7 - protein, 147 aa, Homo sapiens
  UBE2D4 E2 domain
MALKRIQKELTDLQRDPPAQCSAGPVGDDLFHWQATIMGPNDSPYQGGVFFLTIHFPTDYPFKPPKVAFTTKIYHPNINSNGSICLDILRSQWSPALTVSKVLLSICSLLCDPNPDDPLVPEIAHTYKADREKYNRLAREWTQKYAM

SEQ ID NO: 8 - protein, 193 aa, Homo sapiens
  UBE2E1 E2 domain
MSDDDSRASTSSSSSSSSNQQTEKETNTPKKKESKVSMSKNSKLLSTSAKRIQKELADITLDPPPNCSAGPKGDNIYEWRSTILGPPGSVYEGGVFFLDITFTPEYPFKPPKVTFRTRIYHCNINSQGVICLDILKDNWSPALTISKVLLSICSLLTDCNPADPLVGSIATQYMTNRAEHDRMARQWTKRYAT

SEQ ID NO: 9 - protein, 201 aa, Homo sapiens
  UBE2E2 E2 domain
MSTEAQRVDDSPSTSGGSSDGDQRESVQQEPEREQVQPKKKEGKISSKTAAKLSTSAKRIQKELAEITLDPPPNCSAGPKGDNIYEWRSTILGPPGSVYEGGVFFLDITFSPDYPFKPPKVTFRTRIYHCNINSQGVICLDILKDNWSPALTISKVLLSICSLLTDCNPADPLVGSIATQYMTNRAEHDRMARQWTKRYAT

SEQ ID NO: 10 - protein, 207 aa, Homo sapiens
  UBE2E3 E2 domain
MSSDRQRSDDESPSTSSGSSDADQRDPAAPEPEEQEERKPSATQQKKNTKLSSKTTAKLSTSAKRIQKELAEITLDPPPNCSAGPKGDNIYEWRSTILGPPGSVYEGGVFFLDITFSSDYPFKPPKVTFRTRIYHCNINSQGVICLDILKDNWSPALTISKVLLSICSLLTDCNPADPLVGSIATQYLTNRAEHDRIARQWTKRYAT

SEQ ID NO: 11 - protein, 185 aa, Homo sapiens
  UBE2F E2 domain
MLTLASKLKRDDGLKGSRTAATASDSTRRVSVRDKLLVKEVAELEANLPCTCKVHFPDPNKLHCFQLTVTPDEGYYQGGKFQFETEVPDAYNMVPPKVKCLTKIWHPNITETGEICLSLLREHSIDGTGWAPTRTLKDVVWGLNSLFTDLLNFDDPLNIEAAEHHLRDKEDFRNKVDDYIKRYAR

SEQ ID NO: 12 - protein, 170 aa, Homo sapiens
  UBE2G1 E2 domain
MTELQSALLLRRQLAELNKNPVEGFSAGLIDDNDLYRWEVLIIGPPDTLYEGGVFKAHLTFPKDYPLRPPKMKFITEIWHPNVDKNGDVCISILHEPGEDKYGYEKPEERWLPIHTVETIMISVISMLADPNGDSPANVDAAKEWREDRNGEFKRKVARCVRKSQETAFE

SEQ ID NO: 13 - protein, 165 aa, Homo sapiens
  UBE2G2 E2 domain
MAGTALKRLMAEYKQLTLNPPEGIVAGPMNEENFFEWEALIMGPEDTCFEFGVFPAILSFPLDYPLSPPKMRFTCEMFHPNIYPDGRVCISILHAPGDDPMGYESSAERWSPVQSVEKILLSVVSMLAEPNDESGANVDASKMWRDDREQFYKIAKQIVQKSLGL

SEQ ID NO: 14 - protein, 183 aa, Homo sapiens
  UBE2H E2 domain
MSSPSPGKRRMDTDVVKLIESKHEVTILGGLNEFVVKFYGPQGTPYEGGVWKVRVDLPDKYPFKSPSIGFMNKIFHPNIDEASGTVCLDVINQTWTALYDLTNIFESFLPQLLAYPNPIDPLNGDAAAMYLHRPEEYKQKIKEYIQKYATEEALKEQEEGTGDSSSESSMSDFSEDEAQDMEL

SEQ ID NO: 15 - protein, 158 aa, Homo sapiens [sequence omitted]

SEQ ID NO: 16 - protein, 318 aa, Homo sapiens
  UBE2J1 E2 domain
METRYNLKSPAVKRLMKEAAELKDPTDHYHAQPLEDNLFEWHFTVRGPPDSDFDGGVYHGRIVLPPEYPMKPPSIILLTANGRFEVGKKICLSISGHHPETWQPSWSIRTALLAIIGFMPTKGEGAIGSLDYTPEERRALAKKSQDFCCEGCGSAMKDVLLPLKSGSDSSQADQEAKELARQISFKAEVNSSGKTISESDLNHSFSLTDLQDDIPTTFQGATASTSYGLQNSSAASFHQPTQPVAKNTSMSPRQRRAQQQSQRRLSTSPDVIQGHQPRDNHTDHGGSAVLIVILTLALAALIFRRIYLANEYIFDFEL

SEQ ID NO: 17 - protein, 259 aa, Homo sapiens
  UBE2J2 E2 domain
MSSTSSKRAPTTATQRLKQDYLRIKKDPVPYICAEPLPSNILEWHYVVRGPEMTPYEGGYYHGKLIFPREFPFKPPSIYMITPNGRFKCNTRLCLSITDFHPDTWNPAWSVSTILTGLLSFMVEKGPTLGSIETSDFTKRQLAVQSLAFNLKDKVFCELFPEVVEEIKQKQKAQDELSSRPQTLPLPDVVPDGETHLVQNGIQLLNGHAPGAVPNLAGLQQANRHHGLLGGALANLFVIVGFAAFAYTVKYVLRSIAQE

SEQ ID NO: 18 - protein, 200 aa, Homo sapiens
  UBE2K E2 domain
MANIAVQRIKREFKEVLKSEETSKNQIKVDLVDENFTELRGEIAGPPDTPYEGGRYQLEIKIPETYPFNPPKVRFITKIWHPNISSVTGAICLDILKDQWAAAMTLRTVLLSLQALLAAAEPDDPQDAVVANQYKQNPEMFKQTARLWAHVYAGAPVSSPEYTKKIENLCAMGFDRNAVIVALSSKSWDVETATELLLSN

SEQ ID NO: 19 - protein, 154 aa, Homo sapiens [sequence omitted]

SEQ ID NO: 20 - protein, 153 aa, Homo sapiens
  UBE2L6 E2 domain
MMASMRVVKELEDLQKKPPPYLRNLSSDDANVLVWHALLLPDQPPYHLKAFNLRISFPPEYPFKPPMIKFTTKIYHPNVDENGQICLPIISSENWKPCTKTCQVLEALNVLVNRPNIREPLRMDLADLLTQNPELFRKNAEEFTLRFGVDRPS

SEQ ID NO: 21 - protein, 183 aa, Homo sapiens
  UBE2M E2 domain
MIKLFSLKQQKKEEESAGGTKGSSKKASAAQLRIQKDINELNLPKTCDISFSDPDDLLNFKLVICPDEGFYKSGKFVFSFKVGQGYPHDPPKVKCETMVYHPNIDLEGNVCLNILREDWKPVLTINSIIYGLQYLFLEPNPEDPLNKEAAEVLQNNRRLFEQNVQRSMRGGYIGSTYFERCLK

SEQ ID NO: 22 - protein, 152 aa, Homo sapiens
  UBE2N E2 domain
MAGLPRRIIKETQRLLAEPVPGIKAEPDESNARYFHVVIAGPQDSPFEGGTFKLELFLPEEYPMAAPKVRFMTKIYHPNVDKLGRICLDILKDKWSPALQIRTVLLSIQALLSAPNPDDPLANDVAEQWKTNEAQAIETARAWTRLYAMNNI

SEQ ID NO: 23 - protein, 153 aa, Homo sapiens
  UBE2NL E2 domain
MAELPHRIIKETQRLLAEPVPGIKAEPDESNARYFHVVIAGESKDSPFEGGTFKRELLLAEEYPMAAPKVRFMTKIYHPNVDKLERISLDILKDKWSPALQIRTVLLSIQALLNAPNPDDPLANDVVEQWKTNEAQAIETARAWTRLYAMNSI

SEQ ID NO: 24 - protein, 1292 aa, Homo sapiens
  UBE2O E2 domain
MADPAAPTPAAPAPAQAPAPAPEAVPAPAAAPVPAPAPASDSASGPSSDSGPEAGSQRLLFSHDLVSGRYRGSVHFGLVRLIHGEDSDSEGEEEGRGSSGCSEAGGAGHEEGRASPLRRGYVRVQWYPEGVKQHVKETKLKLEDRSVVPRDVVRHMRSTDSQCGTVIDVNIDCAVKLIGTNCIIYPVNSKDLQHIWPFMYGDYIAYDCWLGKVYDLKNQIILKLSNGARCSMNTEDGAKLYDVCPHVSDSGLFFDDSYGFYPGQVLIGPAKIFSSVQWLSGVKPVLSTKSKFRVVVEEVQVVELKVTWITKSFCPGGTDSVSPPPSVITQENLGRVKRLGCFDHAQRQLGERCLYVFPAKVEPAKIAWECPEKNCAQGEGSMAKKVKRLLKKQVVRIMSCSPDTQCSRDHSMEDPDKKGESKTKSEAESASPEETPDGSASPVEMQDEGAEEPHEAGEQLPPFLLKEGRDDRLHSAEQDADDEAADDTDDTSSVTSSASSTTSSQSGSGTSRKKSIPLSIKNLKRKHKRKKNKITRDFKPGDRVAVEVVTTMTSADVMWQDGSVECNIRSNDLFPVHHLDNNEFCPGDFVVDKRVQSCPDPAVYGVVQSGDHIGRTCMVKWFKLRPSGDDVELIGEEEDVSVYDIADHPDFRFRTTDIVIRIGNTEDGAPHKEDEPSVGQVARVDVSSKVEVVWADNSKTIILPQHLYNIESEIEESDYDSVEGSTSGASSDEWEDDSDSWETDNGLVEDEHPKIEEPPIPPLEQPVAPEDKGVVISEEAATAAVQGAVAMAAPMAGLMEKAGKDGPPKSFRELKEAIKILESLKNMTVEQLLTGSPTSPTVEPEKPTREKKFLDDIKKLQENLKKTLDNVAIVEEEKMEAVPDVERKEDKPEGQSPVKAEWPSETPVLCQQCGGKPGVTFTSAKGEVFSVLEFAPSNHSFKKIEFQPPEAKKFFSTVRKEMALLATSLPEGIMVKTFEDRMDLFSALIKGPTRTPYEDGLYLFDIQLPNIYPAVPPHFCYLSQCSGRLNPNLYDNGKVCVSLLGTWIGKGTERWTSKSSLLQVLISIQGLILVNEPYYNEAGFDSDRGLQEGYENSRCYNEMALIRVVQSMTQLVRRPPEVFEQEIRQHFSTGGWRLVNRIESWLETHALLEKAQALPNGVPKASSSPEPPAVAELSDSGQQEPEDGGPAPGEASQGSDSEGGAQGLASASRDHTDQTSETAPDASVPPSVKPKKRRKSYRSFLPEKSGYPDIGFPLFPLSKGFIKSIRGVLTQFRAALLEAGMPECTEDK

SEQ ID NO: 25 - protein, 422 aa, Homo sapiens
  UBE2Q1 E2 domain
MQQPQPQGQQQPGPGQQLGGQGAAPGAGGGPGGGPGPGPCLRRELKLLESIFHRGHERFRIASACLDELSCEFLLAGAGGAGAGAAPGPHLPPRGSVPGDPVRIHCNITESYPAVPPIWSVESDDPNLAAVLERLVDIKKGNTLLLQHLKRIISDLCKLYNLPQHPDVEMLDQPLPAEQCTQEDVSSEDEDEEMPEDTEDLDHYEMKEEEPAEGKKSEDDGIGKENLAILEKIKKNQRQDYLNGAVSGSVQATDRLMKELRDIYRSQSFKGGNYAVELVNDSLYDWNVKLLKVDQDSALHNDLQILKEKEGADFILLNFSFKDNFPFDPPFVRVVSPVLSGGYVLGGGAICMELLTKQGWSSAYSIESVIMQISATLVKGKARVQFGANKSQYSLTRAQQSYKSLVQIHEKNGWYTPPKEDG

SEQ ID NO: 26 - protein, 375 aa, Homo sapiens
  UBE2Q2 E2 domain
MSVSGLKAELKFLASIFDKNHERFRIVSWKLDELHCQFLVPQQGSPHSLPPPLTLHCNITESYPSSSPIWFVDSEDPNLTSVLERLEDTKNNNLLRQQLKWLICELCSLYNLPKHLDVEMLDQPLPTGQNGTTEEVTSEEEEEEEEMAEDIEDLDHYEMKEEEPISGKKSEDEGIEKENLAILEKIRKTQRQDHLNGAVSGSVQASDRLMKELRDIYRSQSYKTGIYSVELINDSLYDWHVKLQKVDPDSPLHSDLQILKEKEGIEYILLNFSFKDNFPFDPPFVRVVLPVLSGGYVLGGGALCMELLTKQGWSSAYSIESVIMQINATLVKGKARVQFGANKNQYNLARAQQSYNSIVQIHEKNGWYTPPKEDG

SEQ ID NO: 27 - protein, 161 aa, Homo sapiens
  UBE2QL E2 domain
MKELQDIARLSDRFISVELVDESLFDWNVKLHQVDKDSVLWQDMKETNTEFILLNLTFPDNFPFSPPFMRVLSPRLENGYVLDGGAICMELLTPRGWSSAYTVEAVMRQFAASLVKGQGRICRKAGKSKKSFSRKEAEATFKSLVKTHEKYGWVTPPVSDG

SEQ ID NO: 28 - protein, 236 aa, Homo sapiens
  UBE2R1 E2 domain
MARPLVPSSQKALLLELKGLQEEPVEGFRVTLVDEGDLYNWEVAIFGPPNTYYEGGYFKARLKFPIDYPYSPPAFRFLTKMWHPNIYETGDVCISILHPPVDDPQSGELPSERWNPTQNVRTILLSVISLLNEPNTFSPANVDASVMYRKWKESKGKDREYTDIIRKQVLGTKVDAERDGVKVPTTLAEYCVKTKAPAPDEGSDLFYDDYYEDGEVEEEADSCFGDDEDDSGTEES

SEQ ID NO: 29 - protein, 238 aa, Homo sapiens
  UBE2R2 E2 domain
MAQQQMTSSQKALMLELKSLQEEPVEGFRITLVDESDLYNWEVAIFGPPNTLYEGGYFKAHIKFPIDYPYSPPTFRFLTKMWHPNIYENGDVCISILHPPVDDPQSGELPSERWNPTQNVRTILLSVISLLNEPNTFSPANVDASVMFRKWRDSKGKDKEYAEIIRKQVSATKAEAEKDGVKVPTTLAEYCIKTKVPSNDNSSDLLYDDLYDDDIDDEDEEEEDADCYDDDDSGNEES

SEQ ID NO: 30 - protein, 222 aa, Homo sapiens
  UBE2S E2 domain
MNSNVENLPPHIIRLVYKEVTTLTADPPDGIKVFPNEEDLTDLQVTIEGPEGTPYAGGLFRMKLLLGKDFPASPPKGYFLTKIFHPNVGANGEICVNVLKRDWTAELGIRHVLLTIKCLLIHPNPESALNEEAGRLLLENYEEYAARARLLTEIHGGAGGPSGRAEAGRALASGTEASSTDPGAPGGPGGAEGPMAKKHAGERDKKLAAKKKTDKKRALRRL

SEQ ID NO: 31 - protein, 197 aa, Homo sapiens
  UBE2T E2 domain
MQRASRLKRELHMLATEPPPGITCWQDKDQMDDLRAQILGGANTPYEKGVFKLEVIIPERYPFEPPQIRFLTPIYHPNIDSAGRICLDVLKLPPKGAWRPSLNIATVLTSIQLLMSEPNPDDPLMADISSEFKYNKPAFLKNARQWTEKHARQKQKADEEEMLDNLPEAGDSRVHNSTQKRKASQLVGIEKKFHPDV

SEQ ID NO: 32 - protein, 321 aa, Homo sapiens
  UBE2U E2 domain
MHGRAYLLLHRDFCDLKENNYKGITAKPVSEDMMEWEVEIEGLQNSVWQGLVFQLTIHFTSEYNYAPPVVKFITIPFHPNVDPHTGQPCIDFLDNPEKWNTNYTLSSILLALQVMLSNPVLENPVNLEAARILVKDESLYRTILRLFNRPLQMKDDSQELPKDPRKCIRPIKTTSFSDYYQTWSRIATSKATEYYRTPLLKVPNFIGQYYKWKKMDLQHQKEWNLKYSVIKCWLARKRMPHEVTHSMEEIKLCPTLIPTTDEIFLESPTAINSITDIYETEEEGWKSDTSLYENDTDEPREEEVEDLISWTNTLNTNTSED

SEQ ID NO: 33 - protein, 147 aa, Homo sapiens
  UBE2V1 E2 domain
MAATTGSGVKVPRNFRLLEELEEGQKGVGDGTVSWGLEDDEDMTLTRWTGMIIGPPRTIYENRIYSLKIECGPKYPEAPPFVRFVTKINMNGVNSSNGVVDPRAISVLAKWQNSYSIKVVLQELRRLMMSKENMKLPQPPEGQCYSN

SEQ ID NO: 34 - protein, 145 aa, Homo sapiens
  UBE2V2 E2 domain
MAVSTGVKVPRNFRLLEELEEGQKGVGDGTVSWGLEDDEDMTLTRWTGMIIGPPRTNYENRIYSLKVECGPKYPEAPPSVRFVTKINMNGINNSSGMVDARSIPVLAKWQNSYSIKVVLQELRRLMMSKENMKLPQPPEGQTYNN

SEQ ID NO: 35 - protein, 151 aa, Homo sapiens
  UBE2W E2 domain
MASMQKRLQKELLALQNDPPPGMTLNEKSVQNSITQWIVDMEGAPGTLYEGEKFQLLFKFSSRYPFDSPQVMFTGENIPVHPHVYSNGHICLSILTEDWSPALSVQSVCLSIISMLSSCKEKRRPPDNSFYVRTCNKNPKKTKWWYHDDTC

SEQ ID NO: 36 - protein, 354 aa, Homo sapiens
  UBE2Z E2 domain
MAESPTEEAATAGAGAAGPGASSVAGVVGVSGSGGGFGPPFLPDVWAAAAAAGGAGGPGSGLAPLPGLPPSAAAHGAALLSHWDPTLSSDWDGERTAPQCLLRIKRDIMSIYKEPPPGMFVVPDTVDMTKIHALITGPFDTPYEGGFFLFVFRCPPDYPIHPPRVKLMTTGNNTVRFNPNFYRNGKVCLSILGTWTGPAWSPAQSISSVLISIQSLMTENPYHNEPGFEQERHPGDSKNYNECIRHETIRVAVCDMMEGKCPCPEPLRGVMEKSFLEYYDFYEVACKDRLHLQGQTMQDPFGEKRGHFDYQSLLMRLGLIRQKVLERLHNENAEMDSDSSSSGTETDLHGSLRV

SEQ ID NO: 37 - protein, 471 aa, Homo sapiens
  UEVLD E2 domain
MEFDCEGLRRLLGKYKFRDLTVEELRNVNVFFPHFKYSMDTYVFKDSSQKDLLNFTGTIPVMYQGNTYNIPIRFWILDSHPFAPPICFLKPTANMGILVGKHVDAQGRIYLPYLQNWSHPKSVIVGLIKEMIAKFQEELPMYSLSSSDEARQVDLLAYIAKITEGVSDTNSKSWANHENKTVNKITVVGGGELGIACTLAISAKGIADRLVLLDLSEGTKGATMDLEIFNLPNVEISKDLSASAHSKVVIFTVNSLGSSQSYLDVVQSNVDMFRALVPALGHYSQHSVLLVASQPVEIMTYVTWKLSTFPANRVIGIGCNLDSQRLQYIITNVLKAQTSGKEVWVIGEQGEDKVLTWSGQEEVVSHTSQVQLSNRAMELLRVKGQRSWSVGLSVADMVDSIVNNKKKVHSVSALAKGYYDINSEVFLSLPCILGTNGVSEVIKTTLKEDTVTEKLQSSASSIHSLQQQLKL

SEQ ID NO: 38 - protein, 4857 aa, Homo sapiens
  BIRC6 E2 domain
MVTGGGAAPPGTVTEPLPSVIVLSAGRKMAAAAAAASGPGCSSAAGAGAAGVSEWLVLRDGCMHCDADGLHSLSYHPALNAILAVTSRGTIKVIDGTSGATLQASALSAKPGGQVKCQYISAVDKVIFVDDYAVGCRKDLNGILLLDTALQTPVSKQDDVVQLELPVTEAQQLLSACLEKVDISSTEGYDLFITQLKDGLKNTSHETAANHKVAKWATVTFHLPHHVLKSIASAIVNELKKINQNVAALPVASSVMDRLSYLLPSARPELGVGPGRSVDRSLMYSEANRRETFTSWPHVGYRWAQPDPMAQAGFYHQPASSGDDRAMCFTCSVCLVCWEPTDEPWSEHERHSPNCPFVKGEHTQNVPLSVTLATSPAQFPCTDGTDRISCFGSGSCPHFLAAATKRGKICIWDVSKLMKVHLKFEINAYDPAIVQQLILSGDPSSGVDSRRPTLAWLEDSSSCSDIPKLEGDSDDLLEDSDSEEHSRSDSVTGHTSQKEAMEVSLDITALSILQQPEKLQWEIVANVLEDTVKDLEELGANPCLTNSKSEKTKEKHQEQHNIPFPCLLAGGLLTYKSPATSPISSNSHRSLDGLSRTQGESISEQGSTDNESCTNSELNSPLVRRTLPVLLLYSIKESDEKAGKIFSQMNNIMSKSLHDDGFTVPQIIEMELDSQEQLLLQDPPVTYIQQFADAAANLTSPDSEKWNSVFPKPGTLVQCLRLPKFAEEENLCIDSITPCADGIHLLVGLRTCPVESLSAINQVEALNNLNKLNSALCNRRKGELESNLAVVNGANISVIQHESPADVQTPLIIQPEQRNVSGGYLVLYKMNYATRIVTLEEEPIKIQHIKDPQDTITSLILLPPDILDNREDDCEEPIEDMQLTSKNGFEREKTSDISTLGHLVITTQGGYVKILDLSNFEILAKVEPPKKEGTEEQDTFVSVIYCSGTDRLCACTKGGELHFLQIGGTCDDIDEADILVDGSLSKGIEPSSEGSKPLSNPSSPGISGVDLLVDQPFTLEILTSLVELTRFETLTPRFSATVPPCWVEVQQEQQQRRHPQHLHQQHHGDAAQHTRTWKLQTDSNSWDEHVFELVLPKACMVGHVDFKFVLNSNITNIPQIQVTLLKNKAPGLGKVNALNIEVEQNGKPSLVDLNEEMQHMDVEESQCLRLCPFLEDHKEDILCGPVWLASGLDLSGHAGMLTLTSPKLVKGMAGGKYRSFLIHVKAVNERGTEEICNGGMRPVVRLPSLKHQSNKGYSLASLLAKVAAGKEKSSNVKNENTSGTRKSENLRGCDLLQEVSVTIRRFKKTSISKERVQRCAMLQFSEFHEKLVNTLCRKTDDGQITEHAQSLVLDTLCWLAGVHSNGPGSSKEGNENLLSKTRKFLSDIVRVCFFEAGRSIAHKCARFLALCISNGKCDPCQPAFGPVLLKALLDNMSFLPAATTGGSVYWYFVLLNYVKDEDLAGCSTACASLLTAVSRQLQDRLTPMEALLQTRYGLYSSPFDPVLFDLEMSGSSCKNVYNSSIGVQSDEIDLSDVLSGNGKVSSCTAAEGSFTSLTGLLEVEPLHFTCVSTSDGTRIERDDAMSSFGVTPAVGGLSSGTVGEASTALSSAAQVALQSLSHAMASAEQQLQVLQEKQQQLLKLQQQKAKLEAKLHQTTAAAAAAASAVGPVHNSVPSNPVAAPGFFIHPSDVIPPTPKTTPLFMTPPLTPPNEAVSVVINAELAQLFPGSVIDPPAVNLAAHNKNSNKSRMNPLGSGLALAISHASHFLQPPPHQSIIIERMHSGARRFVTLDFGRPILLTDVLIPTCGDLASLSIDIWTLGEEVDGRRLVVATDISTHSLILHDLIPPPVCRFMKITVIGRYGSTNARAKIPLGFYYGHTYILPWESELKLMHDPLKGEGESANQPEIDQHLAMMVALQEDIQCRYNLACHRLETLLQSIDLPPLNSANNAQYFLRKPDKAVEEDSRVFSAYQDCIQLQLQLNLAHNAVQRLKVALGASRKMLSETSNPEDLIQTSSTEQLRTIIRYLLDTLLSLLHASNGHSVPAVLQSTFHAQACEELFKHLCISGTPKIRLHTGLLLVQLCGGERWWGQFLSNVLQELYNSEQLLIFPQDRVFMLLSCIGQRSLSNSGVLESLLNLLDNLLSPLQPQLPMHRRTEGVLDIPMISWVVMLVSRLLDYVATVEDEAAAAKKPLNGNQWSFINNNLHTQSLNRSSKGSSSLDRLYSRKIRKQLVHHKQQLNLLKAKQKALVEQMEKEKIQSNKGSSYKLLVEQAKLKQATSKHFKDLIRLRRTAEWSRSNLDTEVTTAKESPEIEPLPFTLAHERCISVVQKLVLFLLSMDFTCHADLLLFVCKVLARIANATRPTIHLCEIVNEPQLERLLLLLVGTDFNRGDISWGGAWAQYSLTCMLQDILAGELLAPVAAEAMEEGTVGDDVGATAGDSDDSLQQSSVQLLETIDEPLTHDITGAPPLSSLEKDKEIDLELLQDLMEVDIDPLDIDLEKDPLAAKVFKPISSTWYDYWGADYGTYNYNPYIGGLGIPVAKPPANTEKNGSQTVSVSVSQALDARLEVGLEQQAELMLKMMSTLEADSILQALTNTSPTLSQSPTGTDDSLLGGLQAANQTSQLIIQLSSVPMLNVCFNKLFSMLQVHHVQLESLLQLWLTLSLNSSSTGNKENGADIFLYNANRIPVISLNQASITSFLTVLAWYPNTLLRTWCLVLHSLTLMTNMQLNSGSSSAIGTQESTAHLLVSDPNLIHVLVKFLSGTSPHGTNQHSPQVGPTATQAMQEFLTRLQVHLSSTCPQIFSEFLLKLIHILSTERGAFQTGQGPLDAQVKLLEFTLEQNFEVVSVSTISAVIESVTFLVHHYITCSDKVMSRSGSDSSVGARACFGGLFANLIRPGDAKAVCGEMTRDQLMFDLLKLVNILVQLPLSGNREYSARVSVTTNTTDSVSDEEKVSGGKDGNGSSTSVQGSPAYVADLVLANQQIMSQILSALGLCNSSAMAMIIGASGLHLTKHENFHGGLDAISVGDGLFTILTTLSKKASTVHMMLQPILTYMACGYMGRQGSLATCQLSEPLLWFILRVLDTSDALKAFHDMGGVQLICNNMVTSTRAIVNTARSMVSTIMKFLDSGPNKAVDSTLKTRILASEPDNAEGIHNFAPLGTITSSSPTAQPAEVLLQATPPHRRARSAAWSYIFLPEEAWCDLTIHLPAAVLLKEIHIQPHLASLATCPSSVSVEVSADGVNMLPLSTPVVTSGLTYIKIQLVKAEVASAVCLRLHRPRDASTLGLSQIKLLGLTAFGTTSSATVNNPFLPSEDQVSKTSIGWLRLLHHCLTHISDLEGMMASAAAPTANLLQTCAALLMSPYCGMHSPNIEVVLVKIGLQSTRIGLKLIDILLRNCAASGSDPTDLNSPLLFGRLNGLSSDSTIDILYQLGTTQDPGTKDRIQALLKWVSDSARVAAMKRSGRMNYMCPNSSTVEYGLLMPSPSHLHCVAAILWHSYELLVEYDLPALLDQELFELLFNWSMSLPCNMVLKKAVDSLLCSMCHVHPNYFSLLMGWMGITPPPVQCHHRLSMTDDSKKQDLSSSLTDDSKNAQAPLALTESHLATLASSSQSPEAIKQLLDSGLPSLLVRSLASFCFSHISSSESIAQSIDISQDKLRRHHVPQQCNKMPITADLVAPILRFLTEVGNSHIMKDWLGGSEVNPLWTALLFLLCHSGSTSGSHNLGAQQTSARSASLSSAATTGLTTQQRTAIENATVAFFLQCISCHPNNQKLMAQVLCELFQTSPQRGNLPTSGNISGFIRRLFLQLMLEDEKVTMFLQSPCPLYKGRINATSHVIQHPMYGAGHKFRTLHLPVSTTLSDVLDRVSDTPSITAKLISEQKDDKEKKNHEEKEKVKAENGFQDNYSVVVASGLKSQSKRAVSATPPRPPSRRGRTIPDKIGSTSGAEAANKIITVPVFHLFHKLLAGQPLPAEMTLAQLLTLLYDRKLPQGYRSIDLTVKLGSRVITDPSLSKTDSYKRLHPEKDHGDLLASCPEDEALTPGDECMDGILDESLLETCPIQSPLQVFAGMGGLALIAERLPMLYPEVIQQVSAPVVTSTTQEKPKDSDQFEWVTIEQSGELVYEAPETVAAEPPPIKSAVQTMSPIPAHSLAAFGLFLRLPGYAEVLLKERKHAQCLLRLVLGVTDDGEGSHILQSPSANVLPTLPFHVLRSLFSTTPLTTDDGVLLRRMALEIGALHLILVCLSALSHHSPRVPNSSVNQTEPQVSSSHNPTSTEEQQLYWAKGTGFGTGSTASGWDVEQALTKQRLEEEHVTCLLQVLASYINPVSSAVNGEAQSSHETRGQNSNALPSVLLELLSQSCLIPAMSSYLRNDSVLDMARHVPLYRALLELLRAIASCAAMVPLLLPLSTENGEEEEEQSECQTSVGTLLAKMKTCVDTYTNRLRSKRENVKTGVKPDASDQEPEGLTLLVPDIQKTAEIVYAATTSLRQANQEKKLGEYSKKAAMKPKPLSVLKSLEEKYVAVMKKLQFDTFEMVSEDEDGKLGFKVNYHYMSQVKNANDANSAARARRLAQEAVTLSTSLPLSSSSSVFVRCDEERLDIMKVLITGPADTPYANGCFEFDVYFPQDYPSSPPLVNLETTGGHSVRFNPNLYNDGKVCLSILNTWHGRPEEKWNPQTSSFLQVLVSVQSLILVAEPYFNEPGYERSRGTPSGTQSSREYDGNIRQATVKWAMLEQIRNPSPCFKEVIHKHFYLKRVEIMAQCEEWIADIQQYSSDKRVGRTMSHHAAALKRHTAQLREELLKLPCPEGLDPDTDDAPEVCRATTGAEETLMHDQVKPSSSKELPSDFQL

SEQ ID NO: 39 - protein, 292 aa, Homo sapiens
  FTS E2 domain
MNPFWSMSTSSVRKRSEGEEKTLTGDVKTSPPRTAPKKQLPSIPKNALPITKPTSPAPAAQSTNGTHASYGPFYLEYSLLAEFTLVVKQKLPGVYVQPSYRSALMWFGVIFIRHGLYQDGVFKFTVYIPDNYPDGDCPRLVFDIPVFHPLVDPTSGELDVKRAFAKWRRNHNHIWQVLMYARRVFYKIDTASPLNPEAAVLYEKDIQLFKSKVVDSVKVCTARLFDQPKIEDPYAISFSPWNPSVHDEAREKMLTQKKPEEQHNKSVHVAGLSWVKPGSVQPFSKEEKTVAT

SEQ ID NO: 40 - protein, 390 aa, Homo sapiens
  TSG101 E2 domain
MAVSESQLKKMVSKYKYRDLTVRETVNVITLYKDLKPVLDSYVFNDGSSRELMNLTGTIPVPYRGNTYNIPICLWLLDTYPYNPPICFVKPTSSMTIKTGKHVDANGKIYLPYLHEWKHPQSDLLGLIQVMIVVFGDEPPVFSRPISASYPPYQATGPPNTSYMPGMPGGISPYPSGYPPNPSGYPGCPYPPGGPYPATTSSQYPSQPPVTTVGPSRDGTISEDTIRASLISAVSDKLRWRMKEEMDRAQAELNALKRTEEDLKKGHQKLEEMVTRLDQEVAEVDKNIELLKKKDEELSSALEKMENQSENNDIDEVIIPTAPLYKQILNLYAEENAIEDTIFYLGEALRRGVIDLDVFLKHVRLLSRKQFQLRALMQKARKTAGLSDLY

SEQ ID NO: 41 - protein, 167 aa, Homo sapiens
  UFC1 E2 domain
MADEATRRVVSEIPVLKTNAGPRDRELWVQRLKEEYQSLIRYVENNKNADNDWFRLESNKEGTRWFGKCWYIHDLLKYEFDIEFDIPITYPTTAPEIAVPELDGKTAKMYRGGKICLTDHFKPLWARNVPKFGLAHLMALGLGPWLAVEIPDLIQKGVIQHKEKCNQ

SEQ ID NO: 42 - protein, 151 aa, Homo sapiens
  UBE2A UBC domain
STPARRRLMRDFKRLQEDPPAGVSGAPSENNIMVWNAVIFGPEGTPFEDGTFKLTIEFTEEYPNKPPTVRFVSKMFHPNVYADGSICLDILQNRWSPTYDVSSILTSIQSLLDEPNPNSPANSQAAQLYQENKREYEKRVSAIVEQSWRDC

SEQ ID NO: 43 - protein, 151 aa, Homo sapiens
  UBE2B UBC domain
STPARRRLMRDFKRLQEDPPVGVSGAPSENNIMQWNAVIFGPEGTPFEDGTFKLVIEFSEEYPNKPPTVRFLSKMFHPNVYADGSICLDILQNRWSPTYDVSSILTSIQSLLDEPNPNSPANSQAAQLYQENKREYEKRVSAIVEQSWNDS

SEQ ID NO: 44 - protein, 178 aa, Homo sapiens
  UBE2C UBC domain
ASQNRDPAATSVAAARKGAEPSGGAARGPVGKRLQQELMTLMMSGDKGISAFPESDNLFKWVGTIHGAAGTVYEDLRYKLSLEFPSGYPYNAPTVKFLTPCYHPNVDTQGNICLDILKEKWSALYDVRTILLSIQSLLGEPNIDSPLNTHAAELWKNPTAFKKYLQETYSKQVTSQEP

SEQ ID NO: 45 - protein, 146 aa, Homo sapiens
  UBE2D1 UBC domain
ALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAM

SEQ ID NO: 46 - protein, 146 aa, Homo sapiens
  UBE2D2 UBC domain
ALKRIHKELNDLARDPPAQCSAGPVGDDMFHWQATIMGPNDSPYQGGVFFLTIHFPTDYPFKPPKVAFTTRIYHPNINSNGSICLDILRSQWSPALTISKVLLSICSLLCDPNPDDPLVPEIARIYKTDREKYNRIAREWTQKYAM

SEQ ID NO: 47 - protein, 146 aa, Homo sapiens
  UBE2D3 UBC domain
ALKRINKELSDLARDPPAQCSAGPVGDDMFHWQATIMGPNDSPYQGGVFFLTIHFPTDYPFKPPKVAFTTRIYHPNINSNGSICLDILRSQWSPALTISKVLLSICSLLCDPNPDDPLVPEIARIYKTDRDKYNRISREWTQKYAM

SEQ ID NO: 48 - protein, 146 aa, Homo sapiens
  UBE2D4 UBC domain
ALKRIQKELTDLQRDPPAQCSAGPVGDDLFHWQATIMGPNDSPYQGGVFFLTIHFPTDYPFKPPKVAFTTKIYHPNINSNGSICLDILRSQWSPALTVSKVLLSICSLLCDPNPDDPLVPEIAHTYKADREKYNRLAREWTQKYAM

SEQ ID NO: 49 - protein, 192 aa, Homo sapiens
  UBE2E1 UBC domain
SDDDSRASTSSSSSSSSNQQTEKETNTPKKKESKVSMSKNSKLLSTSAKRIQKELADITLDPPPNCSAGPKGDNIYEWRSTILGPPGSVYEGGVFFLDITFTPEYPFKPPKVTFRTRIYHCNINSQGVICLDILKDNWSPALTISKVLLSICSLLTDCNPADPLVGSIATQYMTNRAEHDRMARQWTKRYAT

SEQ ID NO: 50 - protein, 189 aa, Homo sapiens
  UBE2E2 UBC domain
STSGGSSDGDQRESVQQEPEREQVQPKKKEGKISSKTAAKLSTSAKRIQKELAEITLDPPPNCSAGPKGDNIYEWRSTILGPPGSVYEGGVFFLDITFSPDYPFKPPKVTFRTRIYHCNINSQGVICLDILKDNWSPALTISKVLLSICSLLTDCNPADPLVGSIATQYMTNRAEHDRMARQWTKRYAT

SEQ ID NO: 51 - protein, 149 aa, Homo sapiens
  UBE2E3 UBC domain
LSTSAKRIQKELAEITLDPPPNCSAGPKGDNIYEWRSTILGPPGSVYEGGVFFLDITFSSDYPFKPPKVTFRTRIYHCNINSQGVICLDILKDNWSPALTISKVLLSICSLLTDCNPADPLVGSIATQYLTNRAEHDRIARQWTKRYAT

SEQ ID NO: 52 - protein, 160 aa, Homo sapiens
  UBE2F UBC domain
STRRVSVRDKLLVKEVAELEANLPCTCKVHFPDPNKLHCFQLTVTPDEGYYQGGKFQFETEVPDAYNMVPPKVKCLTKIWHPNITETGEICLSLLREHSIDGTGWAPTRTLKDVVWGLNSLFTDLLNFDDPLNIEAAEHHLRDKEDFRNKVDDYIKRYAR

SEQ ID NO: 53 - protein, 153 aa, Homo sapiens
  UBE2G1 UBC domain
LLLRRQLAELNKNPVEGFSAGLIDDNDLYRWEVLIIGPPDTLYEGGVFKAHLTFPKDYPLRPPKMKFITEIWHPNVDKNGDVCISILHEPGEDKYGYEKPEERWLPIHTVETIMISVISMLADPNGDSPANVDAAKEWREDRNGEFKRKVARC

SEQ ID NO: 54 - protein, 164 aa, Homo sapiens
  UBE2G2 UBC domain
AGTALKRLMAEYKQLTLNPPEGIVAGPMNEENFFEWEALIMGPEDTCFEFGVFPAILSFPLDYPLSPPKMRFTCEMFHPNIYPDGRVCISILHAPGDDPMGYESSAERWSPVQSVEKILLSVVSMLAEPNDESGANVDASKMWRDDREQFYKIAKQIVQKSLGL

SEQ ID NO: 55 - protein, 159 aa, Homo sapiens
  UBE2H UBC domain
SSPSPGKRRMDTDVVKLIESKHEVTILGGLNEFVVKFYGPQGTPYEGGVWKVRVDLPDKYPFKSPSIGFMNKIFHPNIDEASGTVCLDVINQTWTALYDLTNIFESFLPQLLAYPNPIDPLNGDAAAMYLHRPEEYKQKIKEYIQKYATEEALKEQEEG

SEQ ID NO: 56 - protein, 157 aa, Homo sapiens
  UBE2I UBC domain
SGIALSRLAQERKAWRKDHPFGFVAVPTKNPDGTMNLMNWECAIPGKKGTPWEGGLFKLRMLFKDDYPSSPPKCKFEPPLFHPNVYPSGTVCLSILEEDKDWRPAITIKQILLGIQELLNEPNIQDPAQAEAYTIYCQNRVEYEKRVRAQAKKFAPS

SEQ ID NO: 57 - protein, 284 aa, Homo sapiens
  UBE2J1 UBC domain
ETRYNLKSPAVKRLMKEAAELKDPTDHYHAQPLEDNLFEWHFTVRGPPDSDFDGGVYHGRIVLPPEYPMKPPSIILLTANGRFEVGKKICLSISGHHPETWQPSWSIRTALLAIIGFMPTKGEGAIGSLDYTPEERRALAKKSQDFCCEGCGSAMKDVLLPLKSGSDSSQADQEAKELARQISFKAEVNSSGKTISESDLNHSFSLTDLQDDIPTTFQGATASTSYGLQNSSAASFHQPTQPVAKNTSMSPRQRRAQQQSQRRLSTSPDVIQGHQPRDNHTDHG

SEQ ID NO: 58 - protein, 184 aa, Homo sapiens
  UBE2J2 UBC domain
SSTSSKRAPTTATQRLKQDYLRIKKDPVPYICAEPLPSNILEWHYVVRGPEMTPYEGGYYHGKLIFPREFPFKPPSIYMITPNGRFKCNTRLCLSITDFHPDTWNPAWSVSTILTGLLSFMVEKGPTLGSIETSDFTKRQLAVQSLAFNLKDKVFCELFPEVVEEIKQKQKAQDELSSRPQTLP

SEQ ID NO: 59 - protein, 199 aa, Homo sapiens
  UBE2K UBC domain
ANIAVQRIKREFKEVLKSEETSKNQIKVDLVDENFTELRGEIAGPPDTPYEGGRYQLEIKIPETYPFNPPKVRFITKIWHPNISSVTGAICLDILKDQWAAAMTLRTVLLSLQALLAAAEPDDPQDAVVANQYKQNPEMFKQTARLWAHVYAGAPVSSPEYTKKIENLCAMGFDRNAVIVALSSKSWDVETATELLLSN

SEQ ID NO: 60 - protein, 153 aa, Homo sapiens
  UBE2L3 UBC domain
AASRRLMKELEEIRKCGMKNFRNIQVDEANLLTWQGLIVPDNPPYDKGAFRIEINFPAEYPFKPPKITFKTKIYHPNIDEKGQVCLPVISAENWKPATKTDQVIQSLIALVNDPQPEHPLRADLAEEYSKDRKKFCKNAEEFTKKYGEKRPVD

SEQ ID NO: 61 - protein, 152 aa, Homo sapiens
  UBE2L6 UBC domain
MASMRVVKELEDLQKKPPPYLRNLSSDDANVLVWHALLLPDQPPYHLKAFNLRISFPPEYPFKPPMIKFTTKIYHPNVDENGQICLPIISSENWKPCTKTCQVLEALNVLVNRPNIREPLRMDLADLLTQNPELFRKNAEEFTLRFGVDRPS

SEQ ID NO: 62 - protein, 157 aa, Homo sapiens
  UBE2M UBC domain
ASAAQLRIQKDINELNLPKTCDISFSDPDDLLNFKLVICPDEGFYKSGKFVFSFKVGQGYPHDPPKVKCETMVYHPNIDLEGNVCLNILREDWKPVLTINSIIYGLQYLFLEPNPEDPLNKEAAEVLQNNRRLFEQNVQRSMRGGYIGSTYFERCLK

SEQ ID NO: 63 - protein, 151 aa, Homo sapiens
  UBE2N UBC domain
AGLPRRIIKETQRLLAEPVPGIKAEPDESNARYFHVVIAGPQDSPFEGGTFKLELFLPEEYPMAAPKVRFMTKIYHPNVDKLGRICLDILKDKWSPALQIRTVLLSIQALLSAPNPDDPLANDVAEQWKTNEAQAIETARAWTRLYAMNNI

SEQ ID NO: 64 - protein, 152 aa, Homo sapiens
  UBE2NL UBC domain
AELPHRIIKETQRLLAEPVPGIKAEPDESNARYFHVVIAGESKDSPFEGGTFKRELLLAEEYPMAAPKVRFMTKIYHPNVDKLERISLDILKDKWSPALQIRTVLLSIQALLNAPNPDDPLANDVVEQWKTNEAQAIETARAWTRLYAMNSI

SEQ ID NO: 65 - protein, 138 aa, Homo sapiens
  UBE2O UBC domain
SNHSFKKIEFQPPEAKKFFSTVRKEMALLATSLPEGIMVKTFEDRMDLFSALIKGPTRTPYEDGLYLFDIQLPNIYPAVPPHFCYLSQCSGRLNPNLYDNGKVCVSLLGTWIGKGTERWTSKSSLLQVLISIQGLILV

SEQ ID NO: 66 - protein, 168 aa, Homo sapiens
  UBE2Q1 UBC domain
SGSVQATDRLMKELRDIYRSQSFKGGNYAVELVNDSLYDWNVKLLKVDQDSALHNDLQILKEKEGADFILLNFSFKDNFPFDPPFVRVVSPVLSGGYVLGGGAICMELLTKQGWSSAYSIESVIMQISATLVKGKARVQFGANKSQYSLTRAQQSYKSLVQIHEKNGW

SEQ ID NO: 67 - protein, 167 aa, Homo sapiens
  UBE2Q2 UBC domain
GAVSGSVQASDRLMKELRDIYRSQSYKTGIYSVELINDSLYDWHVKLQKVDPDSPLHSDLQILKEKEGIEYILLNFSFKDNFPFDPPFVRVVLPVLSGGYVLGGGALCMELLTKQGWSSAYSIESVIMQINATLVKGKARVQFGANKNQYNLARAQQSYNSIVQIHE

SEQ ID NO: 68 - protein, 160 aa, Homo sapiens
  UBE2QL UBC domain
KELQDIARLSDRFISVELVDESLFDWNVKLHQVDKDSVLWQDMKETNTEFILLNLTFPDNFPFSPPFMRVLSPRLENGYVLDGGAICMELLTPRGWSSAYTVEAVMRQFAASLVKGQGRICRKAGKSKKSFSRKEAEATFKSLVKTHEKYGWVTPPVSDG

SEQ ID NO: 69 - protein, 178 aa, Homo sapiens
  UBE2R1 UBC domain
PSSQKALLLELKGLQEEPVEGFRVTLVDEGDLYNWEVAIFGPPNTYYEGGYFKARLKFPIDYPYSPPAFRFLTKMWHPNIYETGDVCISILHPPVDDPQSGELPSERWNPTQNVRTILLSVISLLNEPNTFSPANVDASVMYRKWKESKGKDREYTDIIRKQVLGTKVDAERDGVKVP

SEQ ID NO: 70 - protein, 178 aa, Homo sapiens
  UBE2R2 UBC domain
TSSQKALMLELKSLQEEPVEGFRITLVDESDLYNWEVAIFGPPNTLYEGGYFKAHIKFPIDYPYSPPTFRFLTKMWHPNIYENGDVCISILHPPVDDPQSGELPSERWNPTQNVRTILLSVISLLNEPNTFSPANVDASVMFRKWRDSKGKDKEYAEIIRKQVSATKAEAEKDGVKVP

SEQ ID NO: 71 - protein, 155 aa, Homo sapiens
  UBE2S UBC domain
NSNVENLPPHIIRLVYKEVTTLTADPPDGIKVFPNEEDLTDLQVTIEGPEGTPYAGGLFRMKLLLGKDFPASPPKGYFLTKIFHPNVGANGEICVNVLKRDWTAELGIRHVLLTIKCLLIHPNPESALNEEAGRLLLENYEEYAARARLLTEIHG

SEQ ID NO: 72 - protein, 166 aa, Homo sapiens
  UBE2T UBC domain
QRASRLKRELHMLATEPPPGITCWQDKDQMDDLRAQILGGANTPYEKGVFKLEVIIPERYPFEPPQIRFLTPIYHPNIDSAGRICLDVLKLPPKGAWRPSLNIATVLTSIQLLMSEPNPDDPLMADISSEFKYNKPAFLKNARQWTEKHARQKQKADEEEMLDNLP

SEQ ID NO: 73 - protein, 149 aa, Homo sapiens
  UBE2U UBC domain
HGRAYLLLHRDFCDLKENNYKGITAKPVSEDMMEWEVEIEGLQNSVWQGLVFQLTIHFTSEYNYAPPVVKFITIPFHPNVDPHTGQPCIDFLDNPEKWNTNYTLSSILLALQVMLSNPVLENPVNLEAARILVKDESLYRTILRLFNRP

SEQ ID NO: 74 - protein, 140 aa, Homo sapiens
  UBE2V1 UBC domain
GVKVPRNFRLLEELEEGQKGVGDGTVSWGLEDDEDMTLTRWTGMIIGPPRTIYENRIYSLKIECGPKYPEAPPFVRFVTKINMNGVNSSNGVVDPRAISVLAKWQNSYSIKVVLQELRRLMMSKENMKLPQPPEGQCYSN

SEQ ID NO: 75 - protein, 144 aa, Homo sapiens
  UBE2V2 UBC domain
AVSTGVKVPRNFRLLEELEEGQKGVGDGTVSWGLEDDEDMTLTRWTGMIIGPPRTNYENRIYSLKVECGPKYPEAPPSVRFVTKINMNGINNSSGMVDARSIPVLAKWQNSYSIKVVLQELRRLMMSKENMKLPQPPEGQTYNN

SEQ ID NO: 76 - protein, 117 aa, Homo sapiens
  UBE2W UBC domain
MASMQKRLQKELLALQNDPPPGMTLNEKSVQNSITQWIVDMEGAPGTLYEGEKFQLLFKFSSRYPFDSPQVMFTGENIPVHPHVYSNGHICLSILTEDWSPALSVQSVCLSIISMLS

SEQ ID NO: 77 - protein, 145 aa, Homo sapiens
  UBE2Z UBC domain
MSIYKEPPPGMFVVPDTVDMTKIHALITGPFDTPYEGGFFLFVFRCPPDYPIHPPRVKLMTTGNNTVRFNPNFYRNGKVCLSILGTWTGPAWSPAQSISSVLISIQSLMTENPYHNEPGFEQERHPGDSKNYNECIRHETIRVAV

SEQ ID NO: 78 - protein, 182 aa, Homo sapiens
  UEVLD UBC domain
MEFDCEGLRRLLGKYKFRDLTVEELRNVNVFFPHFKYSMDTYVFKDSSQKDLLNFTGTIPVMYQGNTYNIPIRFWILDSHPFAPPICFLKPTANMGILVGKHVDAQGRIYLPYLQNWSHPKSVIVGLIKEMIAKFQEELPMYSLSSSDEARQVDLLAYIAKITEGVSDTNSKSWANHENKTV

SEQ ID NO: 79 - protein, 203 aa, Homo sapiens
  BIRC6 UBC domain
ANQEKKLGEYSKKAAMKPKPLSVLKSLEEKYVAVMKKLQFDTFEMVSEDEDGKLGFKVNYHYMSQVKNANDANSAARARRLAQEAVTLSTSLPLSSSSSVFVRCDEERLDIMKVLITGPADTPYANGCFEFDVYFPQDYPSSPPLVNLETTGGHSVRFNPNLYNDGKVCLSILNTWHGRPEEKWNPQTSSFLQVLVSVQSLIL

SEQ ID NO: 80 - protein, 202 aa, Homo sapiens
  FTS UBC domain
PPRTAPKKQLPSIPKNALPITKPTSPAPAAQSTNGTHASYGPFYLEYSLLAEFTLVVKQKLPGVYVQPSYRSALMWFGVIFIRHGLYQDGVFKFTVYIPDNYPDGDCPRLVFDIPVFHPLVDPTSGELDVKRAFAKWRRNHNHIWQVLMYARRVFYKIDTASPLNPEAAVLYEKDIQLFKSKVVDSVKVCTARLFDQPKIED

SEQ ID NO: 81 - protein, 144 aa, Homo sapiens
  TSG101 UBC domain
AVSESQLKKMVSKYKYRDLTVRETVNVITLYKDLKPVLDSYVFNDGSSRELMNLTGTIPVPYRGNTYNIPICLWLLDTYPYNPPICFVKPTSSMTIKTGKHVDANGKIYLPYLHEWKHPQSDLLGLIQVMIVVFGDEPPVFSRP

SEQ ID NO: 82 - protein, 166 aa, Homo sapiens
  UFC1 UBC domain
ADEATRRVVSEIPVLKTNAGPRDRELWVQRLKEEYQSLIRYVENNKNADNDWFRLESNKEGTRWFGKCWYIHDLLKYEFDIEFDIPITYPTTAPEIAVPELDGKTAKMYRGGKICLTDHFKPLWARNVPKFGLAHLMALGLGPWLAVEIPDLIQKGVIQHKEKCNQ

SEQ ID NO: 83 - DNA, 453 bp, Homo sapiens
  UBE2A UBC domain
tccaccccggctcggcggcgcctcatgcgggacttcaagaggttgcaggaggatcctccagccggagtcagcggggctccgtccgagaacaacataatggtgtggaacgcggtcattttcgggcctgaagggaccccgtttgaggatggaacatttaaacttacaatagaattcactgaagaatatccaaataaaccacctacagttagatttgtctctaagatgttccatccaaatgtctatgcagatggtagtatatgtctggacatacttcagaaccgttggagtccaacctatgatgtgtcttccattctaacatccatacagtctctgttggatgaacccaatcccaatagtccagcaaacagccaggctgctcagctgtaccaggagaacaaacgggaatatgaaaagcgtgtttctgcaatagtagaacaaagctggcgtgattgt

SEQ ID NO: 84 - DNA, 453 bp, Homo sapiens
  UBE2B UBC domain
agcacaccagctcggcggagactgatgagagacttcaagcggctgcaagaggaccctcctgtgggagtttctggcgcccctagcgagaacaacatcatgcagtggaacgccgtgatcttcggccctgagggcaccccttttgaggacggcaccttcaagctggtcatcgagttcagcgaggaataccccaacaagcctcctaccgtgcggttcctgagcaagatgtttcaccccaacgtgtacgccgacggcagcatctgtctggacatcctgcagaacagatggtccccaacctacgatgtgtccagcatcctgaccagcatccagagcctgctggacgagcccaatcctaacagccctgccaattctcaggccgctcagctgtaccaagagaacaagcgcgagtacgagaagcgggtgtccgccatcgttgagcagagctggaatgacagc

SEQ ID NO: 85 - DNA, 534 bp, Homo sapiens
  UBE2C UBC domain
gccagccagaacagagatcctgccgccacatctgtggccgctgctagaaaaggcgctgagccttctggcggagctgccagaggacctgtgggaaagagactgcagcaagagctgatgaccctgatgatgagcggcgacaagggcatctccgcctttccagagagcgacaacctgttcaaatgggtcggaaccatccacggcgctgccggcacagtgtatgaggacctgagatacaagctgagcctggaatttcccagcggctacccctacaatgcccctaccgtgaagtttctgaccccttgctatcaccccaacgtggacacccagggcaacatctgcctggacatcctgaaagagaagtggagcgccctgtacgatgtgcggacaatcctgctgagcatccagtctctgctgggcgagcccaacatcgacagccctctgaatactcacgccgccgagctgtggaagaaccccaccgcctttaagaagtacctgcaagagacatacagcaagcaagtgaccagccaagagcct

SEQ ID NO: 86 - DNA, 438 bp, Homo sapiens
  UBE2D1 UBC domain
gccctgaagagaatccagaaagagctgagcgacctgcagagagatcctcctgctcactgttctgctggccctgtgggagatgacctgttccactggcaagccaccatcatgggccctccagatagcgcctatcaaggcggcgtgttcttcctgaccgtgcacttccccacagactaccccttcaagcctcctaagatcgccttcaccaccaagatctatcaccccaacatcaacagcaacggcagcatctgcctggacatcctgagaagccaatggtcccctgctctgaccgtgtccaaggtgctgctgagcatctgcagcctgctgtgcgaccccaatcctgacgatcctctggtgcctgatatcgcccagatctacaagagcgacaaagagaagtacaaccggcacgccagagagtggacccagaaatacgctatg

SEQ ID NO: 87 - DNA, 438 bp, Homo sapiens
  UBE2D2 UBC domain
gctctgaagagaatccacaaagagctgaacgacctggccagagatcctcctgctcagtgttctgctggccctgtgggcgacgacatgtttcactggcaagccaccatcatgggccccaacgacagcccttatcaaggcggcgtgttcttcctgaccattcacttccccacagactaccccttcaagcctcctaaggtggccttcaccaccagaatctatcaccccaacatcaacagcaacggcagcatctgcctggacatcctgagaagccaatggtcccctgctctgaccatcagcaaggtgctgctgagcatctgcagcctgctgtgcgaccccaatcctgacgatcctctggtgcctgagatcgcccggatctacaagaccgacagagagaagtacaaccggatcgccagagagtggacccagaaatacgccatg

SEQ ID NO: 88 - DNA, 438 bp, Homo sapiens
  UBE2D3 UBC domain
gctctgaagcggatcaacaaagagctgagcgacctggccagagatcctcctgctcagtgttctgctggccctgtgggcgacgacatgtttcactggcaagccaccatcatgggccccaacgacagcccttatcaaggcggcgtgttcttcctgaccattcacttccccacagactaccccttcaagcctcctaaggtggccttcaccaccagaatctatcaccccaacatcaacagcaacggcagcatctgcctggacatcctgagaagccaatggtcccctgctctgaccatcagcaaggtgctgctgagcatctgcagcctgctgtgcgaccccaatcctgacgatcctctggtgcctgagatcgcccggatctacaagaccgaccgggacaagtacaaccggatcagcagagagtggacccagaaatacgccatg

SEQ ID NO: 89 - DNA, 438 bp, Homo sapiens
  UBE2D4 UBC domain
gctctgaagagaatccagaaagagctgaccgacctgcagcgggaccctcctgctcaatgttctgctggacccgtgggcgacgacctgtttcattggcaagccaccatcatgggccccaacgacagcccttatcaaggcggcgtgttcttcctgaccattcacttccccacagactaccccttcaagcctcctaaggtggccttcaccaccaagatctatcaccccaacatcaacagcaacggcagcatctgcctggacatcctgagaagccaatggtcccctgctctgaccgtgtccaaggtgctgctgagcatctgcagcctgctgtgcgaccccaatcctgacgatcctctggtgcctgagatcgcccacacctacaaggccgacagagagaagtacaaccggctggccagagagtggacccagaaatacgctatg

SEQ ID NO: 90 - DNA, 576 bp, Homo sapiens
  UBE2E1 UBC domain
agcgacgatgacagcagagccagcacctctagcagcagcagctccagcagcaatcagcagaccgagaaagagacaaacacgcccaagaaaaaagaaagcaaggtgtccatgagcaagaacagcaagctgctgagcaccagcgccaagagaatccagaaagagctggccgacatcacactggaccctccacctaattgcagcgccggacctaagggcgacaacatctatgagtggcggagcaccatcctgggaccacctggctctgtttatgaaggcggcgtgttcttcctggacatcaccttcacacctgagtaccccttcaagcctcctaaagtgaccttccggaccagaatctaccactgcaacatcaacagccagggcgtgatctgcctggatatcctgaaggacaattggagccccgctctgaccatcagcaaggtgctgctgtccatctgcagcctgctgaccgactgcaatcctgccgatcctctcgtgggctctatcgccacacagtacatgaccaacagagccgagcacgaccggatggccagacagtggacaaagagatacgccaca

SEQ ID NO: 91 - DNA, 567 bp, Homo sapiens
  UBE2E2 UBC domain
agcacatctggcggaagctccgatggcgatcagagggaatctgtgcagcaagagcccgagcgagaacaggtgcagcccaagaagaaagagggcaagatcagcagcaagaccgccgccaagctgagcacaagcgccaagagaatccagaaagagctggccgagatcacactggaccctccacctaattgcagcgccggacctaagggcgacaacatctatgagtggcggagcaccatcctgggaccacctggctctgtttatgaaggcggcgtgttcttcctggacatcacattcagccctgactaccccttcaagcctcctaaagtgaccttccggaccagaatctaccactgcaacatcaacagccagggcgtgatctgcctggatatcctgaaggacaattggagccccgctctgaccatcagcaaggtgctgctgagcatctgcagcctgctgaccgactgcaatcctgccgatcctctcgtgggctctatcgccacacagtacatgaccaacagagccgagcacgaccggatggccagacagtggacaaagagatacgccaca

SEQ ID NO: 92 - DNA, 447 bp, Homo sapiens
  UBE2E3 UBC domain
ttatccactagtgctaaaagaattcagaaggagctagctgaaataacccttgatcctcctcctaattgcagtgctgggcctaaaggagataacatttatgaatggagatcaactatacttggtccaccgggttctgtatatgaaggtggtgtgttttttctggatatcacattttcatcagattatccatttaagccaccaaaggttactttccgcaccagaatctatcactgcaacatcaacagtcagggagtcatctgtctggacatccttaaagacaactggagtcccgctttgactatttcaaaggttttgctgtctatttgttcccttttgacagactgcaaccctgcggatcctctggttggaagcatagccactcagtatttgaccaacagagcagaacacgacaggatagccagacagtggaccaagagatacgcaaca

SEQ ID NO: 93 - DNA, 480 bp, Homo sapiens
  UBE2F UBC domain
agcaccagacgggtgtcagtgcgggataagctgctggtcaaagaggtggccgagctggaagccaacctgccttgtacatgcaaggtgcacttccccgatcctaacaagctgcactgctttcagctgaccgtgacacccgacgagggctactatcaaggcggcaagttccagttcgagacagaggtgcccgacgcctacaacatggtgcctccaaaagtgaagtgcctgaccaagatctggcaccccaacatcaccgagacaggcgagatctgtctgagcctgctgagagagcacagcatcgacggaacaggatgggcccctaccagaacactgaaggatgttgtgtggggcctgaactccctgttcaccgacctgctgaacttcgacgaccctctgaatatcgaggccgccgagcaccacctgagagacaaagaggacttccggaacaaggtggacgactacatcaagagatacgcccgg

SEQ ID NO: 94 - DNA, 459 bp, Homo sapiens
  UBE2G1 UBC domain
ctgctgctgagaaggcagctggccgagctgaacaagaaccccgtggaaggcttttctgccggcctgatcgacgacaacgacctgtacagatgggaagtgctgatcatcggccctccagacacactgtatgaaggcggcgtgttcaaggcccacctgacattccccaaggactaccctctgcggcctcctaagatgaagttcatcaccgagatctggcaccccaacgtggacaagaatggcgacgtgtgcatcagcatcctgcacgagcctggcgaggataagtacggctacgagaagcccgaggaacggtggctgcctatccacaccgtggaaaccatcatgatcagcgtgatctccatgctggccgatcctaacggcgacagccctgccaatgtggatgccgccaaagagtggcgcgaggacagaaacggcgagttcaagagaaaggtggcccggtgt

SEQ ID NO: 95 - DNA, 492 bp, Homo sapiens
  UBE2G2 UBC domain
gccggaacagccctgaaaagactgatggccgagtacaagcagctgaccctgaatcctcctgagggcattgtggctggccctatgaacgaagagaacttcttcgagtgggaagccctgatcatgggccccgaggatacctgctttgagttcggagtgttccccgctatcctgagcttccctctggactaccctctgagccctcctaagatgcggtttacctgcgagatgtttcaccccaacatctaccccgacggcagagtgtgcatcagcattctgcatgcccctggcgacgaccctatgggctatgaatcttctgccgagcggtggtcccctgtgcagagcgtggaaaagatcctgctgagcgtggtgtccatgctggccgagcctaatgatgagagcggcgccaatgtggacgccagcaaaatgtggcgggacgacagagagcagttctacaagatcgccaagcagatcgtgcagaagtccctgggattg

SEQ ID NO: 96 - DNA, 477 bp, Homo sapiens
  UBE2H UBC domain
agctctccatctcctggcaagcggagaatggacaccgacgtggtcaagctgatcgagagcaagcacgaagtgaccatcctcggcggcctgaacgagttcgtcgtgaagttctatggaccccagggcaccccttatgaaggcggcgtttggaaagtgcgcgtggacctgcctgacaagtacccctttaagagccccagcatcggcttcatgaacaagatctttcaccccaacatcgacgaggccagcggcaccgtttgtctggacgtgatcaaccagacctggacagccctgtacgacctgaccaatatcttcgagagcttcctgcctcagctgctggcttaccccaatcctatcgaccctctgaatggcgacgccgctgccatgtatctgcacagacccgaagagtacaagcagaagatcaaagagtacatccagaagtacgccaccgaggaagccctgaaagagcaagaggaaggc

SEQ ID NO: 97 - DNA, 471 bp, Homo sapiens
  UBE2I UBC domain
agcggaattgccctgagcagactggcccaagagagaaaggcctggcggaaggatcaccccttcggctttgtggccgtgcctaccaagaatcccgacggcaccatgaacctgatgaactgggagtgcgctatccccggcaagaagggcacaccttgggaaggcggactgttcaagctgcggatgctgttcaaggacgactaccctagcagccctcctaagtgcaagttcgagcctcctctgtttcaccccaacgtgtaccctagcggcaccgtgtgtctgagcatcctggaagaggacaaggattggaggcccgccatcaccatcaagcagatcctgctgggcatccaagagctgctgaacgagcccaacattcaggaccctgctcaggccgaggcctacaccatctactgccagaacagagtggaatacgagaagagagtcagagcccaggccaagaagttcgccccatct

SEQ ID NO: 98 - DNA, 666 bp, Homo sapiens
  UBE2J1 UBC domain
gagacccgctacaacctgaagagtccggctgttaaacgtttaatgaaagaagcggcagaattgaaagatccaacagatcattaccatgcgcagcctttagaggataacctttttgaatggcacttcacggttagagggcccccagactccgattttgatggaggagtttatcacgggcggatagtactgccaccagagtatcccatgaaaccaccaagcattattctcctaacggctaatggtcgatttgaagtgggcaagaaaatctgtttgagcatctcaggccatcatcctgaaacttggcagccttcgtggagtataaggacagcattattagccatcattgggtttatgccaacaaaaggagagggagccataggttctctagattacactcctgaggaaagaagagcacttgccaaaaaatcacaagatttctgttgtgaaggatgtggctctgccatgaaggatgtcctgttgcctttaaaatctggaagcgattcaagccaagctgaccaagaagccaaagaactggctaggcaaataagctttaaggcagaagtcaattcatctggaaagactatctctgagtcagacttaaaccactctttttcactaactgatttacaagatgatatacctacaacattccagggtgctacggcc

SEQ ID NO: 99 - DNA, 552 bp, Homo sapiens
  UBE2J2 UBC domain
agcagcaccagctctaagagagcccctacaaccgccacacagcggctgaagcaggactacctgcggatcaagaaagaccccgtgccttacatctgcgccgagcctctgcctagcaacatcctggaatggcactacgtcgtgcggggacctgagatgacaccttacgaaggcggctactaccacggcaagctgatcttccccagagagttcccattcaagccacctagcatctacatgatcacccctaacggccggttcaagtgcaacaccagactgtgcctgagcatcaccgactttcaccccgacacctggaatcccgcttggagcgtgtccacaatcctgacaggcctgctgagcttcatggtggaaaagggccctacactgggcagcatcgagacaagcgacttcaccaagagacagctggccgtgcagagcctggccttcaacctgaaggacaaggtgttctgcgagctgttccccgaggtggtggaagagatcaagcagaagcagaaggcccaggacgagctgagcagcagacctcaaacactgcct

SEQ ID NO: 100 - DNA, 597 bp, Homo sapiens
  UBE2K UBC domain
gccaatatcgccgtgcagagaatcaagcgcgagttcaaagaggtgctgaagtccgaggaaaccagcaagaaccagatcaaggtggacctggtggacgagaacttcaccgagctgagaggcgagattgccggacctcctgacacaccttatgaaggcggcagataccagctggaaatcaagatccccgagacataccccttcaatcctccaaaagtgcggttcatcaccaagatctggcaccccaacatcagcagcgtgaccggcgccatctgtctggacatcctgaaggatcagtgggccgctgccatgacactgagaacagttctgctgagtctgcaggccctgctggctgctgctgaacctgatgatccacaggatgccgtggtggccaaccagtacaagcagaaccccgagatgttcaagcagaccgccagactgtgggcccatgtgtatgctggtgctcccgttagcagccccgagtacaccaagaaaatcgagaacctgtgcgccatgggcttcgacagaaacgccgtgattgtggccctgagcagcaagagctgggatgtcgaaacagccaccgaactgctgctgtccaac

SEQ ID NO: 101 - DNA, 459 bp, Homo sapiens
  UBE2L3 UBC domain
gccgctagtcggagactgatgaaggaactggaagagatccggaagtgcggcatgaagaacttccggaacatccaggtggacgaggccaacctgctgacatggcagggactgatcgtgcccgacaatcctccttacgacaagggcgccttcagaatcgagatcaacttccccgccgagtatcccttcaagcctcctaagatcaccttcaagaccaagatctatcaccccaacatcgacgagaagggccaagtgtgcctgcctgtgatcagcgccgagaattggaagcctgccaccaagaccgaccaagtgatccagtctctgatcgccctggtcaacgaccctcagcctgaacatcctctgagagccgatctggccgaagagtacagcaaggaccggaagaagttctgcaagaacgctgaagagttcaccaagaagtacggcgagaagcggcccgtggat

SEQ ID NO: 102 - DNA, 456 bp, Homo sapiens
  UBE2L6 UBC domain
atggccagcatgcgggtcgtgaaagagctggaagatctgcagaagaagcctcctccttacctgcggaacctgagcagcgacgatgccaatgtgcttgtgtggcatgccctgctgctgcccgatcagcctccttatcacctgaaggcctttaacctgcggatcagcttcccacctgagtaccccttcaagcctcctatgatcaagttcaccaccaagatctatcaccccaacgtggacgagaacggccagatctgcctgcctatcatcagcagcgagaactggaagccctgcaccaagacctgccaggtgctggaagctctgaacgtgctggtcaacagacccaacatccgcgagcctctgagaatggacctggccgatctgctgacacagaaccccgagctgttccggaagaacgccgaagagttcaccctgagattcggcgtggacagacctagc

SEQ ID NO: 103 - DNA, 471 bp, Homo sapiens
  UBE2M UBC domain
gcctctgctgcccagctgagaatccagaaggacatcaacgagctgaacctgcctaagacctgcgacatcagcttcagcgaccccgacgacctgctgaacttcaagctggtcatctgccccgacgagggcttctacaagagcggcaagttcgtgttcagcttcaaagtcggccagggctaccctcacgaccctccaaaagtgaagtgcgagacaatggtgtatcaccccaacatcgacctggaaggcaacgtgtgcctgaacatcctgcgcgaggattggaagcccgtgctgaccatcaacagcatcatctacggcctgcagtacctgttcctggaacctaatcctgaggaccctctgaacaaagaggccgccgaagtcctgcagaacaacagaaggctgttcgagcagaacgtgcagcggtctatgagaggcggctacattggcagcacctacttcgagagatgcctgaag

SEQ ID NO: 104 - DNA, 453 bp, Homo sapiens
  UBE2N UBC domain
gccgggctgccccgcaggatcatcaaggaaacccagcgtttgctggcagaaccagttcctggcatcaaagccgaaccagatgagagcaacgcccgttattttcatgtggtcattgctggccctcaggattccccctttgagggagggacttttaaacttgaactattccttccagaagaatacccaatggcagcccctaaagtacgtttcatgaccaaaatttatcatcctaatgtagacaagttgggaagaatatgtttagatattttgaaagataagtggtccccagcactgcagatccgcacagttctgctatcgatccaggccttgttaagtgctcccaatccagatgatccattagcaaatgatgtagcggagcagtggaagaccaacgaagcccaagccatagaaacagctagagcatggactaggctatatgccatgaataatatt

SEQ ID NO: 105 - DNA, 456 bp, Homo sapiens
  UBE2NL UBC domain
gccgagctgccccacaggatcatcaaggaaacccagcgtttgctggcagagccagttcctggcatcaaagcagaaccagatgaaagcaacgcccgttattttcatgtggtcattgctggggaatcaaaggattccccctttgagggagggacttttaaacgtgaactattacttgcagaagaatacccaatggcagcccctaaagtacgtttcatgaccaaaatttatcatccaaatgtagacaagttggaaagaataagtttagatattttgaaagataagtggtccccagccctgcagatccgcacagttctgctatcgatccaggccttgttaaatgctcccaatccagatgatccattagcaaatgatgtagtggagcagtggaagaccaacgaagcccaagccattgaaacagctagagcatggactaggctatatgccatgaatagtatt

SEQ ID NO: 106 - DNA, 414 bp, Homo sapiens
  UBE2O UBC domain
agcaaccacagcttcaagaagatcgagttccagccacctgaggccaagaaattcttcagcaccgtgcggaaagagatggccctgctggctacatctctgcccgagggcatcatggtcaagaccttcgaggaccggatggacctgttcagcgccctgatcaagggccccaccagaacaccttatgaggacggcctgtacctgttcgacatccagctgcctaacatctaccccgccgtgcctccacacttctgctacctgtctcagtgcagcggcagactgaaccccaacctgtacgacaacggcaaagtgtgcgtgtccctgctcggcacatggatcggaaagggcaccgagagatggaccagcaagtctagcctgctgcaggtcctgatcagcatccagggactgatcctggtg

SEQ ID NO: 107 - DNA, 504 bp, Homo sapiens
  UBE2Q1 UBC domain
agcggatctgtgcaggccaccgaccggcttatgaaggaactgcgggacatctacagaagccagagcttcaaaggcggcaactacgccgtggaactggtcaacgacagcctgtacgactggaacgtgaagctgctgaaggtggaccaggatagcgccctgcacaacgacctgcagatcctgaaagagaaagagggcgccgacttcatcctgctgaacttcagcttcaaggacaacttccccttcgatcctccattcgtgcgcgtggtgtctcctgttctgtctggcggatatgtgcttggcggcggagccatctgtatggaactgctgacaaagcaaggctggtccagcgcctacagcatcgagagcgtgatcatgcagatcagcgccacactggtcaagggcaaagccagagtgcagttcggcgccaacaagagccagtacagcctgacaagagcccagcagagctacaagtccctggtgcagatccacgagaagaacggctgg

SEQ ID NO: 108 - DNA, 501 bp, Homo sapiens
  UBE2Q2 UBC domain
ggcgctgtgtctggatctgtgcaggcctctgaccggctgatgaaggaactgcgggacatctacagaagccagagctacaagaccggcatctacagcgtggaactgatcaacgacagcctgtacgactggcacgtgaagctgcagaaggtggaccctgatagccctctgcacagcgacctgcagatcctgaaagagaaagagggcatcgagtacatcctgctgaacttcagcttcaaggacaacttccccttcgatcctccattcgtgcgcgtggtgctgcctgttctgtctggcggatatgtgcttggaggcggagccctgtgtatggaactgctgacaaagcaaggctggtccagcgcctacagcatcgagagcgtgatcatgcagatcaacgccacactggtcaagggcaaagccagagtgcagttcggcgccaacaagaaccagtacaacctggctagagcccagcagtcctacaacagcatcgtgcagatccacgaa

SEQ ID NO: 109 - DNA, 480 bp, Homo sapiens
  UBE2QL UBC domain
aaggagctgcaggacatcgcgcgccttagcgaccgcttcatctccgtggagctggtggacgagagcctgttcgactggaacgtgaagctgcaccaggtggacaaggactcggtgctgtggcaggacatgaaggagaccaacaccgagttcatcctgctcaacctcaccttccccgacaacttccccttctcgccgcccttcatgcgggtgctcagcccgcgcctggagaacggctacgtgctggacggcggcgccatctgcatggagctgctcacgccgcgcggctggtccagcgcctacaccgtggaggccgtcatgcgccagttcgcagccagcctggtcaagggccagggacggatctgtagaaaagctggcaaatcaaaaaagtccttcagtcgcaaggaagctgaagctacctttaagagtttggtgaagacgcatgaaaaatatggttgggtcaccccgcccgtgtccgacggc

SEQ ID NO: 110 - DNA, 534 bp, Homo sapiens
  UBE2R1 UBC domain
cctagctctcagaaggccctgctgctggaactgaagggcctgcaagaggaacccgtggaaggcttcagagtgaccctggtggatgagggcgacctgtacaattgggaagtcgccatcttcggccctccaaacacctactacgaaggcggctacttcaaggcccggctgaagttccccatcgactacccttatagccctcctgccttccggttcctgaccaagatgtggcaccccaacatctacgagacaggcgacgtgtgcatcagcattctgcaccctccagtggacgatcctcagtctggcgagctgccaagcgagagatggaaccccacacagaacgtgcggaccatcctgctgagcgtgatcagcctgctgaacgagcccaacacattcagccccgccaatgtggatgccagcgtgatgtaccggaagtggaaagagtccaagggcaaagacagagagtacaccgacatcatccggaaacaggtgctgggcacaaaggtggacgccgagcgagatggcgtgaaagttcct

SEQ ID NO: 111 - DNA, 573 bp, Homo sapiens
  UBE2R2 UBC domain
accagctcgcagaaggccctgatgctcgagctgaaatccctgcaggaggaaccggtggagggcttccggatcaccctggtggacgagtccgacctctacaactgggaggtggccatcttcggaccccccaacaccctctacgaaggcggctacttcaaggcgcatattaaatttcctattgactacccctattcaccacctaccttcagattcttgaccaaaatgtggcaccccaacatttatgagaatggagatgtatgcatttcgattcttcatccgcctgtagatgacccacagagtggagaactgccttctgaaaggtggaatcctactcagaatgtgaggactatcctattaagtgtaatctcactgcttaatgagcccaacaccttctccccagccaatgtcgatgcttcagttatgttcaggaaatggagagacagtaaaggaaaagacaaagaatatgctgaaattattaggaaacaagtttcagccactaaggccgaagcagaaaaggatggagtgaaggtccccacaaccctggcggaatactgcatcaaaactaaagtgcct

SEQ ID NO: 112 - DNA, 465 bp, Homo sapiens
  UBE2S UBC domain
aacagcaacgtggaaaacctgcctcctcacatcatccggctggtgtacaaagaagtgaccacactgaccgccgatcctccagacggcatcaaggtgttccccaacgaagaggacctgaccgacctgcaagtgaccatcgaaggccctgagggcacaccttatgctggcggcctgttcagaatgaagctgctgctgggcaaagacttccccgcatctcctccaaagggctacttcctgaccaagatctttcaccccaacgtgggcgccaacggcgagatctgtgtgaacgtgctgaagagagactggaccgccgagctgggaatcagacacgtgctgctgaccatcaagtgcctgctgattcaccctaatcctgagagcgccctgaacgaggaagctggcagactgctgctcgaaaactacgaggaatacgccgccagagccaggctgctgacagagattcatgga

SEQ ID NO: 113 - DNA, 498 bp, Homo sapiens
  UBE2T UBC domain
cagagagccagcagactgaagcgcgagctgcatatgctggccacagaacctccacctggcatcacctgttggcaggacaaggaccagatggacgacctgagagcccagattctcggcggagccaacacaccttatgagaagggcgtgttcaagctggaagtgatcatccccgagagataccccttcgagcctcctcagatccggtttctgacccctatctatcaccccaacatcgacagcgccggcagaatctgtctggacgtgctgaagctgcctcctaaaggcgcttggaggcccagcctgaatatcgccacagtgctgaccagcatccagctgctgatgagcgagcccaatcctgacgatcccctgatggccgatatcagcagcgagttcaagtacaacaagcccgccttcctgaagaacgccagacagtggacagagaagcacgcccggcagaagcagaaggccgacgaggaagagatgctggacaacctgcct

SEQ ID NO: 114 - DNA, 447 bp, Homo sapiens
  UBE2U UBC domain
cacggcagagcctatctgctgctgcacagagatttctgcgacctgaaagagaacaactacaagggcatcaccgccaagcctgtgtccgaggacatgatggaatgggaagtcgagatcgagggcctgcagaactctgtgtggcagggccttgtgttccagctgaccatccacttcaccagcgagtacaactacgcccctcctgtggtcaagttcatcacaatcccttttcaccccaacgtggaccctcacaccggccagccttgcatcgactttctggacaaccccgagaagtggaacaccaactacaccctgagcagcatcctgctggccctgcaagtgatgctgagcaaccccgtgctggaaaaccccgtgaatctggaagccgccagaatcctggtcaaggacgagagcctgtaccggaccatcctgcggctgttcaacagacct

SEQ ID NO: 115 - DNA, 420 bp, Homo sapiens
  UBE2V1 UBC domain
ggagtaaaagtccctcgcaatttccgactgttggaagaactcgaagaaggccagaaaggagtaggagatggcacagttagctggggtctagaagatgacgaagacatgacacttacaagatggacagggatgataattgggcctccaagaacaatttatgaaaaccgaatatacagccttaaaatagaatgtggacctaaatacccagaagcacccccctttgtaagatttgtaacaaaaattaatatgaatggagtaaatagttctaatggagtggtggacccaagagccatatcagtgctagcaaaatggcagaattcatatagcatcaaagttgtcctgcaagagcttcggcgcctaatgatgtctaaagaaaatatgaaactccctcagccgcccgaaggacagtgttacagcaat

SEQ ID NO: 116 - DNA, 432 bp, Homo sapiens
  UBE2V2 UBC domain
gccgtgtctacaggcgtgaaggtgcccagaaacttccggctgctggaagaactggaagagggccagaaaggcgtcggagatggcacagtgtcttggggcctcgaagatgacgaggacatgaccctgaccagatggaccggcatgatcatcggccctccacggaccaactacgagaaccggatctactccctgaaggtggaatgcggccctaagtaccctgaggctcctcctagcgtcagattcgtgaccaagatcaacatgaacggcatcaacaacagcagcggcatggtggacgccagatctattcctgtgctggccaagtggcagaacagctacagcatcaaggtggtgctgcaagagctgcggcggctgatgatgagcaaagaaaacatgaagctgccccagccacctgagggacagacctacaacaat

SEQ ID NO: 117 - DNA, 348 bp, Homo sapiens
  UBE2W UBC domain
gccagcatgcagaagcggctgcagaaagaactgctggccctgcagaacgatcctcctcctggcatgaccctgaacgagaagtccgtgcagaacagcatcacccagtggatcgtggacatggaaggcgcccctggcacactgtatgagggcgagaaattccagctgctgttcaagttcagcagcagataccccttcgacagccctcaagtgatgttcaccggcgagaacatccctgtgcaccctcacgtgtacagcaacggccacatctgcctgagcatcctgaccgaggattggagccctgctctgagcgtgcagagcgtgtgtctgagcatcatctccatgctgagc

SEQ ID NO: 118 - DNA, 435 bp, Homo sapiens
  UBE2Z UBC domain
atgtccatttataaggagcctcctccaggaatgttcgttgtacctgatactgttgacatgactaagattcatgcattgatcacaggcccatttgacactccttatgaagggggtttcttcctgttcgtgtttcggtgtccgcccgactatcccatccacccacctcgggtcaaactgatgacaacgggcaataacacagtgaggtttaaccccaacttctaccgcaatgggaaagtctgcttgagtattctaggtacatggactggacctgcctggagcccagcccagagcatctcctcagtgctcatctctatccagtccctgatgactgagaacccctatcacaatgagcccggctttgaacaggagagacatccaggagacagcaaaaactataatgaatgtatccggcacgagaccatcagagttgcagtc

SEQ ID NO: 119 - DNA, 1059 bp, Homo sapiens
  UEVLD UBC domain
gagttcgactgcgagggcctgagacggctgcttggcaagtacaagttcagggacctaactgtggaagaactaaggaatgtaaatgtatttttcccacatttcaaatattccatggacacctatgtttttaaagatagttctcagaaagacctgctgaattttactggcacaattcctgtgatgtatcagggtaatacatataacataccaattcgtttctggattttggattctcaccctttcgctccccctatttgcttcttgaagccaactgcaaatatgggaatcttagtcggaaaacatgtggatgctcaaggcagaatatatttgccctatctccaaaactggagccatcctaaatctgtcattgttggattaattaaagaaatgattgccaagtttcaagaggaacttcccatgtattctctatcatcatctgatgaggcacggcaggtagacttgctagcctatattgcaaaaatcactgaaggtgtttcagatacaaattcaaagagctgggcaaatcatgagaataaaacagtcaataaaattactgtggttggaggtggagaactcggtattgcctgcacattagcaatttcagcaaagggtattgcagacaggcttgtcctcttagacctctcagaagggactaaaggagccacgatggaccttgaaatcttcaaccttcctaatgtggagatcagcaaagatttgtctgcctctgctcattccaaggtggtgatcttcacagtcaactctttgggtagttctcagtcgtaccttgatgtggtacagagcaatgtggatatgttcagagcccttgtcccagctctgggacattatagtcaacacagtgtcctgctcgttgcatctcaaccagtggaaatcatgacctatgtaacatggaaactgagtacatttcctgcaaatcgagtgatcggaattggatgtaatctggattcacagagattacagtatattattacaaatgttttgaaggcacagacttcaggcaaagaagtatgggttattggcgagcaaggagaagacaaagtg

SEQ ID NO: 120 - DNA, 609 bp, Homo sapiens
  BIRC6 UBC domain
gccaatcaagagaagaagctgggcgagtacagcaagaaagccgccatgaagcccaagcctctgagcgtgctgaagtccctggaagagaaatacgtggccgtgatgaagaagctccagttcgacaccttcgagatggtgtccgaggacgaggatggcaagctgggcttcaaagtgaactaccactacatgagccaagtgaagaacgccaacgacgccaactctgccgccagagctagaaggctggctcaagaagccgtgactctgagcacaagcctgccactgagcagcagcagctccgtgttcgtcagatgtgacgaggaacggctggacatcatgaaggtgctgatcacaggccctgccgacacaccttacgccaatggctgcttcgagttcgacgtgtacttccctcaagactaccctagcagccctcctctggtcaacctggaaacaacaggcggacacagcgtgcggttcaaccccaacctgtacaacgacggcaaagtgtgcctgagcatcctgaacacctggcacggcagacccgaggaaaagtggaatcctcagaccagcagcttcctgcaggtcctggtgtctgtgcagagcctgattctg

SEQ ID NO: 121 - DNA, 606 bp, Homo sapiens
  FTS UBC domain
cctccacgaactgcaccaaagaaacagctgccttctattcccaaaaatgctttgcccataactaagcctacatctcctgccccagcagcacagtcaacaaatggcacgcatgcgtcctatggacccttctacctggaatactctcttcttgcagaatttaccttggttgtgaagcagaagctaccaggcgtctatgtgcagccatcttatcgctctgcattaatgtggtttggagtaatattcatacggcatggactttaccaagatggcgtatttaagtttacagtttacatccctgataactatccagatggtgactgtccacgcttggtgttcgatattcctgtctttcacccgctagttgatcccacctcaggtgagctggatgtgaagagagcatttgcaaaatggaggcggaaccataatcatatttggcaggtattaatgtatgcaaggagagttttctacaagattgatacagcaagccccctgaacccagaggctgcagtactgtatgaaaaagatattcagctttttaaaagtaaagttgttgacagtgttaaggtgtgcactgctcgtttgtttgaccaacctaaaatagaagac

SEQ ID NO: 122 - DNA, 432 bp, Homo sapiens
  TSG101 UBC domain
gccgtgtctgagagccagctgaagaaaatggtgtccaagtacaagtaccgggacctgaccgtgcgggaaaccgtgaatgtgatcaccctgtacaaggacctgaagcctgtgctggacagctacgtgttcaacgatggcagcagccgcgagctgatgaacctgacaggcacaatccccgtgccttaccggggcaacacctacaacatccccatctgcctgtggctgctggacacatacccctacaatcctccaatctgcttcgtgaagcccaccagcagcatgaccatcaagaccggcaaacacgtggacgccaacggcaagatctacctgccttacctgcacgagtggaagcaccctcagtctgatctgctgggcctgatccaagtgatgatcgtggtgttcggcgacgagcctcctgtgttcagtagacct

SEQ ID NO: 123 - DNA, 498 bp, Homo sapiens
  UFC1 UBC domain
gccgatgaggccacacgaagagtggtgtctgagatccccgtgctgaaaacaaacgctggccccagagacagagaactgtgggtgcagcggctgaaagaggaataccagagcctgatccgctacgtcgagaacaacaagaacgccgacaacgactggttccggctggaaagcaacaaagaaggcacccgttggttcggcaagtgctggtacatccacgacctgctgaagtacgagttcgacatcgagttcgatatccccatcacataccccaccaccgctccagagattgccgtgcctgagctggatggcaagaccgccaagatgtacagaggcggcaagatctgcctgaccgaccacttcaaacccctgtgggccagaaacgtgcccaagtttggactggcccatctgatggccctcggacttggaccttggctggccgtggaaatccccgacctgattcagaaaggcgtgatccagcacaaagagaagtgcaaccag

SEQ ID NO: 124 - protein, 9 aa, Artificial Sequence
  Synthetic; Haemagglutinin tag
YPYDVPDYA

SEQ ID NO: 125 - protein, 10 aa, Artificial Sequence
  Synthetic; Glu-Glu epitope tag
CEEEEYMPME

SEQ ID NO: 126 - protein, 94 aa, Artificial Sequence
  Synthetic; aCS3 targeting domain
VSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 127 - protein, 98 aa, Artificial Sequence
  Synthetic; aCS3_full
EFAMVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 128 - protein, 94 aa, Artificial Sequence
  Synthetic; aCS3(V33R) targeting domain
VSSVPTKLEVVAATPTSLLISWDAPAVTVDYYRITYGETGHWPWVWQEFEVPGSKSTATISGLKPGVDYTITVYAGSYSSYYYYGSPISINYRT

SEQ ID NO: 129 - protein, 94 aa, Artificial Sequence
  Synthetic; aCS3(K7Q) targeting domain
VSSVPTQLEVVAATPTSLLISWDAPAVTVDYYVITYGETGHWPWVWQEFEVPGSKSTATISGLKPGVDYTITVYAGSYSSYYYYGSPISINYRT

SEQ ID NO: 130 - protein, 94 aa, Artificial Sequence
  Synthetic; aCS3(K55Y) targeting domain
VSSVPTKLEVVAATPTSLLISWDAPAVTVDYYVITYGETGHWPWVWQEFEVPGSYSTATISGLKPGVDYTITVYAGSYSSYYYYGSPISINYRT

SEQ ID NO: 131 - protein, 94 aa, Artificial Sequence
  Synthetic; aCS3(K64H) targeting domain
VSSVPTKLEVVAATPTSLLISWDAPAVTVDYYVITYGETGHWPWVWQEFEVPGSYSTATISGLHPGVDYTITVYAGSYSSYYYYGSPISINYRT

SEQ ID NO: 132 - protein, 94 aa, Artificial Sequence
  Synthetic; aCS3(K7Q/K55Y) targeting domain
VSSVPTQLEVVAATPTSLLISWDAPAVTVDYYVITYGETGHWPWVWQEFEVPGSYSTATISGLKPGVDYTITVYAGSYSSYYYYGSPISINYRT

SEQ ID NO: 133 - protein, 94 aa, Artificial Sequence
  Synthetic; aCS3(K7Q/K64H) targeting domain
VSSVPTQLEVVAATPTSLLISWDAPAVTVDYYVITYGETGHWPWVWQEFEVPGSYSTATISGLHPGVDYTITVYAGSYSSYYYYGSPISINYRT

SEQ ID NO: 134 - protein, 94 aa, Artificial Sequence; identical to SEQ ID NO: 133
  Synthetic; aCS3(K55Y/K64H) targeting domain

SEQ ID NO: 135 - protein, 94 aa, Artificial Sequence; identical to SEQ ID NO: 133
  Synthetic; ACS3 (K7Q/K55Y/K64H) targeting domain

SEQ ID NO: 136-137 - skipped (2 entries)

SEQ ID NO: 138 - protein, 156 aa, Artificial Sequence
  Synthetic; K19 targeting domain
DLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKL

SEQ ID NO: 139 - protein, 154 aa, Artificial Sequence
  Synthetic; E3_5 targeting domain
DLGKKLLEAARAGQDDEVRILMANGADVNATDNDGYTPLHLAASNGHLEIVEVLLKNGADVNASDLTGITPLHLAAATGHLEIVEVLLKHGADVNAYDNDGHTPLHLAAKYGHLEIVEVLLKHGADVNAQDKFGKTAFDISIDNGNEDLAEILQ

SEQ ID NO: 140 - skipped

SEQ ID NO: 141 - protein, 9 aa, Artificial Sequence
  Synthetic; Linker 1 (Short)
LEGGGGSSR

SEQ ID NO: 142 - protein, 19 aa, Artificial Sequence
  Synthetic; Linker 2 (Long)
LEGGGGSGGGGSGGGGSSR

SEQ ID NO: 143 - protein, 20 aa, Artificial Sequence
  Synthetic; Linker 3
AAAGGGGSGGGGSGGGGSGT

SEQ ID NO: 144 - protein, 5 aa, Artificial Sequence
  Synthetic; Linker 4
GGGGG

SEQ ID NO: 145 - protein, 15 aa, Artificial Sequence
  Synthetic; Linker 5
GGGGSGGGGSGGGGS

SEQ ID NO: 146 - protein, 5 aa, Artificial Sequence
  Synthetic; Linker 6
GGGGS

SEQ ID NO: 147 - protein, 214 aa, Artificial Sequence
  Synthetic; VHL regulation domain
AMPRRAENWDEAEVGAEEAGVEEYGPEEDGGEESGAEESGPEESGPEELGAEEEMEVGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 148 - protein, 146 aa, Artificial Sequence
  Synthetic; UBE2D1(C85A) regulation domain
ALKRIQKELSDLQRDPPAHCSAGPVGDDLFHWQATIMGPPDSAYQGGVFFLTVHFPTDYPFKPPKIAFTTKIYHPNINSNGSIALDILRSQWSPALTVSKVLLSICSLLCDPNPDDPLVPDIAQIYKSDKEKYNRHAREWTQKYAM

SEQ ID NO: 149 - protein, 107 aa, Artificial Sequence
  Synthetic; HA_aCS3 control
YPYDVPDYAEFAMVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 150 - protein, 165 aa, Artificial Sequence
  Synthetic; HA_K19 control
YPYDVPDYADLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKL

SEQ ID NO: 151 - protein, 163 aa, Artificial Sequence
  Synthetic; HA_E3_5 control
YPYDVPDYADLGKKLLEAARAGQDDEVRILMANGADVNATDNDGYTPLHLAASNGHLEIVEVLLKNGADVNASDLTGITPLHLAAATGHLEIVEVLLKHGADVNAYDNDGHTPLHLAAKYGHLEIVEVLLKHGADVNAQDKFGKTAFDISIDNGNEDLAEILQ

SEQ ID NO: 152 - protein, 326 aa, Artificial Sequence
  Synthetic; HA_VHL_Linker 1_aCS3
YPYDVPDYAAMPRRAENWDEAEVGAEEAGVEEYGPEEDGGEESGAEESGPEESGPEELGAEEEMEVGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGDLEGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 153 - protein, 336 aa, Artificial Sequence
  Synthetic; HA_VHL_Linker 2_aCS3
YPYDVPDYAAMPRRAENWDEAEVGAEEAGVEEYGPEEDGGEESGAEESGPEESGPEELGAEEEMEVGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGDLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 154 - protein, 326 aa, Artificial Sequence
  Synthetic; HA_VHL_Linker 4_aCS3
YPYDVPDYAAMPRRAENWDEAEVGAEEAGVEEYGPEEDGGEESGAEESGPEESGPEELGAEEEMEVGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGDGGGGGEFAMVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 155 - protein, 336 aa, Artificial Sequence
  Synthetic; HA_VHL_Linker 2_aCS3(V33R)
YPYDVPDYAAMPRRAENWDEAEVGAEEAGVEEYGPEEDGGEESGAEESGPEESGPEELGAEEEMEVGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGDLEGGGGSGGGGSGGGGSSRVSSVPTKLEVVAATPTSLLISWDAPAVTVDYYRITYGETGHWPWVWQEFEVPGSKSTATISGLKPGVDYTITVYAGSYSSYYYYGSPISINYRT

SEQ ID NO: 156 - protein, 273 aa, Artificial Sequence
  Synthetic; HA_UBE2B_Linker 2_aCS3
YPYDVPDYASTPARRRLMRDFKRLQEDPPVGVSGAPSENNIMQWNAVIFGPEGTPFEDGTFKLVIEFSEEYPNKPPTVRFLSKMFHPNVYADGSICLDILQNRWSPTYDVSSILTSIQSLLDEPNPNSPANSQAAQLYQENKREYEKRVSAIVEQSWNDSLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 157 - protein, 300 aa, Artificial Sequence
  Synthetic; HA_UBE2C_Linker 2_aCS3
YPYDVPDYAASQNRDPAATSVAAARKGAEPSGGAARGPVGKRLQQELMTLMMSGDKGISAFPESDNLFKWVGTIHGAAGTVYEDLRYKLSLEFPSGYPYNAPTVKFLTPCYHPNVDTQGNICLDILKEKWSALYDVRTILLSIQSLLGEPNIDSPLNTHAAELWKNPTAFKKYLQETYSKQVTSQEPLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 158 - protein, 258 aa, Artificial Sequence
  Synthetic; HA_UBE2D1_Linker 1_aCS3
YPYDVPDYAALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMLEGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 159 - protein, 268 aa, Artificial Sequence
  Synthetic; HA_UBE2D1_Linker 2_aCS3
YPYDVPDYAALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 160 - protein, 268 aa, Artificial Sequence
  Synthetic; HA_UBE2D1_Linker 2_aCS3(V33R)
YPYDVPDYAALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMLEGGGGSGGGGSGGGGSSRVSSVPTKLEVVAATPTSLLISWDAPAVTVDYYRITYGETGHWPWVWQEFEVPGSKSTATISGLKPGVDYTITVYAGSYSSYYYYGSPISINYRT

SEQ ID NO: 161 - protein, 268 aa, Artificial Sequence
  Synthetic; HA_UBE2D1_Linker 2_aCS3 (K7Q)
YPYDVPDYAALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMLEGGGGSGGGGSGGGGSSRVSSVPTQLEVVAATPTSLLISWDAPAVTVDYYVITYGETGHWPWVWQEFEVPGSKSTATISGLKPGVDYTITVYAGSYSSYYYYGSPISINYRT

SEQ ID NO: 162 - protein, 268 aa, Artificial Sequence
  Synthetic; HA_UBE2D1_Linker 2_aCS3 (K55Y)
YPYDVPDYAALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMLEGGGGSGGGGSGGGGSSRVSSVPTKLEVVAATPTSLLISWDAPAVTVDYYVITYGETGHWPWVWQEFEVPGSYSTATISGLKPGVDYTITVYAGSYSSYYYYGSPISINYRT

SEQ ID NO: 163 - protein, 268 aa, Artificial Sequence
  Synthetic; HA_UBE2D1_Linker 2_aCS3 (K64H)
YPYDVPDYAALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMLEGGGGSGGGGSGGGGSSRVSSVPTKLEVVAATPTSLLISWDAPAVTVDYYVITYGETGHWPWVWQEFEVPGSKSTATISGLHPGVDYTITVYAGSYSSYYYYGSPISINYRT

SEQ ID NO: 164 - protein, 268 aa, Artificial Sequence
  Synthetic; HA_UBE2D1_Linker 2_aCS3 (K7Q, K55Y)
YPYDVPDYAALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMLEGGGGSGGGGSGGGGSSRVSSVPTQLEVVAATPTSLLISWDAPAVTVDYYVITYGETGHWPWVWQEFEVPGSYSTATISGLKPGVDYTITVYAGSYSSYYYYGSPISINYRT

SEQ ID NO: 165 - protein, 268 aa, Artificial Sequence
  Synthetic; HA_UBE2D1_Linker 2_aCS3 (K7Q, K64H)
YPYDVPDYAALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMLEGGGGSGGGGSGGGGSSRVSSVPTQLEVVAATPTSLLISWDAPAVTVDYYVITYGETGHWPWVWQEFEVPGSKSTATISGLHPGVDYTITVYAGSYSSYYYYGSPISINYRT

SEQ ID NO: 166 - protein, 268 aa, Artificial Sequence
  Synthetic; HA_UBE2D1_Linker 2_aCS3 (K55Y, K64H)
YPYDVPDYAALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMLEGGGGSGGGGSGGGGSSRVSSVPTKLEVVAATPTSLLISWDAPAVTVDYYVITYGETGHWPWVWQEFEVPGSYSTATISGLHPGVDYTITVYAGSYSSYYYYGSPISINYRT

SEQ ID NO: 167 - protein, 268 aa, Artificial Sequence
  Synthetic; HA_UBE2D1_Linker 2_aCS3 (K7Q, K55Y, K64H)
YPYDVPDYAALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMLEGGGGSGGGGSGGGGSSRVSSVPTQLEVVAATPTSLLISWDAPAVTVDYYVITYGETGHWPWVWQEFEVPGSYSTATISGLHPGVDYTITVYAGSYSSYYYYGSPISINYRT

SEQ ID NO: 168 - protein, 223 aa, Artificial Sequence
  Synthetic; HA_VHL control
YPYDVPDYAAMPRRAENWDEAEVGAEEAGVEEYGPEEDGGEESGAEESGPEESGPEELGAEEEMEVGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 169 - protein, 155 aa, Artificial Sequence
  Synthetic; HA_UBE2D1 control
YPYDVPDYAALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAM

SEQ ID NO: 170 - protein, 268 aa, Artificial Sequence
  Synthetic; HA_UBE2D1(C85A)_Linker 2_aCS3
YPYDVPDYAALKRIQKELSDLQRDPPAHCSAGPVGDDLFHWQATIMGPPDSAYQGGVFFLTVHFPTDYPFKPPKIAFTTKIYHPNINSNGSIALDILRSQWSPALTVSKVLLSICSLLCDPNPDDPLVPDIAQIYKSDKEKYNRHAREWTQKYAMLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 171 - protein, 268 aa, Artificial Sequence
  Synthetic; HA_UBE2D2_Linker 2_aCS3
YPYDVPDYAALKRIHKELNDLARDPPAQCSAGPVGDDMFHWQATIMGPNDSPYQGGVFFLTIHFPTDYPFKPPKVAFTTRIYHPNINSNGSICLDILRSQWSPALTISKVLLSICSLLCDPNPDDPLVPEIARIYKTDREKYNRIAREWTQKYAMLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 172 - protein, 268 aa, Artificial Sequence
  Synthetic; HA_UBE2D3_Linker 2_aCS3
YPYDVPDYAALKRINKELSDLARDPPAQCSAGPVGDDMFHWQATIMGPNDSPYQGGVFFLTIHFPTDYPFKPPKVAFTTRIYHPNINSNGSICLDILRSQWSPALTISKVLLSICSLLCDPNPDDPLVPEIARIYKTDRDKYNRISREWTQKYAMLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 173 - protein, 314 aa, Artificial Sequence
  Synthetic; HA_UBE2E1_Linker 2_aCS3
YPYDVPDYASDDDSRASTSSSSSSSSNQQTEKETNTPKKKESKVSMSKNSKLLSTSAKRIQKELADITLDPPPNCSAGPKGDNIYEWRSTILGPPGSVYEGGVFFLDITFTPEYPFKPPKVTFRTRIYHCNINSQGVICLDILKDNWSPALTISKVLLSICSLLTDCNPADPLVGSIATQYMTNRAEHDRMARQWTKRYATLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 174 - protein, 282 aa, Artificial Sequence
  Synthetic; HA_UBE2F_Linker 2_aCS3
YPYDVPDYASTRRVSVRDKLLVKEVAELEANLPCTCKVHFPDPNKLHCFQLTVTPDEGYYQGGKFQFETEVPDAYNMVPPKVKCLTKIWHPNITETGEICLSLLREHSIDGTGWAPTRTLKDVVWGLNSLFTDLLNFDDPLNIEAAEHHLRDKEDFRNKVDDYIKRYARLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 175 - protein, 275 aa, Artificial Sequence
  Synthetic; HA_UBE2G1_Linker 2_aCS3
YPYDVPDYALLLRRQLAELNKNPVEGFSAGLIDDNDLYRWEVLIIGPPDTLYEGGVFKAHLTFPKDYPLRPPKMKFITEIWHPNVDKNGDVCISILHEPGEDKYGYEKPEERWLPIHTVETIMISVISMLADPNGDSPANVDAAKEWREDRNGEFKRKVARCLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 176 - protein, 286 aa, Artificial Sequence
  Synthetic; HA_UBE2G2_Linker 2_aCS3
YPYDVPDYAAGTALKRLMAEYKQLTLNPPEGIVAGPMNEENFFEWEALIMGPEDTCFEFGVFPAILSFPLDYPLSPPKMRFTCEMFHPNIYPDGRVCISILHAPGDDPMGYESSAERWSPVQSVEKILLSVVSMLAEPNDESGANVDASKMWRDDREQFYKIAKQIVQKSLGLLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 177 - protein, 281 aa, Artificial Sequence
  Synthetic; HA_UBE2H_Linker 2_aCS3
YPYDVPDYASSPSPGKRRMDTDVVKLIESKHEVTILGGLNEFVVKFYGPQGTPYEGGVWKVRVDLPDKYPFKSPSIGFMNKIFHPNIDEASGTVCLDVINQTWTALYDLTNIFESFLPQLLAYPNPIDPLNGDAAAMYLHRPEEYKQKIKEYIQKYATEEALKEQEEGLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 178 - protein, 279 aa, Artificial Sequence
  Synthetic; HA_UBE2I_Linker 2_aCS3
YPYDVPDYASGIALSRLAQERKAWRKDHPFGFVAVPTKNPDGTMNLMNWECAIPGKKGTPWEGGLFKLRMLFKDDYPSSPPKCKFEPPLFHPNVYPSGTVCLSILEEDKDWRPAITIKQILLGIQELLNEPNIQDPAQAEAYTIYCQNRVEYEKRVRAQAKKFAPSLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 179 - protein, 306 aa, Artificial Sequence
  Synthetic; HA_UBE2J2_Linker 2_aCS3
YPYDVPDYASSTSSKRAPTTATQRLKQDYLRIKKDPVPYICAEPLPSNILEWHYVVRGPEMTPYEGGYYHGKLIFPREFPFKPPSIYMITPNGRFKCNTRLCLSITDFHPDTWNPAWSVSTILTGLLSFMVEKGPTLGSIETSDFTKRQLAVQSLAFNLKDKVFCELFPEVVEEIKQKQKAQDELSSRPQTLPLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 180 - protein, 321 aa, Artificial Sequence
  Synthetic; HA_UBE2K_Linker 2_aCS3
YPYDVPDYAANIAVQRIKREFKEVLKSEETSKNQIKVDLVDENFTELRGEIAGPPDTPYEGGRYQLEIKIPETYPFNPPKVRFITKIWHPNISSVTGAICLDILKDQWAAAMTLRTVLLSLQALLAAAEPDDPQDAVVANQYKQNPEMFKQTARLWAHVYAGAPVSSPEYTKKIENLCAMGFDRNAVIVALSSKSWDVETATELLLSNLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 181 - protein, 275 aa, Artificial Sequence
  Synthetic; HA_UBE2L3_Linker 2_aCS3
YPYDVPDYAAASRRLMKELEEIRKCGMKNFRNIQVDEANLLTWQGLIVPDNPPYDKGAFRIEINFPAEYPFKPPKITFKTKIYHPNIDEKGQVCLPVISAENWKPATKTDQVIQSLIALVNDPQPEHPLRADLAEEYSKDRKKFCKNAEEFTKKYGEKRPVDLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 182 - protein, 274 aa, Artificial Sequence
  Synthetic; HA_UBE2L6_Linker 2_aCS3
YPYDVPDYAMASMRVVKELEDLQKKPPPYLRNLSSDDANVLVWHALLLPDQPPYHLKAFNLRISFPPEYPFKPPMIKFTTKIYHPNVDENGQICLPIISSENWKPCTKTCQVLEALNVLVNRPNIREPLRMDLADLLTQNPELFRKNAEEFTLRFGVDRPSLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 183 - protein, 279 aa, Artificial Sequence
  Synthetic; HA_UBE2M_Linker 2_aCS3
YPYDVPDYAASAAQLRIQKDINELNLPKTCDISFSDPDDLLNFKLVICPDEGFYKSGKFVFSFKVGQGYPHDPPKVKCETMVYHPNIDLEGNVCLNILREDWKPVLTINSIIYGLQYLFLEPNPEDPLNKEAAEVLQNNRRLFEQNVQRSMRGGYIGSTYFERCLKLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 184 - protein, 260 aa, Artificial Sequence
  Synthetic; HA_UBE2O_Linker 2_aCS3
YPYDVPDYASNHSFKKIEFQPPEAKKFFSTVRKEMALLATSLPEGIMVKTFEDRMDLFSALIKGPTRTPYEDGLYLFDIQLPNIYPAVPPHFCYLSQCSGRLNPNLYDNGKVCVSLLGTWIGKGTERWTSKSSLLQVLISIQGLILVLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 185 - protein, 290 aa, Artificial Sequence
  Synthetic; HA_UBE2Q1_Linker 2_aCS3
YPYDVPDYASGSVQATDRLMKELRDIYRSQSFKGGNYAVELVNDSLYDWNVKLLKVDQDSALHNDLQILKEKEGADFILLNFSFKDNFPFDPPFVRVVSPVLSGGYVLGGGAICMELLTKQGWSSAYSIESVIMQISATLVKGKARVQFGANKSQYSLTRAQQSYKSLVQIHEKNGWLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 186 - protein, 289 aa, Artificial Sequence
  Synthetic; HA_UBE2Q2_Linker 2_aCS3
YPYDVPDYAGAVSGSVQASDRLMKELRDIYRSQSYKTGIYSVELINDSLYDWHVKLQKVDPDSPLHSDLQILKEKEGIEYILLNFSFKDNFPFDPPFVRVVLPVLSGGYVLGGGALCMELLTKQGWSSAYSIESVIMQINATLVKGKARVQFGANKNQYNLARAQQSYNSIVQIHELEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 187 - protein, 300 aa, Artificial Sequence
  Synthetic; HA_UBE2R1_Linker 2_aCS3
YPYDVPDYAPSSQKALLLELKGLQEEPVEGFRVTLVDEGDLYNWEVAIFGPPNTYYEGGYFKARLKFPIDYPYSPPAFRFLTKMWHPNIYETGDVCISILHPPVDDPQSGELPSERWNPTQNVRTILLSVISLLNEPNTFSPANVDASVMYRKWKESKGKDREYTDIIRKQVLGTKVDAERDGVKVPLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 188 - protein, 277 aa, Artificial Sequence
  Synthetic; HA_UBE2S_Linker 2_aCS3
YPYDVPDYANSNVENLPPHIIRLVYKEVTTLTADPPDGIKVFPNEEDLTDLQVTIEGPEGTPYAGGLFRMKLLLGKDFPASPPKGYFLTKIFHPNVGANGEICVNVLKRDWTAELGIRHVLLTIKCLLIHPNPESALNEEAGRLLLENYEEYAARARLLTEIHGLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 189 - protein, 288 aa, Artificial Sequence
  Synthetic; HA_UBE2T_Linker 2_aCS3
YPYDVPDYAQRASRLKRELHMLATEPPPGITCWQDKDQMDDLRAQILGGANTPYEKGVFKLEVIIPERYPFEPPQIRFLTPIYHPNIDSAGRICLDVLKLPPKGAWRPSLNIATVLTSIQLLMSEPNPDDPLMADISSEFKYNKPAFLKNARQWTEKHARQKQKADEEEMLDNLPLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 190 - protein, 271 aa, Artificial Sequence
  Synthetic; HA_UBE2U_Linker 2_aCS3
YPYDVPDYAHGRAYLLLHRDFCDLKENNYKGITAKPVSEDMMEWEVEIEGLQNSVWQGLVFQLTIHFTSEYNYAPPVVKFITIPFHPNVDPHTGQPCIDFLDNPEKWNTNYTLSSILLALQVMLSNPVLENPVNLEAARILVKDESLYRTILRLFNRPLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 191 - protein, 239 aa, Artificial Sequence
  Synthetic; HA_UBE2W_Linker 2_aCS3
YPYDVPDYAMASMQKRLQKELLALQNDPPPGMTLNEKSVQNSITQWIVDMEGAPGTLYEGEKFQLLFKFSSRYPFDSPQVMFTGENIPVHPHVYSNGHICLSILTEDWSPALSVQSVCLSIISMLSLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 192 - protein, 325 aa, Artificial Sequence
  Synthetic; HA_BIRC6_Linker 2_aCS3
YPYDVPDYAANQEKKLGEYSKKAAMKPKPLSVLKSLEEKYVAVMKKLQFDTFEMVSEDEDGKLGFKVNYHYMSQVKNANDANSAARARRLAQEAVTLSTSLPLSSSSSVFVRCDEERLDIMKVLITGPADTPYANGCFEFDVYFPQDYPSSPPLVNLETTGGHSVRFNPNLYNDGKVCLSILNTWHGRPEEKWNPQTSSFLQVLVSVQSLILLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 193 - protein, 288 aa, Artificial Sequence
  Synthetic; HA_UFC1_Linker 2_aCS3
YPYDVPDYAADEATRRVVSEIPVLKTNAGPRDRELWVQRLKEEYQSLIRYVENNKNADNDWFRLESNKEGTRWFGKCWYIHDLLKYEFDIEFDIPITYPTTAPEIAVPELDGKTAKMYRGGKICLTDHFKPLWARNVPKFGLAHLMALGLGPWLAVEIPDLIQKGVIQHKEKCNQLEGGGGSGGGGSGGGGSSRVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 194 - protein, 254 aa, Artificial Sequence
  Synthetic; HA_UBE2D1_Linker 4_aCS3
YPYDVPDYAALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMGGGGGVSSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 195 - protein, 272 aa, Artificial Sequence
  Synthetic; HA_aCS3(V33R)_Linker 2_UBE2D1
YPYDVPDYAEFAMVSSVPTKLEVVAATPTSLLISWDAPAVTVDYYRITYGETGHWPWVWQEFEVPGSKSTATISGLKPGVDYTITVYAGSYSSYYYYGSPISINYRTLEGGGGSGGGGSGGGGSSRALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAM

SEQ ID NO: 196 - protein, 330 aa, Artificial Sequence
  Synthetic; HA_aCS3_Linker 1_VHL
YPYDVPDYAEFAMVSSVPTKLEVVAATPTSLLISWDAPAVTVDYYVITYGETGHWPWVWQEFEVPGSKSTATISGLKPGVDYTITVYAGSYSSYYYYGSPISINYRTLEGGGGSSRAMPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 197 - protein, 340 aa, Artificial Sequence
  Synthetic; HA_aCS3_ Linker 2_VHL
YPYDVPDYAEFAMVSSVPTKLEVVAATPTSLLISWDAPAVTVDYYVITYGETGHWPWVWQEFEVPGSKSTATISGLKPGVDYTITVYAGSYSSYYYYGSPISINYRTLEGGGGSGGGGSGGGGSSRAMPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 198 - protein, 397 aa, Artificial Sequence
  Synthetic; HA_K19_linker 3_VHL
YPYDVPDYADLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLAAAGGGGSGGGGSGGGGSGTPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 199 - protein, 395 aa, Artificial Sequence
  Synthetic; HA_E3_5_linker 3_VHL
YPYDVPDYADLGKKLLEAARAGQDDEVRILMANGADVNATDNDGYTPLHLAASNGHLEIVEVLLKNGADVNASDLTGITPLHLAAATGHLEIVEVLLKHGADVNAYDNDGHTPLHLAAKYGHLEIVEVLLKHGADVNAQDKFGKTAFDISIDNGNEDLAEILQAAAGGGGSGGGGSGGGGSGTPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 200 - protein, 326 aa, Artificial Sequence
  Synthetic; HA_aCS3_Linker 4_VHL
YPYDVPDYAEFAMVSSVPTKLEVVAATPTSLLISWDAPAVTVDYYVITYGETGHWPWVWQEFEVPGSKSTATISGLKPGVDYTITVYAGSYSSYYYYGSPISINYRTGGGGGAMPRRAENWDEAEVGAEEAGVEEYGPEEDGGEESGAEESGPEESGPEELGAEEEMEVGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 201 - protein, 340 aa, Artificial Sequence
  Synthetic; HA_aCS3 (V33R)_ Linker 2_VHL
YPYDVPDYAEFAMVSSVPTKLEVVAATPTSLLISWDAPAVTVDYYRITYGETGHWPWVWQEFEVPGSKSTATISGLKPGVDYTITVYAGSYSSYYYYGSPISINYRTLEGGGGSGGGGSGGGGSSRAMPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 202 - protein, 262 aa, Artificial Sequence
  Synthetic; HA_aCS3_ Linker 1_UBE2D1
YPYDVPDYAEFAMVSSVPTKLEVVAATPTSLLISWDAPAVTVDYYVITYGETGHWPWVWQEFEVPGSKSTATISGLKPGVDYTITVYAGSYSSYYYYGSPISINYRTLEGGGGSSRALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAM

SEQ ID NO: 203 - protein, 272 aa, Artificial Sequence
  Synthetic; HA_aCS3_ Linker 2_UBE2D1
YPYDVPDYAEFAMVSSVPTKLEVVAATPTSLLISWDAPAVTVDYYVITYGETGHWPWVWQEFEVPGSKSTATISGLKPGVDYTITVYAGSYSSYYYYGSPISINYRTLEGGGGSGGGGSGGGGSSRALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAM

SEQ ID NO: 204 - protein, 331 aa, Artificial Sequence
  Synthetic; HA_K19_linker 3_UBE2D1
YPYDVPDYADLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLAAAGGGGSGGGGSGGGGSGTALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAM

SEQ ID NO: 205 - protein, 329 aa, Artificial Sequence
  Synthetic; HA_E3_5__linker 3_UBE2D1
YPYDVPDYADLGKKLLEAARAGQDDEVRILMANGADVNATDNDGYTPLHLAASNGHLEIVEVLLKNGADVNASDLTGITPLHLAAATGHLEIVEVLLKHGADVNAYDNDGHTPLHLAAKYGHLEIVEVLLKHGADVNAQDKFGKTAFDISIDNGNEDLAEILQAAAGGGGSGGGGSGGGGSGTALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAM

SEQ ID NO: 206 - protein, 5 aa, Artificial Sequence
  Synthetic; PxxxP motif
  VARIANT 2..5: Wherein Xaa is any amino acid
PXXXP

SEQ ID NO: 207 - protein, 5 aa, Artificial Sequence
  Synthetic; PxxPP motif
  VARIANT 2..3: Wherein Xaa is any amino acid
PXXPP

SEQ ID NO: 208 - protein, 6 aa, Artificial Sequence
  Synthetic; TPNGRF motif
TPNGRF

SEQ ID NO: 209 - protein, 6 aa, Artificial Sequence
  Synthetic; TANGRF motif
TANGRF

SEQ ID NO: 210 - protein, 6 aa, Artificial Sequence
  Synthetic; TxNGRF motif
  VARIANT 2: Wherein Xaa is any amino acid
TXNGRF

SEQ ID NO: 211 - protein, 6 aa, Artificial Sequence
  Linker 7
LEGGSR

SEQ ID NO: 212 - protein, 11 aa, Artificial Sequence
  Linker 8
LEGGGSGGSSR

SEQ ID NO: 213 - protein, 13 aa, Artificial Sequence
  Linker 9
LEGGGGSGGGSSR

SEQ ID NO: 214 - protein, 16 aa, Artificial Sequence
  Linker 10
LEGGGSGGGSGGGSSR

SEQ ID NO: 215 - protein, 19 aa, Artificial Sequence
  Linker 11
LEGGGGSGPSGGGGPSGSR

SEQ ID NO: 216 - protein, 23 aa, Artificial Sequence
  Linker 12
LESNGGGGSPAPAPGGGGSGSSR

SEQ ID NO: 217 - protein, 24 aa, Artificial Sequence
  Linker 13
LEGGGGSYPYDVPDYASGGGGSSR

SEQ ID NO: 218 - protein, 26 aa, Artificial Sequence
  Linker 14
TGGSAGGSGGSAGGSGGSAGGSGGSA

SEQ ID NO: 219 - protein, 28 aa, Artificial Sequence
  Linker 15
AGSGGSTGSGGSPTPSTSGGSTGSGGAS

SEQ ID NO: 220 - protein, 28 aa, Artificial Sequence
  Linker 16
AGSGGSGGSGGSGNSSTSGGSGGSGGAS

SEQ ID NO: 221 - protein, 28 aa, Artificial Sequence
  Linker 17
GGSPVPSTPGGGSGGGSGGSPVPSTPGS

SEQ ID NO: 222 - protein, 28 aa, Artificial Sequence
  Linker 18
SPGTGSPGTGSPGTGSPGTGSPGTGSPG

SEQ ID NO: 223 - DNA, 774 bp, Artificial Sequence
  UBE2D1_Linker 7_aCS3(K7Q,K55Y,K64H)_HA
gccttgaagcggattcagaaagagctgagcgacctgcagagggaccctcctgctcactgttctgctggacccgtgggagatgacctgttccactggcaagccaccatcatgggccctccagatagcgcctatcaaggcggcgtgttcttcctgaccgtgcacttccccacagactaccccttcaagcctcctaagatcgccttcaccaccaagatctatcaccccaacatcaacagcaacggcagcatctgcctggacatcctgagaagccaatggtcccctgctctgaccgtgtccaaggtgctgctgagcatctgcagcctgctgtgcgaccccaatcctgacgatcctctggtgcctgatatcgcccagatctacaagagcgacaaagagaagtacaaccggcacgccagagagtggacccagaaatacgctatgctggaaggcggctctagagtgtccagcgtgcccacacagctggaagtggttgccgccacacctacaagcctgctgatctcttgggatgcccctgccgtgacagtggactactacgtgatcacctacggcgagacaggccactggccttgggtctggcaagagtttgaagtgcccggcagctacagcaccgccacaatttctggactgcaccccggcgtggactacaccatcacagtgtacgccggctcctacagcagctactactactatggcagccccatcagcatcaactaccggacaggcggctacccctacgacgtgccagattatgcttga

SEQ ID NO: 224 - DNA, 789 bp, Artificial Sequence
  UBE2D1_ Linker 8_aCS3 (K7Q,K55Y,K64H)_HA
gccttgaagcggattcagaaagagctgagcgacctgcagagggaccctcctgctcactgttctgctggacccgtgggagatgacctgttccactggcaagccaccatcatgggccctccagatagcgcctatcaaggcggcgtgttcttcctgaccgtgcacttccccacagactaccccttcaagcctcctaagatcgccttcaccaccaagatctatcaccccaacatcaacagcaacggcagcatctgcctggacatcctgagaagccaatggtcccctgctctgaccgtgtccaaggtgctgctgagcatctgcagcctgctgtgcgaccccaatcctgacgatcctctggtgcctgatatcgcccagatctacaagagcgacaaagagaagtacaaccggcacgccagagagtggacccagaaatacgctatgctggaaggcggcggaagcggcggatcttctagagtgtccagcgtgcccacacagctggaagtggttgccgccacacctacaagcctgctgatctcttgggatgcccctgccgtgacagtggactactacgtgatcacctacggcgagacaggccactggccttgggtctggcaagagtttgaagtgcccggcagctacagcaccgccacaatttctggactgcaccccggcgtggactacaccatcacagtgtacgccggctcctacagcagctactactactatggcagccccatcagcatcaactaccggacaggcggctacccctacgacgtgccagattatgcttga

SEQ ID NO: 225 - DNA, 795 bp, Artificial Sequence
  UBE2D1_ Linker 9_aCS3 (K7Q,K55Y,K64H)_HA
gccttgaagcggattcagaaagagctgagcgacctgcagagggaccctcctgctcactgttctgctggacccgtgggagatgacctgttccactggcaagccaccatcatgggccctccagatagcgcctatcaaggcggcgtgttcttcctgaccgtgcacttccccacagactaccccttcaagcctcctaagatcgccttcaccaccaagatctatcaccccaacatcaacagcaacggcagcatctgcctggacatcctgagaagccaatggtcccctgctctgaccgtgtccaaggtgctgctgagcatctgcagcctgctgtgcgaccccaatcctgacgatcctctggtgcctgatatcgcccagatctacaagagcgacaaagagaagtacaaccggcacgccagagagtggacccagaaatacgctatgctggaaggcggcggaggatctggtggtggatcttctagagtgtccagcgtgcccacacagctggaagtggttgccgccacacctacaagcctgctgatctcttgggatgcccctgccgtgacagtggactactacgtgatcacctacggcgagacaggccactggccttgggtctggcaagagtttgaagtgcccggcagctacagcaccgccacaatttctggactgcaccccggcgtggactacaccatcacagtgtacgccggctcctacagcagctactactactatggcagccccatcagcatcaactaccggacaggcggctacccctacgacgtgccagattatgcttga

SEQ ID NO: 226 - DNA, 804 bp, Artificial Sequence
  UBE2D1_ Linker 10_aCS3(K7Q,K55Y,K64H)_HA
gccttgaagcggattcagaaagagctgagcgacctgcagagggaccctcctgctcactgttctgctggacccgtgggagatgacctgttccactggcaagccaccatcatgggccctccagatagcgcctatcaaggcggcgtgttcttcctgaccgtgcacttccccacagactaccccttcaagcctcctaagatcgccttcaccaccaagatctatcaccccaacatcaacagcaacggcagcatctgcctggacatcctgagaagccaatggtcccctgctctgaccgtgtccaaggtgctgctgagcatctgcagcctgctgtgcgaccccaatcctgacgatcctctggtgcctgatatcgcccagatctacaagagcgacaaagagaagtacaaccggcacgccagagagtggacccagaaatacgctatgctggaaggcggcggaagcggcggagggagcggtggtggatcttctagagtgtccagcgtgcccacacagctggaagtggttgccgccacacctacaagcctgctgatctcttgggatgcccctgccgtgacagtggactactacgtgatcacctacggcgagacaggccactggccttgggtctggcaagagtttgaagtgcccggcagctacagcaccgccacaatttctggactgcaccccggcgtggactacaccatcacagtgtacgccggctcctacagcagctactactactatggcagccccatcagcatcaactaccggacaggcggctacccctacgacgtgccagattatgcttga

SEQ ID NO: 227 - DNA, 813 bp, Artificial Sequence
  UBE2D1_ Linker 2_aCS3(K7Q,K55Y,K64H)_HA
gccttgaagcggattcagaaagagctgagcgacctgcagagggaccctcctgctcactgttctgctggacccgtgggagatgacctgttccactggcaagccaccatcatgggccctccagatagcgcctatcaaggcggcgtgttcttcctgaccgtgcacttccccacagactaccccttcaagcctcctaagatcgccttcaccaccaagatctatcaccccaacatcaacagcaacggcagcatctgcctggacatcctgagaagccaatggtcccctgctctgaccgtgtccaaggtgctgctgagcatctgcagcctgctgtgcgaccccaatcctgacgatcctctggtgcctgatatcgcccagatctacaagagcgacaaagagaagtacaaccggcacgccagagagtggacccagaaatacgctatgctggaaggcggcggaggatctggcggaggcggtagcggtggtggtggatcttctagagtgtccagcgtgcccacacagctggaagtggttgccgccacacctacaagcctgctgatctcttgggatgcccctgccgtgacagtggactactacgtgatcacctacggcgagacaggccactggccttgggtctggcaagagtttgaagtgcccggcagctacagcaccgccacaatttctggactgcaccccggcgtggactacaccatcacagtgtacgccggctcctacagcagctactactactatggcagccccatcagcatcaactaccggacaggcggctacccctacgacgtgccagattatgcttga

SEQ ID NO: 228 - DNA, 813 bp, Artificial Sequence
  UBE2D1_ Linker 11_aCS3(K7Q,K55Y,K64H)_HA
gccttgaagcggattcagaaagagctgagcgacctgcagagggaccctcctgctcactgttctgctggacccgtgggagatgacctgttccactggcaagccaccatcatgggccctccagatagcgcctatcaaggcggcgtgttcttcctgaccgtgcacttccccacagactaccccttcaagcctcctaagatcgccttcaccaccaagatctatcaccccaacatcaacagcaacggcagcatctgcctggacatcctgagaagccaatggtcccctgctctgaccgtgtccaaggtgctgctgagcatctgcagcctgctgtgcgaccccaatcctgacgatcctctggtgcctgatatcgcccagatctacaagagcgacaaagagaagtacaaccggcacgccagagagtggacccagaaatacgctatgctggaaggcggcggaggctctggaccttctggtggcggaggaccatctggctctagagtgtctagcgtgcccacacagctggaagtggtggccgctacacctacaagcctgctgatctcttgggatgcccctgccgtgacagtggactactacgtgatcacctacggcgagacaggccactggccttgggtctggcaagagtttgaagtgcccggcagctacagcaccgccacaatttctggactgcaccccggcgtggactacaccatcacagtgtacgccggctcctacagcagctactactactatggcagccccatcagcatcaactaccggacaggcggctacccctacgacgtgccagattatgcttga

SEQ ID NO: 229 - DNA, 825 bp, Artificial Sequence
  UBE2D1_ Linker 12_aCS3(K7Q,K55Y,K64H)_HA
gccctgaagagaatccagaaagagctgagcgacctgcagcgggatcctcctgctcactgttctgctggacccgtgggagatgacctgttccactggcaagccaccatcatgggccctccagatagcgcctatcaaggcggcgtgttcttcctgaccgtgcacttccccacagactaccccttcaagcctcctaagatcgccttcaccaccaagatctatcaccccaacatcaacagcaacggcagcatctgcctggacatcctgagaagccaatggtcccctgctctgaccgtgtccaaggtgctgctgagcatctgcagcctgctgtgcgaccccaatcctgacgatcctctggtgcctgatatcgcccagatctacaagagcgacaaagagaagtacaaccggcacgccagagagtggacccagaaatacgccatgctggaaagcaatggcggcggaggatctccagctcctgctcctggcggaggcggatctggatcttctagagtgtccagcgtgcccacacagctggaagtggttgccgccacacctacaagcctgctgatctcttgggatgcccctgccgtgacagtggactactacgtgatcacctacggcgagacaggccactggccttgggtctggcaagagtttgaagtgcccggcagctacagcaccgccacaatttctggactgcaccccggcgtggactacaccatcacagtgtacgccggctcctacagcagctactactactatggcagccccatcagcatcaactaccggacaggcggctacccctacgacgtgccagattatgcttga

SEQ ID NO: 230 - DNA, 840 bp, Artificial Sequence
  UBE2D1_ Linker 13_aCS3(K7Q,K55Y,K64H)_HA
gccttgaagcggattcagaaagagctgagcgacctgcagagggaccctcctgctcactgttctgctggacccgtgggagatgacctgttccactggcaagccaccatcatgggccctccagatagcgcctatcaaggcggcgtgttcttcctgaccgtgcacttccccacagactaccccttcaagcctcctaagatcgccttcaccaccaagatctatcaccccaacatcaacagcaacggcagcatctgcctggacatcctgagaagccaatggtcccctgctctgaccgtgtccaaggtgctgctgagcatctgcagcctgctgtgcgaccccaatcctgacgatcctctggtgcctgatatcgcccagatctacaagagcgacaaagagaagtacaaccggcacgccagagagtggacccagaaatacgccatgtctcccggaacaggctctcctggcacaggcagtcctggaactggatcaccaggcacaggttctccaggcactggaagccctggtgtcagctctgtgcctacacagctggaagtggtggccgccacacctacaagcctgctgatctcttgggatgcccctgccgtgacagtggactactacgtgatcacctacggcgagacaggccactggccttgggtctggcaagagtttgaagtgcccggcagctacagcaccgccacaatttctggactgcaccccggcgtggactacaccatcacagtgtacgccggctcctacagcagctactactactatggcagccccatcagcatcaactaccggacaggcggctacccctacgacgtgccagattatgcttga

SEQ ID NO: 231 - DNA, 834 bp, Artificial Sequence
  UBE2D1_ Linker 14_aCS3(K7Q,K55Y,K64H)_HA
gccttgaagcggattcagaaagagctgagcgacctgcagagggaccctcctgctcactgttctgctggacccgtgggagatgacctgttccactggcaagccaccatcatgggccctccagatagcgcctatcaaggcggcgtgttcttcctgaccgtgcacttccccacagactaccccttcaagcctcctaagatcgccttcaccaccaagatctatcaccccaacatcaacagcaacggcagcatctgcctggacatcctgagaagccaatggtcccctgctctgaccgtgtccaaggtgctgctgagcatctgcagcctgctgtgcgaccccaatcctgacgatcctctggtgcctgatatcgcccagatctacaagagcgacaaagagaagtacaaccggcacgccagagagtggacccagaaatacgctatgacaggcggatctgccggcggaagcggaggtagtgctggtggatctggtggttcagctggtggcagcggaggatctgctgtcagctctgtgcctacacagctggaagtggtggccgccacacctacaagcctgctgatctcttgggatgcccctgccgtgacagtggactactacgtgatcacctacggcgagacaggccactggccttgggtctggcaagagtttgaagtgcccggcagctacagcaccgccacaatttctggactgcaccccggcgtggactacaccatcacagtgtacgccggctcctacagcagctactactactatggcagccccatcagcatcaactaccggacaggcggctacccctacgacgtgccagattatgcttga

SEQ ID NO: 232 - DNA, 877 bp, Artificial Sequence
  UBE2D1_ Linker 15_aCS3(K7Q,K55Y,K64H)_HA
tattcactcgaggccgccaccatggccttgaagcggattcagaaagagctgagcgacctgcagagggaccctcctgctcactgttctgctggacccgtgggagatgacctgttccactggcaagccaccatcatgggccctccagatagcgcctatcaaggcggcgtgttcttcctgaccgtgcacttccccacagactaccccttcaagcctcctaagatcgccttcaccaccaagatctatcaccccaacatcaacagcaacggcagcatctgcctggacatcctgagaagccaatggtcccctgctctgaccgtgtccaaggtgctgctgagcatctgcagcctgctgtgcgaccccaatcctgacgatcctctggtgcctgatatcgcccagatctacaagagcgacaaagagaagtacaaccggcacgccagagagtggacccagaaatacgctatggccggctctggcggctctacaggatctggtggaagccctacacctagcacatctggcggaagcacaggttctggcggagcctctgttagcagcgtgccaacacagctggaagtggtggccgccacacctacaagcctgctgatctcttgggatgcccctgccgtgacagtggactactacgtgatcacctacggcgagacaggccactggccttgggtctggcaagagtttgaagtgcccggcagctacagcaccgccacaatttctggactgcaccccggcgtggactacaccatcacagtgtacgccggctcctacagcagctactactactatggcagccccatcagcatcaactaccggacaggcggctacccctacgacgtgccagattatgcttgactagtgcatcaca

SEQ ID NO: 233 - DNA, 840 bp, Artificial Sequence
  UBE2D1_ Linker 16_aCS3(K7Q,K55Y,K64H)_HA
gccttgaagcggattcagaaagagctgagcgacctgcagagggaccctcctgctcactgttctgctggacccgtgggagatgacctgttccactggcaagccaccatcatgggccctccagatagcgcctatcaaggcggcgtgttcttcctgaccgtgcacttccccacagactaccccttcaagcctcctaagatcgccttcaccaccaagatctatcaccccaacatcaacagcaacggcagcatctgcctggacatcctgagaagccaatggtcccctgctctgaccgtgtccaaggtgctgctgagcatctgcagcctgctgtgcgaccccaatcctgacgatcctctggtgcctgatatcgcccagatctacaagagcgacaaagagaagtacaaccggcacgccagagagtggacccagaaatacgctatggccggctctggtggcagcggaggttctggtggatctggcaatagcagcacaagcggcggatctggcggaagtggcggagcctctgttagctctgtgcccacacagctggaagtggtggccgccacacctacaagcctgctgatctcttgggatgcccctgccgtgacagtggactactacgtgatcacctacggcgagacaggccactggccttgggtctggcaagagtttgaagtgcccggcagctacagcaccgccacaatttctggactgcaccccggcgtggactacaccatcacagtgtacgccggctcctacagcagctactactactatggcagccccatcagcatcaactaccggacaggcggctacccctacgacgtgccagattatgcttga

SEQ ID NO: 234 - DNA, 840 bp, Artificial Sequence
  UBE2D1_ Linker 17_aCS3(K7Q,K55Y,K64H)_HA
gccttgaagcggattcagaaagagctgagcgacctgcagagggaccctcctgctcactgttctgctggacccgtgggagatgacctgttccactggcaagccaccatcatgggccctccagatagcgcctatcaaggcggcgtgttcttcctgaccgtgcacttccccacagactaccccttcaagcctcctaagatcgccttcaccaccaagatctatcaccccaacatcaacagcaacggcagcatctgcctggacatcctgagaagccaatggtcccctgctctgaccgtgtccaaggtgctgctgagcatctgcagcctgctgtgcgaccccaatcctgacgatcctctggtgcctgatatcgcccagatctacaagagcgacaaagagaagtacaaccggcacgccagagagtggacccagaaatacgctatgggcggcagccctgtgccttctacacctggcggaggaagcggcggaggatctggtggatctccagtgcctagtacaccaggcagcgtgtcctctgtgcccacacagctggaagtggtggccgccacacctacaagcctgctgatctcttgggatgcccctgccgtgacagtggactactacgtgatcacctacggcgagacaggccactggccttgggtctggcaagagtttgaagtgcccggcagctacagcaccgccacaatttctggactgcaccccggcgtggactacaccatcacagtgtacgccggctcctacagcagctactactactatggcagccccatcagcatcaactaccggacaggcggctacccctacgacgtgccagattatgcttga

SEQ ID NO: 235 - DNA, 840 bp, Artificial Sequence; identical to SEQ ID NO: 230
  UBE2D1_ Linker 18_aCS3(K7Q,K55Y,K64H)_HA

SEQ ID NO: 236 - protein, 257 aa, Artificial Sequence
  UBE2D1_Linker 7_aCS3(K7Q,K55Y,K64H)_HA
ALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMLEGGSRVSSVPTQLEVVAATPTSLLISWDAPAVTVDYYVITYGETGHWPWVWQEFEVPGSYSTATISGLHPGVDYTITVYAGSYSSYYYYGSPISINYRTGGYPYDVPDYA

SEQ ID NO: 237 - protein, 262 aa, Artificial Sequence
  UBE2D1_ Linker 8_aCS3 (K7Q,K55Y,K64H)_HA
ALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMLEGGGSGGSSRVSSVPTQLEVVAATPTSLLISWDAPAVTVDYYVITYGETGHWPWVWQEFEVPGSYSTATISGLHPGVDYTITVYAGSYSSYYYYGSPISINYRTGGYPYDVPDYA

SEQ ID NO: 238 - protein, 264 aa, Artificial Sequence
  UBE2D1_ Linker 9_aCS3 (K7Q,K55Y,K64H)_HA
ALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMLEGGGGSGGGSSRVSSVPTQLEVVAATPTSLLISWDAPAVTVDYYVITYGETGHWPWVWQEFEVPGSYSTATISGLHPGVDYTITVYAGSYSSYYYYGSPISINYRTGGYPYDVPDYA

SEQ ID NO: 239 - protein, 267 aa, Artificial Sequence
  UBE2D1_ Linker 10_aCS3(K7Q,K55Y,K64H)_HA
ALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMLEGGGSGGGSGGGSSRVSSVPTQLEVVAATPTSLLISWDAPAVTVDYYVITYGETGHWPWVWQEFEVPGSYSTATISGLHPGVDYTITVYAGSYSSYYYYGSPISINYRTGGYPYDVPDYA

SEQ ID NO: 240 - protein, 270 aa, Artificial Sequence
  UBE2D1_ Linker 2_aCS3(K7Q,K55Y,K64H)_HA
ALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMLEGGGGSGGGGSGGGGSSRVSSVPTQLEVVAATPTSLLISWDAPAVTVDYYVITYGETGHWPWVWQEFEVPGSYSTATISGLHPGVDYTITVYAGSYSSYYYYGSPISINYRTGGYPYDVPDYA

SEQ ID NO: 241 - protein, 270 aa, Artificial Sequence
  UBE2D1_ Linker 11_aCS3(K7Q,K55Y,K64H)_HA
ALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMLEGGGGSGPSGGGGPSGSRVSSVPTQLEVVAATPTSLLISWDAPAVTVDYYVITYGETGHWPWVWQEFEVPGSYSTATISGLHPGVDYTITVYAGSYSSYYYYGSPISINYRTGGYPYDVPDYA

SEQ ID NO: 242 - protein, 274 aa, Artificial Sequence
  UBE2D1_ Linker 12_aCS3(K7Q,K55Y,K64H)_HA
ALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMLESNGGGGSPAPAPGGGGSGSSRVSSVPTQLEVVAATPTSLLISWDAPAVTVDYYVITYGETGHWPWVWQEFEVPGSYSTATISGLHPGVDYTITVYAGSYSSYYYYGSPISINYRTGGYPYDVPDYA

SEQ ID NO: 243 - protein, 264 aa, Artificial Sequence
  UBE2D1_ Linker 13_aCS3(K7Q,K55Y,K64H)
ALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMLEGGGGSYPYDVPDYASGGGGSSRVSSVPTQLEVVAATPTSLLISWDAPAVTVDYYVITYGETGHWPWVWQEFEVPGSYSTATISGLHPGVDYTITVYAGSYSSYYYYGSPISINYRT

SEQ ID NO: 244 - protein, 277 aa, Artificial Sequence
  UBE2D1_ Linker 14_aCS3(K7Q,K55Y,K64H)_HA
ALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMTGGSAGGSGGSAGGSGGSAGGSGGSAVSSVPTQLEVVAATPTSLLISWDAPAVTVDYYVITYGETGHWPWVWQEFEVPGSYSTATISGLHPGVDYTITVYAGSYSSYYYYGSPISINYRTGGYPYDVPDYA

SEQ ID NO: 245 - protein, 279 aa, Artificial Sequence
  UBE2D1_ Linker 15_aCS3(K7Q,K55Y,K64H)_HA
ALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMAGSGGSTGSGGSPTPSTSGGSTGSGGASVSSVPTQLEVVAATPTSLLISWDAPAVTVDYYVITYGETGHWPWVWQEFEVPGSYSTATISGLHPGVDYTITVYAGSYSSYYYYGSPISINYRTGGYPYDVPDYA

SEQ ID NO: 246 - protein, 279 aa, Artificial Sequence
  UBE2D1_ Linker 16_aCS3(K7Q,K55Y,K64H)_HA
ALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMAGSGGSGGSGGSGNSSTSGGSGGSGGASVSSVPTQLEVVAATPTSLLISWDAPAVTVDYYVITYGETGHWPWVWQEFEVPGSYSTATISGLHPGVDYTITVYAGSYSSYYYYGSPISINYRTGGYPYDVPDYA

SEQ ID NO: 247 - protein, 279 aa, Artificial Sequence
  UBE2D1_ Linker 17_aCS3(K7Q,K55Y,K64H)_HA
ALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMGGSPVPSTPGGGSGGGSGGSPVPSTPGSVSSVPTQLEVVAATPTSLLISWDAPAVTVDYYVITYGETGHWPWVWQEFEVPGSYSTATISGLHPGVDYTITVYAGSYSSYYYYGSPISINYRTGGYPYDVPDYA

SEQ ID NO: 248 - protein, 279 aa, Artificial Sequence
  UBE2D1_ Linker 18_aCS3(K7Q,K55Y,K64H)_HA
ALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMSPGTGSPGTGSPGTGSPGTGSPGTGSPGVSSVPTQLEVVAATPTSLLISWDAPAVTVDYYVITYGETGHWPWVWQEFEVPGSYSTATISGLHPGVDYTITVYAGSYSSYYYYGSPISINYRTGGYPYDVPDYA

SEQ ID NO: 249 - DNA, 993 bp, Artificial Sequence
  HA_K19_linker_UBE2D1
tatccctacgacgtgcccgattatgccgacctgggcaagaagctgctggaagctgctagagccggccaggacgatgaagtgcggattctgatggccaacggcgccgatgtgaacgcctctgatagatggggatggacccctctgcatctggctgcttggtggggacacctggaaatcgtggaagtgctgctgaagagaggggccgacgtttcagccgctgatctgcatggacagagcccactgcatctcgccgccatggttggacaccttgagattgtcgaggtcctgctgaagtatggcgctgacgtgaacgctaaggacaccatgggagccacaccactgcacctggcagctagatctggccatctggaaattgtcgaagaactgctcaagaatggggcagacatgaacgcccaggacaagttcggcaagaccaccttcgacatcagcaccgacaacggcaacgaggacctggccgagatcctgcagaaactgctcgagggaggcggaggatctggcggaggtggaagtggcggaggcggctcttctagagccctgaagagaatccagaaagagctgagcgacctgcagagagatcctcctgctcactgttctgctggccctgtgggagatgacctgttccactggcaagccaccatcatgggccctccagatagcgcctatcaaggcggcgtgttcttcctgaccgtgcacttccccacagactaccccttcaagcctcctaagatcgccttcaccaccaagatctatcaccccaacatcaacagcaacggcagcatctgcctggacatcctgagaagccaatggtcccctgctctgaccgtgtccaaggtgctgctgagcatctgcagcctgctgtgcgaccccaatcctgacgatcctctggtgcctgatatcgcccagatctacaagagcgacaaagagaagtacaaccggcacgccagagagtggacccagaaatacgctatgtga

SEQ ID NO: 250 - DNA, 993 bp, Artificial Sequence
  HA_UBE2D1_linker_K19
tatccctacgacgtgcccgattatgccgccctgaagagaatccagaaagagctgagcgacctgcagagagatcctcctgctcactgttctgctggccctgtgggagatgacctgttccactggcaagccaccatcatgggccctccagatagcgcctatcaaggcggcgtgttcttcctgaccgtgcacttccccacagactaccccttcaagcctcctaagatcgccttcaccaccaagatctatcaccccaacatcaacagcaacggcagcatctgcctggacatcctgagaagccaatggtcccctgctctgaccgtgtccaaggtgctgctgagcatctgcagcctgctgtgcgaccccaatcctgacgatcctctggtgcctgatatcgcccagatctacaagagcgacaaagagaagtacaaccggcacgccagagagtggacccagaaatacgctatgctcgagggaggcggaggatctggcggaggtggaagtggcggaggcggctcttctagagacctgggcaagaagctgctggaagctgctagagccggccaggacgatgaagtgcggattctgatggccaacggcgccgatgtgaacgcctctgatagatggggatggacccctctgcatctggctgcttggtggggacacctggaaatcgtggaagtgctgctgaagagaggggccgacgtttcagccgctgatctgcatggacagagcccactgcatctcgccgccatggttggacaccttgagattgtcgaggtcctgctgaagtatggcgctgacgtgaacgctaaggacaccatgggagccacaccactgcacctggcagctagatctggccatctggaaattgtcgaagaactgctcaagaatggggcagacatgaacgcccaggacaagttcggcaagaccaccttcgacatcagcaccgacaacggcaacgaggacctggccgagatcctgcagaaactgtga

SEQ ID NO: 251 - DNA, 1008 bp, Artificial Sequence
  HA_K19_linker_UBE2B
tatccctacgacgtgcccgattatgccgacctgggcaagaagctgctggaagccgctagagccggccaggatgatgaagtgcggatcctgatggccaacggcgccgatgtgaatgcctctgatagatggggctggacccctctgcatctggctgcttggtggggacacctggaaatcgtggaagtgctgctgaagagaggggccgacgtttcagccgctgatctgcatggacagagcccactgcatctcgccgccatggttggacaccttgagattgtcgaggtcctgctgaagtatggcgctgacgtgaacgccaaggacaccatgggagccacaccacttcatctggccgctagatctggccatctggaaattgtcgaagaactgctcaagaatggggccgatatgaacgcccaggataagttcggcaagaccaccttcgacatcagcaccgacaacggcaacgaggacctggccgagatcctgcagaaactgcttgaaggcggcggaggatctggcggaggtggaagcggaggcggtggaagctctagatctacccctgctcggcggagactgatgcgggacttcaagagactgcaagaggaccctcctgtgggagtgtctggcgcccctagcgagaacaacatcatgcagtggaacgccgtgatcttcggccctgagggcaccccttttgaggacggcaccttcaagctggtcatcgagttcagcgaggaataccccaacaagcctcctaccgtgcggttcctgagcaagatgtttcaccccaacgtgtacgccgacggcagcatctgtctggacatcctccagaacagatggtccccaacctacgatgtgtccagcatcctgaccagcatccagagcctgctggacgagcctaatcctaacagccccgccaattctcaggccgctcagctgtaccaagagaacaagcgcgagtacgagaagcgggtgtccgccatcgttgagcagagctggaacgacagctga

SEQ ID NO: 252 - DNA, 1008 bp, Artificial Sequence
  HA_UBE2B_linker_K19
tatccctacgacgtgcccgattatgccagcacaccagctcgtcggagactgatgcgggacttcaagcggctgcaagaggatccacctgtgggagtttctggcgcccctagcgagaacaacatcatgcagtggaacgccgtgatcttcggccctgagggcaccccttttgaggacggcaccttcaagctggtcatcgagttcagcgaggaataccccaacaagcctcctaccgtgcggttcctgagcaagatgtttcaccccaacgtgtacgccgacggcagcatctgtctggacatcctgcagaacagatggtccccaacctacgatgtgtccagcatcctgaccagcatccagagcctgctggacgagcccaatcctaacagccctgccaattctcaggccgctcagctgtaccaagagaacaagcgcgagtacgagaagcgggtgtccgccatcgttgagcagagctggaacgatagccttgaaggcggcggaggatctggcggaggtggaagcggaggcggtggatcttctagagatctgggcaagaagctgctggaagccgctagagccggccaggatgatgaagtgcggatcctgatggccaacggcgccgatgtgaatgcctctgatagatggggctggacccctctgcatctggctgcttggtggggacacctggaaatcgtggaagtgctgctgaagagaggggccgacgtttcagccgctgatctgcatggacagagccctctgcaccttgccgccatggttggacaccttgagattgtcgaggtcctgctgaagtatggcgctgacgtgaacgccaaggacaccatgggagccacaccacttcatctggccgccagaagcggccatctggaaattgtcgaagaactgctcaagaatggggccgatatgaacgcccaggataagttcggcaagaccaccttcgacatcagcaccgacaacggcaatgaggacctggccgagatcctgcaaaagctctga

SEQ ID NO: 253 - protein, 330 aa, Artificial Sequence
  HA_K19_linker_UBE2D1
YPYDVPDYADLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLLEGGGGSGGGGSGGGGSSRALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAM

SEQ ID NO: 254 - protein, 330 aa, Artificial Sequence
  HA_UBE2D1_linker_K19
YPYDVPDYAALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMLEGGGGSGGGGSGGGGSSRDLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKL

SEQ ID NO: 255 - protein, 335 aa, Artificial Sequence
  HA_K19_linker_UBE2B
YPYDVPDYADLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLLEGGGGSGGGGSGGGGSSRSTPARRRLMRDFKRLQEDPPVGVSGAPSENNIMQWNAVIFGPEGTPFEDGTFKLVIEFSEEYPNKPPTVRFLSKMFHPNVYADGSICLDILQNRWSPTYDVSSILTSIQSLLDEPNPNSPANSQAAQLYQENKREYEKRVSAIVEQSWNDS

SEQ ID NO: 256 - protein, 335 aa, Artificial Sequence
  HA_UBE2B_linker_K19
YPYDVPDYASTPARRRLMRDFKRLQEDPPVGVSGAPSENNIMQWNAVIFGPEGTPFEDGTFKLVIEFSEEYPNKPPTVRFLSKMFHPNVYADGSICLDILQNRWSPTYDVSSILTSIQSLLDEPNPNSPANSQAAQLYQENKREYEKRVSAIVEQSWNDSLEGGGGSGGGGSGGGGSSRDLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKL

SEQ ID NO: 257 - protein, 94 aa, Artificial Sequence
  aCS3 (K7Q/K55Y/K64H/V33R)
VSSVPTQLEVVAATPTSLLISWDAPAVTVDYYRITYGETGHWPWVWQEFEVPGSYSTATISGLHPGVDYTITVYAGSYSSYYYYGSPISINYRT

SEQ ID NO: 258 - DNA, 810 bp, Artificial Sequence
  UBE2D1(C85A)_ Linker2_aCS3(K7Q,K55Y,K64H)_HA
gccttgaagcggattcagaaagagctgagcgacctgcagagggaccctcctgctcactgttctgctggacccgtgggagatgacctgttccactggcaagccaccatcatgggccctccagatagcgcctatcaaggcggcgtgttcttcctgaccgtgcacttccccacagactaccccttcaagcctcctaagatcgccttcaccaccaagatctatcaccccaacatcaacagcaacggcagcatcgccctggacatcctgagaagccaatggtcccctgctctgaccgtgtccaaggtgctgctgagcatctgcagcctgctgtgcgaccccaatcctgacgatcctctggtgcctgatatcgcccagatctacaagagcgacaaagagaagtacaaccggcacgccagagagtggacccagaaatacgctatgctggaaggcggcggaggatctggcggaggcggtagcggtggtggtggatcttctagagtgtccagcgtgcccacacagctggaagtggttgccgccacacctacaagcctgctgatctcttgggatgcccctgccgtgacagtggactactacgtgatcacctacggcgagacaggccactggccttgggtctggcaagagtttgaagtgcccggcagctacagcaccgccacaatttctggactgcaccccggcgtggactacaccatcacagtgtacgccggctcctacagcagctactactactatggcagccccatcagcatcaactaccggacaggcggctacccctacgacgtgccagattatgct

SEQ ID NO: 259 - DNA, 810 bp, Artificial Sequence
  UBE2D1_ Linker2_aCS3(K7Q,K55Y,K64H,V33R) _HA
gccttgaagcggattcagaaagagctgagcgacctgcagagggaccctcctgctcactgttctgctggacccgtgggagatgacctgttccactggcaagccaccatcatgggccctccagatagcgcctatcaaggcggcgtgttcttcctgaccgtgcacttccccacagactaccccttcaagcctcctaagatcgccttcaccaccaagatctatcaccccaacatcaacagcaacggcagcatctgcctggacatcctgagaagccaatggtcccctgctctgaccgtgtccaaggtgctgctgagcatctgcagcctgctgtgcgaccccaatcctgacgatcctctggtgcctgatatcgcccagatctacaagagcgacaaagagaagtacaaccggcacgccagagagtggacccagaaatacgctatgctggaaggcggcggaggatctggcggaggcggtagcggtggtggtggatcttctagagtgtccagcgtgcccacacagctggaagtggttgccgccacacctacaagcctgctgatctcttgggatgcccctgccgtgacagtggactactaccggatcacctacggcgagacaggccactggccttgggtctggcaagagtttgaagtgcccggcagctacagcaccgccacaatttctggactgcaccccggcgtggactacaccatcacagtgtacgccggctcctacagcagctactactactatggcagccccatcagcatcaactaccggacaggcggctacccctacgacgtgccagattatgct

SEQ ID NO: 260 - DNA, 810 bp, Artificial Sequence
  UBE2D1(C85A)_ Linker2_aCS3 (K7Q,K55Y,K64H,V33R)_HA
gccttgaagcggattcagaaagagctgagcgacctgcagagggaccctcctgctcactgttctgctggacccgtgggagatgacctgttccactggcaagccaccatcatgggccctccagatagcgcctatcaaggcggcgtgttcttcctgaccgtgcacttccccacagactaccccttcaagcctcctaagatcgccttcaccaccaagatctatcaccccaacatcaacagcaacggcagcatcgccctggacatcctgagaagccaatggtcccctgctctgaccgtgtccaaggtgctgctgagcatctgcagcctgctgtgcgaccccaatcctgacgatcctctggtgcctgatatcgcccagatctacaagagcgacaaagagaagtacaaccggcacgccagagagtggacccagaaatacgctatgctggaaggcggcggaggatctggcggaggcggtagcggtggtggtggatcttctagagtgtccagcgtgcccacacagctggaagtggttgccgccacacctacaagcctgctgatctcttgggatgcccctgccgtgacagtggactactaccggatcacctacggcgagacaggccactggccttgggtctggcaagagtttgaagtgcccggcagctacagcaccgccacaatttctggactgcaccccggcgtggactacaccatcacagtgtacgccggctcctacagcagctactactactatggcagccccatcagcatcaactaccggacaggcggctacccctacgacgtgccagattatgct

SEQ ID NO: 261 - DNA, 810 bp, Artificial Sequence
  UBE2D1(F62A)_ Linker2_aCS3(K7Q,K55Y,K64H)_HA
gccttgaagcggattcagaaagagctgagcgacctgcagagggaccctcctgctcactgttctgctggacccgtgggagatgacctgttccactggcaagccaccatcatgggccctccagatagcgcctatcaaggcggcgtgttcttcctgaccgtgcacttccccacagactaccccgccaagcctcctaagatcgccttcaccaccaagatctatcaccccaacatcaacagcaacggcagcatctgcctggacatcctgagaagccaatggtcccctgctctgaccgtgtccaaggtgctgctgagcatctgcagcctgctgtgcgaccccaatcctgacgatcctctggtgcctgatatcgcccagatctacaagagcgacaaagagaagtacaaccggcacgccagagagtggacccagaaatacgctatgctggaaggcggcggaggatctggcggaggcggtagcggtggtggtggatcttctagagtgtccagcgtgcccacacagctggaagtggttgccgccacacctacaagcctgctgatctcttgggatgcccctgccgtgacagtggactactacgtgatcacctacggcgagacaggccactggccttgggtctggcaagagtttgaagtgcccggcagctacagcaccgccacaatttctggactgcaccccggcgtggactacaccatcacagtgtacgccggctcctacagcagctactactactatggcagccccatcagcatcaactaccggacaggcggctacccctacgacgtgccagattatgct

SEQ ID NO: 262 - DNA, 825 bp, Artificial Sequence
  UBE2B_ Linker2_aCS3(K7Q,K55Y,K64H)_HA
agcacaccagctcggcggagactgatgagagacttcaagcggctgcaagaggaccctcctgtgggagtttctggcgcccctagcgagaacaacatcatgcagtggaacgccgtgatcttcggccctgagggcaccccttttgaggacggcaccttcaagctggtcatcgagttcagcgaggaataccccaacaagcctcctaccgtgcggttcctgagcaagatgtttcaccccaacgtgtacgccgacggcagcatctgtctggacatcctgcagaacagatggtccccaacctacgatgtgtccagcatcctgaccagcatccagagcctgctggacgagcccaatcctaacagccctgccaattctcaggccgctcagctgtaccaagagaacaagcgcgagtacgagaagcgggtgtccgccatcgttgagcagagctggaatgacagcctggaaggcggcggaggatctggcggaggcggtagcggtggtggtggatcttctagagtgtccagcgtgcccacacagctggaagtggttgccgccacacctacaagcctgctgatctcttgggatgcccctgccgtgacagtggactactacgtgatcacctacggcgagacaggccactggccttgggtctggcaagagtttgaagtgcccggcagctacagcaccgccacaatttctggactgcaccccggcgtggactacaccatcacagtgtacgccggctcctacagcagctactactactatggcagccccatcagcatcaactaccggacaggcggctacccctacgacgtgccagattatgct

SEQ ID NO: 263 - DNA, 825 bp, Artificial Sequence
  UBE2B(C88A)_ Linker2_aCS3(K7Q,K55Y,K64H)_HA
agcacaccagctcggcggagactgatgagagacttcaagcggctgcaagaggaccctcctgtgggagtttctggcgcccctagcgagaacaacatcatgcagtggaacgccgtgatcttcggccctgagggcaccccttttgaggacggcaccttcaagctggtcatcgagttcagcgaggaataccccaacaagcctcctaccgtgcggttcctgagcaagatgtttcaccccaacgtgtacgccgacggcagcatcgcgctggacatcctgcagaacagatggtccccaacctacgatgtgtccagcatcctgaccagcatccagagcctgctggacgagcccaatcctaacagccctgccaattctcaggccgctcagctgtaccaagagaacaagcgcgagtacgagaagcgggtgtccgccatcgttgagcagagctggaatgacagcctggaaggcggcggaggatctggcggaggcggtagcggtggtggtggatcttctagagtgtccagcgtgcccacacagctggaagtggttgccgccacacctacaagcctgctgatctcttgggatgcccctgccgtgacagtggactactacgtgatcacctacggcgagacaggccactggccttgggtctggcaagagtttgaagtgcccggcagctacagcaccgccacaatttctggactgcaccccggcgtggactacaccatcacagtgtacgccggctcctacagcagctactactactatggcagccccatcagcatcaactaccggacaggcggctacccctacgacgtgccagattatgct

SEQ ID NO: 264 - DNA, 825 bp, Artificial Sequence
  UBE2B_ Linker2_aCS3(K7Q,K55Y,K64H,V33R)_HA
agcacaccagctcggcggagactgatgagagacttcaagcggctgcaagaggaccctcctgtgggagtttctggcgcccctagcgagaacaacatcatgcagtggaacgccgtgatcttcggccctgagggcaccccttttgaggacggcaccttcaagctggtcatcgagttcagcgaggaataccccaacaagcctcctaccgtgcggttcctgagcaagatgtttcaccccaacgtgtacgccgacggcagcatctgtctggacatcctgcagaacagatggtccccaacctacgatgtgtccagcatcctgaccagcatccagagcctgctggacgagcccaatcctaacagccctgccaattctcaggccgctcagctgtaccaagagaacaagcgcgagtacgagaagcgggtgtccgccatcgttgagcagagctggaatgacagcctggaaggcggcggaggatctggcggaggcggtagcggtggtggtggatcttctagagtgtccagcgtgcccacacagctggaagtggttgccgccacacctacaagcctgctgatctcttgggatgcccctgccgtgacagtggactactaccggatcacctacggcgagacaggccactggccttgggtctggcaagagtttgaagtgcccggcagctacagcaccgccacaatttctggactgcaccccggcgtggactacaccatcacagtgtacgccggctcctacagcagctactactactatggcagccccatcagcatcaactaccggacaggcggctacccctacgacgtgccagattatgct

SEQ ID NO: 265 - DNA, 825 bp, Artificial Sequence
  UBE2B(C88A)_ Linker2_aCS3(K7Q,K55Y,K64H, V33R)_HA
agcacaccagctcggcggagactgatgagagacttcaagcggctgcaagaggaccctcctgtgggagtttctggcgcccctagcgagaacaacatcatgcagtggaacgccgtgatcttcggccctgagggcaccccttttgaggacggcaccttcaagctggtcatcgagttcagcgaggaataccccaacaagcctcctaccgtgcggttcctgagcaagatgtttcaccccaacgtgtacgccgacggcagcatcgcgctggacatcctgcagaacagatggtccccaacctacgatgtgtccagcatcctgaccagcatccagagcctgctggacgagcccaatcctaacagccctgccaattctcaggccgctcagctgtaccaagagaacaagcgcgagtacgagaagcgggtgtccgccatcgttgagcagagctggaatgacagcctggaaggcggcggaggatctggcggaggcggtagcggtggtggtggatcttctagagtgtccagcgtgcccacacagctggaagtggttgccgccacacctacaagcctgctgatctcttgggatgcccctgccgtgacagtggactactaccggatcacctacggcgagacaggccactggccttgggtctggcaagagtttgaagtgcccggcagctacagcaccgccacaatttctggactgcaccccggcgtggactacaccatcacagtgtacgccggctcctacagcagctactactactatggcagccccatcagcatcaactaccggacaggcggctacccctacgacgtgccagattatgct

SEQ ID NO: 266 - protein, 270 aa, Artificial Sequence
  UBE2D1(C85A)_ Linker2_aCS3(K7Q,K55Y,K64H)_HA
ALKRIQKELSDLQRDPPAHCSAGPVGDDLFHWQATIMGPPDSAYQGGVFFLTVHFPTDYPFKPPKIAFTTKIYHPNINSNGSIALDILRSQWSPALTVSKVLLSICSLLCDPNPDDPLVPDIAQIYKSDKEKYNRHAREWTQKYAMLEGGGGSGGGGSGGGGSSRVSSVPTQLEVVAATPTSLLISWDAPAVTVDYYVITYGETGHWPWVWQEFEVPGSYSTATISGLHPGVDYTITVYAGSYSSYYYYGSPISINYRTGGYPYDVPDYA

SEQ ID NO: 267 - protein, 270 aa, Artificial Sequence
  UBE2D1_Linker2_aCS3(K7Q,K55Y,K64H,V33R) _HA
ALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAMLEGGGGSGGGGSGGGGSSRVSSVPTQLEVVAATPTSLLISWDAPAVTVDYYRITYGETGHWPWVWQEFEVPGSYSTATISGLHPGVDYTITVYAGSYSSYYYYGSPISINYRTGGYPYDVPDYA

SEQ ID NO: 268 - protein, 270 aa, Artificial Sequence
  UBE2D1(C85A)_ Linker2_aCS3 (K7Q,K55Y,K64H,V33R)_HA
ALKRIQKELSDLQRDPPAHCSAGPVGDDLFHWQATIMGPPDSAYQGGVFFLTVHFPTDYPFKPPKIAFTTKIYHPNINSNGSIALDILRSQWSPALTVSKVLLSICSLLCDPNPDDPLVPDIAQIYKSDKEKYNRHAREWTQKYAMLEGGGGSGGGGSGGGGSSRVSSVPTQLEVVAATPTSLLISWDAPAVTVDYYRITYGETGHWPWVWQEFEVPGSYSTATISGLHPGVDYTITVYAGSYSSYYYYGSPISINYRTGGYPYDVPDYA

SEQ ID NO: 269 - protein, 270 aa, Artificial Sequence
  UBE2D1(F62A)_ Linker2_aCS3(K7Q,K55Y,K64H)_HA
ALKRIQKELSDLQRDPPAHCSAGPVGDDLFHWQATIMGPPDSAYQGGVFFLTVHFPTDYPAKPPKIAFTTKIYHPNINSNGSICLDILRSQWSPALTVSKVLLSICSLLCDPNPDDPLVPDIAQIYKSDKEKYNRHAREWTQKYAMLEGGGGSGGGGSGGGGSSRVSSVPTQLEVVAATPTSLLISWDAPAVTVDYYVITYGETGHWPWVWQEFEVPGSYSTATISGLHPGVDYTITVYAGSYSSYYYYGSPISINYRTGGYPYDVPDYA

SEQ ID NO: 270 - protein, 275 aa, Artificial Sequence
  UBE2B_ Linker2_aCS3(K7Q,K55Y,K64H)_HA
STPARRRLMRDFKRLQEDPPVGVSGAPSENNIMQWNAVIFGPEGTPFEDGTFKLVIEFSEEYPNKPPTVRFLSKMFHPNVYADGSICLDILQNRWSPTYDVSSILTSIQSLLDEPNPNSPANSQAAQLYQENKREYEKRVSAIVEQSWNDSLEGGGGSGGGGSGGGGSSRVSSVPTQLEVVAATPTSLLISWDAPAVTVDYYVITYGETGHWPWVWQEFEVPGSYSTATISGLHPGVDYTITVYAGSYSSYYYYGSPISINYRTGGYPYDVPDYA

SEQ ID NO: 271 - protein, 275 aa, Artificial Sequence
  UBE2B(C88A)_Linker2_aCS3(K7Q,K55Y,K64H)_HA
STPARRRLMRDFKRLQEDPPVGVSGAPSENNIMQWNAVIFGPEGTPFEDGTFKLVIEFSEEYPNKPPTVRFLSKMFHPNVYADGSIALDILQNRWSPTYDVSSILTSIQSLLDEPNPNSPANSQAAQLYQENKREYEKRVSAIVEQSWNDSLEGGGGSGGGGSGGGGSSRVSSVPTQLEVVAATPTSLLISWDAPAVTVDYYVITYGETGHWPWVWQEFEVPGSYSTATISGLHPGVDYTITVYAGSYSSYYYYGSPISINYRTGGYPYDVPDYA

SEQ ID NO: 272 - protein, 275 aa, Artificial Sequence
  UBE2B_Linker2_aCS3(K7Q,K55Y,K64H,V33R)_HA
STPARRRLMRDFKRLQEDPPVGVSGAPSENNIMQWNAVIFGPEGTPFEDGTFKLVIEFSEEYPNKPPTVRFLSKMFHPNVYADGSICLDILQNRWSPTYDVSSILTSIQSLLDEPNPNSPANSQAAQLYQENKREYEKRVSAIVEQSWNDSLEGGGGSGGGGSGGGGSSRVSSVPTQLEVVAATPTSLLISWDAPAVTVDYYRITYGETGHWPWVWQEFEVPGSYSTATISGLHPGVDYTITVYAGSYSSYYYYGSPISINYRTGGYPYDVPDYA

SEQ ID NO: 273 - protein, 275 aa, Artificial Sequence
  UBE2B(C88A)_Linker2_aCS3(K7Q,K55Y,K64H,V33R)_HA
STPARRRLMRDFKRLQEDPPVGVSGAPSENNIMQWNAVIFGPEGTPFEDGTFKLVIEFSEEYPNKPPTVRFLSKMFHPNVYADGSIALDILQNRWSPTYDVSSILTSIQSLLDEPNPNSPANSQAAQLYQENKREYEKRVSAIVEQSWNDSLEGGGGSGGGGSGGGGSSRVSSVPTQLEVVAATPTSLLISWDAPAVTVDYYRITYGETGHWPWVWQEFEVPGSYSTATISGLHPGVDYTITVYAGSYSSYYYYGSPISINYRTGGYPYDVPDYA

SEQ ID NO: 274 - protein, 333 aa, Artificial Sequence
  HA_E3_5_Linker2_UBE2B
YPYDVPDYADLGKKLLEAARAGQDDEVRILMANGADVNATDNDGYTPLHLAASNGHLEIVEVLLKNGADVNASDLTGITPLHLAAATGHLEIVEVLLKHGADVNAYDNDGHTPLHLAAKYGHLEIVEVLLKHGADVNAQDKFGKTAFDISIDNGNEDLAEILQLEGGGGSGGGGSGGGGSSRSTPARRRLMRDFKRLQEDPPVGVSGAPSENNIMQWNAVIFGPEGTPFEDGTFKLVIEFSEEYPNKPPTVRFLSKMFHPNVYADGSICLDILQNRWSPTYDVSSILTSIQSLLDEPNPNSPANSQAAQLYQENKREYEKRVSAIVEQSWNDS

SEQ ID NO: 275 - protein, 328 aa, Artificial Sequence
  HA_E3_5_Linker2_UBE2D1
YPYDVPDYADLGKKLLEAARAGQDDEVRILMANGADVNATDNDGYTPLHLAASNGHLEIVEVLLKNGADVNASDLTGITPLHLAAATGHLEIVEVLLKHGADVNAYDNDGHTPLHLAAKYGHLEIVEVLLKHGADVNAQDKFGKTAFDISIDNGNEDLAEILQLEGGGGSGGGGSGGGGSSRALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAM

SEQ ID NO: 276 - protein, 396 aa, Artificial Sequence
  HA_VHL_Linker2_E3_5
YPYDVPDYAAMPRRAENWDEAEVGAEEAGVEEYGPEEDGGEESGAEESGPEESGPEELGAEEEMEVGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGDLEGGGGSGGGGSGGGGSSRDLGKKLLEAARAGQDDEVRILMANGADVNATDNDGYTPLHLAASNGHLEIVEVLLKNGADVNASDLTGITPLHLAAATGHLEIVEVLLKHGADVNAYDNDGHTPLHLAAKYGHLEIVEVLLKHGADVNAQDKFGKTAFDISIDNGNEDLAEILQ

SEQ ID NO: 277 - protein, 398 aa, Artificial Sequence
  HA_VHL_Linker2_K19
YPYDVPDYAAMPRRAENWDEAEVGAEEAGVEEYGPEEDGGEESGAEESGPEESGPEELGAEEEMEVGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGDLEGGGGSGGGGSGGGGSSRDLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKL

SEQ ID NO: 278 - protein, 396 aa, Artificial Sequence
  HA_E3_5_Linker2_VHL
YPYDVPDYADLGKKLLEAARAGQDDEVRILMANGADVNATDNDGYTPLHLAASNGHLEIVEVLLKNGADVNASDLTGITPLHLAAATGHLEIVEVLLKHGADVNAYDNDGHTPLHLAAKYGHLEIVEVLLKHGADVNAQDKFGKTAFDISIDNGNEDLAEILQLEGGGGSGGGGSGGGGSSRAMPRRAENWDEAEVGAEEAGVEEYGPEEDGGEESGAEESGPEESGPEELGAEEEMEVGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 279 - protein, 398 aa, Artificial Sequence
  HA_K19_Linker2_VHL
YPYDVPDYADLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLLEGGGGSGGGGSGGGGSSRAMPRRAENWDEAEVGAEEAGVEEYGPEEDGGEESGAEESGPEESGPEELGAEEEMEVGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 280 - protein, 151 aa, Artificial Sequence
  UBE2B (C88A)
STPARRRLMRDFKRLQEDPPVGVSGAPSENNIMQWNAVIFGPEGTPFEDGTFKLVIEFSEEYPNKPPTVRFLSKMFHPNVYADGSIALDILQNRWSPTYDVSSILTSIQSLLDEPNPNSPANSQAAQLYQENKREYEKRVSAIVEQSWNDS

SEQ ID NO: 281 - protein, 144 aa, Artificial Sequence
  UBE2D1 (F62A)
ALKRIQKELSDLQRDPPAHCSAGPVGDDLFHWQATIMGPPDSAYQGGVFFLTVHFPTDYPAKPPKIAFTTKIYHPNINSNGSICLDILRSQWSPALTVSKVLLSICSLLCDPNPDDPLVPDIAQIYKSDKEKYNRHAREWTQKY

(19) United States
(12) Patent Application Publication
(10) Pub. No.: US 2025/0197819 A1
(43) Pub. Date: Jun. 19, 2025

(54) FUSION PROTEINS COMPRISING AN E2 UBIQUITIN OR UBIQUITIN-LIKE CONJUGATING DOMAIN AND A TARGETING DOMAIN FOR SPECIFIC PROTEIN DEGRADATION

(71) Applicant: MEDIMMUNE LIMITED, CAMBRIDGE (GB)

(72) Inventors: SANDRINE LEGG, CAMBRIDGE (GB); JAMES HUNT, CAMBRIDGE (GB); LARS GREDSTED, CAMBRIDGE (GB); RALPH RAYMOND MINTER, CAMBRIDGE (GB)

(21) Appl. No.: 18/253,665

(22) PCT Filed: Nov. 20, 2020
(86) PCT No.: PCT/IB2020/060978

§ 371 Date: May 19, 2023

(51) Int. Cl. C12N 9/10; A61K 38/00; C07K 16/46

(52) U.S. Cl. CPC C12N 9/104; C07K 16/46; A61K 38/00; C07K 2319/09; C12Y 203/02

(57) ABSTRACT

The disclosure provides a molecule comprising a regulation domain comprising an E2 ubiquitin or ubiquitin-like conjugating domain which has an amino acid sequence having at least 80% sequence identity to a human E2 ubiquitin or ubiquitin-like domain, and a targeting domain capable of targeting the regulation domain to a substrate. Also provided are polynucleotides encoding such molecules, methods of identifying and producing the same, and related pharmaceutical compositions and kits suitable for use, among other things, in treating or preventing a disease and/or condition in a subject that is mediated by a dysregulated substrate.

### BACKGROUND

Ubiquitination is characterised by a rapid and reversible posttranslational covalent binding of ubiquitin to proteins. The mechanism plays an important role in targeting proteins for degradation, and in modulating their sub-cellular localisation, intracellular signalling, and interactions with other proteins (Glickman and Ciechanover, Physiol Rev 2002, 82(2):373-428; Mukhopadhyay and Riezman, Science 2007, 315(5809):201-5; and Schnell and Hicke, J Biol Chem 2003, 278(38):35857-60).

Ubiquitin (Ub) is a small (76 amino acid; 8.6 kDa) regulatory protein. The addition of ubiquitin to a protein is called ubiquitination. Ubiquitination involves activation of the ubiquitin C-terminus. For this to occur, E1 Ub-activating enzymes form a thioester bond with Ub in an adenosine triphosphate-dependent reaction. This is followed by conjugation to an E2 ubiquitin-conjugating enzyme (and potentially to a HECT-style E3 ubiquitin ligase as an E3˜Ub intermediate), and ligation to the substrate protein. Ubiquitin can be bound to a target substrate by (i) lysine residues via an isopeptide bond, (ii) cysteine residues by a thioester bond, (iii) serine and threonine residues by an ester bond, or (iv) the amino group of the protein's N-terminus via a peptide bond.

A single ubiquitin protein (monoubiquitination) or a chain of ubiquitin (polyubiquitination) can be added to the substrate. In polyubiquitin chains, the secondary ubiquitin molecules are linked to one of the seven lysine residues (for example K48 or K63) or the N-terminal methionine of the previous ubiquitin molecule. The addition of ubiquitin to proteins allows modulation of proteins by either labelling them for degradation via the proteasome, altering their cellular location, affecting their activity, and promoting or preventing protein interactions.

Several proteins similar to ubiquitin, termed ubiquitin-like proteins (Ubls), can be used in similar mechanisms. The human genome encodes at least eight families of ubiquitin-like proteins, not including ubiquitin itself, that are considered Type I Ubls. These are small ubiquitin-like modifier (SUMO), neural precursor cell expressed developmentally down-regulated 8 (NEDD8), autophagy-related protein 8 (ATG8), autophagy-related protein 12 (ATG12), ubiquitin-related modifier 1 (URM1), ubiquitin-fold modifier 1 (UFM1), ubiquitin-like protein FAT10, and interferon-stimulated gene 15 (ISG15). Type I Ubls are capable of covalent conjugation. Covalent conjugation occurs through one to two glycine residues at the C-terminus. Humans also encode fau ubiquitin-like protein (FUBl), a Type 2 Ubl, which is not capable of covalent conjugation. Proteins tagged by SUMO or NEDD8 are not recognised for degradation; however, they play a role in gene transcription activation, protein localisation and stabilisation. Each target-function combination has its own unique combination of E1, E2 and E3 enzymes.

E2 enzymes act to transfer ubiquitin to a target substrate and all share a core catalytic domain of around 150 amino acids called the ubiquitin core catalytic domain (UBC domain). Generally, the domain adopts an α/β-fold typically with four α-helices and a 4 stranded β-sheet (Stewart et al., Cell Res 2016, 26:423). Important loop regions form part of the E3-binding site and the E2 active site. This surface is involved in binding to both RING-E3 and HECT-E3 domains and overlaps with the region recognised by E1 enzymes. UBC domains are approximately 14-16 kDa and are ˜35% conserved among different family members (Dikic et al., Nat Rev Mol Cell Biol 2009, 10:659-671). E2s have a common fold that has been adapted for specific systems. Although most E2s encompass only a single structural UBC domain, many have short N- and/or C-terminal extensions that can impart important E2-specific functionality, such as recognising bound ubiquitin for chain-building E2s. Some E2s have functionally important insertions, including Ube2R1 and Ube2G2 and some E2s have additional structured domain linked to the UBC domain (e.g. Ube2K) or are part of large multi domain proteins (Ube2O or BIRC6).

E2 enzymes primarily engage in two types of reactions for the transfer of ubiquitin from a E2˜Ub conjugate to the substrate: (1) transthiolation (transfer from a thioester to a thiol group—e.g. transfer of ubiquitin to the active site cysteine residue of a HECT-type E3 ligase) and (2) aminolysis (transfer from a thioester to an amino group), but others have also been reported. (Stewart et al., Cell Res 2016, 26: 423).

All E2s interact with an E1 enzyme and one or more E3s. In addition, E2s may directly engage a target protein (e.g. those termed E3/E2 hybrid such as BIRC6 and UBE2O are E3-independent, as are able to interact and ubiquitinate their substrates without assistance from E3s) and so play a role in the determination of where and how a target is modified by ubiquitin. To date, there are no examples where an E3 alters the chemical reactivity profile of an E2, so the intrinsic reactivity of a given E2 will likely be predictive of the nature of its products.

On the basis of their mechanistic strategies, E3s have been classified into three families, namely RING, HECT and RING-between-RINGS (RBR). RING/U-box E3 ubiquitin ligases are able to bind to both a substrate and a E2-Ub conjugate. HECT/RBR domain E3 ligases must additionally be able to form an intermediate thioester with ubiquitin (E3-Ub). Some E2s can function with multiple types of E3.

HECT domain-containing E3 ubiquitin ligases form intermediate thioesters with Ub (E3˜Ub) at their active site cysteine before transferring Ub to substrates, whereas most RING finger domain-containing E3 enzymes act as scaffolds that bind to E2 enzymes and substrates simultaneously.

RING E3s (most E3s) are not involved directly in the chemical transfer of Ub to substrates. They bind substrates and an E2˜Ub conjugate to facilitate Ub transfer directly from the E2 active site to the substrate. RING E3s function as a protein co-factor for E2˜Ub conjugates. RING E3/E2˜Ub complexes are dynamic; however, interactions with RING E3s increase the intrinsic reactivity of many (but not all) E2˜Ub conjugates towards aminolysis. For example, the Ube2D family of E2s react with lysine slowly in the absence of an E3, but rapidly in the presence of a RING domain.

E2˜Ub generally adopt a “closed state” upon RING E3 binding. A conserved RING (allosteric linchpin) residue, usually an arginine, lysine or asparagine, donates a hydrogen bond to an E2 backbone carbonyl in loop 7 and one or more backbone groups in the tail of Ub. This E2˜Ub closed state is thought of as an activated state for aminolysis. Transthiolation reactions can readily occur in absence of E3s. Therefore, it is hypothesised that E3s that progress with a E3˜Ub conjugate intermediate (e.g. HECT E3s) do not need to promote E2˜Ub closed states.

It will be appreciated that the ability to regulate a specific target via its modification with ubiquitin or ubiquitin-like proteins has potential utility in studying protein function and in combating disease.

PROteolysis-TArgeting Chimeras (PROTACs) are engineered chemical entities that make use of the ubiquitin-proteasome pathway and allow for temporally controlled elimination of proteins in a post-translational manner, operating through simultaneous binding of a target protein and an E3 ligase. A PROTAC molecule brings a target protein into contact with an E3 ubiquitin ligase, prompting transfer of ubiquitin from an E2 ubiquitin conjugating enzyme, leading to ubiquitination of the target protein and degradation by the proteasome. PROTACs have significant potential to target previously ‘undruggable’ proteins for applications in drug discovery and the development of new therapies (Schneekloth et al, Bioorg Med Chem Letter 2008, 18(22):5904-08). The first generation PROTACs were peptide-based PROTACs that contain a phosphopeptide that binds to the E3 ligase beta-TRCP, and a small-molecule Ovalicin that targets MetAP-2 (Schneekloth et al., Bioorg Med Chem Letter 2008, 18(22):5904-08). Since then, small-molecule PROTACs, MDM2-based PROTACs, IAP-based PROTACs, CRBN-based PROTACs and VHL-based PROTACs have been developed. More than thirty small molecule PROTACs have been reported, which target, for example, the androgen receptor (Olson et al., Nat Chem Biol 2018, 14(2):163-70), cyclin dependent kinase 9 (Robb et al., Chem Commun 2017, 53(54):7577-80; and Burslem et al., Cell Chem Biol 2018, 25(1):67-77), and c-Met, with 10 degradation of the target protein providing several advantages over inhibition, in terms of potency, selectivity and drug resistance (Pan et al., Oncotarget 2016, 7(28):44299-44309).

A range of biological PROTACs has also been developed. For example, Portnoff et al (J Biol Chem, 289(11):7844-5) describe so-called ubiquibodies, which are engineered protein chimeras that combine the activity of E3 ubiquitin ligase with designer binding proteins such as single-chain Fv intrabodies or a fibronectin type III domain (FN3) monobody. Pan et al (Oncotarget 7(28):44299-44309) have developed a recombinant chimeric protein that specifically induces mutant KRAS degradation and potently inhibits pancreatic tumour growth. The chimeric protein comprises the Ras binding domain (RBD) of Raf1 and an E3 adaptor protein. Fulcher et al (Open Biol 7:170066) describe an affinity-directed protein missile (AdPROM system) that harbours the von Hippel-Lindau (VHL) protein, the substrate receptor of the Cullin2 (CUL2) E3 ligase complex, tethered to polypeptide binders that selectively bind and recruit endogenous target proteins to the CUL2-E3 ligase complex for ubiquitination and proteasomal degradation. Another biological based degradation system is the so-called Trim-Away technology developed by Clift et al (Cell 2017, 171(7):1692-1706), which involves TRIM21, an E3 ubiquitin ligase that binds with high affinity to the Fc domain of antibodies.

However, various problems remain in existence before PROTAC technology can mature for clinical applications such as off-target effects, in vivo metabolic stability, cellular permeability, and large molecular weight. It is also difficult to synthesise and optimise such bifunctional molecules, which are important obstacles in research and manufacture. Thus, there remains a need for further such bifunctional molecules.

### SUMMARY

Despite all known biological PROTACs requiring a E3 ubiquitin ligase, surprisingly and unexpectedly, the present inventors have identified a novel class of molecules that contain E2 enzymes which can nevertheless provide for targeted degradation or regulation of proteins via the ubiquitin or ubiquitin-like protein pathways. Such molecules are simpler to produce, smaller, easier to deliver, less reliant on endogenous proteins, easier to modularise and may reach a broader set of targets.

Provided herein is a molecule comprising (a) a regulation domain comprising an E2 ubiquitin or ubiquitin-like conjugating domain which has an amino acid sequence having at least 80% sequence identity to a human E2 enzyme or a functional part thereof, and (b) a targeting domain capable of targeting the regulation domain to a substrate. In certain embodiments, the molecule does not comprise an E3 ubiquitin or ubiquitin-like ligase or functional part thereof. In some embodiments, the molecule is a fusion polypeptide. In some embodiments, the regulation domain is N-terminal to the targeting domain. In other embodiments, the regulation domain is C-terminal to the targeting domain.

Further provided herein is a compound comprising the foregoing molecule and a targeting moiety capable of targeting the molecule to a cell. Further provided herein is use of a compound comprising (i) the foregoing molecule and (ii) a targeting moiety capable of targeting the molecule to a cell, in the manufacture of a medicament for delivering the molecule in an individual.

The present disclosure further provides a polynucleotide encoding the foregoing molecule or the foregoing compound. Further provided herein is a vector comprising the foregoing polynucleotide, such as an adeno-associated virus (AAV) vector or a lentiviral vector. Further provided herein is a host cell comprising the polynucleotide or the vector. Further provided herein is a composition comprising the foregoing molecule or the foregoing compound and a further therapeutic agent.

Further provided herein is a pharmaceutical composition comprising the foregoing molecule, the foregoing compound, the foregoing polynucleotide, the foregoing vector, the foregoing host cell, or the foregoing composition, and one or more pharmaceutically acceptable carrier, diluent or excipient.

Another aspect of the disclosure provides a method of delivering the foregoing molecule to a cell in an individual, the method comprising: administering to the individual a compound comprising (i) the molecule and (ii) a targeting moiety capable of targeting the molecule to the cell; or administering to the individual the foregoing polynucleotide or the foregoing vector, wherein the polynucleotide or vector encodes the molecule in the cell.

Another aspect of the disclosure provides a kit of parts comprising: (a) a regulation domain comprising an E2 ubiquitin or ubiquitin-like conjugating domain which has an amino acid sequence having at least 80% sequence identity to a human E2 enzyme or a functional part thereof, and (b) a targeting domain capable of targeting the regulation domain to the substrate; optionally wherein the kit does not comprise an E3 ubiquitin or ubiquitin-like ligase or a functional part thereof.

Further provided herein is a kit of parts comprising: (a) the foregoing molecule; and (b) a targeting moiety that is capable of targeting to cells that contain the substrate to be regulated, optionally wherein the targeting moiety is a binding partner such as an antibody. Further provided herein is a method of preventing or treating a disease or condition mediated by an aberrant level of a substrate or form thereof in a subject, the method comprising administering the foregoing molecule, the foregoing compound, the foregoing polynucleotide, the foregoing vector, the foregoing host cell, the foregoing pharmaceutical composition, or the foregoing composition to the subject. Further provided herein is the foregoing molecule, the foregoing compound, the foregoing polynucleotide, the foregoing vector, the foregoing host cell, the foregoing pharmaceutical composition, or the foregoing composition for use in preventing or treating a disease or condition mediated by an aberrant level of a substrate or form thereof in a subject. In some embodiments, the disease or condition is cancer, diabetes, autoimmune disease, Alzheimer's disease, Parkinson's disease, pain, viral disease, bacterial disease, prionic disease, fungal disease, parasitic disease, arthritis, immunodeficiency, or inflammatory disease.

Further provided herein is a method of regulating a substrate, the method comprising contacting the substrate with the foregoing molecule under conditions effective for the molecule to regulate the substrate. In some embodiments, the regulating involves the substrate being degraded, or the substrate being prevented from being degraded, or the subcellular location of the substrate being altered, or one or more activities of the substrate being modulated (e.g. increased or decreased), or the degree of post-translational modification of the substrate being modulated.

Further provided herein is a method of identifying a substrate as a potential drug target, the method comprising: (a) providing a cell, tissue or organ comprising the substrate; (b) contacting the cell, tissue or organ with the foregoing molecule, the foregoing compound, the foregoing polynucleotide, or the foregoing vector; and (c) assessing the effect of the molecule, compound, polynucleotide or vector on one or more properties of the cell, tissue or organ, wherein identification of an effect that is correlated with a particular disease status is indicative that the substrate is a potential drug target for the particular disease.

Further provided herein is a method of assessing the function of a substrate, the method comprising: (a) providing a cell, tissue or organ comprising the substrate; (b) contacting the cell, tissue or organ with the foregoing molecule, the foregoing compound, the foregoing polynucleotide, or the foregoing vector; and (c) assessing the effect of the molecule, compound, polynucleotide or vector on one or more properties of the cell, tissue or organ.

Further provided herein is a method of identifying a test agent that may be useful in preventing or treating a disease or condition mediated by an aberrant level of a substrate or form thereof, the method comprising: providing the substrate; providing a test agent comprising (a) a regulation domain comprising an E2 ubiquitin or ubiquitin-like conjugating domain which has an amino acid sequence having at least 80% sequence identity to a human E2 ubiquitin or ubiquitin-like domain, and (b) a targeting domain capable of targeting the regulation domain to a substrate, optionally wherein the test agent does not comprise an E3 ubiquitin or ubiquitin-like ligase or part thereof; contacting the substrate and test agent under conditions effective for the test agent to facilitate regulation of the substrate; and determining whether the test agent regulates the substrate. In some embodiments, the method further comprises the step of testing the test agent in an assay of the disease or condition.

### BRIEF DESCRIPTION OF THE DRAWINGS

FIG. 1. Degradation of SHP2 protein in MDA-MB-231 cells comparing an E3 fusion polypeptide and an E2 fusion polypeptide. (FIG. 1A) Western blot of MDA-MB-231 cell lysates following lentiviral transduction of encoded control and fusion polypeptide constructs. SHP2 and loading control alpha tubulin are shown. Two replicate samples of E2 fusion polypeptide UBE2D1_aCS3, where SHP2 protein levels are reduced. The black box identifies lysates from cells transduced with E3 ligase fusion polypeptides and the grey box identifies E2 fusion polypeptide constructs. (FIG. 1B) Graph displaying densitometry of western blot signals. Band density of SHP2 protein levels were normalised to alpha tubulin loading control band density and then represented as a percentage of control MDA-MB-231 cell SHP2 levels. Data is representative of multiple replicates in multiple cell lines.

FIG. 2. Degradation of SHP2 protein in MDA-MB-231 cells comparing orientation and linker length in E3 ligase and E2 biological fusion polypeptides. (FIG. 2A) Western blot of MDA-MB-231 cell lysates following lentiviral transduction of encoded control and fusion polypeptide constructs. SHP2 protein and GAPDH loading control are shown. The black box identifies lysates from cells transduced with E3 ligase fusion polypeptides and the grey box identifies E2 fusion polypeptide constructs. (FIG. 2B) Graph displaying densitometry of western blot signals. Band density of SHP2 protein levels were normalised to GAPDH loading control band density and then represented as a percentage of control MDA-MB-231 cell SHP2 levels. The UBE2D1 regulation domain constructs have used the shorter name E2D1. Within the sample names, ‘short’ and ‘long’ refer to the different linker lengths of 9 and 19 amino acids respectively.

FIG. 3. Degradation of SHP2 protein in U20S cells comparing orientation and linker length in E3 ligase and E2 biological fusion polypeptides. (FIG. 3A) Western blot of U20S cell lysates following lentiviral transduction of encoded control and fusion polypeptide constructs. SHP2 protein and GAPDH loading control are shown. The black box identifies lysates from cells transduced with E3 ligase fusion polypeptides and the grey box identifies E2 fusion polypeptide constructs. (FIG. 3B) Graph displaying densitometry of western blot signals. Band density of SHP2 protein levels were normalised to GAPDH loading control band density and then represented as a percentage of control U20S cell SHP2 levels. The UBE2D1 regulation domain constructs have used the shorter name E2D1. Within the sample names, ‘short’ and ‘long’ refer to the different linker lengths of 9 and 19 amino acids respectively. (FIG. 3C) Graph comparing different linker lengths and efficiency of SHP2 degradation using E2D1_Linker_aCS3 (UBE2D1_Linker_aCS3) constructs based on fluorescence signals following imaging with a Cytation 5 (Biotek®). Fluorescence intensity of SHP2 and HA tag protein levels were probed using antibodies specific for these epitopes, and SHP2 levels were normalised to the range 0-100% based on SHP2 levels found within untreated cells within each experiment. Data correspond to n=3 or more biological replicates. The number of residues for the linker length refers to the number of amino acids in the linker sequence.

FIG. 4. Degradation of SHP2 protein in MDA-MB-231 cells comparing high and low affinity variants of the SHP2-binding monobody as binding domain. Standard aCS3 monobody with high affinity to SHP2 (SHP2 C-SH2 domain Kd=4-9.1 nM) was compared to V33R aCS3 mutant with lower affinity (SHP2 C-SH2 domain Kd=1.2 uM; Sha et al., Proc. Natl. Acad. Sci. USA, 2013 110(37):14924-9 and supplementary information). Note: monobody aCS3 is also referred to as CS3 herein. (FIG. 4A) Western blot of MDA-MB-231 cell lysates following lentiviral transduction of encoded control and fusion polypeptide constructs with aCS3 and aCS3 V33R mutant binding domains. SHP2 protein and GAPDH loading control are shown. The black box identifies lysates from cells transduced with E3 ligase fusion polypeptides and the grey box identifies E2 fusion polypeptide constructs. There are two replicate samples of E2D1_long_aCS3 in the western blot. (FIG. 4B) Graph displaying densitometry of western blot signals. Band density of SHP2 protein levels were normalised to GAPDH loading control band density and then represented as a percentage of control MDA-MB-231 cell SHP2 levels. The UBE2D1 regulation domain constructs have used the shorter name E2D1. Within the sample names, ‘long’ refers to the 19 amino acid linker between the regulation and binding domains.

FIG. 5. Degradation of SHP2 protein in U20S cells comparing high and low affinity variants of the SHP2-binding monobody as binding domain. Standard aCS3 monobody with high affinity to SHP2 (SHP2 C-SH2 domain Kd=4-9.1 nM) was compared to V33R aCS3 mutant with lower affinity (SHP2 C-SH2 domain Kd=1.2 uM; Sha et al., Proc. Nat. Acad. Sci. USA, 2013 110(37):14924-9 and supplementary information). Note: monobody aCS3 is also referred to as CS3 herein. (FIG. 5A) Western blot of U20S cell lysates following lentiviral transduction of encoded control and fusion polypeptide constructs with aCS3 and aCS3 V33R mutant binding domains. SHP2 protein and GAPDH loading control are shown. The black box identifies lysates from cells transduced with E3 ligase fusion polypeptides and the grey box identifies E2 fusion polypeptide constructs. (FIG. 5B) Graph displaying densitometry of western blot signals. Band density of SHP2 protein levels were normalised to GAPDH loading control band density and then represented as a percentage of control U20S cell SHP2 levels. The UBE2D1 regulation domain constructs have used the shorter name E2D1. Within the sample names, ‘long’ refers to the 19 amino acid linker between the regulation and binding domains.

FIG. 6. Comparison of the degradation of KRas using K19 DARPin_E2 fusion and K19 DARPin_E3 fusion polypeptides. DARPin K19 binds to both GDP- and GTP-bound KRAS (Bery et al, Nat Commun 201910(1):2607) whereas E3_5 is a negative control (non-binding) DARPin. The DARPin fusion polypeptide constructs were tested in both MDA-MB-231 and Ad293 cell lines. (FIG. 6A) Western blot of MDA-MB-231 and Ad293 cell lysates following lentiviral transduction of encoded control and fusion polypeptide constructs. KRas protein and alpha tubulin loading control protein are shown. The black box identifies lysates from cells transduced with E3 ligase fusion polypeptides and the grey box identifies E2 fusion polypeptide constructs. (FIG. 6B) Graph displaying densitometry of western blot signals. Band density of KRAS protein levels were normalised to alpha tubulin loading control band density and then represented as a percentage of control MDA-MB-231 or Ad293 cell KRAS levels respectively.

FIG. 7. Schematic of E3 and E2 fusion polypeptides. (FIG. 7A) Example of an E3 biological fusion polypeptide comprising E3 ligase VHL fused to a binding domain via a linker. As previously described by Fulcher et al (Fulcher et al, 2017, Open Biol 7:170066). The binding domain (for example a monobody, nanobody or antibody mimetic) can recruit a target protein (an endogenous protein or a non-endogenous or ectopically expressed protein such as a viral protein) in the cell to the EloB/C/CUL2/RBX1 E3 ligase machinery. This complex then binds an E2 conjugating enzyme which allows the transfer of ubiquitin to the target protein. The addition of multiple ubiquitin molecules, forming a chain, is termed polyubiquitination and labels the target protein for degradation by the proteasome. Alternative E3 ligase fusion polypeptides may require the involvement of different proteins to allow ubiquitination of the target protein. (FIG. 7B) Example of an E2 biological fusion polypeptide comprising an E2 ubiquitin conjugating domain directly fused to a binding domain via a linker. The binding domain (for example a monobody, nanobody or antibody mimetic) can bind a target protein (an endogenous protein or a non-endogenous or ectopically expressed protein such as a viral protein) in the cell allowing the E2 ubiquitin conjugating domain to transfer of ubiquitin to the target protein. Polyubiquitination of the target protein will result in degradation of the target protein by the proteasome. In these examples, if the E2 ubiquitin conjugating domain was replaced by a ubiquitin-like conjugating domain, then rather than ubiquitin transfer to the target protein, a ubiquitin-like molecule would be transferred to the target protein (for example, SUMO, NEDD8, RUB1, ATG8, ATG12, ISG15, FAU, or URM1).

FIG. 8. Investigating the effect on SHP2 protein expression of a panel of E2 ubiquitin conjugating enzyme and E2 ubiquitin-like conjugating enzyme core domains fused to the aCS3 binding domain, in MDA-MB-231 cells. 26 different E2 ubiquitin conjugating enzyme and E2 ubiquitin-like conjugating enzyme core domains were encoded on lentiviral plasmids as fusion proteins in the following format, HA tag_E2_linker_aCS3. Lentiviral particles were then produced and used to transduce MDA-MB-231 cells. (FIG. 8A) Western blots of MDA-MB-231 cell lysates following lentiviral transduction of encoded control and panel of E2 fusion constructs. Western blots were probed with antibodies to SHP2, HA tag (indicating expression levels of fusion proteins) and alpha tubulin (as a loading control). The protein lysate from MDA-MB-231 cells transduced with lentiviral particles encoding UBE2D1_aCS3 fusion protein were run on each separate gel for comparison purposes. (FIG. 8B) Graph displaying the amount of SHP2 protein observed for each E2 core domain fusion protein relative to SHP2 degradation observed with UBE2D1_aCS3 fusion polypeptide. Values were calculated using densitometry of western blot signals. Band density of SHP2 protein levels were normalised to alpha tubulin loading control band density and then represented as a percentage of SHP2 levels observed for cells transduced with lentiviral particles encoding the UBE2D1_aCS3 fusion polypeptide. Core domains of interest were those able to reduce SHP2 protein levels to a similar or greater extent than UBE2D1_aCS3.

FIG. 9. Investigating the effect on SHP2 protein expression of a panel of E2 ubiquitin conjugating enzyme and E2 ubiquitin-like conjugating enzyme core domains fused to the aCS3 binding domain, in U20S cells. 26 different E2 ubiquitin conjugating enzyme and E2 ubiquitin-like conjugating enzyme core domains were encoded on lentiviral plasmids as fusion proteins in the following format, HA tag_E2_linker_aCS3. Lentiviral particles were then produced and used to transduce U20S cells. (FIG. 9A) Western blots of U20S cell lysates following lentiviral transduction of encoded control and panel of E2 fusion constructs. Western blots were probed with antibodies to SHP2, HA tag (indicating expression levels of fusion proteins) and alpha tubulin (as a loading control). The protein lysate from U20S cells transduced with lentiviral particles encoding UBE2D1_aCS3 fusion protein were run on each separate gel for comparison purposes. (FIG. 9B) Graph displaying the amount of SHP2 protein observed for each E2 core domain fusion protein relative to SHP2 degradation observed with UBE2D1_aCS3 fusion polypeptide. Values were calculated using densitometry of western blot signals. Band density of SHP2 protein levels were normalised to alpha tubulin loading control band density and then represented as a percentage of SHP2 levels observed for cells transduced with lentiviral particles encoding the UBE2D1_aCS3 fusion polypeptide. Core domains of interest were those able to reduce SHP2 protein levels to a similar or greater extent than UBE2D1_aCS3.

FIG. 10. Investigating the effect of mutating the lysine residues within the aCS3 binding domain on SHP2 degradation and fusion polypeptide expression level. The aCS3 monobody contains 3 lysine residues (K7, K55 and K64), making it liable for (self) ubiquitination and degradation when expressed as a fusion protein with an E2 ubiquitin conjugating enzyme. The three aCS3 lysine residues were mutated individually and in combination and expressed in cells as the binding domain of an UBE2D1 fusion polypeptide in the HA tag_E2D1_linker_aCS3 format. Structural modelling performed in-house indicated which amino acid residue changes should retain monobody stability. Lysine residue K7 was mutated to glutamine (K7Q). Lysine residue K55 was mutated to tyrosine (K55Y) and Lysine residue K64 was mutated to histidine (K64H). The effects on SHP2 degradation and fusion polypeptide expression in cells expressing fusion polypeptides containing these aCS3 variants was measured by western blot probing for SHP2 protein and HA tag expression levels, respectively. (FIG. 10A) Western blot of U20S cell lysates following lentiviral transduction of encoded control and lysine-mutated aCS3 variant fusion polypeptide constructs. Western blots were probed with antibodies to SHP2, HA tag (indicating expression levels of fusion proteins) and alpha tubulin (as a loading control). (FIG. 10B) Graph displaying densitometry of western blot signals. Band density of SHP2 protein levels were normalised to alpha tubulin loading control band density and then represented as a percentage of control U20S cell SHP2 levels. (FIG. 10C) Graph displaying densitometry of western blot signals. Band density of HA-tagged fusion polypeptide levels were normalised to alpha tubulin loading control band density and then represented as a percentage of HA-tagged UBE2D1_aCS3 (WT) levels.

FIG. 10 continued. Mutating the catalytic site of UBE2D1 or UBE2B Regulation domains of fusion polypeptides or reducing the Binding domain affinity to the target protein reduces target protein degradation. (FIG. 10D) U20S cells were transfected with mRNA encoding variants of SHP2 targeted fusion polypeptides (using the aCS3 binding domain with all lysines removed i.e. K7Q, K55Y, K64H) and target SHP2 protein levels were determined by Western blotting following 24 hours incubation. U20S cells were transfected with mRNA encoding EGFP as a control (non-degrading mRNA). Variants included (i) mutating the catalytic cysteine residue of regulation domains e.g. UBE2D1 (C85A) and UBE2B (C88A), (ii) reducing binding domain affinity for SHP2 with V33R mutation of aCS3 and (iii) mutating UBE2D1 residue involved in interactions with E3 ligases to determine effects on activity (i.e. F62A). Quantitation of SHP2 expression levels from densitometry measurements of Western blot band intensity relative to loading control levels and normalised to SHP2 levels in U20S cells transfected with EGFP for (FIG. 10E) UBE2D1 and (FIG. 10F) UBE2B fusion polyproteins.

FIG. 11. Investigating the degradation of human antigen R (HuR) in MDA-MB-231 cells using UBE2D1 fusions comprising V_{HH} nanobody (HuR8 and HuR17) binding domains targeting HuR. HuR is a predominantly nuclear protein. Control UBE2D1 fusion proteins with a Cas9 V_{HH} nanobody binding domain are included. Cas9 is a bacterial protein, and therefore is not endogenously expressed in mammalian cells. Hence, the Cas9 V_{HH} nanobody should not selectively bind to any proteins in mammalian cells. The effects on HuR protein levels was explored for fusion constructs in both orientations. (FIG. 11A) Western blot of MDA-MB-231 cell lysates following lentiviral transduction of encoded control (UBE2D1_Cas9 V_{HH}) and HuR-binding variant fusion polypeptide constructs, UBE2D1_HuR17 and UBE2D1_HuR8. Western blots were probed with antibodies to HuR and alpha tubulin (as a loading control). (FIG. 11B) Graph displaying densitometry of western blot signals. Band density of HuR protein levels were normalised to alpha tubulin loading control band density and then represented as a percentage of HuR levels observed for cells expressing UBE2D1_Cas9 V_{HH}. (FIG. 11C) Western blot of MDA-MB-231 cell lysates following lentiviral transduction of encoded control (Cas9 V_{HH}_UBE2D1) and HuR-binding variant fusion polypeptide constructs, HuR17_UBE2D1 and HuR8_UBE2D1. Western blots were probed with antibodies to HuR, and alpha tubulin (as a loading control). (FIG. 11D) Graph displaying densitometry of western blot signals. Band density of HuR protein levels were normalised to alpha tubulin loading control band density and then represented as a percentage of HuR levels observed for cells expressing Cas9 V_{HH}_UBE2D1.

FIG. 12. Investigating the degradation of human antigen R (HuR) in U20S cells using UBE2D1 fusions comprising of V_{HH} nanobody (HuR8 and HuR17) binding domains targeting HuR. HuR is a predominantly nuclear protein. Control UBE2D1 fusion proteins with a Cas9 V_{HH} nanobody binding domain are included. Cas9 is a bacterial protein, and therefore is not endogenously expressed in mammalian cells. Hence, the Cas9 V_{HH} nanobody should not selectively bind to any proteins in mammalian cells. The effects on HuR protein levels was explored for fusion constructs in both orientations. (FIG. 12A) Western blot of U20S cell lysates following lentiviral transduction of encoded control (UBE2D1_Cas9 V_{HH}) and HuR-binding variant fusion polypeptide constructs, UBE2D1_HuR17 and UBE2D1_HuR8. Western blots were probed with antibodies to HuR and alpha tubulin (as a loading control). (FIG. 12B) Graph displaying densitometry of western blot signals. Band density of HuR protein levels were normalised to alpha tubulin loading control band density and then represented as a percentage of HuR levels observed for cells expressing UBE2D1 Cas9 V_{HH}. (FIG. 12C) Western blot of U20S cell lysates following lentiviral transduction of encoded control (Cas9 V_{HH}_UBE2D1) and HuR-binding variant fusion polypeptide constructs, HuR17_UBE2D1 and HuR8_UBE2D1. Western blots were probed with antibodies to HuR and alpha tubulin (as a loading control). (FIG. 12D) Graph displaying densitometry of western blot signals. Band density of HuR protein levels were normalised to alpha tubulin loading control band density and then represented as a percentage of HuR levels observed for cells expressing Cas9 V_{HH}_UBE2D1.

FIG. 13. Graph comparing different degradation domains for the degradation of KRas in HPAC cell line. HPAC pancreatic cancer cell lines were transduced with lentivirus encoding PROTACs targeting KRas (using the KRas binding DARPin K19). PROTACs containing the following degradation domains were investigated; UBE2D1 (E2D1), UBE2B (E2B) and VHL. KRas targeted PROTACs were tested in both “Binding domain_Regulation domain” and “Regulation domain_Binding domain” orientations. Negative control DARPin E3_5 was used as a negative control binding domain in combination with the various degradation domains in the following orientation: E3_5_Regulation domain. (FIG. 13A) Western blot of KRas and loading control alpha tubulin expression in cell lysates transduced with lentiviral PROTAC constructs. (FIG. 13B) Graph quantitating KRas expression using western blot densitometry of KRas and alpha tubulin expression. Data is shown relative to untreated cells alone control and normalised to alpha tubulin loading control levels.

### DETAILED DESCRIPTION

The present disclosure relates to targeted protein regulation using molecules comprising targeting moieties and regulation domains, and the use of such regulation to study protein function and to combat disease. In particular, a first aspect of the disclosure provides a molecule comprising: (a) a regulation domain comprising an E2 ubiquitin or ubiquitin-like conjugating domain which has an amino acid sequence having at least 80%, 85%, 90%, 95%, or 98% sequence identity to a human E2 enzyme or a functional part thereof, and (b) a targeting domain capable of targeting the regulation domain to a substrate.

By the term ‘molecule’, we include the meaning of any entity possessing a regulation domain and a targeting domain as defined herein. In a preferred embodiment, the molecule is a polypeptide. In some embodiments, the regulation and targeting domains are attached via a polypeptide linker. Preferably, the molecule is a polypeptide, and the regulation domain and targeting domain are attached via a polypeptide linker, as further described herein.

It will be appreciated that reference to a regulation domain and a targeting domain simply refers to discrete portions of the molecule having the respective functions of regulation and targeting as explained herein. In this way, the molecules of the disclosure can be considered to be a bifunctional molecule, which typically comprises two protein binding domains joined by a linker of appropriate length. Given the different respective functions of the domains, it will be understood that the molecule is generally heterobifunctional.

By regulation domain, we include the meaning of the portion of the molecule of the disclosure that is capable of facilitating the regulation of a target substrate, such as regulating one or more activities of a target substrate and/or regulating the cellular location of a target substrate and/or regulating the stability of a target substrate and/or regulating the degree of post-translational modification of a target substrate. A regulation domain may result in regulation of the target by any means; however, since the regulation domain contains an E2 ubiquitin or ubiquitin-like conjugating domain, it will be appreciated that the regulation is typically mediated by conjugating a ubiquitin or ubiquitin-like protein to the target substrate. The skilled person will be aware that different ubiquitin or ubiquitin-like proteins conjugated to a target substrate will exert different effects on one or more activities of a target substrate and/or on the cellular location of a target substrate and/or on the stability of a target substrate, depending on which ubiquitin or ubiquitin-like protein is conjugated to it. Such effects are reviewed in Herrman et al (Circ Res 2007, 100(9):1276-1291). Also, the conjugation of a ubiquitin or ubiquitin-like protein to a target substrate may regulate the target substrate's activity by steric effects, such as slowing the rate of a chemical reaction and/or preventing downstream signalling, for example by steric hindrance. Adding ubiquitin or ubiquitin-like-molecule to a substrate may directly impede the interaction of the substrate with a binding partner due to the size of the ubiquitin/ubiquitin-like protein addition (e.g. RAS:RAF binding could be blocked, thereby halting signalling). For the avoidance of doubt, all such effects are encompassed by the term “regulation” as used herein.

In one embodiment, the regulation includes the target substrate being degraded, or the increased stability of the target substrate, or the subcellular location of the target substrate being altered, or one or more activities of the target substrate being modulated (e.g. increased or decreased), or the degree of post-translational modification of the target substrate being modulated.

In a specific embodiment, the regulation domain comprises an E2 ubiquitin-conjugating domain which is capable of conjugating ubiquitin to the target substrate, such that the target substrate is thereby degraded. In this way, when the regulation domain acts to degrade a target substrate, it will be appreciated that it may be referred to as a degradation domain.

In another specific embodiment, the regulation comprises an E2 ubiquitin-like conjugating domain which is capable of modulating the subcellular location of a target substrate or modulating one or more activities of the target substrate.

Thus, depending on the identity of the regulation that is exerted by the domain, it will be understood that the regulation domain can be considered to be a degradation domain, a localisation domain, an activation domain or a deactivation domain. In a preferred embodiment, the regulation domain is a degradation domain.

In a preferred embodiment, the regulation domain acts to degrade the target substrate by virtue of it containing an E2 ubiquitin conjugating domain, in which case it may be termed a degradation domain.

By degradation we include the meaning that the amount of the target substrate is decreased by virtue of the target substrate being degraded in the proteasome. The amount of target substrate may be decreased when in the presence of the molecule of the disclosure compared to the amount of the target substrate when in the absence of the molecule of the disclosure. For example, in the presence of the molecule of the disclosure, the amount of the target substrate may be decreased by at least 10%, 20%, 30%, 40%, 50%, 60%, 70%, 80%, 90%, 95%, or 99% compared to the amount of the target substrate in the absence of the molecule of the disclosure. Similarly, when a cell contains a molecule of the disclosure, it will be appreciated that the molecule may decrease the amount of target substrate in the cell, for example, by at least 10%, 20%, 30%, 40%, 50%, 60%, 70%, 80%, 90%, 95% or 99% compared to the level of the target substrate in the cell in the absence of the molecule of the disclosure. Preferably, the amount of the target substrate is decreased to an undetectable level. In an embodiment, the molecule of the present disclosure results in the degradation of 30-100% of the amount of target substrate in the absence of the molecule of the disclosure, such as 40-100%, 50-100%, 60-100%, 70-100%, 80-100% or 90-100%. It is understood that substrates may be degraded in the absence of the molecule of the disclosure at a background level, for example as part of normal protein turnover. In this case, the regulation may act to degrade the target substrate to a greater extent than the background degradation rate.

For intracellular target substrates, the extent of degradation can be assessed by measuring the level of the target substrates in a cell that contains the molecule of the disclosure, and measuring the level of the target substrate in a cell that is otherwise substantially the same but does not contain a molecule of the disclosure. By “substantially the same”, we include the meaning that the cells are of the same type (e.g. express substantially the same cell surface markers), and/or are from the same tissue, and/or are in the same stage of the cell cycle. Alternatively, one may measure the starting amount of target substrate in the cell in the absence of the molecule of the disclosure, and then measure the amount of target substrate following addition of the molecule of the disclosure to the cell. As yet another alternative, the amount of the target substrate in a cell in which a molecule of the disclosure is present may also be compared to a negative control. By a “negative control” we include the meaning of a cell in which an inactive version of the molecule of the disclosure is present, for example a molecule lacking a targeting domain and/or a regulation domain, or including a non-functional targeting and/or regulation domain. For example, the inactive version may lack a binding domain for the substrate or may have an irrelevant binding domain. Similarly, the inactive version may contain an inactive regulation domain, for example one that cannot interact with one or more binding partners that are required to mediate the regulation. For example, as described further below, including in Example 6, a variant of the E2 enzyme, UBE2D1, containing the mutation F62A, completely abrogated regulation activity. Without wishing to be bound by any theory, the inventors believe that this is due to the F62 residue being involved in the interaction between UBE2D1 and Ring-type E3 ligases, such as RNF4. Hence, it will be appreciated that the negative control may be one in which the E2 protein is unable to interact with an E3 protein, such as one comprising a mutation at the position corresponding to F62 in the E2 protein UBE2D1 (e.g. F62A). In another example, the inactive regulation domain may comprise one or more mutations at the position of a catalytic cysteine residue, thereby abrogating its catalytic activity. Example mutations in the catalytic cysteine residue of regulation domains include C85A for UBE2D1 and C88A for UBE2B, which abrogate regulation activity. The disclosure provides examples of such negative control fusion proteins in Table 12A below. Again, it is preferred if the cell of the negative control is otherwise substantially the same as the cell containing the molecule of the disclosure. It will be appreciated, therefore, that references to at least 10%, 20%, 30%, 40%, 50%, 60%, 70%, 80%, or 90% etc. degradation compared to the amount of substrate in the absence of the molecule of the disclosure, includes the meaning of compared to the amount of the substrate in a cell that is otherwise substantially the same but which does not contain a molecule of the disclosure, or compared to the amount of the substrate in the cell prior to the addition of the molecule of the disclosure in the cell, or compared to the amount of the substrate in a cell containing an inactive version of the molecule of the disclosure.

Assessing the level of target substrate in the presence and absence of the molecule of the disclosure can be done using well-known techniques in the art. For example, assessing the levels of protein is standard practice in the art and any suitable method may be used. For example, immunoassays such as ELISA or a radio-immunoassay, immunofluorescence, HPLC, gel electrophoresis and capillary electrophoresis (followed, for example, by UV or fluorescent detection), may be used to detect and quantify a target substrate. Methods of measuring levels of target substrates by mass spectrometry are well known in the art and any suitable form of mass spectrometry may be used. Western blotting, immunoprecipitation, immunohistochemistry on paraffin, immunofluorescence, fluorescence in situ hybridisation and flow cytometry may also be used. As described further in the Examples, western blotting and densitometry are a convenient way of detecting and quantifying target substrates.

As described above, in some embodiments, depending on which ubiquitin or ubiquitin-like protein becomes attached to the target substrate, the regulation domain may be considered a localisation domain, an activation domain or a deactivation domain.

By a localisation domain, we include the meaning of a domain that acts to direct the target substrate so that it preferentially resides in a particular cellular location (e.g. one or more particular subcellular locations such as organelles). For example, in the presence of the molecule of the disclosure, the localisation domain of the molecule may result in a higher proportion of the target substrate within the cell residing in particular subcellular locations, compared to the proportion of target substrate that would residue in those particular subcellular locations in the absence of the molecule of the disclosure. Methods of assessing cellular localisation of target substrates are well known in the art and any suitable method may be used such as immunohistochemistry techniques. Examples of such localisation domains and the effects of the conjugation of ubiquitin-like proteins to the target substrate in the context of localisation include those demonstrated by Embabe et al (“Mdm2-mediated NEDDylation of HuR controls the nuclear localization of HuR and protects it from degradation,” Hepatology 2012, 55(4):1237-48), Wen et al (“SUMOylation Promotes Nuclear Import and Stabilization of Polo-like Kinase 1 to Support Its Mitotic Function,” Cell Rep 2017 21, 2147-59), Matunis et al (“A novel ubiquitin-like modification modulates the partitioning of the Ran-GTPase-activating protein RanGAP1 between the cytosol and the nuclear pore complex,” J Cell Biol 1996, 135(6 Pt 1):1457-70), and Mahajan et al (“A small ubiquitin-related polypeptide involved in targeting RanGAP1 to nuclear pore complex protein RanBP2,” Cell 1997 88(1):97-107), all of which are incorporated herein by reference.

By an activation domain, we include the meaning of a domain that acts to increase one or more activities of the target substrate compared to a reference level of the one or more activities in the absence of the molecule of the disclosure. The one or more activities may include binding interactions with cellular entities such as proteins and/or nucleic acids, or enzymatic activities or signalling activities. The one or more activities may be increased by 1.5-fold, 2-fold, 3-fold, 4-fold, 5-fold, 6-fold, 7-fold, 8-fold, 9-fold or 10-fold relative to the one or more activities in the absence of the molecule of the disclosure. Such activities can be assayed using well-known techniques in the art, such as ELISA, and the skilled person would be able to tailor the assays to the target substrate in question by interrogating the scientific literature. Examples of such activation domains and the effects of the conjugation of ubiquitin-like proteins to the target substrate in the context of activation include those demonstrated by Soucy et al (“Cullin-RING ubiquitin E3 ligases require NEDD8 modification to be activated,” Clin Cancer Res 2009, 15(12):3912-16) and Noh et al (“NEDDylation increases RCAN1 binding to calcineurin,” PLoS ONE 2012, 7(10):e48315), all of which are incorporated herein by reference.

By a deactivation domain, we include the meaning of a domain that acts to decrease one or more activities of the target substrate compared to a reference level of the one or more activities in the absence of the molecule of the disclosure. The one or more activities may include binding interactions with cellular entities such as proteins and/or nucleic acids, or enzymatic activities or signalling activities. The one or more activities may be decreased by 1.5-fold, 2-fold, 3-fold, 4-fold, 5-fold, 6-fold, 7-fold, 8-fold, 9-fold or 10-fold relative to the one or more activities in the absence of the molecule of the disclosure, and are preferably decreased to an undetectable level. Examples of such deactivation domains and the effects of the conjugation of ubiquitin-like proteins to the target substrate in the context of deactivation include those demonstrated by Kamynina and Stover (“SREBP SUMOylation inhibits SREBP transcriptional activity indirectly through the recruitment of a co-repressor complex that includes histone deacetylase 3 (HDAC3),” Adv Exp Med Biol 2017, 963:143-68) and Yang et al (“SUMOylation Inhibits SF-1 Activity by Reducing CDK7-Mediated Serine 203 Phosphorylation,” Mol Cell Biol 2009, 29(3):613-25), all of which are incorporated herein by reference.

By an ‘E2 ubiquitin or ubiquitin-like conjugating domain’, we include the meaning of a domain which is capable of conjugating ubiquitin or a ubiquitin-like protein to a target substrate. For example, the regulation domain may contain an E2 ubiquitin conjugating domain that is capable of binding to ubiquitin and transferring ubiquitin to the target substrate. Alternatively, the regulation domain may contain an E2 ubiquitin-like conjugating domain that is capable of binding to a ubiquitin-like protein and transferring the ubiquitin-like protein to a target substrate, such as any one of SUMO, NEDD8, ATG8, ATG12, ISG15, UFM1, FAT10, URM1, and FUBl.

In some embodiments, the capability of the E2 ubiquitin or ubiquitin-like conjugating domain to conjugate a target substrate, may be assessed when the E2 ubiquitin or ubiquitin-conjugating domain is part of the molecule of the disclosure alongside the targeting domain that selectively targets the target substrate in question. Hence, in an embodiment, the E2 ubiquitin or ubiquitin-like conjugating domain, within the molecule of the disclosure, is capable of conjugating ubiquitin or a ubiquitin-like protein to the target substrate, such that at least 10% of the target substrate is conjugated to the ubiquitin or ubiquitin-like protein. Preferably, at least 20%, 30%, 40%, 50%, 60%, 70%, 80%, 90%, 95% or 99% of the target substrate is conjugated to the ubiquitin or ubiquitin-like protein. The assessment may be carried out in vivo or in vitro. For example, the assessment may be carried out by a recombinant biochemical assay or in a cell.

It will be appreciated that the conjugating of ubiquitin or ubiquitin-like protein to a target substrate may be assessed either directly or indirectly using routine methods in the art. For example, the conjugation for ubiquitin or a ubiquitin-like protein to a target substrate may be measured directly by detecting changes to the molecular weight of the target substrate as a marker of ubiquitin or ubiquitin-like protein conjugation (e.g. by SDS PAGE separation), or by using western blots and immunoassays e.g. based on antibodies that are specific for ubiquitin or ubiquitin-like protein. Alternatively, the conjugation of ubiquitin or ubiquitin-like protein to a target substrate may be measured indirectly, for example by assessing the downstream effect of the conjugation, namely degradation of the target substrate or another regulation as described herein. Again, any suitable technique can be used for such indirect measurement as are well-known in the art, and as described herein and in the Examples. It will be appreciated that such assays may be in vivo or in vitro. Specific examples of ways of measuring ubiquitin or ubiquitin-like conjugation include a cell assay, such as a quantitative live-cell assay (see, for example, Richting et al (“Quantitative live-cell kinetic degradation and mechanistic profiling of PROTAC mode of action,” ACS Chem Biol 2018, 13(9):2758-70), a biotinylation assay, such as an in vivo biotinylation assay (see, for example, Pirone et al “A comprehensive platform for the analysis of ubiquitin-like protein modifications using in vivo biotinylation,” Sci Rep 2017, 7:40756), mass spectrometry and/or immunostaining. Activity may also be measured using recombinant assays, such as a recombinant assay (see, for example, those provided by Abcam (Cambridge, UK)).

Humans have ˜41 E2 enzymes, and the amino acid sequences (and the nucleotide sequences of the cDNAs which encode them) are available by reference to GenBank or UniProt. The amino acid sequences and nucleotide sequences encoding various human E2 enzymes are also included in Tables 7-9 below. It will be appreciated that human E2s will be compatible with therapeutic use in human cells, and are unlikely to elicit immunogenic responses in humans.

Various classifications exist for E2 enzymes. For example, Michelle et al (J Mol Evol 2009, 68:616-628) have classified the enzymes into 17 families, Families 1-17, based on a phylogenetic analysis, and all such families are included in the scope of this disclosure. Thus, by E2 enzyme described herein, we include the meaning of any of an E2 enzyme selected from any one of a Family 1 E2 enzyme, a Family 2 E2 enzyme, a Family 3 E2 enzyme, a Family 4 E2 enzyme, a Family 5 E2 enzyme, a Family 6 E2 enzyme, a Family 7 E2 enzyme, a Family 8 E2 enzyme, a Family 9 E2 enzyme, a Family 10 E2 enzyme, a Family 11 E2 enzyme, a Family 12 E2 enzyme, a Family 13 E2 enzyme, a Family 14 E2 enzyme, a Family 15 E2 enzyme, a Family 16 E2 enzyme, and a Family 17 E2 enzyme. Hormaechea-Agulla et al (Mol Cell 2018, 41(3):168-178) have classified the enzymes into four classes, Classes I-IV. Class I contains only the UBC domain, Classes II and III have either N- or C-terminal extensions, respectively, and Class IV E2s have both N- and C-terminal extensions. Again, for the avoidance of doubt, all such classes of E2 are included in the scope of the disclosure, and so by E2 enzyme described herein, we include the meaning of a Class I E2, a Class II E2, a Class III E2 and a Class IV E2.

By “has an amino acid sequence having at least 80% sequence identity to a human E2 enzyme or a functional part thereof” we include the meaning that the E2 ubiquitin or ubiquitin-like conjugation domain must have an amino acid sequence that has at least 80% sequence identity to any human E2 enzyme or a functional part thereof (such as any of the human E2 enzymes listed in Tables 3-9) for example at least 81%, 82%, 83%, 84%, 85%, 86%, 87%, 88%, 89%, 90%, 91%, 92%, 93%, 94%, 95%, 96%, 97%, 98%, or 99% sequence identity to any human E2 enzyme or a functional part thereof (such as any of the human E2 enzymes listed in Tables 3-9).

By a “functional part”, we include the meaning of a portion of the human E2 enzyme that has the ubiquitin or ubiquitin-like conjugating capacity, for example as described above.

Typically, the functional part is at least 20 amino acids in length, such as at least 30, 40, 50, 60, 70, 80, 90, 100, 110, 120, 130, 140 or 150 amino acids in length. Preferably, the functional part is 50-150 or 80-150 amino acids in length such as 100-150 amino acids in length. For example, a functional part of a human E2 enzyme includes a portion of the human E2 enzyme which is capable of conjugating ubiquitin or ubiquitin-like protein to a target substrate, for example when the functional part is part of the molecule of the disclosure alongside the targeting domain that selectively targets the target substrate in question. As will become clearer below, the functional part is preferably the UBC domain, but it will be appreciated that it may even be a portion of the UBC domain provided that the portion is still capable of conjugating ubiquitin or ubiquitin-like protein to a target substrate.

In an embodiment, the E2 ubiquitin or ubiquitin-like conjugating domain is derived from an E2 enzyme or a functional part thereof or is synthetic.

All E2 enzymes have a core catalytic domain called the UBC domain. Thus, in one embodiment, the E2 ubiquitin or ubiquitin-like conjugating domain comprises a ubiquitin core catalytic (UBC) domain of a human E2 enzyme, or a variant of a UBC domain of a human E2 enzyme that still nevertheless has the ubiquitin or ubiquitin-like conjugating capacity, for example as described above. Thus, the UBC domain used in the present disclosure may be naturally occurring, for example derived from a human E2 enzyme, or it may be synthetic. Synthetic variants may be designed according to the consensus sequences within the UBC domain, as described in further detail below.

The amino acid sequences of the UBC domains of human E2 enzymes are provided in Table 8 below. It will be appreciated that the amino acid sequences of UBC domains of further E2 enzymes can also be identified by the skilled person, for example by searching for the sequence corresponding to one of the UBC domains in Table 8 using standard alignment techniques such as MacVector and Clustal W. The UBC domains are generally composed of four alpha helices and a four stranded beta-sheet.

The length of the UBC in human E2 enzymes ranges from 117 amino acids to 284 amino acids, and so in an embodiment, the UBC domain comprises 110-290 amino acids, such as 117-284 amino acids or 140-192 amino acids.

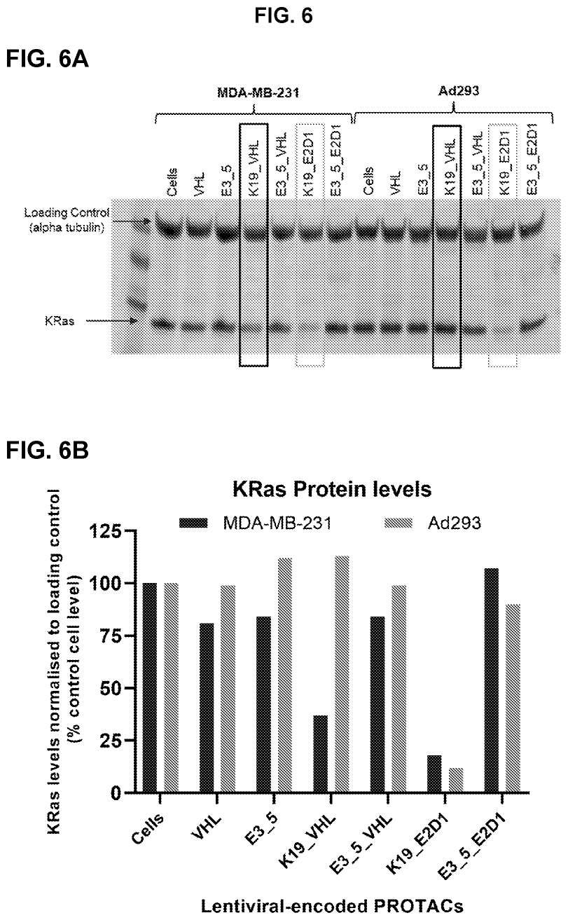

Comparison of UBC domains has identified key conserved regions or consensus sequences within it, for example as described by Michelle et al (“What was the set of ubiquitin and ubiquitin-like conjugating enzymes in the eukaryotic common ancestor?” J Mol Evol 2009, 68:616-628; see, in particular, FIG. 6 thereof). For instance, the general signature motif is a HxN tripeptide (e.g. HPN or histidine-proline-asparagine) and an active cysteine residue generally located at the eighth amino acid on the C-terminal side of this canonical motif. Also conserved is a PxxxP (SEQ ID NO: 206) motif and a tryptophan residue 26-43 amino acids from the C-terminal end of the PxxxP motif (SEQ ID NO: 206).

Accordingly, in one embodiment the E2 ubiquitin or ubiquitin-like conjugating domain comprises a UBC domain that contains a conserved catalytic cysteine residue. It will, however, be understood that the UBC domain does not necessarily require the catalytic cysteine residue. For example, UBE2V1 and UBE2V2 lack the conserved cysteine residue, but they nevertheless interact with Ube2N to allow lysine 63 (K63) polyubiquitin chain formation. In other words, it will be appreciated that the UBC domain may be one that becomes active in a cellular environment, for example through interaction with other E2 proteins. Nevertheless, it is preferred that the E2 ubiquitin or ubiquitin-like conjugating domain is one that is catalytic and has the conserved cysteine residue.

In another embodiment, the E2 ubiquitin or ubiquitin-like conjugating domain comprises a UBC domain that comprises a HxN peptide motif such as a HPN tripeptide. It will be appreciated, therefore, that the UBC domain may contain a HxN peptide motif (a HPN tripeptide) and a conserved cysteine residue generally located at the eighth amino acid on the C-terminal side of this canonical motif.

Using the characterisation of E2s into 17 families as described by Michelle et al (J Mol Evol 2009, 68:616-628), Family 5 (human E2s in this family are UBE2J1 and UBE2J2) missed the canonical tripeptide HxN, which was replaced by TPNGRF (SEQ ID NO: 208) or TANGRF (SEQ ID NO: 209). Thus, in a further embodiment, the UBC domain may contain a TxNGRF (SEQ ID NO: 210) peptide motif, for example TPNGRF (SEQ ID NO: 208) or TANGRF (SEQ ID NO: 209), in place of the HxN motif. Hence, the UBC may comprise a TxNGRF (SEQ ID NO: 210) peptide motif, for example TPNGRF (SEQ ID NO: 208) or TANGRF (SEQ ID NO: 209), and a conserved cysteine residue.

In a further embodiment, the E2 ubiquitin or ubiquitin-like conjugating domain comprises a UBC domain that comprises a PxxxP (SEQ ID NO: 206) peptide motif, such as a PxxPP (SEQ ID NO: 207) motif.

In a further embodiment, the E2 ubiquitin or ubiquitin-like conjugating domain comprises a UBC domain that comprises a conserved tryptophan residue. Preferably, this is between 26-43 amino acids from the C-terminal end of the PxxxP motif (SEQ ID NO: 206), such as a PxxPP (SEQ ID NO: 207) motif.

In yet a further embodiment, the E2 ubiquitin or ubiquitin-like conjugating domain comprises a UBC domain that comprises (i) a conserved cysteine residue; and/or (ii) a HxN peptide motif such as HPN, or a TxNGRF (SEQ ID NO: 210) peptide motif, for example TPNGRF (SEQ ID NO: 208) or TANGRF (SEQ ID NO: 209); and/or (iii) a PxxxP (SEQ ID NO: 206) peptide motif, such as a PxxPP (SEQ ID NO: 207) motif; and/or (iv) a conserved tryptophan residue.

In still a further embodiment, the E2 ubiquitin or ubiquitin-like conjugating domain comprises a UBC domain that comprises (i) a conserved cysteine residue; (ii) a HxN peptide motif such as HPN, or a TxNGRF (SEQ ID NO: 210) peptide motif, for example TPNGRF (SEQ ID NO: 208) or TANGRF (SEQ ID NO: 209); (iii) a PxxxP (SEQ ID NO: 206) peptide motif, such as a PxxPP (SEQ ID NO: 207) motif; and (iv) a conserved tryptophan residue, wherein the conserved tryptophan residue is 26-34 amino acids from the C-terminal end of the PxxxP motif (SEQ ID NO: 2206), and the conserved cysteine residue is within eight amino acids to the C-terminus of the HxN or TxNGRF motif.

In an embodiment, the ubiquitin or ubiquitin-like conjugating domain comprises a UBC domain that is a variant of a UBC of a human E2 enzyme, which variant shares at least 80% sequence identity with the UBC of a human E2 enzyme. For example, the variant may have an amino acid sequence of at least 80% sequence identity (such as at least 81%, 82%, 83%, 84%, 85%, 86%, 87%, 88%, 89%, 90%, 91%, 92%, 93%, 94%, 95%, 96%, 97%, 98%, or 99% sequence identity) to the UBC domain of any one of UBE2A (hHR6A), UBE2B (hHR6B), UBE2C (UbcH10), UBE2D1 (UbcH5A), UBE2D2 (UbcH5B), UBE2D3 (UbcH5C), UBE2D4 (HBUCE1), UBE2E1 (UbcH6), UBE2E2, UBE2E3 (UbcH9), UBE2F (NCE2), UBE2G1 (UBE2G), UBE2G2 (UBC7), UBE2H (UBCH), UBE2I (Ubc9), UBE2J1 (NCUBE1), UBE2J2 (NCUBE2), UBE2K (HIP2), UBE2L3 (UbcH7), UBE2L6 (UbcH8), UBE2M (Ubc12), UBE2N (Ubc13), UBE2NL, UBE20 (E2-230K), UBE2Q1 (NICE-5), UBE2Q2, UBE2QL, UBE2R1 (CDC34), UBE2R2 (CDC34B), UBE2S (E2-EPF), UBE2T (HSPC150), UBE2U, UBE2V1 (UEV-1A), UBE2V2 (MMS2), UBE2W, UBE2Z (Use1), UVELD (UEV3), BIRC6 (apollon), FTS (AKTIP), TSG101, and UFC1 (SEQ ID NOs: 42-82, respectively).

The percent sequence identity between two polypeptides may be determined using any suitable computer program, for example the GAP program of the University of Wisconsin Genetic Computing Group and it will be appreciated that percent identity is calculated in relation to polypeptides whose sequence has been aligned optimally. The alignment may alternatively be carried out using the Clustal W program (Thompson et al Nucleic Acids Res 1994, 22(22):4673-80). The parameters used may be as follows: Fast pairwise alignment parameters: K-tuple(word) size; 1, window size; 5, gap penalty; 3, number of top diagonals; 5. Scoring method: x percent. Multiple alignment parameters: gap open penalty; 10, gap extension penalty; 0.05. Scoring matrix: BLOSUM.

Typically, such variants sharing at least 80% sequence identity with the UBC of a human E2 enzyme would still retain the conserved sequences of the UBC domain as described above. Thus, the variant of a UBC of a human E2 enzyme preferably comprises (i) a conserved cysteine residue; and/or (ii) a HxN peptide motif such as HPN, or a TxNGRF (SEQ ID NO: 210) peptide motif, for example TPNGRF (SEQ ID NO: 208) or TANGRF (SEQ ID NO: 209); and/or (iii) a PxxxP (SEQ ID NO: 206) peptide motif, such as a PxxPP (SEQ ID NO: 207) motif; and/or (iv) a conserved tryptophan residue. Most preferably, the variant of a UBC of a human E2 enzyme comprises (i) a conserved cysteine residue; (ii) a HxN peptide motif such as HPN, or a TxNGRF (SEQ ID NO: 210) peptide motif, for example TPNGRF (SEQ ID NO: 208) or TANGRF (SEQ ID NO: 209); (iii) a PxxxP (SEQ ID NO: 206) peptide motif, such as a PxxPP (SEQ ID NO: 207) motif; and (iv) a conserved tryptophan residue, wherein the conserved tryptophan residue is 26-34 amino acids from the C-terminal end of the PxxxP motif (SEQ ID NO: 206), and the conserved cysteine residue is within eight amino acids to the C-terminus of the HxN or TxNGRF motif (SEQ ID NO: 210).

By variant we include variants of the UBC domain whose amino acid sequence comprises one or more deletions; and/or one or more amino acid substitutions; and/or one or more insertions compared to the amino acid sequence of the parent human E2 enzyme UBC.

The variants may be produced in any suitable way. Conventional site-directed mutagenesis may be employed, or polymerase chain reaction-based procedures well known in the art may be used.

Typically, it is preferred that the amino acid substitutions of the variants disclosed herein are conservative amino acid substitutions, for example where an amino acid residue is replaced with an amino acid residue having a similar side chain. Conservative amino acid substitutions are well known in the art and include (original residue ↔Substitution) Ala (A) ↔Val, Gly or Pro; Arg (R)↔Lys or His; Asn (N)↔Gln; Asp (D)↔Glu; Cys (C)↔Ser; Gln (Q)↔Asn; Glu (G)↔Asp; Gly (G)↔Ala; His (H)↔Arg; IIe (I)↔Leu; Leu (L)↔Ile, Val or Met; Lys (K)↔Arg; Met (M)↔Leu; Phe (F)↔Tyr; Pro (P)↔Ala; Ser (S)↔Thr or Cys; Thr (T)↔Ser; Trp (W)↔Tyr; Tyr (Y)↔Phe or Trp; and Val (V)↔Leu or Ala.

In a preferred embodiment, the ubiquitin or ubiquitin-like conjugating domain comprises the UBC domain of a human E2 enzyme such as any one of UBE2A (hHR6A), UBE2B (hHR6B), UBE2C (UbcH10), UBE2D1 (UbcH5A), UBE2D2 (UbcH5B), UBE2D3 (UbcH5C), UBE2D4 (HBUCE1), UBE2E1 (UbcH6), UBE2E2, UBE2E3 (UbcH9), UBE2F (NCE2), UBE2G1 (UBE2G), UBE2G2 (UBC7), UBE2H (UBCH), UBE2I (Ubc9), UBE2J1 (NCUBE1), UBE2J2 (NCUBE2), UBE2K (HIP2), UBE2L3 (UbcH7), UBE2L6 (UbcH8), UBE2M (Ubc12), UBE2N (Ubc13), UBE2NL, UBE20 (E2-230K), UBE2Q1 (NICE-5), UBE2Q2, UBE2QL, UBE2R1 (CDC34), UBE2R2 (CDC34B), UBE2S (E2-EPF), UBE2T (HSPC150), UBE2U, UBE2V1 (UEV-1A), UBE2V2 (MMS2), UBE2W, UBE2Z (Use1), UVELD (UEV3), BIRC6 (apollon), FTS (AKTIP), TSG101, and UFC1, the amino acid sequence of which UBC domains are specified in SEQ ID NOs: 42-82, respectively.

It will be understood that BIRC6 and UBE20 have been described in the art as E2/E3 hybrid enzymes as they are E3-independent E2 ubiquitin-conjugating enzymes (see Bartke et al Mol Cell 2004 and Ullah et al FEBS J2018). For the avoidance of doubt, such enzymes are included in the definition of E2 enzyme herein.

For the avoidance of doubt by “human E2” herein, we include the meaning of “derived from” human E2 such that the cDNA or gene expressing the enzyme was originally obtained using genetic material from human, but that the protein may be expressed in any host cell subsequently. Thus, it will be plain that a human E2 may be expressed in a prokaryotic host cell, such as E. coli, but would nevertheless be considered to be a human E2.

In a further embodiment, the regulation domain comprises an E2 enzyme, which in turn comprises the E2 ubiquitin or ubiquitin-like conjugating domain. Thus, it will be appreciated that the regulation domain may contain a full-length E2 enzyme, and not just the UBC domain thereof or another functional part thereof.

When the regulation domain comprises an E2 enzyme, the E2 enzyme is one that has an amino acid sequence having at least 80% sequence identity to any human E2 enzyme, such as one listed in Tables 3-8 below, for example an amino acid sequence with at least 81%, 82%, 83%, 84%, 85%, 86%, 87%, 88%, 89%, 90%, 91%, 92%, 93%, 94%, 95%, 96%, 97%, 98%, or 99% sequence identity to a human E2 enzyme. Preferably, the E2 enzyme has at least 80% sequence identity to a human E2 enzyme selected from the group consisting of UBE2A (hHR6A), UBE2B (hHR6B), UBE2C (UbcH10), UBE2D1 (UbcH5A), UBE2D2 (UbcH5B), UBE2D3 (UbcH5C), UBE2D4 (HBUCE1), UBE2E1 (UbcH6), UBE2E2, UBE2E3 (UbcH9), UBE2F (NCE2), UBE2G1 (UBE2G), UBE2G2 (UBC7), UBE2H (UBCH), UBE2I (Ubc9), UBE2J1 (NCUBE1), UBE2J2 (NCUBE2), UBE2K (HIP2), UBE2L3 (UbcH7), UBE2L6 (UbcH8), UBE2M (Ubc12), UBE2N (Ubc13), UBE2NL, UBE20 (E2-230K), UBE2Q1 (NICE-5), UBE2Q2, UBE2QL, UBE2R1 (CDC34), UBE2R2 (CDC34B), UBE2S (E2-EPF), UBE2T (HSPC150), UBE2U, UBE2V1 (UEV-1A), UBE2V2 (MMS2), UBE2W, UBE2Z (Use1), UVELD (UEV3), BIRC6 (apollon), FTS (AKTIP), TSG101, and UFC1, the amino acid sequences of which are provided in SEQ ID NOs: 1-41 respectively (see Table 7). Most preferably, the E2 enzyme is UBE2D1 (UbcH5A), UBE2E2, UBE2L3 (UbcH7), UBE20 (E2-230K), UBE2Q2, or UBE2R2.

Hence, it will be appreciated that the E2 enzyme may be a variant form of any of the human E2 enzymes described herein (see, for example Tables 3-9) having at least 80% sequence identity to any one of the human E2 enzymes, e.g. as provided in SEQ ID NOs: 1-41. By variant we include the meaning of the amino acid sequence of the human E2 enzyme containing one or more deletions; and/or one or more amino acid substitutions; and/or one or more insertions. Amino acid substitutions are preferred, and include conservative amino acid substitutions as mentioned above. Thus, it will be appreciated that the regulation domain may comprise a human E2 enzyme or a UBC domain of a human E2 enzyme (such as any of those described herein, for example in Tables 3-9) wherein up to 1, 2, 3, 4, 5, 6, 7, 8, 9, 10, 15 or 20, 25 or up to 30 amino acids are added, deleted and/or substituted (e.g. conservative substitutions) by other amino acids. Generally, the variations will be confined to outside the conserved regions described herein, including the cysteine residue important for catalytic activity, the HxN motif, the PxxxP motif (SEQ ID NO: 206) as being important for catalytic activity, such as the cysteine residue. Similarly, the variations will generally not interfere with an interaction between the E2 enzyme and one or more binding partners which interaction is involved in mediating the regulation function of the E2 enzyme. For example, as described in Example 6, a variant of the E2 enzyme, UBE2D1, containing the mutation F62A, completely abrogated regulation activity, which was thought to be due to the F62 residue being involved in the interaction between UBE2D1 and endogenous RING-type E3 ligases, such as RNF4. Hence, typically, the variant form of the E2 enzyme will still be able to interact with an E3 enzyme (e.g. the E3 protein that it naturally binds to in order to carry out the desired regulatory function). By “still be able to interact with an E3 enzyme”, we include the meaning that the variant form of the E2 enzyme demonstrates at least 50% of the binding to the E3 enzyme, such as 60%, 70%, 80% or 90% of the binding, and more preferably 95% or 99% of the binding to the E3 enzyme, as the level of binding between the E2 enzyme without the variation and the E3 enzyme. Methods for assessing protein-protein interactions are standard practice in the art, including for E2:E3 binding pairs (see, for example, Gundogdu and Walden, Protein Science. 2019; 28:1758-1770; Ning Zheng and Nitzan Shabek. Annual Rev Biochemistry, Vol. 86:129-157, 2017; and Turek et al., JBC 293, 16324-16336, 2018).

Also, to minimise auto-ubiquitination of the molecule of the disclosure, it may be desirable to modify human E2 enzymes or functional parts thereof (e.g. UBC domains), for example by modifying any one or more lysine residues within the human E2 enzymes or functional parts thereof. By ‘modification’ we include the meaning of one or more amino acid substitutions (e.g. conservative substitutions), and/or deletions and/or additions. This can be done using standard recombinant technology such as conventional site-directed mutagenesis or by use of PCR. Such modified human E2 enzymes may also be considered as variants. Such modifications (e.g. where one or more lysine residues are substituted for another amino acid) may also increase the stability of the resulting protein, for example when the modification is a stabilising modification, e.g. based on modelling predictions from protein crystal structures.

Additionally or alternatively, to increase the stability of the molecule of the disclosure, it may be desirable to modify human E2 enzymes or functional parts thereof (e.g. UBC domains), for example by modifying any one or more amino acid residues within the human E2 enzymes or functional parts thereof, which modifications are known to be stabilising, for example based on modelling predictions from protein crystal structures. By ‘modification’ we again include the meaning of one or more amino acid substitutions (e.g. conservative substitutions), and/or deletions and/or additions. Such modified human E2 enzymes may also be considered as variants.

It will be appreciated, therefore, that the regulation domain may be one that comprises a variant of one of the amino acid sequences of any one of SEQ ID NOs: 1-82 (i.e. any of the human E2 enzymes or UBC domains thereof in Tables 3-9) which contains up to 30 amino acid modifications, for example 1, or up to 2, 3, 4, 5, 6, 7, 8, 9, 10, 11, 12, 13, 14 or up to 15, 20, 25 or up to 30 amino acid modifications. The modifications may, for example, be ones that minimise auto-ubiquitination and/or increase stability.

In a preferred embodiment, the regulation domain comprises an E2 enzyme selected from the group consisting of UBE2A (hHR6A), UBE2B (hHR6B), UBE2C (UbcH10), UBE2D1 (UbcH5A), UBE2D2 (UbcH5B), UBE2D3 (UbcH5C), UBE2D4 (HBUCE1), UBE2E1 (UbcH6), UBE2E2, UBE2E3 (UbcH9), UBE2F (NCE2), UBE2G1 (UBE2G), UBE2G2 (UBC7), UBE2H (UBCH), UBE2I (Ubc9), UBE2J1 (NCUBE1), UBE2J2 (NCUBE2), UBE2K (HIP2), UBE2L3 (UbcH7), UBE2L6 (UbcH8), UBE2M (Ubc12), UBE2N (Ubc13), UBE2NL, UBE20 (E2-230K), UBE2Q1 (NICE-5), UBE2Q2, UBE2QL, UBE2R1 (CDC34), UBE2R2 (CDC34B), UBE2S (E2-EPF), UBE2T (HSPC150), UBE2U, UBE2V1 (UEV-1A), UBE2V2 (MMS2), UBE2W, UBE2Z (Use1), UVELD (UEV3), BIRC6 (apollon), FTS (AKTIP), TSG101, and UFC1, the amino acid sequences of which E2 enzymes are specified in SEQ ID NOs: 1-41, respectively.

It will be appreciated that any of the examples of possible regulation domains described above and listed in SEQ ID NOs: 1-82 may contain up to 5 amino acids at either or both termini (e.g. up to 2 amino acids) which may arise from the cloning strategy adopted to express them. However, it will be understood that these amino acids should not alter the function of the regulation domain. For example, the regulation domain von Hippel Lindau (VHL) protein in SEQ ID NO: 147 contains the two amino acids alanine and methionine at the N-terminus which derive from the cloning strategy, while in SEQ ID NO: 199, the VHL regulation domain is absent these two amino acids. In both cases, however, the regulation domains possess the relevant functionality.

By targeting domain we include the meaning of any domain or moiety that is capable of targeting to a target substrate. Preferably, the targeting domain is capable of targeting selectively to the substrate. For example, it is preferred if the targeting domain targets the substrate to a greater extent than any other substrate, and preferably only targets the substrate.

In one embodiment, the targeting domain binds to the substrate, and preferably binds to the substrate specifically. For example, it is preferred if the targeting domain binds to the substrate to a greater extent than any other substrate in a cell in which the molecule of the disclosure is intended to be used (e.g. the cell containing the substrate to be regulated). For example, it is preferred if the targeting domain has a Kd value (dissociation constant) which is at least five or ten times lower (i.e. higher affinity) than for at least one other substrate within the cell, and preferably more than 100 or 500 times lower. More preferably, the targeting domain of the substrate has a Kd value more than 1000 or 5000 times lower than for at least one other substrate within the cell. Kd values can be determined readily using methods well known in the art.

The targeting domain is typically a polypeptide (e.g. one that selectively binds to the substrate), such as any one of a monobody, nanobody, antibody, antibody fragment, scFv, intrabody, minibody, scaffold protein such as a designed ankyrin repeat protein (DARPin), peptide binder, or a ligand binding domain, preferably one that binds specifically (e.g. a ligand binding domain which can bind with high (e.g. nanomolar Kd or better) affinity to a target substrate).

As used herein, the term “antibody” includes but is not limited to polyclonal, monoclonal, chimeric, single chain, Fab fragments, fragments produced by a Fab expression library and bispecific antibodies. Such fragments include fragments of whole antibodies which retain their binding activity for a target substance, Fv, F(ab′) and F(ab′)2 fragments, as well as single chain antibodies (scFv), fusion proteins and other synthetic proteins which comprise the antigen-binding site of the antibody. A targeting domain comprising only part of an antibody may be advantageous by virtue of optimising the rate of clearance from the blood and may be less likely to undergo non-specific binding due to the Fc part. Also included are domain antibodies (dAbs), diabodies, camelid antibodies and engineered camelid antibodies. Furthermore, for administration to humans, the antibodies and fragments thereof may be humanised antibodies, which are now well known in the art (Janeway et al., 2001, Immunobiology., 5th ed., Garland Publishing; An et al., 2009, Therapeutic Monoclonal Antibodies: From Bench to Clinic, ISBN: 978-0-470-11791-0).

Also included are monobodies, nanobodies, intrabodies, unibodies, asymmetric IgG-like antibodies (eg triomab/quadroma, Trion Pharma/Fresenius Biotech; knobs-into-holes, Genentech; Cross MAbs, Roche; electrostatically matched antibodies, AMGEN; LUZ-Y, Genentech; strand exchange engineered domain (SEED) body, EMD Serono; biolonic, Merus; and Fab-exchanged antibodies, Genmab), symmetric IgG-like antibodies (eg dual targeting (DT)-Ig, GSK/Domantis; two-in-one antibody, Genentech; crosslinked MAbs, karmanos cancer center; mAb2, F-star; and Cov X-body, Cov X/Pfizer), IgG fusions (eg dual variable domain (DVD)-Ig, Abbott; IgG-like bispecific antibodies, Eli Lilly; Ts2Ab, Medimmune/AZ; BsAb, ZymoGenetics; HERCULES, Biogen Idec; TvAb, Roche) Fc fusions (eg ScFv/Fc fusions, Academic Institution; SCORPION, Emergent BioSolutions/Trubion, ZymoGenetics/BMS; dual affinity retargeting technology (Fc-DART), MacroGenics; dual (ScFv)2-Fab, National Research Center for Antibody Medicine) Fab fusions (eg F(ab)2, Medarex/AMGEN; dual-action or Bis-Fab, Genentech; Dock-and-Lock (DNL), ImmunoMedics; bivalent bispecific, Biotechnol; and Fab-Fv, UCB-Celltech), ScFv- and diabody-based antibodies (eg bispecific T cell engagers (BiTEs), Micromet; tandem diabodies (Tandab), Affimed; DARTs, MacroGenics; Single-chain diabody, Academic; TCR-like antibodies, AIT, Receptor Logics; human serum albumin ScFv fusion, Merrimack; and COMBODIES, Epigen Biotech), IgG/non-IgG fusions (eg immunocytokins, EMDSerono, Philogen, ImmunGene, ImmunoMedics; superantigen fusion protein, Active Biotech; and immune mobilising mTCR Against Cancer, ImmTAC) and oligoclonal antibodies (e.g. Symphogen and Merus).

The antibody may possess any of the antibody-like scaffolds described by Carter (“Potent antibody therapeutics by design”, Nat Rev Immunol 2006, 6(5):343-57, and Carter (“Introduction to current and future protein therapeutics: a protein engineering perspective”, Exp Cell Res 2011, 317(9):1261-9), incorporated herein by reference, together with the specificity determining regions described herein. Thus, the term “antibody” also includes affibodies and non-immunoglobulin-based frameworks. Examples include adnectins, anticalins, affilins, trans-bodies, DARPins, Tn3 molecules, trimerX, microproteins, fynomers, avimers, centgrins and kalbitor (ecallantide).

Suitable targeting domains for a given target substrate can be made by the skilled person using technology long-established in the art. For example, methods of preparation of monoclonal antibodies and antibody fragments are well known in the art and include hybridoma technology (Kohler & Milstein, “Continuous cultures of fused cells secreting antibody of predefined specificity” Nature 1975, 256:495-497); antibody phage display (Winter et al, “Making antibodies by phage display technology” Annu Rev Immunol 1994, 12:433-455); ribosome display (Schaffitzel et al., “Ribosome display: an in vitro method for selection and evolution of antibodies from libraries” J Immunol Methods 1999, 231:119-135); and iterative colony filter screening (Giovannoni et al., “Isolation of anti-angiogenesis antibodies from a large combinatorial repertoire by colony filter screening” Nucleic Acids Res 2001, 29:E27). Further, antibodies and antibody fragments suitable for use in the present disclosure are described, for example, in the following publications: “Monoclonal Hybridoma Antibodies: Techniques and Application”, Hurrell (CRC Press, 1982); “Monoclonal Antibodies: A Manual of Techniques”, H. Zola, CRC Press, 1987, ISBN: 0-84936-476-0; “Antibodies: A Laboratory Manual” 1st Edition, Harlow & Lane, Eds, Cold Spring Harbor Laboratory Press, New York, 1988. ISBN 0-87969-314-2; “Using Antibodies: A Laboratory Manual” 2nd Edition, Harlow & Lane, Eds, Cold Spring Harbor Laboratory Press, New York, 1999. ISBN 0-87969-543-9; and “Handbook of Therapeutic Antibodies” Stefan Dubel, Ed., 1st Edition, —Wiley-VCH, Weinheim, 2007. ISBN: 3-527-31453-9.

The targeting domains of the present disclosure can be monospecific, bispecific, trispecific or of greater multispecificity. Multispecific targeting domains can be specific for different epitopes of a substrate or can be specific for both a substrate polypeptide of the present disclosure as well as for heterologous compositions, such as a heterologous polypeptide or solid support material. It will be appreciated that such multispecific targeting domains may have value for targeting more complex multidomain substrates.

To minimise ubiquitination of the targeting domain of the molecule of the disclosure, it may be desirable to minimise the number of lysine residues in the targeting domain. Thus, the targeting domain may be modified by replacing lysine amino acids with, for example, arginine residues. Techniques for doing so are well known in the art.

Particular examples of suitable targeting domains have been exemplified in the Examples and include the monobody aCS3 which selectively binds to the C-SH2 domain of Src-homology 2 (SH2) domain-containing phosphatase 2 (SHP2), HuR8 and HuR17 which are nanobodies that bind to human antigen R, the DARPin K19 which binds to KRas protein, and Cas9 which selectively binds to the bacterial Cas9 protein utilised herein as an example of a negative control (as it is not expressed in mammals). The amino acid sequences of these targeting domains, as well as lysine variants of aCS3, are included in Table 10, and it will be appreciated that any such targeting domains may be used in the context of the disclosure. Thus, in one embodiment, the targeting domain has the amino acid sequence of any one of SEQ ID NOs: 126-135, 138-139, 257 or a variant thereof having up to 20 amino acid modifications, for example up to 1, 2, 3, 4, 5, 6, 7, 8, 9, or up to 10, 15, or 20 amino acid modifications. By ‘modification’ we include the meaning of one or more amino acid substitutions (e.g. conservative substitutions), and/or additions and/or deletions. In another embodiment, the targeting domain has the amino acid sequence of any one of SEQ ID NOs: 126-135, 138-139, 257 or a variant thereof having at least 80% sequence identity to any one of SEQ ID NOs: 126-135, 138-139, 257 for example at least 85% or 90% sequence identity, for example at least 91%, 92%, 93%, 94%, 95%, 96%, 97%, 98%, or 99% sequence identity to any one of SEQ ID NOs: 126-135, 138-139, 257. It will be appreciated that the variants of the targeting domain may be ones which have been modified to minimise ubiquitination of the targeting domain, for example by modifying one or more lysine residues (e.g. by substituting one or more of them to another amino acid residue and/or deleting one or more of them) and/or to increase the stability of the targeting domain, for example by making one or more modifications known to increase stability, for example based on modelling predictions from protein crystal structures.

In some embodiments, the present disclosure provides a molecule wherein: the targeting domain is a variant of the amino acid sequence of any one of SEQ ID NOs: 126-135, 138-139, 257 in which one or more of the lysine residues has been substituted with another amino acid and/or deleted; and/or the regulation domain is a variant of the amino acid sequence of any one of SEQ ID NOs: 42-82 in which one or more lysine residues has been substituted with another amino acid and/or deleted.

By substrate (or target substrate) we include the meaning of any substrate that can be targeted by the molecule of the disclosure, and thereby become conjugated to ubiquitin or a ubiquitin-like protein and thereby be regulated (e.g. degraded).

Preferably, the target substrate is a polypeptide, and is typically an intracellular polypeptide by which we include the meaning of any polypeptide with at least one portion being within the cell. Thus, the substrate may be an intracellular polypeptide that resides in the cytosol and/or an organelle within the cell, or it may be a membrane polypeptide, such as a transmembrane polypeptide (e.g. GPCR) that has at least an intracellular portion. However, ubiquitin is found both in intra- and extracellular fluids and is involved in regulation of numerous cellular processes. Extracellular ubiquitin has been linked to the modulation of immune response (Sujashvili, “Advantages of extracellular ubiquitin in modulation of immune responses,” Mediators Inflamm 2016, Epub 2016:4190390); and Baska et al suggest that constituents of ubiquitin-proteasome pathway are secreted in the mammalian epididymal fluid (EF) (Baska et al., “Mechanism of extracellular ubiquitination in the mammalian epididymis,” J Cell Physiol 2008, 215(3):684-96). Thus, it will be appreciated that in particular contexts the molecule of the disclosure can be used to regulate extracellular target substrates. Preferably, however, the substrate is an intracellular polypeptide.

In an embodiment, the substrate is localised in one or more of the plasma membrane, cytoplasm, nucleus, mitochondria, endosome, endoplasmic reticulum, mitochondria and Golgi apparatus.

Examples of possible target substrates include an oncogenic protein, a signalling protein, a GPCR, a post-translationally modified protein, an adhesion protein, a receptor, a cell-cycle protein, a checkpoint protein, a viral protein, a prion protein, a bacterial protein, a parasitic protein, a fungal protein, a DNA binding protein, a structural protein, an enzyme, an immunogen, an antigen, and a pathogenic protein. It will be appreciated that the target substrate may be any potential therapeutic target, whether conventionally druggable or presently undruggable.

In a particular embodiment, the substrate is selected from the group consisting of Ras, KRas and SHP2. Other possible target substrates include human rhino virus (HRV) protease 3C, muscarinic acetylcholine receptor 2 (M2R), Beta-2 adrenergic receptor (β2-AR), Crossover junction endonuclease MUS81 (MUS81) and Human antigen R (HuR).

In some embodiments, the regulation domain and the targeting domain are joined by a linker. By linker, we include the meaning of a chemical moiety that attaches the regulation domain to the targeting domain. It is preferred if the regulation domain is covalently bound to the targeting domain, for example by a linker.

Thus, the regulation domain and targeting domain may be linked by any of the conventional ways of cross-linking molecules, such as those generally described in O'Sullivan et al (“Comparison of two methods of preparing enzyme-antibody conjugates: Application of these conjugates for enzyme immunoassay,” Anal Biochem 1979, 100:100-8). For example, one of the regulation domain or targeting domain may be enriched with thiol groups and the other reacted with a bifunctional agent capable of reacting with those thiol groups, for example the N-hydroxysuccinimide ester of iodoacetic acid (NHIA) or N-succinimidyl-3-(2-pyridyldithio)propionate (SPDP), a heterobifunctional cross-linking agent which incorporates a disulphide bridge between the conjugated species. Amide and thioether bonds, for example achieved with m-maleimidobenzoyl-N-hydroxysuccinimide ester, are generally more stable in vivo than disulphide bonds. It is known that bis-maleimide reagents allow the attachment of a thiol group (e.g. thiol group of a cysteine residue of an antibody) to another thiol-containing moiety (e.g. thiol group of a T cell antigen or a linker intermediate), in a sequential or concurrent fashion. Other functional groups besides maleimide, which are reactive with a thiol group include iodoacetamide, bromoacetamide, vinyl pyridine, disulfide, pyridyl disulfide, isocyanate, and isothiocyanate.

In a particularly preferred embodiment, the regulation domain and the targeting domain are polypeptides and the regulation domain is attached to the targeting domain, either directly without a linker, or indirectly through a linker.

Thus, it will be appreciated that the regulation domain and targeting domain may be constituent parts of a fusion polypeptide that may be encoded by a nucleic acid molecule. Hence, in a particularly preferred embodiment, the molecule of the disclosure is a fusion polypeptide comprising (a) a regulation domain comprising an E2 ubiquitin or ubiquitin-like conjugating domain which has an amino acid sequence having at least 80% sequence identity to a human E2 enzyme or a functional part thereof, and (b) a targeting domain capable of targeting the regulation domain to a substrate. The regulation domain may be at the N-terminus of the targeting domain, or the targeting domain may be at the N-terminus of the regulation domain. By fusion polypeptide, we include the meaning of a protein or polypeptide that has an amino acid sequence derived from two or more proteins, for example two heterologous domains as indicated above, namely the regulation domain and the targeting domain. The fusion protein may also include linking regions of amino acids between amino acid portions derived from separate proteins.

Suitably, the regulation domain and the targeting domain are joined so that both domains retain their respective activities such that the molecule can be targeted to a target substrate, and the substrate can be consequentially regulated. It may, therefore, be desirable for the fusion polypeptide to contain a peptide linker between the regulation domain and the targeting domain, for example so as to prevent steric disruption between the targeting substrate and the target substrate. Suitable linker peptides are those that typically adopt a random coil conformation, and so the linkers may comprise glycine, serine or a mixture of glycine plus serine residues. Other amino acids that the linkers may contain include any one or more of leucine, glutamate, arginine, proline, alanine, asparagine, tyrosine, aspartate, valine and threonine. Preferably, the linker contains between from 1 and 45 amino acid residues, such as between from 5 and 28 amino acid residues in length, more preferably from 1 and 20 amino acid residues, or from 4 and 20 amino acid residues, such as from 5 and 19 amino acid residues in length. Most preferably, the linker contains between from 6 and 20 amino acid residues, such as between from 9 and 19 amino acid residues in length. Particular lengths of linker include lengths of 5, 6, 7, 8, 9, 10, 11, 12, 13, 14, 15, 16, 17, 18, 19, 20, 21, 22, 23, 24, 25, 26, 27 or 28 amino acid residues.

Examples of particularly preferred linkers that may be used are listed in Table 10, which includes the peptide GGGGS (SEQ ID NO: 146) or GGGGSGGGGSGGGGS (SEQ ID NO: 145) or LEGGGGSSR (SEQ ID NO: 141) or LEGGGGSGGGGSGGGGSSR (SEQ ID NO: 142) AAAGGGGSGGGGSGGGGSGT (SEQ ID NO: 143) or GGGGG (SEQ ID NO: 144) or LEGGSR (SEQ ID NO: 211) or LEGGGSGGSSR (SEQ ID NO: 212) or LEGGGGSGGGSSR (SEQ ID NO: 213) or LEGGGSGGGSGGGSSR (SEQ ID NO: 214) or LEGGGGSGPSGGGGPSGSR (SEQ ID NO: 215) or LESNGGGGSPAPAPGGGGSGSSR (SEQ ID NO: 216) or LEGGGGSYPYDVPDYASGGGGSSR (SEQ ID NO: 217) or TGGSAGGSGGSAGGSGGSAGGSGGSA (SEQ ID NO: 218) or AGSGGSTGSGGSPTPSTSGGSTGSGGAS (SEQ ID NO: 219) or AGSGGSGGSGGSGNSSTSGGSGGSGGAS (SEQ ID NO: 220) or GGSPVPSTPGGGSGGGSGGSPVPSTPGS (SEQ ID NO: 221), or SPGTGSPGTGSPGTGSPGTGSPGTGSPG (SEQ ID NO: 222). It will be appreciated that “LE” and “SR” in these examples are present due to restriction cloning sites introduced into the nucleotide sequences. It will further be appreciated that the one or more serine residues in any of these example linkers may be substituted for a glycine residue.

Polynucleotides which encode suitable targeting domains are known in the art or can be readily designed from known sequences such as from sequences of proteins known to interact with target substrates or contained in nucleotide sequence databases such as the GenBank, EMBL and dbEST databases. Polynucleotides which encode suitable regulation domains are known in the art or else can readily be designed from known E2 enzyme sequences and made.

Polynucleotides which encode suitable linker peptides can readily be designed from linker peptide sequences and made.

Thus, polynucleotides which encode the fusion polypeptides of the disclosure can readily be constructed using well known genetic engineering techniques.

The nucleic acid is then expressed in a suitable host to produce a molecule of the disclosure, e.g. fusion polypeptide. Thus, the nucleic acid encoding the fusion polypeptide of the disclosure may be used in accordance with known techniques, appropriately modified in view of the teachings contained herein, to construct an expression vector, which is then used to transform an appropriate host cell for the expression and production of the fusion polypeptide of the disclosure.

It is appreciated that the nucleic acid encoding the polypeptide of the disclosure may be joined to a wide variety of other nucleic acid sequences for introduction into an appropriate host. The companion nucleic acid will depend upon the nature of the host, the manner of the introduction of the nucleic acid into the host, and whether episomal maintenance or integration is desired, as is well known in the art.

As mentioned above and demonstrated in the Examples, the inventors have found that it is possible to provide for targeted regulation of target substrates by molecules comprising a regulation domain that contains an E2 ubiquitin or ubiquitin-like conjugating domain, and not an E3 ligase. Hence, in one embodiment, the molecule or fusion polypeptide of the disclosure does not comprise an E3 ubiquitin or a ubiquitin-like ligase or a functional part thereof. By a functional part of a E3 ubiquitin or a ubiquitin-like ligase, we include a part of a E3 ubiquitin or ubiquitin-like ligase that is still capable of assisting in the transfer of ubiquitin or a ubiquitin-like protein to a substrate, for example either directly (as with HECT E3 ubiquitin ligases) or indirectly (as with RING E3 ubiquitin ligases). Assaying E3 ubiquitin or ubiquitin-like ligase activity can be done using any suitable technique known in the art, and may involve testing whether the E3 ubiquitin or ubiquitin-like ligase is capable of binding to the substrate and to E2-Ub or E2-Ubl (see, for example, the ternary complex formation assays described by Richting et al. (“Quantitative live-cell kinetic 15 degradation and mechanistic profiling of PROTAC mode of action,” ACS Chem Biol 2018, 13(9):2758-70)).. By “does not comprise an E3 ubiquitin or ubiquitin-like ligase” we include the meaning that the molecule or polypeptide of the disclosure is not covalently attached to a E3 ubiquitin or ubiquitin-like ligase. For example, when the molecule of the disclosure is a fusion polypeptide, the nucleotide sequence encoding that fusion polypeptide does not also encode a E3 ubiquitin or ubiquitin-like ligase.

In an embodiment, the molecule of the disclosure (e.g. polypeptide of the disclosure), does not comprise a E3 ubiquitin or ubiquitin-like ligase or functional part thereof, which E3 ubiquitin or ubiquitin-like ligase or functional part thereof is one that comprises one or more domains selected from the group consisting of a RING (Really Interesting New Gene) domain, a U-box domain, a HECT (homologous to E6-AP carboxyl terminus) domain, and an RBR domain. In an embodiment, the molecule of the disclosure comprises a subcellular localisation signal, such as a nuclear localisation signal, a mitochondrial localisation signal or an endosomal localisation signal.

Examples of fusion polypeptides of the disclosure include those listed in Table 12A, and so in a preferred embodiment, the molecule of the disclosure is any one of the fusion polypeptides listed in Table 12A, having respective SEQ ID NOs: 156-167, 170-195, 202-205, 236-248, 253-256 and 266-275, more preferably wherein the molecules of the disclosure have the amino acid sequence of any one of SEQ ID NOs: 156-167, 171-195, 202-204, 236-248, 253-256, 267, 270 and 272. Also included are variants of the polypeptides of SEQ ID NOs: 156-167, 170-195, 202-205, 236-248, 253-256 and 266-275, preferably variants of the polypeptides of any one of SEQ ID NOs: 156-167, 171-195, 202-204, 236-248, 253-256, 267, 270 and 272, for example variants having up to 50 amino acid modifications (e.g. amino acid substitutions (preferably conservative substitutions) and/or additions and/or deletions, such as up to 45, 40, 35, 30, 25, or 20 modifications, for example up to 19, 18, 17, 16, 15, 14, 13, 12, 11, 10, 9, 8, 7, 6, 5, 4, 3, 2 or 1 amino acid modification(s), or variants having at least 50% sequence identity to any one of the fusion polypeptides listed in Table 12A having respectively SEQ ID NOs: 156-167, 170-195, 202-205, 236-248, 253-256 and 266-275, preferably SEQ ID NOs: 156-167, 171-195, 202-204, 236-248, 253-256, 267, 270 and 272, for example at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity. It will be appreciated that the variant should be one in which the regulation domain and the targeting domain are still able to perform their respective functions, such that the molecule can be targeted to a target substrate, and the substrate can be consequentially regulated. It will also be appreciated that the E2 ubiquitin or ubiquitin-like conjugating domain of the regulation domain within the variant must still have at least 80% sequence identity to a human E2 enzyme or a functional part thereof. It is yet further appreciated that the variants of the fusion polypeptides listed in Table 12A may be ones in which the targeting domain (e.g. aCS3 or K19 or variants thereof) are substituted for another targeting domain, such as aCS3 or K19 or a variant thereof as the case may be, and/or the regulation domain is substituted for another regulation domain.

In some embodiments, the molecule of the disclosure includes a detectable marker, for example to allow the identification or selection of cells that contain the molecule of the disclosure. By a “detectable marker” we include the meaning of a marker which, when within the molecule of the disclosure, may be detected either directly or indirectly, so that the presence of the molecule can be likewise detected. For example, it may be desirable to include a detectable marker in the fusion polypeptide of the disclosure in order to determine if the fusion polypeptide is being expressed. Accordingly, in an embodiment, the molecule of the disclosure further comprises a detectable marker. Examples of detectable markers include affinity tags, such as a hemagglutinin A epitope tag (YPYDVPDYA; SEQ ID NO: 124), the Glu-Glu tag (CEEEEYMPME; SEQ ID NO: 125), and the FLAG tag (which binds with anti-FLAG antibodies). Other examples of markers that may be used are radiolabels, fluorescent labels, enzymatic labels or other amino-acid based labels. Any suitable marker may be used, although to minimise self-ubiquitination which may result in proteolytic degradation of the molecule of the disclosure, markers that do not contain lysine residues are preferred. Nucleic acid molecules encoding such peptide markers are available, for example, from Sigma-Aldrich Corporation (St. Louis, Mo., USA). It will be appreciated that the detectable marker may be present at the N-terminus of the fusion polypeptide or at the C-terminus of the fusion polypeptide, or if a peptide linker is present between the regulation domain and the targeting domain, the detectable marker may be present within the linker of the fusion polypeptide. Examples of constructs with a detectable marker in various positions can be found in Table 12A.

In a further embodiment, the molecule of the disclosure may comprise an additional localisation moiety that may be used to direct the molecule of the disclosure to a particular subcellular location. By localisation moiety we include the meaning of a moiety that targets the molecule of the disclosure to a particular subcellular location and thereby increases the concentration the molecule of the disclosure at that subcellular location, as compared to the concentration of the molecule of the disclosure at that subcellular location in the absence of the localisation moiety. The subcellular location may be one in which the target substrate predominantly resides. For example, if the target substrate resides predominantly in the nucleus, it may be desirable to include a localisation moiety that directs the molecule to the nucleus. Equally, it will be appreciated that the molecule of the disclosure may be used to regulate (e.g. degrade) target substrates selectively in particular subcellular locations. For example, the molecule may be used to regulate (e.g. degrade) target substrates that reside in the mitochondria but not those same substrates that reside in the nucleus.

Means of assessing subcellular localisation are well known to those skilled in the art. For example, this could be tested by immunofluorescent staining and high content imaging. The target protein could be stained for using a specific antibody and the presence of the molecule of the disclosure by staining with an anti-tag antibody. By using fluorescently tagged secondary antibodies, this may be detected by high-content confocal imaging. Additionally, the cell nucleus, mitochondria or other organelles can be stained with specific dyes. Various localisation motifs are well known to the skilled person, including nuclear localisation sequences (NLS) which localise to the nucleus (Lange et al., J Biol Chem 2007, 282(8):5101-05), and CAAX motifs or palmitoylation sites to localise to the plasma membrane (Michaelson et al., Mol Biol Cell 2005, 16:1606-16; Guan and Fierke, Sci China Chem 2011, 54(12):1888-97; Aicart-Ramos et al, Biochim Biophys Acta—Biomembranes 2011, 1808(12):298194). Any such localisation motif may be included in the molecule of the disclosure.

While the fusion polypeptides listed in Table 12A are shown in a particular orientation (e.g. ‘regulation domain—linker—targeting domain’ from N-terminus to C-terminus), for the avoidance of doubt the reverse orientation is also included in the scope of the disclosure. For example, the fusion polypeptide of SEQ ID NO: 193 (HA_UFC1 Linker 2 aCS3) is in the orientation ‘regulation domain—linker—targeting domain’, however, it will be understood that the reverse orientation ‘targeting domain—linker—regulation domain’ is also included in the scope of the disclosure.

Thus, it will be appreciated that the disclosure provides a fusion polypeptide comprising a regulation domain, a targeting domain, optionally a peptide linker between the regulation domain and targeting domain, and optionally a detectable marker and/or a localisation domain. For example, the disclosure includes a fusion polypeptide comprising a regulation domain, a targeting domain, a peptide linker between the regulation domain and targeting domain, and optionally a detectable marker and/or a localisation domain.

In a preferred embodiment, the first aspect of the disclosure includes a fusion polypeptide comprising an E2 enzyme having an amino acid sequence with at least 80% sequence identity to a human E2 enzyme (e.g. as listed in any of Tables 3-9 below) and a targeting domain (e.g. a monobody or nanobody) capable of targeting the E2 enzyme to a substrate.

In a preferred embodiment, the first aspect of the disclosure includes a fusion polypeptide comprising a E2 ubiquitin or ubiquitin-like conjugation domain having an amino acid sequence with at least 80% sequence identity to a functional part of a human E2 enzyme (e.g. as listed in any of Tables 3-9 below) and a targeting domain (e.g. a monobody or nanobody) capable of targeting the E2 enzyme to a substrate. Preferably, the functional part is the UBC domain.

A second aspect of the disclosure provides a compound comprising (i) a molecule according to the first aspect of the disclosure, and (ii) a targeting moiety capable of targeting the molecule to a cell.

Preferences for the molecule according to the first aspect of the disclosure include those described above. For example, the compound may comprise a fusion polypeptide of the first aspect of the disclosure and a targeting moiety capable of targeting the molecule to the cell.

It will be appreciated that the cell is one which contains the target substrate that the molecule or polypeptide of the disclosure is capable of regulating. Thus, the cell may contain a substrate which is desirable to degrade, and the molecule of the disclosure comprises a regulation domain that is a degradation domain.

By targeting moiety, we include the meaning of any moiety that is capable of targeting to a cell that contains a substrate which is desirable to regulate (e.g. degrade). By a cell that contains a substrate which is desirable to regulate (e.g. degrade), we include the meaning of all cells that contain that substrate, or a subset of cells that contain the substrate where it is desirable to regulate (e.g. degrade) the substrate only in that subset of cells. Preferably, the targeting domain is capable of targeting selectively to the cell that contains the substrate which is desirable to regulate (e.g. degrade). For example, it is preferred if the targeting moiety targets the cell to a great extent than it does any other type of cell, and most preferably only targets the cell that contains the substrate which is desirable to regulate (e.g. degrade).

In one embodiment, the targeting moiety is a specific binding partner of an entity expressed by or associated with the cell that contains the substrate which is desirable to regulate (e.g. degrade). Typically, the expressed entity is expressed selectively on the cell. For example, the abundance of the expressed entity is typically 10 or 100 or 500 or 1000 or 5000 or 10000 higher on the cell that contains the substrate which is desirable to regulate (e.g. degrade) than on other cells, for example within the individual to be treated.

By “binding partner” we include the meaning of a molecule that binds to an entity expressed by a particular cell. Preferably, the binding partner binds selectively to that entity. For example, it is preferred if the binding partner has a Kd value (dissociation constant) which is at least five or ten times lower (i.e. higher affinity) than for at least one other entity expressed by another cell (e.g. cell not containing a substrate that is desirable to regulate (e.g. degrade) or a cell that contains the substrate but wherein it is not desirable to regulate (e.g. degrade) it in that cell), and preferably more than 100 or 500 times lower. More preferably, the binding partner of that entity has a Kd value more than 1000 or 5000 times lower than for at least one other entity expressed by another cell (e.g. cell not containing a substrate that is desirable to regulate (e.g. degrade) or a cell that contains the substrate but wherein it is not desirable to regulate (e.g. degrade) it in that cell).

Typically, the binding partner is one that binds to an entity that is present or accessible to the binding partner in significantly greater concentrations in or on a cell that contains the substrate which is desirable to regulate (e.g. degrade) than in any other cells of the host. Thus, the binding partner may bind to a surface molecule or antigen on the cell that contains the substrate which is desirable to regulate (e.g. degrade), that is expressed in considerably higher amounts than on other cells. Similarly, the binding partner may bind to an entity that has been secreted into the extracellular fluid by the cells that contain the substrate which is desirable to regulate (e.g. degrade) to a greater extent than by other cells. For example, if the target substrate resides in a cancer cell, the binding partner may bind to a tumour associated antigen which is expressed on the cell membrane or which has been secreted into tumour extracellular fluid.

In a preferred embodiment, the binding partner is one that binds to an entity that is present or accessible to the cell that contains the substrate which is desirable to regulate (e.g. degrade). Preferably, the entity is one which when bound by the binding partner is one that leads to the internalisation of the binding partner (and any associated molecule, e.g. the compound of the second aspect of the disclosure) into the cell. It will be appreciated that when intracellular delivery relies on the endocytic pathway as the main uptake mechanism, it may be desirable to include within the compound (e.g. the molecule of the first aspect of the disclosure) a means to escape the endosome or lysosome, or any other vesicle it may be contained within, or otherwise engage another means (i.e. external to the compound) to mediate such escape. Methods for enhancing endosomal escape are well known to the skilled person and include those reviewed in Hum Gen Ther 2011, 22(10):A14-A14 and in Lönn et al (Sci Rep 2016, 6:32301). For example, the compound of the second aspect of the disclosure (and/or the molecule of the first aspect of the disclosure) may contain an endosomal escape domain.

The targeting moiety may be any of a polypeptide, a peptide, a small molecule or a peptidomimetic. Typically, the targeting moiety is a polypeptide such as any one of a monobody, nanobody, antibody, antibody fragment, scFv, intrabody, minibody, novel scaffold, peptide binder, or ligand binding domain.

In a preferred embodiment, the targeting moiety is a binding partner such as an antibody. The antibody may be one that binds to an antigen expressed by the cell that contains the substrate which is desirable to regulate (e.g. degrade), for example an antigen expressed on the surface of the cell. Preferably, the antigen is one which when bound by the antibody leads to the internalisation of the compound of the second aspect of the disclosure into the cell, for example by receptor-mediated endocytosis.

Wherein the molecule of the disclosure and the targeting moiety are polypeptides, it will be appreciated that the compound of the second aspect of the disclosure may also constitute a fusion polypeptide comprising a regulation domain, a targeting domain and a targeting moiety. Thus, the targeting moiety may be a polypeptide that itself is fused to the fusion polypeptide comprising the targeting domain and regulation domain.

It is appreciated that a person skilled in the art can readily select suitable binding partners for any given cell, for example by identifying surface antigens or molecules specific for that cell and finding a binding partner for that antigen or molecule. Considerable research has already been carried out on antibodies and fragments thereof to tumour-associated antigens, immune cell antigens and infectious agents. Thus, in some embodiments, selecting an appropriate targeting moiety for a given cell type typically involves searching the literature, guided by, for example, the reviews of Muro (“Challenges in design and characterisation of ligand-targeted drug delivery systems,” J Control Release 2012, 164(2):125-37) and Carter et al. (“Identification and validation of cell surface antigens for antibody targeting in oncology,” Endocr-relat Cancer 2004, 11:659-87). Alternatively, a cell may be taken from a patient (e.g. by biopsy), and antibodies directed against the cell prepared. Such ‘tailor-made’ antibodies are already known. It has been demonstrated that antibodies confer binding to tumour cells not only from the patient they have been obtained from but also for a large number of other patients. Thus, a plurality of such antibodies has become commercially available. Other methods of identifying suitable binding partners for a given unwanted cell include genetic approaches (e.g. microarray), proteomic approaches (e.g. differential Mass spectrometry), immunological approaches (e.g. immunising animals with tumour cells and identifying antibody-secreting clones which specifically target malignant cells), phage display selections using antibody libraries on the diseased cell itself (phenotypic screening; see Rust et al., Mol Cancer 2013, 12:11, Sandercock et al., Mol Cancer 2015, 14:147 and Williams et al., Oncotarget 2016, 7(42)68278-91) and in silico approaches wherein targets are identified using a systems biology approach.

It will be understood that the targeting domain is one that typically functions inside a cell to direct the regulation domain to a target substrate (e.g. intracellular polypeptide), whereas the targeting moiety typically functions outside of the cell to target the regulation domain and targeting domain to that cell.

Antibody-drug conjugates, such as for cancer therapy are reviewed by Carter & Senter (Cancer J 2008, 14(3):154-69), and Chari et al (Angewandte Chemie International Edition 2014, 53:3751), and it will be appreciated that the compounds of this aspect of the disclosure may considered such antibody drug conjugates (see also U.S. Pat. Nos. 5,773,001; 5,767,285; 5,739,116; 5,693,762; 5,585,089; US 2006/0088522; US 2011/0008840; U.S. Pat. No. 7,659,241; Hughes 2010 Nat Drug Discov 9: 665, Lash 2010; In vivo: The Business & Medicine Report 32-38; Mahato et al 2011, Adv Drug Deliv Rev 63:659; Jeffrey et al. 2006, BMCL 16: 358; Drugs R D 11(1): 85-95). ADCs generally comprise a monoclonal antibody against a target present on a tumour cell, a cytotoxic drug, and a linker that attaches the antibody to the drug. Thus, the compound of the second aspect of the disclosure may be an ADC comprising a targeting moiety that is an antibody, a regulation domain and a targeting domain. Preferences for the regulation domain and targeting domain include those described above in relation to the first aspect of the disclosure.

The targeting moiety may be attached to the molecule of the first aspect of the disclosure in known ways. For example, if the targeting moiety is a polypeptide such as an antibody, and the molecule of the first aspect of the disclosure is a fusion polypeptide, the targeting moiety, regulation domain and targeting domain can be expressed as a fusion polypeptide, as is well known in the art and as described above. Alternatively, the targeting moiety may be attached to the molecule of the first aspect of the disclosure by any other known means, either covalently or non-covalently.

In some embodiments, the targeting moiety is joined to the molecule of the disclosure by a linker. By linker we include the meaning of a chemical moiety that attaches the targeting moiety to the molecule of the first aspect of the disclosure. The attachment may be covalent or non-covalent. Preferably, it is covalent. Thus, the targeting moiety and molecule of the first aspect of the disclosure may be linked by any of the conventional ways of cross-linking molecules, for example as described above in relation to the first aspect of the disclosure. It will be understood that a large number of homobifunctional and heterobifunctional crosslinking chemistries would be appropriate to join the targeting moiety to the T cell antigen, and any such chemistry may be used.

In some embodiments, the molecule of the first aspect of the disclosure and the compound of the second aspect of the disclosure is encoded by a suitable nucleic acid molecule and expressed in a suitable host cell. Accordingly, a third aspect of the disclosure provides a polynucleotide encoding a molecule of the first aspect of the disclosure or the compound of the second aspect of the disclosure. Thus, when the molecule of the first aspect of the disclosure or the compound of second aspect of the disclosure are fusion polypeptides, it will be appreciated that the disclosure includes polynucleotides encoding such fusion polypeptides. Preferences for the molecule of the first aspect of the disclosure and the compound of the second aspect of the disclosure include those described above in relation to their respective aspects of the disclosure.

The polynucleotide may be DNA or it may be RNA. It may comprise deoxyribonucleotides, ribonucleotides, modified nucleotides or bases, and/or their analogues, or any substrate that can be incorporated into a polymer by DNA or RNA polymerase, or by a synthetic reaction. A polynucleotide may comprise modified nucleotides, such as methylated nucleotides and their analogues. If present, modification to the nucleotide structure may be imparted before or after assembly of the polymer. The sequence of nucleotides may be interrupted by non-nucleotide components.

Suitable nucleic acid molecules encoding the molecule of the first aspect of the disclosure or the compound of the second aspect of the disclosure may be made using standard cloning techniques, site-directed mutagenesis and PCR as is well known in the art. Molecular biological methods for cloning and engineering genes and cDNAs, for mutating DNA, and for expressing polypeptides from polynucleotides in host cells are well known in the art, as exemplified in “Molecular cloning, a laboratory manual”, third edition, Sambrook, J. & Russell, D. W. (eds), Cold Spring Harbor Laboratory Press, Cold Spring Harbor, NY, incorporated herein by reference. Examples of suitable polynucleotides include those in Table 12C below, which have been assigned SEQ ID NOs: 223-235, 249-252 and 258-265, preferably wherein the polynucleotide is any one of SEQ ID NOs: 223-235, 249-252, 259, 262 and 264.

A fourth aspect of the disclosure provides a vector comprising the polynucleotide of the third aspect of the disclosure. The vector can be of any type, for example a recombinant vector such as an expression vector. The expression vectors contain elements (e.g., promoter, signals of initiation and termination of translation, as well as appropriate regions of regulation of transcription) which allow the expression and/or the secretion of the polypeptides in a host cell. Suitable expression systems include constitutive or inducible expression systems. Particularly, the vector may be a viral vector, e.g. a lentivirus or adenovirus or retrovirus. Most particularly, the vector may be a lentivirus or adeno-associated virus (AAV) vector. Other vectors include oncolytic viruses.

It is appreciated that in certain embodiments the nucleic acid molecule and the vector may be used in the treatment aspects of the disclosure via a gene therapy approach using formulations and methods described below and known in the art.

A fifth aspect of the disclosure provides a host cell comprising the polynucleotide of the third aspect of the disclosure or a polynucleotide of the fourth aspect of the disclosure. Any of a variety of host cells can be used, such as a prokaryotic cell, for example, E. coli, or a eukaryotic cell, for example a mammalian cell, a human cell, a yeast, insect or plant cell. The host cell may be a cell line, such as a cancer cell line. Suitable examples of cells include Ad293, MDA-MB-231, U20S, HCT116, HeLa and HEK 293 cells. Many suitable vectors and host cells are very well known in the art. Preferably, the host cell is a stable cell line. Alternatively, the host cell may be a cell obtained from a patient.

The disclosure also includes methods for making a molecule of the first aspect of the disclosure or compound of the second aspect of the disclosure. For example, the disclosure comprises expressing in a suitable host cell a recombinant vector encoding the molecule or the first aspect of the disclosure or the compound of the second aspect of the disclosure, and recovering the molecule or compound. Methods for expressing and purifying polypeptides are very well known in the art.

The disclosure also provides a method of producing a cell comprising introducing a polynucleotide molecule according to the third aspect of the disclosure or a vector according to the fourth aspect of the disclosure. Suitable methods of introducing polynucleotide molecules and/or vectors include those described above, and are generally known in the art.

In addition to a host cell being used in a method to produce a molecule or compound of the disclosure, the host cell itself may be used directly in therapy, for example in cell mediated therapy. For example, it may be useful to selectively modulate or degrade disease-causing proteins in particular post-translational states (e.g. phosphorylation states) while still maintaining the expression of other post-translational states. Thus, the disclosure provides a method of treatment, comprising administering a host cell according to the disclosure to the subject, for example for use in medicine or for preventing or treating a disease or condition mediated by an aberrant level of a substrate or form thereof in a subject. Accordingly, the disclosure also provides a host cell according to the fifth aspect of the disclosure for use in medicine, for example for use in the prevention or treating of a disease or condition mediated by an aberrant level of a substrate or form thereof in a subject. The disclosure also provides for the use of said host cell in the manufacture of a medicament for use in medicine, for example for use in the prevention or treating of a disease or condition mediated by an aberrant level of a substrate or form thereof in a subject. Foight et al, “Multi-input chemical control of protein dimerization for programming graded cellular responses,” Nat Biotechnol 2019, 37(10):1209-16 describes the use of PROTACs in cell therapy. Further discussion of how the various agents (e.g. molecules, compounds, polynucleotides, vectors, and compositions) of the disclosure may be used in therapy is provided below.

As is explained below, while the molecule of the disclosure or compound of the disclosure may be clinically effective in the absence of any other therapeutic agent (e.g. anti-cancer compound), it may be advantageous to administer the molecule or compound (or polynucleotide encoding said molecule or compound) in conjunction with a further therapeutic agent.

Accordingly, a sixth aspect of the disclosure provides a composition comprising the first aspect of the disclosure, a compound according to the second aspect of the disclosure, a polynucleotide according to the third aspect of the disclosure, a vector according to the fourth aspect of the disclosure, or a cell according to the fifth aspect of the disclosure, and a further therapeutic agent.

In an embodiment, the further therapeutic agent is selected from the group consisting of an anti-cancer agent, an anti-viral agent, an anti-diabetic agent, an immunotherapeutic agent, an anti-inflammatory agent, an antibiotic, and any combination thereof. Examples of such agents are well known in the art and can readily be identified by the skilled person.

Preferably, the further therapeutic agent is an anti-cancer agent. The further anticancer agent may be selected from alkylating agents including nitrogen mustards such as mechlorethamine (HN2), cyclophosphamide, ifosfamide, melphalan (L-sarcolysin) and chlorambucil; ethylenimines and methylmelamines such as hexamethylmelamine, thiotepa; alkyl sulphonates such as busulphan; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin); natural products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; and biological response modifiers such as interferon alphenomes; miscellaneous agents including platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MIH); and adrenocortical suppressant such as mitotane (o,p′-DDD) and aminoglutethimide; taxol and analogues/derivatives; cell cycle inhibitors; proteosome inhibitors such as Bortezomib (Velcade®); signal transductase (e.g. tyrosine kinase) inhibitors such as Imatinib (Glivec®), COX-2 inhibitors, and hormone agonists/antagonists such as flutamide and tamoxifen. Particularly, tirapazamine may be utilised.

A seventh aspect of the disclosure provide a molecule according to the first aspect of the disclosure, a compound according to the second aspect of the disclosure, a polynucleotide according to the third aspect of the disclosure, a vector according to the fourth aspect of the disclosure, a cell according to the fifth aspect of the disclosure or a composition according to the sixth aspect of the disclosure, for use in medicine.

An eighth aspect of the disclosure provides a pharmaceutical composition comprising a molecule according to the first aspect of the disclosure, a compound according to the second aspect of the disclosure, a polynucleotide according to the third aspect of the disclosure, a vector according to the fourth aspect of the disclosure, a cell according to the fifth aspect of the disclosure or a composition according to the sixth aspect of the disclosure, and one or more pharmaceutically acceptable carrier, diluent or excipient.

While it is possible for the molecule according to the first aspect of the disclosure, a compound according to the second aspect of the disclosure, a polynucleotide according to the third aspect of the disclosure, a vector according to the fourth aspect of the disclosure, a cell according to the fifth aspect of the disclosure or a composition according to the sixth aspect of the disclosure to be administered alone, it is preferable to present it as a pharmaceutical formulation, together with one or more acceptable carriers, diluents or excipients. By “pharmaceutically acceptable” is included that the formulation is sterile and pyrogen free. Suitable pharmaceutical carriers, diluents and excipients are well known in the art of pharmacy. The carrier(s) must be “acceptable” in the sense of being compatible with the inhibitor and not deleterious to the recipients thereof. Typically, the carriers will be water or saline which will be sterile and pyrogen free; however, other acceptable carriers may be used.

Where appropriate, the formulations may be presented in unit dosage form and may be prepared by any of the methods well known in the art of pharmacy. Such methods include the step of bringing into association the active ingredient (e.g. molecule, compound, polynucleotide, vector, or composition of the disclosure) with the carrier which constitutes one or more accessory ingredients. In general, the formulations are prepared by uniformly and intimately bringing into association the active ingredient with liquid carriers or finely divided solid carriers or both, and then, if necessary, shaping the product.

Formulations in accordance with the present disclosure suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

In some embodiments, the unit dosage formulations are those containing a daily dose or unit, daily sub-dose or an appropriate fraction thereof, of an active ingredient. It should be understood that in addition to the ingredients particularly mentioned above the formulations of this disclosure may include other agents conventional in the art having regard to the type of formulation in question, for example those suitable for oral administration may include flavouring agents.

The amount of the agent of the disclosure which is administered to the individual is an amount effective to combat the particular individual's condition. The amount may be determined by the physician.

Preferably, in the context of any medical use described herein, the subject to be treated is a human. Alternatively, the subject may be an animal, for example a domesticated animal (for example a dog or cat), laboratory animal (for example laboratory rodent, for example mouse, rat or rabbit) or an animal important in agriculture (i.e. livestock), for example horses, cattle, sheep or goats.

It will be appreciated that the molecule of the disclosure can be delivered to a cell (e.g. a cell that contains the substrate which is desirable to regulate (e.g. degrade)) in various ways. For example, the molecule of the disclosure may be a fusion polypeptide that can be targeted to a cell in an individual by virtue of it being attached to a separate targeting moiety as described above in relation to the compound of the second aspect of the disclosure. In this way, the fusion polypeptide is brought into the vicinity of the cell and may be delivered to the cell, for example by internalisation following the binding of the targeting moiety to an entity on the cell (e.g. on the cell surface). Alternatively, the molecule of the disclosure may be a fusion polypeptide and it is delivered to a cell by introducing a polynucleotide or vector encoding the fusion polypeptide into the cell.

Accordingly, a ninth aspect of the disclosure provides a method of delivering a molecule according to the first aspect of the disclosure to a cell (e.g. a cell that contains the substrate which is desirable to regulate (e.g. degrade)) in an individual, the method comprising: administering to the individual a compound of the second aspect of the disclosure or administering to the individual a polynucleotide of the third aspect of the disclosure or a vector of the fourth aspect of the disclosure, wherein the polynucleotide or vector encodes the molecule in the cell.

The molecule, compound, polynucleotide or vector may be administered orally or by any parenteral route, for example in the form of a pharmaceutical formulation comprising the active ingredient, optionally in the form of a non-toxic organic, or inorganic, acid, or base, addition salt, in a pharmaceutically acceptable dosage form. The active ingredient may be administered at varying doses.

The disclosure also provides a compound according to the second aspect of the disclosure, a polynucleotide according to the third aspect of the disclosure or a vector according to the fourth aspect of the disclosure for use in delivering a molecule according to the first aspect of the disclosure to a cell (e.g. a cell that contains the substrate which is desirable to regulate (e.g. degrade)) in an individual.

Similarly, the disclosure also provides the use of a compound according to the second aspect of the disclosure, a polynucleotide according to the third aspect of the disclosure or a vector according to the fourth aspect of the disclosure, in the manufacture of a medicament for delivering a molecule according to the first aspect of the disclosure to a cell (e.g. a cell that contains the substrate which is desirable to regulate (e.g. degrade)) in an individual.

A tenth aspect of the disclosure provides a kit of parts comprising: (a) a regulation domain comprising an E2 ubiquitin or ubiquitin-like conjugating domain which has an amino acid sequence having at least 80% sequence identity to a human E2 enzyme or a functional part thereof, and (b) a targeting domain capable of targeting the regulation domain to a substrate; optionally wherein the kit does not comprise an E3 ubiquitin or ubiquitin-like ligase or a functional part thereof.

Preferences for the regulation domain, E2 ubiquitin or ubiquitin-like conjugating domain, targeting domain, substrate and E3 ubiquitin or ubiquitin-like ligase or functional part thereof, include those described above in relation to the first aspect of the disclosure.

In an embodiment, the kit further comprises a linking means suitable for linking the regulation domain to the targeting domain. Any suitable linking means including linkers as described elsewhere herein may be used. Thus, the kit may further comprise a linker that is capable of joining the regulation domain to the targeting domain. The linking may be covalent or non-covalent.

In an additional embodiment, the kit further comprises a targeting moiety that is capable of targeting to a cell that contains a substrate to be regulated (e.g. degraded). Hence, it will be appreciated that the kit may be useful in a “plug and play” context, wherein appropriate regulation domains, targeting domains and targeting moieties are selected and then combined to form a tailored treatment for a given individual. It will be appreciated that such kits are suitable for use in the treatment aspects of the disclosure described herein and below. For example, if a cancer was shown to be dependent on the expression or activity of a particular oncogene, that oncogene could be targeted for degradation or other regulation. This may be achieved using a promiscuous E2 enzyme or a functional part or variant thereof, but if the oncogene is known to be a substrate protein for a specific E2 enzyme, that E2 enzyme may be selected. Preferences for the targeting moiety include those described above in relation to the second aspect of the disclosure. It is preferred if the targeting moiety is an antibody.

An eleventh aspect of the disclosure provides a kit of parts comprising: (a) a molecule of the first aspect of the disclosure; and (b) a targeting moiety that is capable of targeting to cells that contain a substrate to be regulated (e.g. degraded). Preferences for the molecule of the first aspect of the disclosure and substrate include those described above in relation to the first aspect of the disclosure, and preferences for the targeting moiety include those described above in relation to the second aspect of the disclosure. It is preferred if the targeting moiety is an antibody. Again, it will be appreciated that such kits are suitable for use in the treatment aspects of the disclosure described herein and below, and may be useful in a “plug and play” context.

In an embodiment, the kit further comprises a linking means suitable for linking the molecule of the first aspect of the disclosure to the targeting moiety. Any suitable linking means including linkers as described elsewhere herein may be used. Thus, the kit may further comprise a linker that is capable of joining the molecule of the first aspect of the disclosure to the targeting moiety. The linking may be covalent or non-covalent.

A twelfth aspect of the disclosure provides a kit of parts comprising: (a) a polynucleotide that encodes a regulation domain comprising an E2 ubiquitin or ubiquitin-like conjugating domain which has an amino acid sequence having at least 80% sequence identity to a a human E2 enzyme or a functional part thereof, and (b) a polynucleotide that encodes a targeting domain capable of targeting the regulation domain to a substrate; optionally wherein the kit does not comprise a polynucleotide that encodes an E3 ubiquitin or ubiquitin-like ligase or a functional part thereof.

Preferences for the regulation domain, E2 ubiquitin or ubiquitin-like conjugating domain, targeting domain, substrate, and E3 ubiquitin or ubiquitin-like ligase or functional part thereof, include those described above in relation to the first aspect of the disclosure.

In an embodiment, the kit comprises one or more promoter sequences capable of directing expression of one or both of the polynucleotides in a cell that contains a substrate to be regulated. The promoters may be constitutively active or they may be inducible, thereby allowing the temporal regulation of expression of the polynucleotides in cells. It may be useful to use tissue specific promoters, so as to target expression to particular cell types or tissues. Such promoters are well known in the art and can be readily sourced or designed, for example based on consulting the scientific literature.

A further kit of parts provided by the disclosure comprises: (a) a polynucleotide encoding a molecule according to the first aspect of the disclosure, and (b) a targeting moiety that is capable of targeting to cells that contain a substrate to be regulated. Preferences of the molecule according to the first aspect of the disclosure and the targeting moiety include those described above. It will be appreciated that such a kit may also be used in a “plug and play” system as described above, wherein a polynucleotide encoding a molecule according to the first aspect of the disclosure may be used to express such a molecule that can then be attached to a suitable targeting moiety, for example depending upon the ultimate therapeutic application.

As discussed below, the agents of the disclosure have utility in preventing or treating a disease or condition mediated by an aberrant level of a substrate in a subject. Thus, it will be appreciated, that before treating the subject, it may be useful to confirm or else ascertain which substrate is at an aberrant level in a cell, for example a cell in a biopsy taken from a subject. Hence, it will be appreciated that it may be useful if any of the kit of parts described above further comprised one or more reagents to assess the expression profile of a cell that contains a substrate to be regulated. Assessing the expression profile of the cell (e.g. in a biopsy sample) may be carried out using routine assays for measuring nucleic acid (e.g. DNA or RNA transcripts) or protein levels. For example, transcriptomic or proteomic techniques may be used. Any suitable reagents may be used, including binding partners of nucleic acid encoding the substrate, binding partners of the substrate itself, and PCR primers. Preferably, the reagent is an antibody that binds to the substrate.

As discussed further below, the agents of the disclosure have utility in assessing the function of a substrate by assessing the effect of the molecule of the disclosure on one or more properties of the cell, tissue or organ. In some embodiments, therefore any of the kits of parts described above may further comprise a means for assessing a property of a cell. In this way, the kits can be used in a screening context, for example to identify substrates that have a particular effect on a given property of a cell.

By a property of the cell we include any of survival, growth, proliferation, differentiation, migration, morphology, signalling, metabolic activity, gene expression, protein translation, and cell-cell interaction. Assessing one or more properties of a cell may be carried out using any suitable method known in the art. For example, any of cell survival, growth, proliferation, differentiation, migration and morphology may be assessed by microscopy or image analysis. Properties may also be detected using appropriate markers. For example, expression of detectably-labelled proteins, reporters and/or single-step labelling of cell components and markers can enable cell architecture, multicellular organisation and other readouts to be directly visualised, for example by fluorescence microscopy (e.g. E-cadherin staining to identify cell-cell contacts). Gene expression may be assessed by functional genomic (eg microarray) techniques, protein translation by proteomic techniques or immunohistochemical techniques, and so on. Any of immunofluorescence, Hoeschst staining or Annexin-V assays may be used. It is appreciated, therefore, that the skilled person may select the appropriate technique to assess a given property, and thereby select an appropriate means. Examples of means include any of antibodies, primers, enzymatic reagents, immunoassay reagents, detectable markers of entities of interest (e.g. proteins or nucleic acids).

A thirteenth aspect of the disclosure provides a method of preventing or treating a disease or condition mediated by an aberrant level of a substrate or form thereof in a subject, the method comprising administering the molecule of the first aspect of the disclosure, the compound of the second aspect of the disclosure, the polynucleotide of the third aspect of the disclosure, the vector of the fourth aspect of the disclosure, the cell according to the fifth aspect of the disclosure, the composition of the sixth aspect of the disclosure, and the pharmaceutical composition of the eighth aspect of the disclosure, to the subject.

Similarly, the disclosure provides a molecule of the first aspect of the disclosure, a compound of the second aspect of the disclosure, a polynucleotide of the third aspect of the disclosure, a vector of the fourth aspect of the disclosure, a cell according to the fifth aspect of the disclosure, a composition of the sixth aspect of the disclosure, and a pharmaceutical composition of the eighth aspect of the disclosure for use in preventing or treating a disease or condition mediated by an aberrant level of a substrate or form thereof in a subject.

Likewise, the disclosure provides the use of a molecule of the first aspect of the disclosure, a compound of the second aspect of the disclosure, a polynucleotide of the third aspect of the disclosure, a vector of the fourth aspect of the disclosure, a composition of the sixth aspect of the disclosure, and a pharmaceutical composition of the eighth aspect of the disclosure in the manufacture of a medicament for preventing or treating a disease or condition mediated by an aberrant level of a substrate or form thereof in a subject.

By preventing or treating a condition we include the meaning of reducing or alleviating symptoms in a patient (i.e. palliative use), preventing symptoms from worsening or progressing, treating the disorder (e.g. by inhibition or elimination of the causative agent), or prevention of the condition or disorder in a subject who is free therefrom.

By a ‘condition mediated by an aberrant level of a substrate or form thereof’, we include the meaning of any biological or medical condition or disorder in which at least part of the pathology is mediated by an aberrant level of substrate or form thereof. The condition may be caused by the aberrant level of the substrate or form thereof or else the aberrant level of the substrate of form thereof may be an effect of the condition. By aberrant level we include the meaning that the substrate or form thereof is present at a higher or lower level than the substrate or form thereof in a normal, non-pathological status. It will be appreciated that the amount of the substrate itself may remain the same as between a pathological and non-pathological condition, but the proportion of the amount of substrate residing in a particular form (e.g. a particular post-translational modified form) may be higher or lower in a pathological state. For the avoidance of doubt, by aberrant level of substrate, we include the meaning of an aberrant level of a form of that substrate, such as a post-translational modified form (e.g. phosphorylated form). Examples of particular conditions include cancer, diabetes, autoimmune disease, Alzheimer's disease, Parkinson's disease, pain, viral disease, bacterial disease, prionic disease, fungal disease, parasitic disease, arthritis, immunodeficiency, and inflammatory disease.

The agent of the disclosure (e.g. a molecule of the first aspect of the disclosure, a compound of the second aspect of the disclosure, a polynucleotide of the third aspect of the disclosure, a vector of the fourth aspect of the disclosure, a cell of the fifth aspect of the disclosure, a composition of the sixth aspect of the disclosure, and a pharmaceutical composition of the eighth aspect of the disclosure) may be formulated in any suitable way, and/or administered to the individual by any suitable route of administration and/or administered to the individual at an appropriate dose, for example as described above, and as determined by a physician.

A fourteenth aspect of the disclosure provides a method of regulating a substrate, the method comprising contacting the substrate with the molecule of the first aspect of the disclosure under conditions effective for the molecule to regulate the substrate. Preferences for the molecule of the first aspect of the disclosure and the substrate include those described above.

By regulating we include the meaning of any of the possible types of regulation that can be mediated by ubiquitin or ubiquitin-like protein, including those described above, for example regulating one or more activities of a target substrate and/or regulating the cellular location of a target substrate and/or regulating the stability of a target substrate. Preferably, the regulating involves degrading the substrate. Thus, in an embodiment, the regulating involves the substrate being degraded, or the substrate being prevented from being degraded, or the subcellular location of the substrate being altered, or one or more activities of the substrate being modulated (e.g. increased or decreased), or the degree of post-translational modification of the substrate being modulated. The method may be performed in vivo or in vitro.

By ‘under conditions effective for the molecule to regulate the substrate’ we include the meaning that the substrate is contacted with the molecule of the disclosure under conditions which allow formation of a complex between the substrate and the molecule, such that ubiquitin or ubiquitin-like protein can be conjugated to the substrate and the substrate thereby regulated. The minimal conditions would be the presence of the E1 protein, ubiquitin or ubiquitin-like protein, and the cellular machinery for the particular regulation mediated by ubiquitin or ubiquitin-like protein. For example, if the particular regulation is ubiquitin-mediated degradation, the conditions effective for the molecule to degrade the substrate would include the cellular machinery necessary for such degradation, e.g. the proteasome, and so on. Typically, the method is carried out within a cell and so the cellular conditions are effective for the molecule to regulate the substrate. However, in vitro ubiquitination assays are known, and so the method may be carried out in vitro, for example to further understanding of the mechanism, kinetics and location of ubiquitin or ubiquitin-like protein additions.

It is appreciated that the agents of the disclosure will have utility in identifying and/or validating substrates as potential drug targets. The agents of the disclosure provide for targeted regulation (e.g. degradation) of cellular substrates, including intracellular substrates, and so the effects of such regulation may be beneficial in a therapeutic setting.

Accordingly, a fifteenth aspect of the disclosure provides method of identifying a substrate as a potential drug target, the method comprising:
(a) providing a cell, tissue or organ comprising the substrate
(b) contacting the cell, tissue or organ with a molecule according to the first aspect of the disclosure or a compound according to the second aspect of the disclosure or a polynucleotide according to the third aspect of the disclosure or a vector according to the fourth aspect of the disclosure; and
(c) assessing the effect of the molecule, compound, polynucleotide or vector on one or more properties of the cell, tissue or organ, wherein identification of an effect that is correlated with a particular disease status is indicative that the substrate is a potential drug target for the particular disease.

Suitable cells, or the tissue/organs they can be derived from, include bone marrow, skin, cartilage, tendon, bone, muscle (including cardiac muscle), blood vessels, corneal, neural, brain, gastrointestinal, renal, liver, pancreatic (including islet cells), lung, pituitary, thyroid, adrenal, lymphatic, salivary, ovarian, testicular, cervical, bladder, endometrial, prostate, vulval and esophageal. Also included are the various cells of the immune system, such as T lymphocytes, B lymphocytes, polymorphonuclear leukocytes, macrophages and dendritic cells. The cells may be stem cells, progenitor cells or somatic cells. Preferably the cells are mammalian cells such as human cells or cells from animals such as mice, rats, rabbits, and the like. It is appreciated that the cells may be derived from a normal or healthy biological tissue, or from a biological tissue afflicted with a disease or illness, such as a tissue or fluid derived from a tumour.

It will be appreciated that the method may be performed in vivo, ex vivo or in vitro. For example, the method may be carried out on tissues or organs ex vivo, in cell cultures in vitro, or on cells, tissue or organs when residing in their natural environment in vivo.

It will be appreciated that the molecule of the first aspect of the disclosure may be delivered to the cells, organ or tissue either by directly contacting with the molecule of the first aspect of the disclosure or compound of the second aspect of the disclosure (e.g. where the compound includes a targeting moiety that binds to an entity on the surface of a cell, leading to internalisation of the compound), or by expressing the polynucleotide of the third aspect of the disclosure or vector of the fourth aspect of the disclosure.

By assessing the effect of the molecule, compound, polynucleotide or vector on one or more properties of the cell, tissue or organ, we include the meaning of assessing the effect on any one or more properties of a cell, tissue or organ, which properties are known to be correlated with a particular disease state. In this way, knowing that regulating (e.g. degrading) a substrate has an effect on the one or more properties, it is possible to identify that substrate as a potential drug target for the particular disease.

Any properties of a cell, tissue or organ can be assessed, and for a given disease or condition, the skilled person would readily be able to identify suitable properties to be assessed. Thus, the one or more properties may be any of the properties of a cell described above in relation to the twelfth aspect of the disclosure, for example properties selected from the group consisting of survival, growth, proliferation, differentiation, migration, morphology, signalling, metabolic activity, gene expression, protein translation, and cell-cell interaction. Properties of tissues and organs include morphology and multicellular organisation. In the context of cancer, the properties to be assessed may include any one or more of cell growth, proliferation, differentiation and migration.

Assessing one or more properties of a cell, tissue or organ may be carried out using any suitable method known in the art, for example as described above in relation to the twelfth aspect of the disclosure. In some embodiments, the method may be carried out using one of the kits of parts of the disclosure described above.

In a similar way to the fifteenth aspect of the disclosure, it will be understood that the agents of the disclosure may be useful in assessing the function of substrates, for example by degrading substrates and assessing the effect, or by otherwise regulating substrates, and assessing the effect of their upregulation.

Accordingly, a sixteenth aspect of the disclosure provides a method of assessing the function of a substrate, the method comprising:
(a) providing a cell, tissue or organ comprising the substrate
(b) contacting the cell, tissue or organ with a molecule according to the first aspect of the disclosure or a compound according to the second aspect of the disclosure or a polynucleotide according to the third aspect of the disclosure or a vector according to the fourth aspect of the disclosure; and
(c) assessing the effect of the molecule, compound, polynucleotide or vector on one or more properties of the cell, tissue or organ.

Preferences for the cell, tissue and organ, and the one or more properties of the cell, tissue or organ, include those described above in relation to the fifteenth aspect of the disclosure. It will be appreciated that the method may be performed in vivo, ex vivo or in vitro. For example, the method may be carried out on tissues or organs ex vivo, in cell cultures in vitro, or on cells, tissue or organs when residing in their natural environment in vivo. It will also be appreciated that this method allows the assessment of the function of cellular gene or protein, for example when the substrate is a protein encoded by a gene. In some embodiments, the method may be carried out using one of the kits of parts of the disclosure described above.

A seventeenth aspect of the disclosure provides a method of identifying an agent that may be useful in preventing or treating a disease or condition mediated by an aberrant level of a substrate or form thereof, the method comprising:
providing the substrate;
providing a test agent comprising (a) a regulation domain comprising an E2 ubiquitin or ubiquitin-like conjugating domain which has an amino acid sequence having at least 80% sequence identity to a human E2 ubiquitin or ubiquitin-like domain, and (b) a targeting domain capable of targeting the regulation domain to a substrate, optionally wherein the test agent does not comprise an E3 ubiquitin or ubiquitin-like ligase or part thereof;
contacting the substrate and test agent under conditions effective for the test agent to facilitate regulation of the substrate; and
determining whether the test agent regulates the substrate.

Preferences for the substrate, and for the disease or condition mediated by an aberrant level of a substrate or form thereof, include those described above. For example, the substrate (e.g. protein) may be an intracellular protein that itself, or a form thereof (e.g. post-translational modified form such as a phosphorylated form), is implicated in a particular disease or condition

It will be appreciated that the test agent may be a molecule according to the first aspect of the disclosure. Hence, the method may be used to assess the efficacy of a candidate molecule of the first aspect of the disclosure to regulate the substrate (e.g. degrade the substrate), and thereby identify the molecule as one that may be useful in combating a disease or condition mediated by an aberrant level of a substrate or form thereof.

It will be appreciated that the method may be performed in vivo, ex vivo or in vitro. For example, the method may be carried out on tissues or organs ex vivo, in cell cultures in vitro, or on cells, tissue or organs when residing in their natural environment in vivo.

By ‘conditions effective for the test agent to facilitate regulation of the substrate’, we include the meaning that the substrate is contacted with the molecule of the disclosure under conditions which allow formation of a complex between the substrate and the molecule, such that ubiquitin or ubiquitin-like protein can be conjugated to the substrate and the substrate thereby regulated. The minimal conditions include those defined above in relation to the fourteenth aspect of the disclosure. It is preferred if the method is carried out within a cell and so the cellular conditions are effective for the test agent to facilitate regulation of the substrate. However, in vitro ubiquitination assays are known, and so the method may be carried out in vitro.

In a preferred embodiment, the test agent is one that degrades the substrate. It is appreciated that in some instances high throughput screening of test agents is preferred and that the method may be used as a “library screening” method, a term well known to those skilled in the art. Thus, the test agent may be a library of test agents. Methodologies for preparing and screening such libraries are known in the art.

The disclosure includes screening methods to identify drugs or lead compounds for use in treating a disease or condition. It is appreciated that screening assays which are capable of high throughput operation are particularly preferred. It is appreciated that the identification of a test agent that regulates (e.g. degrades) a substrate may be an initial step in a drug screening pathway, and the agent may be further selected e.g. based on its efficacy in an assay of the disease or condition in question, and/or further modified. Thus, the method may further comprise the step of testing the test agent in an assay of the disease or condition in questions. Assays for various diseases and conditions are known in the art.

The method may comprise the further step of synthesising and/or purifying the identified agent or the modified agent. The disclosure may further comprise the step of synthesising, purifying and/or formulating the identified test agent. Agents may also be subjected to other tests, for example toxicology or metabolism tests, as is well known to those skilled in the art. The disclosure includes the use of a molecule of the first aspect of the disclosure or the compound of the second aspect of the disclosure or the polynucleotide of the third aspect of the disclosure or the vector of the fourth aspect of the disclosure in drug target validation or in drug discovery.

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order, and as appropriate, any combination of two or more steps may be conducted simultaneously.

All of the documents referred to herein are incorporated herein, in their entirety, by reference. The listing or discussion of an apparently prior-published document in this specification should not necessarily be taken as an acknowledgement that the document is part of the state of the art or is common general knowledge.

### EXAMPLES

### Example 1—Degradation of SHP2 Protein in MDA-MB-231 Cells Comparing an E3 Fusion Polypeptide and an E2 Fusion Polypeptide

### Introduction

The aim of this experiment was to determine whether a biological PROTAC (herein referred to as a fusion polypeptide) capable of degrading a target protein could be produced by using an E2 ubiquitin conjugating enzyme as the ‘regulation’ or ‘degradation’ domain rather than the standard E3 ligase ‘degradation’ domain. Previous studies have demonstrated the ability of biological PROTACs using E3 ‘degradation’ domains to degrade target proteins (Portnoff et al, J. Biol. Chem., 2014 289(11):7844-5; Pan et al, Oncotarget, 2016 7(28):44299-44309; Fulcher et al, Open Biol, 2017 7(5). pii:170066). For this experiment, MDA-MB-231 breast cancer cells were transduced with lentiviral constructs encoding fusion polypeptides and control proteins. The UBE2D1 E2 ubiquitin conjugating enzyme was selected to be incorporated into a fusion protein as an N-terminal ‘degradation’ domain upstream of a linker and SHP2-binding monobody aCS3 (Sha et al., Proc Natl Acad Sci USA, 2013 110(37):14924-9). Controls include an N-terminal and C-terminal E3 ligase (VHL; von Hippel-Lindau) polypeptide fusion to aCS3, aCS3 monobody alone, VHL alone, UBE2D1 alone and un-transduced control cells. The degree of target SHP2 degradation will be determined by western blot analysis and by densitometry of western blot bands.

### Material and Methods

Lentiviral particles were produced as described in ‘Generation of lentiviral particles’ section of the main ‘Methods’ section. Lentiviral particles encoding the following fusion polypeptides (or individual components) were produced in HEK293FT cells: HA_aCS3 (SEQ ID NO: 149), HA_VHL (SEQ ID NO: 168), HA_VHL_Linker 4_aCS3 (SEQ ID NO: 154), HA_aCS3_Linker 4_VHL (SEQ ID NO: 200), HA_UBE2D1 (SEQ ID NO: 169), and HA_UBE2D1_Linker 4_aCS3 (SEQ ID NO: 194).

MDA-MB-231 cells were transduced according to the methods described in ‘Transduction of cells with Lentivirus’ and prepared for Western blot analysis as described in ‘Western blot analysis and quantification’ within the main Methods section. An MDA-MB-231 un-transduced control (‘cells’) lysate is also included. Western blot analysis of sample lysates is performed using rabbit anti-SHP2 (CST #3397; 1:1000 dilution) with secondary Goat anti-Rabbit IRDye800 (Licor #925-32211; 1:15,000 dilution); and mouse anti-alpha tubulin (Licor #926-42213; 1:10,000 dilution) with secondary Goat anti-mouse IRDye680RD (Licor #926-68070; 1:15,000 dilution). Blots are then visualised on the Odyssey system and densitometry of western blot bands is performed using Image Studio software. For each sample, the densitometry values for SHP2 protein bands are divided by the respective densitometry value for the loading control (alpha tubulin). These values are then given as a percentage of the SHP2/alpha-tubulin value observed for control (un-transduced) MDA-MB-231 cells.

### Results

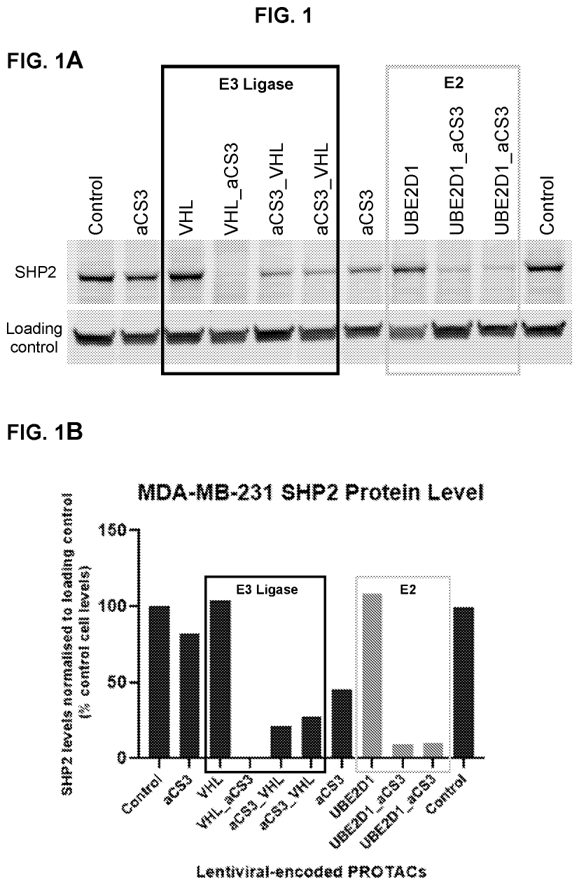

FIG. 1A shows a western blot with SHP2 protein and alpha-tubulin loading control. The two replicate samples for HA_UBE2D1 Linker 4 aCS3 (labelled in the figure as “UBE2D1_aCS3”) indicate that SHP2 protein levels are reduced as compared to control samples. The densitometry quantification suggests SHP2 protein levels are reduced by 90% (FIG. 1B). The HA_VHL_Linker 4 aCS3 (labelled in the figure as “VHL_aCS3”) expressing sample demonstrates no detectable SHP2, whilst the inverse orientation HA_aCS3 Linker 4 VHL samples (labelled in the figure as “aCS3_VHL”) resulted in approximately 70-80% reduction in SHP2 levels (FIGS. 1A and 1B). HA_VHL alone (labelled in the figure as “VHL”) and HA_UBE2D1 (labelled in the figure as “UBE2D1”) alone controls did not appear to negatively affect SHP2 expression levels; however, the HA_aCS3 monobody sample replicates did demonstrate some variability in SHP2 protein levels.

### Conclusion

These data suggest a fusion polypeptide comprising an E2 ubiquitin conjugating enzyme is capable of reducing target protein expression. The most likely method for this reduction is by target ubiquitination and subsequent proteasomal degradation. The data observed with examining the VHL fusion constructs in different orientations suggest that the orientation of the binding and degradation domains relative to each other may influence the effectiveness of target ubiquitination and hence degradation with an E3 ligase.

### Example 2A—Investigating Fusion Polypeptide Domain Orientation and Linker Length in E3 Ligase and E2 Fusion Polypeptides

### Introduction

The aim of this experiment was to investigate the length of the linker between the ‘targeting’ domain and the ‘regulation/degradation’ domain as well as the orientation of these domains with respect to each other and determine how these variables affected fusion polypeptide-mediated changes in target expression. Data shown in FIG. 1 suggest with the VHL (E3 ligase) degradation domain, an N-terminal position resulted in more target SHP2 degradation. This is the orientation reported by Fulcher et al (Open Biol, 2017 (5). pii: 170066). For this experiment, MDA-MB-231 breast cancer and U20S bone osteosarcoma cells were transduced with lentiviral constructs encoding fusion polypeptide and control proteins comparing a short (9 amino acid linker) to a long (19 amino acid linker) in both orientations. The UBE2D1 E2 ubiquitin conjugating enzyme was selected as an E2 fusion polypeptide ‘regulation/degradation’ domain and VHL as an E3 fusion polypeptide ‘degradation domain’. SHP2-binding monobody aCS3 (Sha et al., Proc Natl Acad Sci USA, 2013 110(37):14924-9) was used as the ‘binding’ domain in all fusion polypeptide constructs. Controls include aCS3 monobody alone, VHL alone, UBE2D1 alone and un-transduced control cells. The degree of target SHP2 degradation was determined by western blot analysis and quantified by densitometry of western blot bands.

### Materials and Methods

Lentiviral particles encoding the following fusion polypeptides (or individual components) were produced in HEK293FT cells: HA_aCS3 (SEQ ID NO: 149), HA_UBE2D1 (SEQ ID NO: 169), HA_UBE2D1 Linker 2 aCS3 (SEQ ID NO: 159), HA_UBE2D1 Linker 1 aCS3 (SEQ ID NO: 158), HA_aCS3 Linker 2 UBE2D1 (SEQ ID NO: 203), HA_aCS3 Linker 1_UBE2D1 (SEQ ID NO: 202), HA_VHL (SEQ ID NO: 168), HA_VHL_Linker 2 aCS3 (SEQ ID NO: 153), HA_VHL_Linker 1 aCS3 (SEQ ID NO: 152), HA_aCS3 Linker 2 VHL (SEQ ID NO: 197), and HA_aCS3 Linker 1 VHL (SEQ ID NO: 196).

MDA-MB-231 and U20S cells were transduced according to the methods described in ‘Transduction of cells with lentivirus’ and prepared for western blot analysis as described in ‘western blot analysis and quantification’ within the main methods section. MDA-MB-231 and U20S un-transduced control (‘cells’) lysates were also included. Western blot analysis of sample lysates was performed using rabbit anti-SHP2 (CST #3397; 1:1000 dilution) with secondary Goat anti-Rabbit IRDye800 (Licor #925-32211; 1:15,000 dilution); and rabbit anti-GAPDH (CST #5174; 1:4,000) with secondary Goat anti-Rabbit IRDye800 (Licor #925-32211; 1:15,000 dilution). Blots were visualised on the Odyssey system and densitometry of western blot bands was performed using Image Studio software. For each sample, the densitometry values for SHP2 protein bands are divided by the respective densitometry value for the loading control (GAPDH). These values are then given as a percentage of the SHP2/GAPDH value observed for control (un-transduced) MDA-MB-231 and U20S cells respectively.

### Results

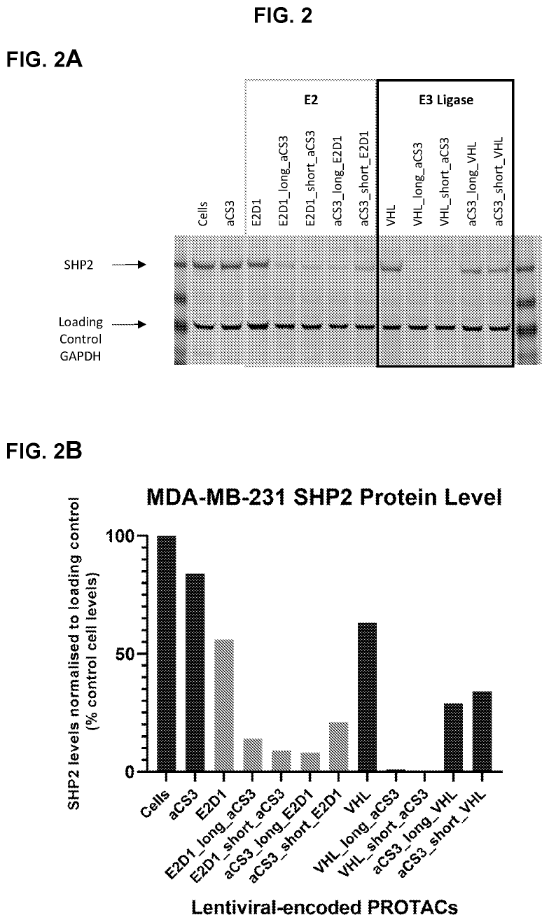

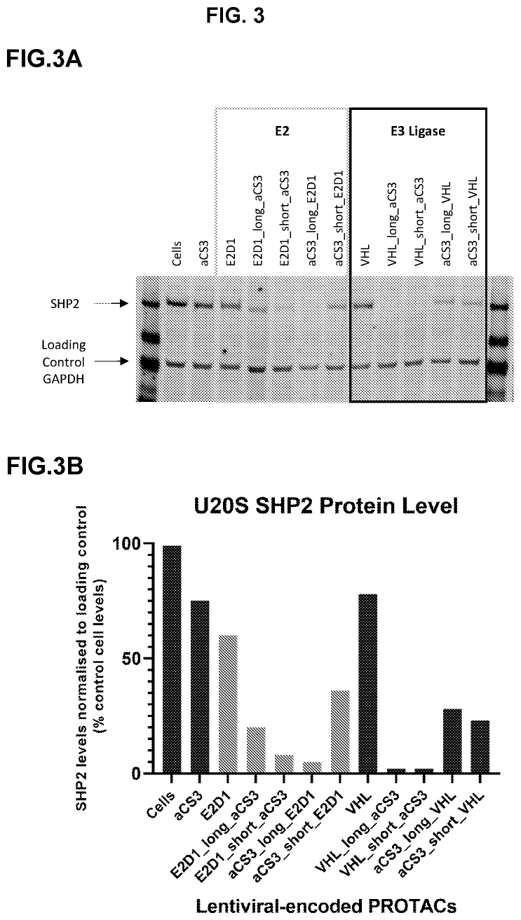

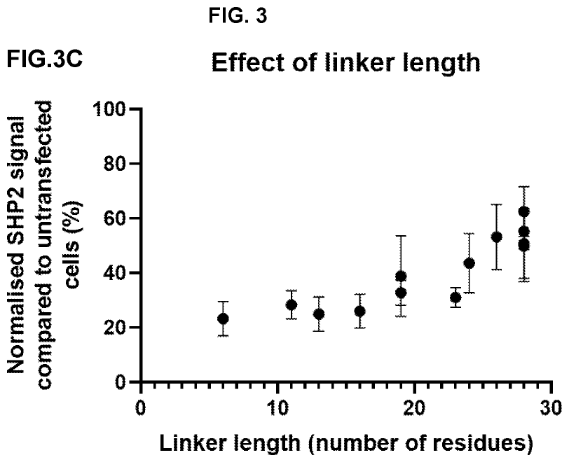

FIGS. 2A and 3A show western blots with SHP2 protein and GAPDH loading control bands. The UBE2D1 ‘regulation/degradation’ domain constructs have used the shorter name E2D1 in FIGS. 2 and 3. In both MDA-MB-231 and U20S cell lines, the UBE2D1 (E2D1) fusion polypeptide constructs resulted in 60-90% reduction in SHP2 protein levels relative to control cells (FIG. 2 and FIG. 3). In MDA-MB-231 cells there was not much variation in the reduction of SHP2 protein between the different linker lengths and orientations (FIG. 2). In U20S cells, there was slightly more variation with the HA_aCS3 Linker 1 UBE2D1 construct (labelled in the figure as “aCS3_short_E2D1”) appearing the least effective format (FIG. 3A). In both the MDA-MB-231 and U20S cells, having the VHL E3 ligase degradation domain in the N-terminal position resulted in the greatest reduction in SHP2 levels irrespective of linker length (FIG. 2 and FIG. 3). Having the aCS3 binding domain N-terminal to the VHL degradation domain resulted in less effective reduction in SHP2 protein levels in both cell lines (FIG. 2 and FIG. 3).

### Conclusion

These data suggest a fusion polypeptide comprising an E2 ubiquitin conjugating enzyme may be less affected by domain orientation than the E3 ligase fusion polypeptides. The data once again showed the E2 fusion polypeptides using UBE2D1 as a ‘regulation/degradation’ domain were capable of reducing target SHP2 protein levels. Some variability was observed between some of the results, which may indicate differences in construct activity or the amount of construct in each cell sample.

### Example 2B—Further Investigating Fusion Polypeptide Domain Linker Length in E2 Fusion Polypeptides

### Introduction

Following a study of the effect of orientation on PROTAC activity, the aim of this experiment was to further investigate the length of the linker between the ‘targeting’ domain and the ‘regulation/degradation’ domain to determine how varying linker length affected fusion polypeptide-mediated changes in target expression. Data shown in FIGS. 2A, 2B, 3A and 3B demonstrate that fusion polypeptides comprising an E2 ubiquitin conjugating enzyme with a 9 amino acid linker or 19 amino acid linker were both capable of reducing target SHP2 protein levels in MDA-MB-231 and U20S cells. For this experiment, further linkers were tested with lengths of 6, 11, 13, 16, 19, 23, 24, 26 and 28 amino acids. The UBE2D1 E2 ubiquitin conjugating enzyme was again selected as an E2 fusion polypeptide ‘regulation/degradation’ domain. SHP2-binding monobody aCS3 (Sha et al., Proc Natl Acad Sci USA, 2013 110(37):14924-9) was again used as the ‘binding’ domain in all fusion polypeptide constructs. Controls include un-transduced control cells. The degree of target SHP2 degradation was determined by western blot analysis and quantified by densitometry of western blot bands.

### Materials and Methods

mRNA synthesis: Linear DNA templates encoding E2D1_aCS3_HA (UBE2D1_aCS3_HA) linker variants and consisting of a T7 promoter, a 5′ UTR, a fusion polypeptide-encoding open reading frame, a 3′ UTR and a polyA tail, were used for in vitro transcription of mRNA as described elsewhere (Vaidyanathan S, et al., Uridine Depletion and Chemical Modification Increase Cas9 mRNA Activity and Reduce Immunogenicity without HPLC Purification. Mol Ther Nucleic Acids 12, 530-542 (2018)).

The fusion polypeptides were created with the following arrangement: UBE2D1_Linker_aCS3_HA, using wild-type UBE2D1 and wherein the linkers used correspond to the following sequences:

| | | Length | |
|---|---|---|---|
| | | (# of | |
| | | amino | SEQ ID |
| Linkers | Amino acid sequence | acids) | NO: |
| Linker 7 | LEGGSR | 6 | SEQ ID |
| | | | NO: 211 |
| Linker 8 | LEGGGSGGSSR | 11 | SEQ ID |
| | | | NO: 212 |
| Linker 9 | LEGGGGSGGGSSR | 13 | SEQ ID |
| | | | NO: 213 |
| Linker 10 | LEGGGSGGGSGGGSSR | 16 | SEQ ID |
| | | | NO: 214 |
| Linker 2 | LEGGGGSGGGGSGGGGSSR | 19 | SEQ ID |
| | | | NO: 142 |
| Linker 11 | LEGGGGSGPSGGGGPSGSR | 19 | SEQ ID |
| | | | NO: 215 |
| Linker 12 | LESNGGGGSPAPAPGGGGSGSSR | 23 | SEQ ID |
| | | | NO: 216 |
| Linker 13 | LEGGGGSYPYDVPDYASGGGGSSR | 24 | SEQ ID |
| | | | NO: 217 |
| Linker 14 | TGGSAGGSGGSAGGSGGSAGGSGGSA | 26 | SEQ ID |
| | | | NO: 218 |
| Linker 15 | AGSGGSTGSGGSPTPSTSGGSTGSGGAS | 28 | SEQ ID |
| | | | NO: 219 |
| Linker 16 | AGSGGSGGSGGSGNSSTSGGSGGSGGAS | 28 | SEQ ID |
| | | | NO: 220 |
| Linker 17 | GGSPVPSTPGGGSGGGSGGSPVPSTPGS | 28 | SEQ ID |
| | | | NO: 221 |
| Linker 18 | SPGTGSPGTGSPGTGSPGTGSPGTGSPG | 28 | SEQ ID |
| | | | NO: 222 |

The fusion polypeptides used in these experiments correspond to the nucleic acid sequences of SEQ ID NOs: 223-235, which encode the amino acid sequences of SEQ ID NOs: 236-248.

Transfection of cells with mRNA: U20S cells were transfected with mRNA using RNAiMAX (Invitrogen) according to manufacturer's instructions. 4×10³ U2OS cells per well were aliquoted onto collagen-coated 96-well plates and incubated for 48 hours at 37° C. Cells were then transfected with 100 ng of each mRNA encoded fusion polypeptide per well (using RNAiMAX as a transfection reagent) and incubated for 24 hours at 37° C.

High content imaging cells to quantitative SHP2 degradation: The cells were then fixed using paraformaldehyde. The levels of SHP2 and HA tag were then probed using antibodies specific for these epitopes and detected using a Cytation 5 High content imaging system. SHP2 levels were normalised to the range 0-100% based on SHP2 levels found within untreated cells within each experiment. Data correspond to n=3 or more biological replicates.

### Results

FIG. 3C shows normalised fluorescence intensity for SHP2 protein for constructs comprising linkers of varying amino acid lengths. The dependence of fusion polypeptide efficacy on the length of the linker between UBE2D1 and the aCS3 binding domain was investigated. In general, shorter linkers (6-20 amino acids in length) consistently showed higher SHP2 degradation activity, as indicated by the lower percentage of normalised SHP2 signal, then longer linkers. However, even longer linkers (e.g. 24-28 amino acids in length) resulted in target degradation activity.

### Conclusion

These data show that all tested lengths of linker resulted in successful target degradation. In addition, different linker compositions were tested for the linkers of length 19 (Linkers 2 and 11) and 28 (Linkers 15-18) amino acids residues long, demonstrating that varying the sequence of the linker does not abrogate target degradation activity. All tested compositions resulted in target degradation. Therefore, target degradation activity can be maintained irrespective of linker length and despite variation in the sequences of the linkers.

### Example 3—Investigating the Effect of Binding Domain Affinity on the Activity of E3 Ligase and E2 Fusion Polypeptides

### Introduction

The aim of this experiment was to investigate the activity of the biological fusion polypeptides, as measured by reduction in target protein levels, using an aCS3 monobody as the binding domain or mutant aCS3 V33R. The fusion polypeptide variants were tested in MDA-MB-231 and U20S cells. Standard aCS3 monobody with a reported high affinity to SHP2 (SHP2 C-SH2 domain Kd=4-9.1 nM) was compared to V33R aCS3 mutant with lower affinity (SHP2 C-SH2 domain Kd=1.2 μM) (Sha et al., Proc Natl Acad Sci USA, 2013 110(37):14924-9 and supplementary information). In the publication by Sha et al., the aCS3 monobody was referred to as CS3. Controls included aCS3 monobody alone, VHL alone, UBE2D1 alone and un-transduced control cells. Fusion polypeptides with N-terminal and C-terminal regulation/degradation domains of UBE2D1 or VHL were tested with either the standard aCS3 binding domain or aCS3 V33R binding domain. All fusion polypeptide constructs tested had a 19 amino acid ‘long’ linker. The degree of target SHP2 degradation was determined by western blot analysis and quantified by densitometry of western blot bands.

### Materials and Methods

Lentiviral particles encoding the following fusion polypeptides (or individual components) were produced in HEK293FT cells: HA_aCS3 (SEQ ID NO: 149), HA_UBE2D1 (SEQ ID NO: 169), HA_UBE2D1 Linker 2 aCS3 (SEQ ID NO: 159), HA_UBE2D1 Linker 2 aCS3(V33R) (SEQ ID NO: 160), HA_aCS3 Linker 2 UBE2D1 (SEQ ID NO: 203), HA_aCS3(V33R) Linker 2 UBE2D1 (SEQ ID NO: 195), HA_VHL (SEQ ID NO: 168), HA_VHL_Linker 2 aCS3 (SEQ ID NO: 153), HA_VHL_Linker 2 aCS3(V33R) (SEQ ID NO: 155), HA_aCS3 Linker 2 VHL (SEQ ID NO: 197), and HA_aCS3(V33R) Linker 2_VHL (SEQ ID NO: 201).

MDA-MB-231 and U20S cells were transduced according to the methods described in ‘transduction of cells with lentivirus’ and prepared for western blot analysis as described in ‘western blot analysis and quantification’ within the main methods section. MDA-MB-231 and U20S un-transduced control (‘cells’) lysates were also included. Western blot analysis of sample lysates was performed using rabbit anti-SHP2 (CST #3397; 1:1000 dilution) with secondary Goat anti-Rabbit IRDye800 (Licor #925-32211; 1:15,000 dilution); and rabbit anti-GAPDH (CST #5174; 1:4,000) with secondary Goat anti-Rabbit IRDye800 (Licor #925-32211; 1:15,000 dilution). Blots were then visualised on the Odyssey system and densitometry of western blot bands was performed using Image Studio software. For each sample, the densitometry values for SHP2 protein bands were divided by the respective densitometry value for the loading control (GAPDH). These values were then provided as a percentage of the SHP2/GAPDH value observed for control (un-transduced) MDA-MB-231 and U20S cells respectively.

### Results

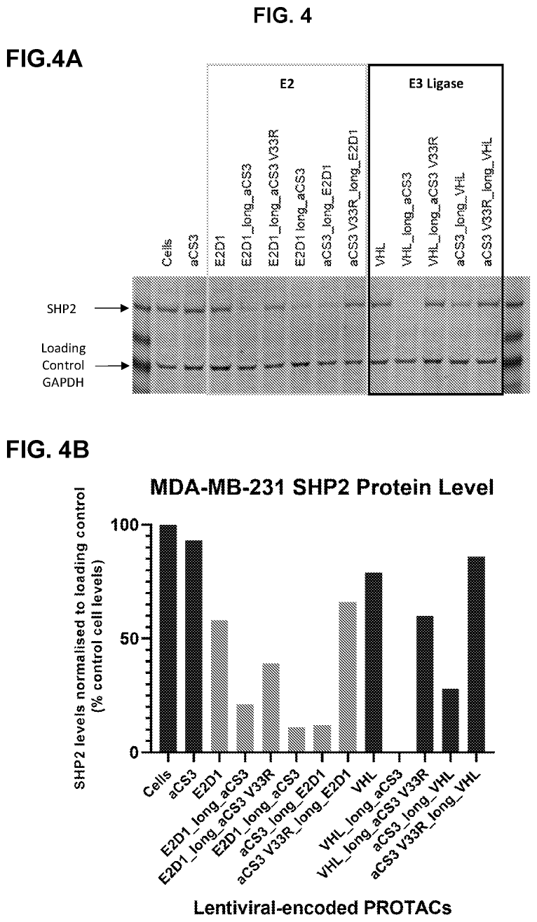

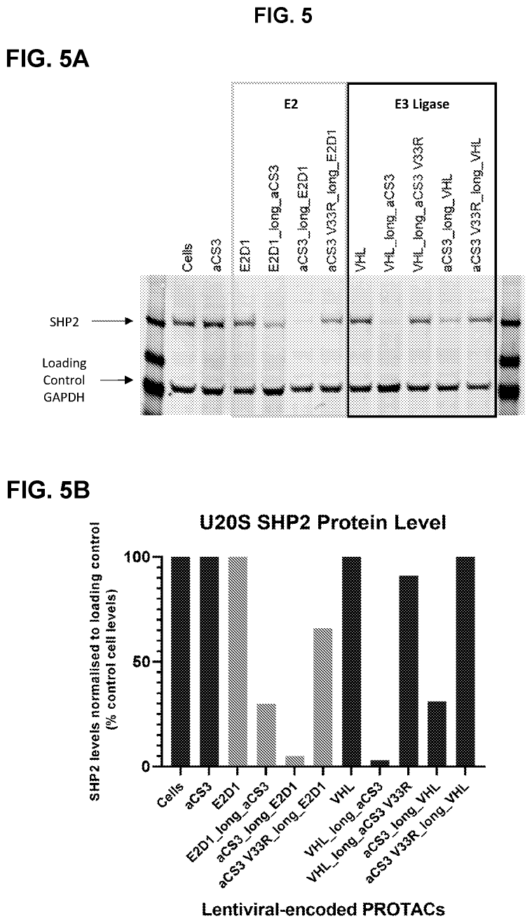

FIGS. 4A and 5A show western blots with SHP2 protein and GAPDH loading control bands. The UBE2D1 ‘regulation/degradation’ domain constructs use the shorter name “E2D1” in FIGS. 4 and 5. In both MDA-MB-231 and U20S, all samples with the standard aCS3 binding domain demonstrated greater reductions in SHP2 protein levels than the mutated, lower affinity variant aCS3(V33R). (FIG. 4 and FIG. 5). In MDA-MB-231 cells, the UBE2D1 (E2D1) fusion polypeptide constructs (in both orientations) with the standard aCS3 binding domain demonstrated approximately 80-90% reductions in SHP2 protein (FIG. 4B). By comparison, the, UBE2D1 (E2D1) fusion polypeptide constructs (in both orientations) with the mutated aCS3(V33R) binding domain demonstrated approximately 35-60% reductions in SHP2 protein (FIG. 4B). These differences were even greater when examining the N-terminal VHL E3 ligase fusion polypeptides, where the SHP2 protein with aCS3 is not detectable. However, with the aCS3(V33R) variant, the reduction of SHP2 protein relative to control cells was 40% (FIG. 4B). In U20S cells (FIG. 5), a similar pattern of results was observed to those described for MDA-MB-231 cells (FIG. 4).

### Conclusion

These data suggest that by reducing the binding affinity of the binding domain, the activity of the fusion polypeptide is reduced and more target protein remains undegraded in the cells. These data suggest that increasing the affinity of the binding domain could increase the amount of target protein degradation. Once again, the data show having an N-terminal VHL E3 ligase ‘degradation’ domain was the most active orientation for the E3 ligase fusion polypeptides tested. The E2 ubiquitin conjugating ‘regulation/degradation’ domain constructs using UBE2D1 and aCS3 demonstrate fairly comparable activity in both orientations tested to date. Some variability was observed between examples which may have been caused by differences in transduction efficiency and lentiviral titre.

### Example 4—Degradation of Endogenous KRas Protein Using an E2 Fusion Polypeptide

### Introduction

The aim of this experiment was to determine if an alternative endogenous target protein could be degraded using a fusion polypeptide comprising an E2 ubiquitin conjugating enzyme as the ‘regulation/degradation’ domain. This was tested in two different cell lines MDA-MB-231 and Ad293 cells. The binding domains of the fusion polypeptide constructs tested were either Designed ankyrin repeat protein (DARPin) K19 or E3_5. K19 binds both GTP- and GDP-bound KRas (Bery et al., Nat Commun. 2019 10(1):2607). E3_5 acted as a negative control unselected DARPin (Binz et al., J Mol Biol, 2003 332(2):489-503). Controls included DARPin E3_5 alone, VHL alone, and un-transduced control cells. All fusion polypeptides comprised an N-terminal ‘binding domain’ of either DARPin K19 or E3_5 and a C-terminal ‘regulation/degradation’ domain of UBE2D1 or VHL. The domains in the constructs were joined by a 20 amino acid linker (‘Linker 3’). The degree of target KRas degradation was determined by western blot analysis and quantified by densitometry of western blot bands.

### Materials and Methods

Lentiviral particles encoding the following fusion polypeptides (or individual components) were produced in HEK293FT cells: HA_VHL (SEQ ID NO: 168), HA_E3_5 (SEQ ID NO: 151), HA_K19 Linker 3 VHL (SEQ ID NO: 198), HA_E3_5 Linker 3 VHL (SEQ ID NO: 199), HA_K19 Linker 3 UBE2D1 (SEQ ID NO: 204), and HA_E3_5 Linker 3 UBE2D1 (SEQ ID NO: 205).

MDA-MB-231 and Ad293 cells were transduced according to the methods described in ‘transduction of cells with lentivirus’ and prepared for western blot analysis as described in ‘western blot analysis and quantification’ within the main methods section. MDA-MB-231 and Ad293 un-transduced control (‘cells’) lysates were also included. Western blot analysis of sample lysates was performed using mouse anti-KRas (LS-Bioscience #LS-C175665; 1:2000 dilution) with secondary Goat anti-mouse IRDye680RD (Licor #926-68070; 1:15,000 dilution); and mouse anti-alpha tubulin (Licor #926-42213; 1:10,000 dilution) with secondary Goat anti-mouse IRDye680RD (Licor #926-68070; 1:15,000 dilution). Blots were then visualised on the Odyssey and densitometry of western blot bands is performed using Image Studio software. For each sample, the densitometry values for KRas protein bands are divided by the respective densitometry value for the loading control (alpha-tubulin). These values are then given as a percentage of the KRas/alpha-tubulin value observed for control (un-transduced) MDA-MB-231 and Ad293 cells respectively.

### Results

Degradation of endogenous KRas by greater than 80% was observed in FIG. 6 western blot in both MDA-MB-231 and Ad293 cells using the E2 ubiquitin conjugating enzyme fusion polypeptide K19_E2D1 (HA_K19 Linker 3 UBE2D1). The UBE2D1 ‘regulation/degradation’ domain constructs use the shorter name “E2D1” in FIG. 6. The negative control fusion polypeptide E3_5_E2D1 (HA_E3_5 Linker 3 UBE2D1) did not result in any KRas degradation in these cells (FIGS. 6A and 6B). In this format, E2 fusion polypeptides (using UBE2D1) were more effective than E3 ligase fusion polypeptide (using VHL) at reducing KRas protein levels in both MDA-MB-231 and Ad293 cells. The K19_VHL E3 ligase fusion polypeptide only demonstrated a reduction in KRas protein levels in MDA-MB-231 cells, but not Ad293 cells.

### Conclusion

These data demonstrate the E2 ubiquitin conjugating enzyme ‘regulation/degradation’ domains are capable of modulating targets other than SHP2. In this case the binding domain (DARPin K19) recruited endogenous KRas, resulting in downstream reductions in KRas protein levels. In the linker and domain orientations tested, the KRas-targeted E2 fusion polypeptides were able to demonstrate activity in both MDA-MB-231 cells and Ad293 cells. Conversely the KRas-targeted E3 fusion polypeptides had some activity in MDA-MB-231 cells but no activity in Ad293 cells.

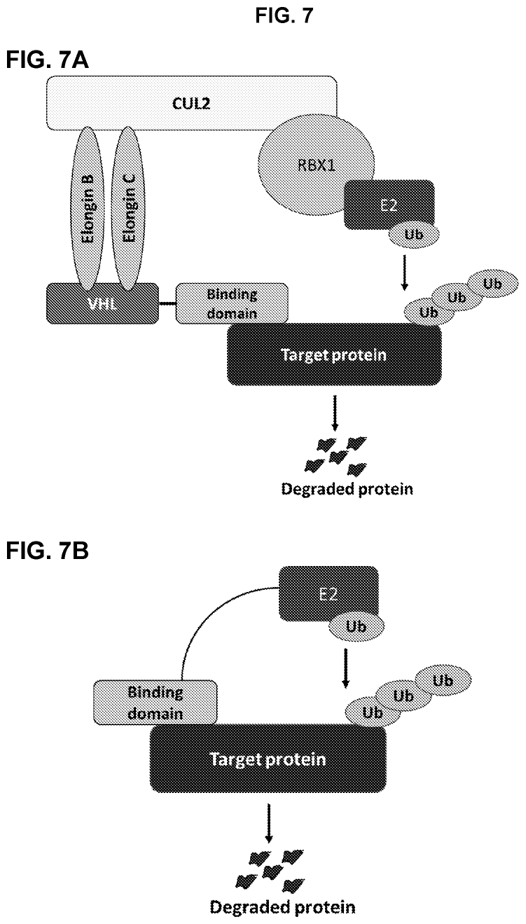

These data suggest either (i) the format tested is sub-optimal for E3 fusion polypeptide activity (as this orientation has previously been less effective for the SHP2-targeted VHL fusion polypeptides; however despite this reduction in SHP2, protein levels were always detected) and/or (ii) the E3 ligase fusion polypeptides may be liable to variable activity depending on cell background, for example, due to the expression levels of certain adaptor proteins required for the EloB/C/CUL2/RBX1 E3 ligase machinery (see FIG. 7). As E2 fusion polypeptides are less reliant on the expression of multiple endogenous proteins to result in target binding and ubiquitin transfer, this is clearly an advantage of using an E2 fusion polypeptide and may allow activity in a larger panel of cell types.

### Example 5A—Investigating a Panel of Core E2 Ubiquitin and Ubiquitin-Like Conjugating Enzymes as ‘Regulation/Degradation’ Domains in SHP2-Targeted Fusion Polypeptide

### Introduction

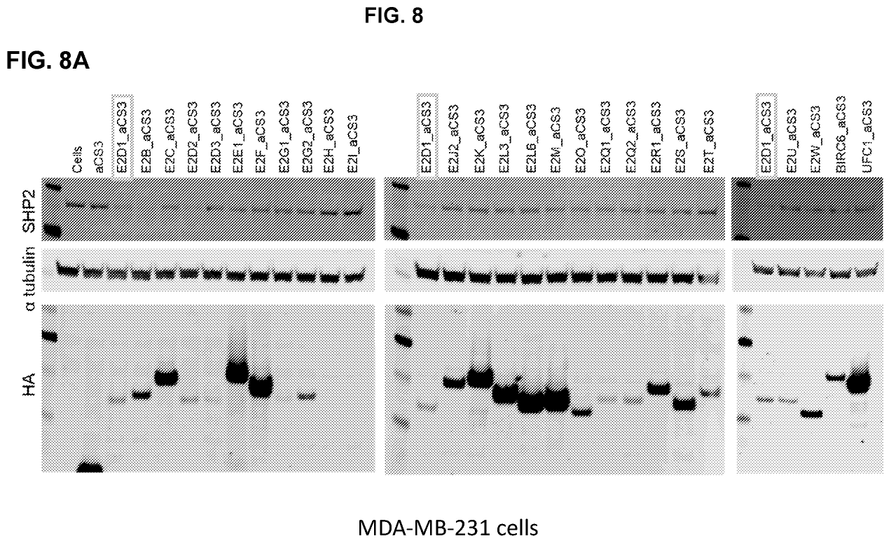

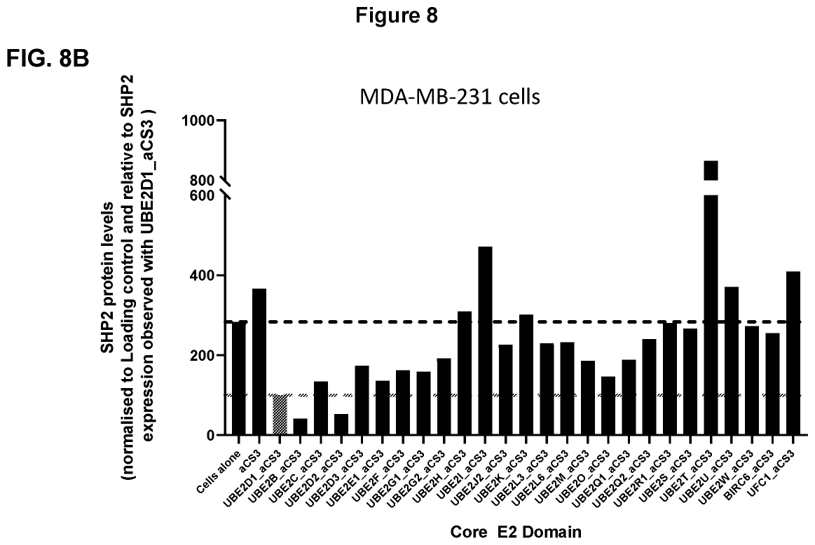

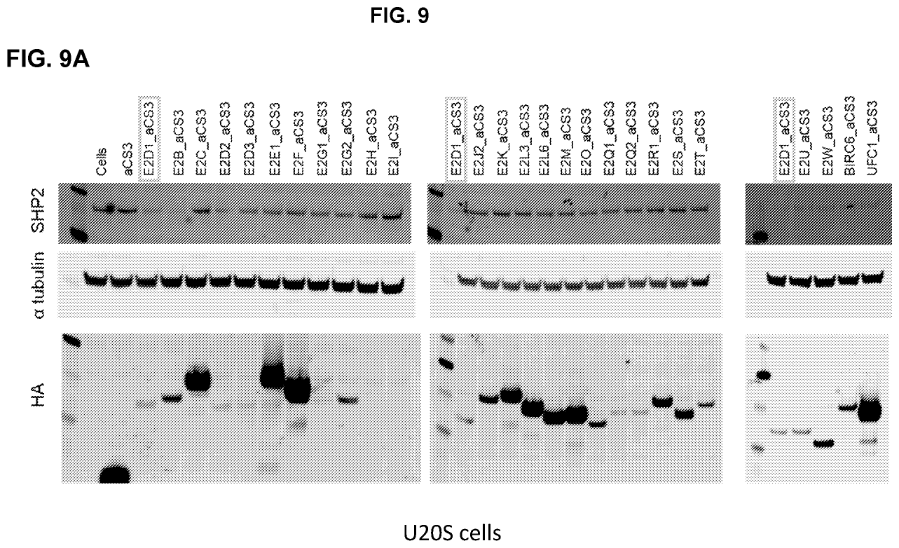

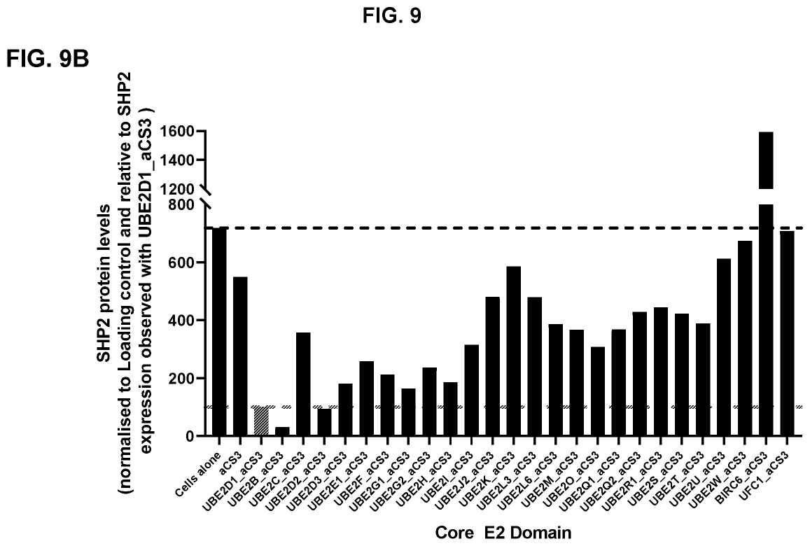

The aim of this experiment was to determine which core E2 ubiquitin or ubiquitin-like conjugating enzyme sequences are able to reduce target protein expression by the greatest extent when expressed in an E2 fusion polypeptide format i.e. core E2_Linker 2_aCS3. 26 different core E2 ubiquitin or ubiquitin-like conjugating enzyme sequences were tested and the expression of the fusion polypeptide constructs, and the resulting SHP2 protein levels were determined by western blot and compared to the E2D1_aCS3 fusion polypeptide used in previous examples. The panel of constructs were tested in MDA-MB-231 and U20S cells. The core E2 domains tested were UBE2D1, UBE2B, UBE2C, UBE2D2, UBE2D3, UBE2E1, UBE2F, UBE2G1, UBE2G2, UBE2H, UBE2I, UBE2J2, UBE2K, UBE2L3, UBEL6, UBE2M, UBE20, UBE2Q1, UBE2Q2, UBE2R1, UBE2S, UBE2T, UBE2U, UBE2W, BIRC6 and UFC1. In western blots, these samples were shown by the shorter nomenclature missing out the first letters ‘UB’ such that UBE2D1 is shown as E2D1 (FIGS. 8A and 9A). Controls included aCS3 monobody alone and un-transduced control cells. The degree of target SHP2 degradation was determined by western blot analysis and quantified by densitometry of western blot bands.

### Materials and Methods

Lentiviral particles encoding the following fusion polypeptides (or individual components) were produced in HEK293FT cells: HA_aCS3 (SEQ ID NO: 149), HA_UBE2D1 Linker 2_aCS3 (SEQ ID NO: 159), HA_UBE2B_Linker 2 aCS3 (SEQ ID NO: 156), HA_UBE2C Linker 2 aCS3 (SEQ ID NO: 157), HA_UBE2D2 Linker 2 aCS3 (SEQ ID NO: 171), HA_UBE2D3 Linker 2 aCS3 (SEQ ID NO: 172), HA_UBE2E1 Linker 2 aCS3 (SEQ ID NO: 173), HA_UBE2F Linker 2 aCS3 (SEQ ID NO: 174), HA_UBE2G1 Linker 2 aCS3 (SEQ ID NO: 175), HA_UBE2G2 Linker 2 aCS3 (SEQ ID NO: 176), HA_UBE2H Linker 2 aCS3 (SEQ ID NO: 177), HA_UBE2I Linker 2 aCS3 (SEQ ID NO: 178), HA_UBE2J2 Linker2 aCS3 (SEQ ID NO: 179), HA_UBE2K Linker2 aCS3 (SEQ ID NO: 180), HA_UBE2L3 Linker2 aCS3 (SEQ ID NO: 181), HA_UBEL6 Linker2 aCS3 (SEQ ID NO: 182), HA_UBE2M Linker 2 aCS3 (SEQ ID NO: 183), HA_UBE20 Linker 2 aCS3 (SEQ ID NO: 184), HA_UBE2Q1 Linker 2 aCS3 (SEQ ID NO: 185), HA_UBE2Q2 Linker 2 aCS3 (SEQ ID NO: 186), HA_UBE2R1 Linker 2 aCS3 (SEQ ID NO: 187), HA_UBE2S Linker 2 aCS3 (SEQ ID NO: 188), HA_UBE2T Linker 2 aCS3 (SEQ ID NO: 189). HA_UBE2U Linker 2 aCS3 (SEQ ID NO: 190), HA_UBE2W Linker 2 aCS3 (SEQ ID NO: 191), HA_BIRC6_Linker 2_aCS3 (SEQ ID NO: 192), and HA_UFC1 Linker 2 aCS3 (SEQ ID NO: 193).

MDA-MB-231 and U20S cells were transduced according to the methods described in ‘transduction of cells with lentivirus’ and prepared for western blot analysis as described in ‘western blot analysis and quantification’ within the main methods section. MDA-MB-231 and U20S un-transduced control (‘cells’) lysates were also included. Western blot analysis of sample lysates was performed using mouse anti-SHP2 (Abcam #ab76285; 1:1000 dilution) with secondary Goat anti-mouse IRDye680RD (Licor #926-68070; 1:15,000 dilution); mouse anti-alpha tubulin (Licor #926-42213; 1:10,000 dilution) with secondary Goat anti-mouse IRDye680RD (Licor #926-68070; 1:15,000 dilution); and rabbit anti-HA tag (Abcam #ab1 37838; 1:1000) with secondary Goat anti-Rabbit IRDye800 (Licor #925-32211; 1:15,000 dilution). Blots were then visualised on the Odyssey system and densitometry of western blot bands was performed using Image Studio software. For each sample, the densitometry values for SHP2 protein bands were divided by the respective densitometry value for the loading control (alpha tubulin). These values are provided as a percentage of the SHP2/alpha tubulin value observed for UBE2D1_aCS3 E2 fusion polypeptide (FIGS. 8B and 9B) to determine if any other core E2 was able to result in a greater effect on target protein levels in MDA-MB-231 and U20S cells respectively.

### Results

In agreement with previous data, degradation of SHP2 protein was observed with HA_UBE2D1 Linker 2 aCS3 (labelled in the figure as “E2D1_aCS3”) in both MDA-MB-231 and U20S cells (FIG. 8 and FIG. 9, respectively). Several of the core E2s tested in this format (N-terminal E2 core domain and C-terminal aCS3 binding domain) resulted in small decreases in SHP2 protein levels as determined by western blot (FIGS. 8A and 9A). Many tested constructs did not appear to reduce SHP2 protein levels. Consistently in both MDA-MB-231 and U20S cells, core HA_UBE2B_Linker 2 aCS3 (labelled in the figure as “E2B_aCS3”) and to a lesser extent HA_UBE2D2 Linker 2 aCS3 (labelled in the figure as “E2D2_aCS3”) reduced SHP2 protein levels to a greater extent than HA_UBED1 Linker 2 aCS3 when the western blot band densitometry was quantified and normalised to the loading control (FIGS. 8B and 9B, respectively). The HA western blot indicates the relative expression levels and/or stability of these HA-tagged fusion polypeptide constructs. Both HA_UBE2D1 Linker 2 aCS3 and HA_UBE2D2 Linker 2 aCS3 showed minimal HA bands, indicating poor construct expression or poor stability of the expressed constructs in the cells. Whereas in both cell lines, the HA_UBE2B_Linker 2 aCS3 construct demonstrated higher HA-tagged protein expression levels than HA_UBE2D1_Linker 2_aCS3 and HA_UBE2D2 Linker 2 aCS3 (FIGS. 8A and 9A). A large proportion of the tested constructs with a diverse array of core E2 ‘regulation/degradation’ domains demonstrated high HA band intensities by western blot indicting high levels of construct expression and/or stability inside the cell.

### Conclusion

These data show that for target regulation resulting in decreased cellular expression levels of target proteins, using HA_UBE2B_Linker 2 aCS3, HA_UBE2D1 Linker 2 aCS3 and HA_UBE2D2 Linker 2 aCS3 constructs resulted in the lowest SHP2 protein levels in both MDA-MB-231 cells. Many of the other core E2 constructs only decreased target expression minimally, if at all. For E2 ubiquitin conjugating enzyme core domain fusion polypeptides, target degradation is considered to be by a ubiquitin-mediated mechanism involving target ubiquitination, polyubiquitination and finally proteasomal degradation. E2 ubiquitin-like conjugating enzyme core fusion polypeptides (e.g. HA_UBE2F Linker 2 aCS3, HA_UBE2I Linker 2 aCS3 and HA_UBE2M Linker 2 aCS3) may post-translationally modify or regulate a target protein in other ways, for example the transfer of ubiquitin-like molecules in the absence of ubiquitin transfer itself.

### Example 5B—Comparing Core E2 Ubiquitin and Ubiquitin-Like Conjugating Enzymes as ‘Regulation/Degradation’ Domains in K19-Targeted Fusion Polypeptide

### Introduction

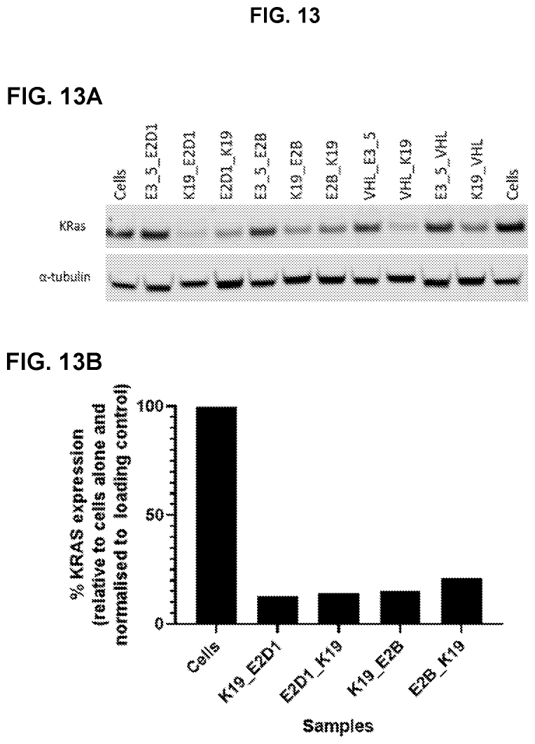

Following the determination that UBE2D1 (E2D1) functions in either orientation for the degradation of different endogenous target proteins (e.g. SHP2 and K19), the aim of this experiment was to determine whether a different core E2 enzyme, UBE2B, is also able to reduce target protein expression irrespective of orientation. The constructs were tested in U20S cells. The core E2 domains tested were UBE2D1 (as a positive control) and UBE2B. In western blots, these samples were shown by the shorter nomenclature missing out the first letters ‘UB’ such that UBE2D1 is shown as E2D1 (FIG. 13). Controls included un-transduced control cells. The degree of target K19 degradation was determined by western blot analysis and quantified by densitometry of western blot bands.

### Materials and Methods

Lentiviral particles encoding the following fusion polypeptides were produced in HEK293FT cells: HA_K19_Linker 2_UBE2D1 (SEQ ID NO: 253), HA_UBE2D1_Linker 2_K19 (SEQ ID NO: 254), HA_K19_Linker 2_UBE2B (SEQ ID NO: 255), and HA_UBE2B_Linker 2_K19 (SEQ ID NO: 256). PROTACs containing the following degradation domains were investigated; UBE2D1 (E2D1), UBE2B (E2B) and VHL. KRas targeted PROTACs were tested in both “Binding domain_Degradation domain” and “Degradation domain_Binding domain” orientations. Negative control DARPin E3_5 was used as a negative control binding domain in combination with the various degradation domains in both orientations (SEQ ID NO: 274-276 and 278). Fusion polypeptides with E3 Degradation domains were also included as controls, as follows: HA_VHL_Linker 2_K19 (SEQ ID NO: 277), and HA_K19_Linker 2_VHL (SEQ ID NO: 279).

HPAC pancreatic cancer cells were transduced according to the methods described in ‘transduction of cells with lentivirus’ and prepared for western blot analysis as described in ‘western blot analysis and quantification’ within the main methods section. HPAC un-transduced control (‘cells’) lysates were also included. Western blot analysis of sample lysates was performed using mouse anti-KRas (LS-Bioscience #LS-C175665; 1:2000 dilution) with secondary Goat anti-mouse IRDye680RD (Licor #926-68070; 1:15,000 dilution); and mouse anti-alpha tubulin (Licor #926-42213; 1:10,000 dilution) with secondary Goat anti-mouse IRDye680RD (Licor #926-68070; 1:15,000 dilution). Blots were then visualised on the Odyssey and densitometry of western blot bands is performed using Image Studio software. For each sample, the densitometry values for KRas protein bands are divided by the respective densitometry value for the loading control (alpha-tubulin). These values are then given as a percentage of the KRas/alpha-tubulin value observed for control (un-transduced) HPAC cells.

### Results

FIG. 13 shows western blots with K19 protein and α-tubulin loading control bands. The UBE2D1 and UBE2B ‘regulation/degradation’ domain constructs have used the shorter name E2D1 and E2B in FIG. 13. In HPAC cells, both orientations of the UBE2D1 (E2D1) fusion polypeptide constructs resulted in reduction in K19 protein levels relative to controls (FIG. 13A). Similarly, both orientations of the VHL fusion polypeptide constructs resulted in reduction in K19 protein levels relative to controls, albeit with the K19_VHL orientation demonstrating a lower level of reduction. These data correlate with that seen in other cell lines, demonstrating that HPAC pancreatic cancer cells are an additional valid model for investigating PROTAC activity. Turning to FIG. 13B, UBE2B (E2B) fusion polypeptide constructs (in both orientations) are compared with UBE2D1 (E2D1) fusion polypeptide constructs (in both orientations), all of which resulted in 70-90% reduction in K19 protein levels (K19_E2D1 87% reduction; E2D1_K19 86% reduction; K19_E2B 85% reduction; and E2B_K19 79% reduction).

### Conclusion

These data show that using the UBE2B degradation domain can result in degradation of KRas protein expression, as well as degradation of SHP2 expression in the alternative construct of Example 5A. Furthermore, both orientations of the PROTAC fusion polypeptides are capable of resulting in target degradation. Altogether, these data demonstrate that multiple E2 ubiquitin or ubiquitin-like conjugating domains fused to multiple target domains result in functional PROTACs irrespective of the orientation for these domains in the fusion polypeptides.

### Example 6A—Mutating the Lysine Residues in aCS3 Binding Domain to Determine if this Will Improve Fusion Polypeptide Activity and Stability in Cells

### Introduction

The aim of this experiment was to determine whether the three lysine residues present in the aCS3 monobody (K7, K55 and K64) ‘binding’ domain within a fusion polypeptide are liable to self-ubiquitination. If these lysine residues are ubiquitinated, this could result in fusion polypeptide degradation, poor stability and reduced activity in cells. The lysine residues were mutated individually and in combination as part of a UBE2D1_aCS3 construct. Structural modelling indicated which amino acid residue changes should retain monobody stability. Lysine residue K7 was mutated to glutamine (K7Q). Lysine residue K55 was mutated to tyrosine (K55Y) and lysine residue K64 was mutated to histidine (K64H). The effects on SHP2 degradation and fusion polypeptide expression in U20S cells expressing fusion polypeptides containing these aCS3 variants was measured by western blot probing for SHP2 protein and HA tag expression levels, respectively. Alpha-tubulin expression levels were determined by western blot as a loading control. Control samples included aCS3 monobody alone, UBE2D1_aCS3 (WT) and un-transduced control cells. The degree of target SHP2 degradation was determined by western blot analysis and quantified by densitometry of western blot bands.

### Materials and Methods

Lentiviral particles encoding the following fusion polypeptides (or individual components) were produced in HEK293FT cells: HA_aCS3 (SEQ ID NO: 149), UBE2D1 Linker2 aCS3 (SEQ ID NO: 159), UBE2D1 Linker 2 aCS3 (K7Q) (SEQ ID NO: 161), UBE2D1 Linker 2 aCS3 (K55Y) (SEQ ID NO: 162), UBE2D1 Linker 2 aCS3 (K64H) (SEQ ID NO: 163), UBE2D1 Linker 2 aCS3 (K7Q, K55Y) (SEQ ID NO: 164), UBE2D1 Linker 2 aCS3 (K7Q, K64H) (SEQ ID NO: 165), UBE2D1 Linker2 aCS3 (K55Y, K64H) (SEQ ID NO: 166), and UBE2D1 Linker 2 aCS3 (K7Q, K55Y, K64H) (SEQ ID NO: 167).

U20S cells were transduced according to the methods described in ‘transduction of cells with lentivirus’ and prepared for western blot analysis as described in ‘western blot analysis and quantification’ within the main methods section. U20S un-transduced control (‘cells’) lysates were also included. Western blot analysis of sample lysates was performed using mouse anti-SHP2 (Abcam #ab76285; 1:1000 dilution) with secondary Goat anti-mouse IRDye680RD (Licor #926-68070; 1:15,000 dilution); mouse anti-alpha tubulin (Licor #926-42213; 1:10,000 dilution) with secondary Goat anti-mouse IRDye680RD (Licor #926-68070; 1:15,000 dilution); and rabbit anti-HA tag (Abcam #ab137838; 1:1000) with secondary Goat anti-Rabbit IRDye800 (Licor #925-32211; 1:15,000 dilution). Blots were then visualised on the Odyssey system and densitometry of western blot bands was performed using Image Studio software. For each sample, the densitometry values for SHP2 protein bands were divided by the respective densitometry value for the loading control (alpha tubulin). These values are provided as a percentage of the SHP2/alpha tubulin value observed for U20S control cells. In addition, for each sample, the densitometry values for HA-tagged protein bands were divided by the respective densitometry value for the loading control (alpha tubulin). These values are provided as a percentage of the HA/alpha tubulin value observed for UBE2D1_aCS3 (WT) to determine if fusion polypeptide protein expression in cells could be improved by removing binding domain lysine residues.

### Results

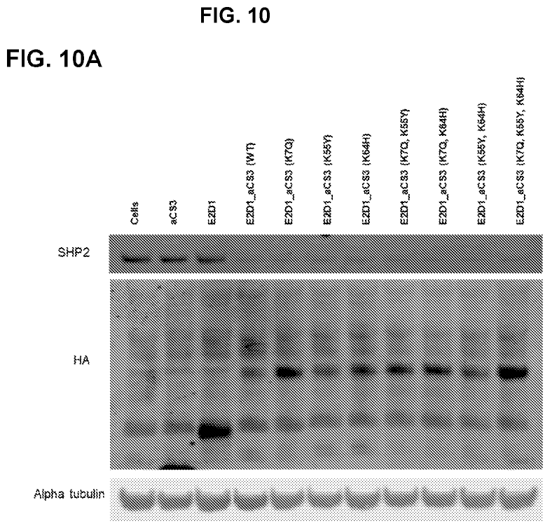

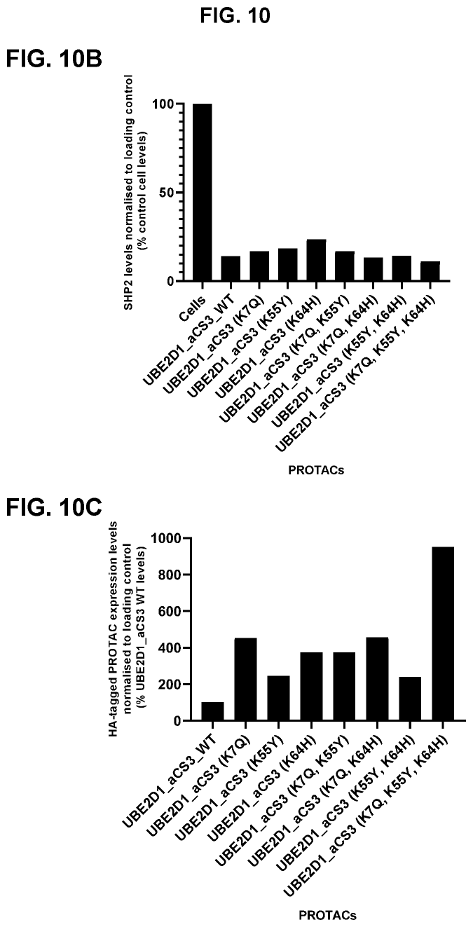

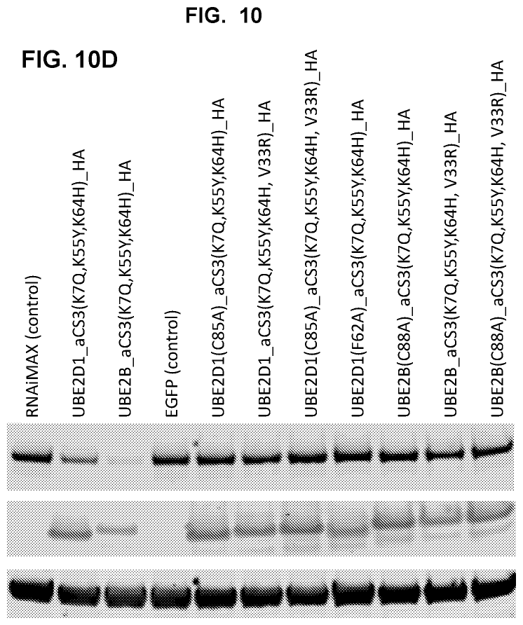

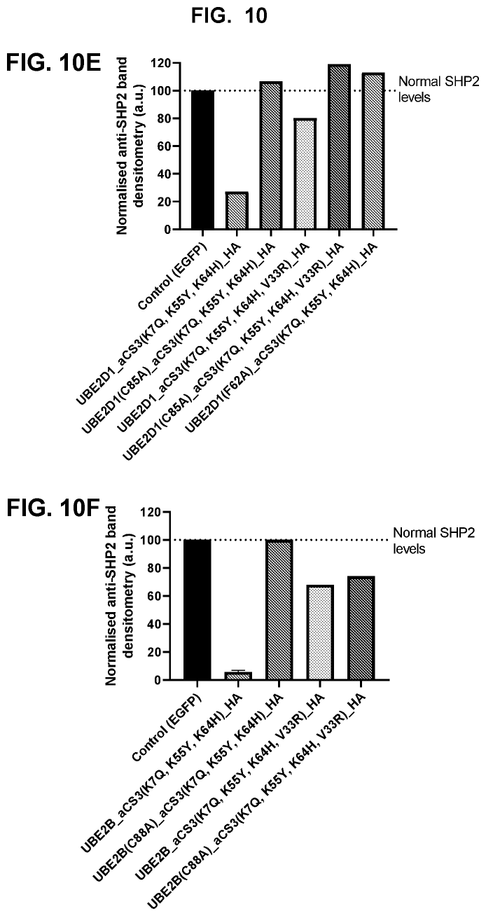

These results show that all HA_UBE2D1 Linker 2 aCS3 (labelled in the figure as “E2D1_aCS3”) samples (wildtype and lysine mutants) were able to result in at least a 75% reduction in SHP2 protein expression (FIGS. 10A and 10B). These results appear comparable between all variants tested. All the lysine mutated variants also demonstrated increased HA expression levels relative to the wildtype aCS3 variant (FIG. 10C). The greatest increases in HA expression appeared to involve the mutation of the lysine at position 7 (K7Q) in isolation or in combination with other lysine mutations, with the highest levels observed for the triple mutant (K7Q, K55Y, K64H; FIG. 10C).

### Conclusions

These data show that the removal of the lysine residues from the aCS3 monobody sequence appeared to increase the level of fusion polypeptide expression in cells while not negatively impacting the extent of target SHP2 degradation in cells. The key residue that appeared to increase HA expression while maintaining the fusion polypeptide's ability to interact with target SHP2 was K7. All variants including this mutation, appeared to show an improved stability and activity profile compared to HA_UBE2D1 Linker 2 aCS3 WT. The triple mutant (K7Q, K55Y, K64H) demonstrated the greatest increase in HA expression and target degradation. These data suggest that, within the E2 fusion polypeptide construct, lysine residues represent a liability for self-ubiquitination which can be resolved by replacing these with alternative residues. In this case, an in-house structural modelling study was performed to select the best mutations to maintain aCS3 monobody stability. These data show that activity appears to be maintained as target SHP2 degradation is comparable or increased in the variants tested.

### Example 6B—Mutating the Catalytic Site of UBE2D1 or UBE2B Regulation Domains of Fusion Polypeptides or Reducing the Binding Domain Affinity to the Target Protein Reduces Target Protein Degradation

### Introduction

The aim of this experiment was to further explore the ability of point mutations to alter the activity of fusion polypeptides. These experiments aimed to determine whether the three lysine residues present in the aCS3 monobody (K7, K55 and K64) ‘binding’ domain within a fusion polypeptide are liable to self-ubiquitination in UBE2D1 and UBE2B, which have different cysteine catalytic sites. These mutants were also tested with the V33R mutation of aCS3 to reduce the affinity of the binding domain for target protein SHP2. Finally, UBE2D1 was further mutated at residue F62, which is involved with interactions with some E3 ligases, to determine its effects on activity.

### Materials and Methods

mRNA synthesis: Linear DNA templates encoding SHP2 targeted fusion polypeptides with various point mutations in Binding Domains and Regulation Domains and consisting of a T7 promoter, a 5′ UTR, a fusion polypeptide-encoding open reading frame, a 3′ UTR and a polyA tail, were used for in vitro transcription of mRNA as described elsewhere (Vaidyanathan S, et al., Uridine Depletion and Chemical Modification Increase Cas9 mRNA Activity and Reduce Immunogenicity without HPLC Purification. Mol Ther Nucleic Acids 12, 530-542 (2018)).

The mRNA sequences used encode the following fusion polypeptides:

(SEQ ID NO: 240)
UBE2D1_Linker2_aCS3(K7Q, K55Y, K64H)_HA,
(SEQ ID NO: 266)
UBE2D1(C85A)_Linker2_aCS3(K7Q, K55Y, K64H)_HA,
(SEQ ID NO: 267)
UBE2D1_Linker2_aCS3(K7Q, K55Y, K64H, V33R)_HA,
(SEQ ID NO: 268)
UBE2D1(C85A)_Linker2_aCS3(K7Q, K55Y, K64H,
V33R)_HA,
(SEQ ID NO: 269)
UBE2D1(F62A)_Linker2_aCS3(K7Q, K55Y, K64H)_HA,
(SEQ ID NO: 270)
UBE2B_Linker2_aCS3(K7Q, K55Y, K64H)_HA,
(SEQ ID NO: 271)
UBE2B(C88A) Linker2_aCS3(K7Q, K55Y, K64H)_HA,
(SEQ ID NO: 272)
UBE2B_Linker2_aCS3(K7Q, K55Y, K64H, V33R)_HA,
and
(SEQ ID NO: 273)
UBE2B(C88A) Linker2_aCS3(K7Q, K55Y, K64H,
V33R)_HA.

Transfection of cells with mRNA: U20S cells were transfected with mRNA using RNAiMAX (Invitrogen) according to manufacturer's instructions. 3.5×10⁵ U20S cells per well were seeded into 6 well plates and incubated for 24 hours at 37° C. Cells were then transfected with 3 μg of each mRNA encoded fusion polypeptide per well (using RNAiMAX as a transfection reagent) and incubated for 24 hours at 37° C.

Western blot analysis and quantification: Media was removed from the cells, prior to washing with PBS. Cells were harvested using accutase (Sigma) and incubating at 37° C. for 3 minutes. The accutase was then neutralised with the addition of complete media. The cell suspension was then collected and centrifuged at 1200 rpm (300×g) for 5 minutes to pellet cells. The cell pellets were washed in PBS and transferred to 1.5 mL Eppendorf tubes. These tubes were then centrifuged at 1200 rpm (300×g) for 5 minutes and the supernatant discarded. The cell pellets are lysed in RIPA Lysis buffer (Thermo Fisher Scientific) containing a 1:100 dilution of protease and phosphatase inhibitor cocktail (Cell Signalling Technology). Lysates were incubated on ice for 30 minutes prior to clarifying, by centrifuging at 15,000 rpm (17,000×g) for 10 minutes at 4° C. The protein concentration of each lysate was determined by BCA assay (Pierce/Thermo Fisher Scientific according to manufacturer's instructions). 40 μg lysate from each cell line then loaded per well on 4-12% BOLT gels (Thermo Fisher Scientific) and run at 200V for 25 minutes prior to transferring to membrane using the iBlot according to manufacturer's instructions (Thermo Fisher Scientific). Membranes were then blocked in Odyssey blocking buffer (Li-cor) and western blot analysis performed using appropriate antibodies (see Table 2 below).

Blots were then visualised on the Odyssey system according to manufacturer's instructions (Li-cor) and densitometry of western blot bands measured using Image Studio software according to manufacturer's instructions (Li-cor).

### Results

To further explore the ability of point mutations to alter the activity of fusion polypeptides, a panel of mutant Binding domain and Regulation domain fusion polypeptides were examined. U20S cells were transfected with mRNA encoding a panel of variant fusion polypeptides targeting SHP2 protein for ubiquitination and subsequent degradation. Cells were transfected using RNAiMAX, incubated for 24 hours and harvested and SHP2 protein levels analysed by Western blot. The Binding domains for the fusion polypeptide variants used in this example contained K7Q, K55Y and K64H point mutations to increase fusion polypeptide expression relative to the non-mutated aCS3 Binding domain (as shown in FIG. 10C).

The additional point mutations explored included:
(i) mutating the catalytic cysteine residue of regulation domains, e.g. UBE2D1 (C85A) and UBE2B (C88A), which resulted in rescuing the SHP2 protein levels to normal (or near normal) by inactivating the catalytic reside of the Regulation domain (see FIGS. 10D, 10E and 10F);
(ii) reducing the affinity of the binding domain for target protein SHP2 by including an additional V33R mutation of aCS3, which resulted in rescuing the SHP2 protein levels to closer to normal expression levels. The SHP2 expression levels were not completely rescued. This may be because the V33R point mutation of aCS3 does not completely abolish target binding but does reduce affinity by approximately 100-fold (Sha et al., Proc. Natl. Acad. Sci. USA, 2013 110(37):14924-9 and supplementary information; see FIGS. 10D, 10E and 10F); and
(iii) mutating UBE2D1 residue F62, involved in interactions with E3 ligases, to determine effects on activity (i.e. F62A), which resulted in a complete rescue of SHP2 protein.

### Conclusion

These data show that by mutating the catalytic cysteine of the E2 Regulation domain (e.g. UBE2D1 or UBE2B) to alanine, the Regulation domain is inactivated and target protein modifications (in this case, ubiquitination and degradation of SHP2) are inhibited. Additionally, by reducing the affinity of the Binding domain to the target protein, the degree of target protein modification is also reduced. In this example, the V33R mutation of aCS3 reduced binding affinity to SHP2 by approximately 100-fold (Sha et al., Proc. Natl. Acad. Sci. USA, 2013 110(37):14924-9 and supplementary information).

Finally, these data suggest that the interaction of an E3 ligase with the E2 Regulation Domain may be key for Regulation domain activity, as the F62A mutation of UBE2D1 appears to abrogate SHP2 degradation. Residue F62 has been shown by structural studies to be involved in interactions between UBE2D1 and E3 ligase RNF4 (Gundogdu and Walden, Protein Science. 2019; 28:1758-1770), which may be involved in catalysing ubiquitination. Mutation of this residue to alanine was hypothesized to prevent E3 interaction with UBE2D1, which appears to prevent the fusion polypeptide from resulting in target SHP2 degradation.

### Example 7—Degradation of a Nuclear Target Using E2 Ubiquitin Conjugating Enzyme Fusion Polypeptides

### Introduction

The aim of this experiment was to determine whether the E2 fusion polypeptide format could be successful in degrading a predominantly nuclear target. Human antigen R was the selected target as single domain antibody/nanobody sequences were available to this target. Human antigen R (HuR/ELAVL1) is an RNA binding protein involved in the stabilisation and translational upregulation of target mRNAs. HuR is predominantly localized in the nucleus but, in response to different stimuli, is exported to the cytoplasm, a process modulated by several posttranslational modifications, which also affect its binding to target mRNAs (Doller et al., Cell Signal., 2008 20:2165-2173). Two separate nanobody sequences were selected for this experiment: HuR8 and HuR17. The binding affinity of HuR8 for target Human antigen R is 2100 nM and that of HuR17 is 30 nM. The control Cas9 V_{HH} nanobody binding domain targeting Cas9 protein was included. Cas9 is a bacterial protein, and therefore is not endogenously expressed in mammalian cells. Hence, the Cas9 V_{HH} nanobody should not selectively bind to any proteins in mammalian cells. These three V_{HH} nanobodies were cloned into UBE2D1 fusions in both orientations: (UB)E2D1_Linker_V_{HH} and V_{HH}_Linker (UB)E2D1. The linker used was the 19 amino acid linker 2 (SEQ ID NO: 142). Lentiviral particles encoding these constructs were transduced into 2 different cell lines (MDA-MB-231 and U20S) and the resulting effects on HuR expression studied by western blot analysis.

### Materials and Methods

Lentiviral particles encoding the following fusion polypeptides (or individual components) were produced in HEK293FT cells: HA_UBE2D1 Linker 2 Cas9. HA_UBE2D1 Linker 2 HuR8, HA_UBE2D1 Linker2 HuR17, HA_Cas9 Linker2 UBE2D1, HA_HuR8 Linker 2 UBE2D1, and HA_HuR17 Linker 2 UBE2D1.

MDA-MB-231 and U20S cells were transduced according to the methods described in ‘transduction of cells with lentivirus’ and prepared for western blot analysis as described in ‘western blot analysis and quantification’ within the main methods section. Western blot analysis of sample lysates was performed using rabbit anti-HuR/ELAVL1 (CST #12582; 1:1000 dilution) with secondary Goat anti-Rabbit IRDye800 (Licor #925-32211; 1:15,000 dilution); and mouse anti-alpha tubulin (Licor #926-42213; 1:10,000 dilution) with secondary Goat anti-mouse IRDye680RD (Licor #926-68070; 1:15,000 dilution. Blots were then visualised on the Odyssey system and densitometry of western blot bands was performed using Image Studio software. For each sample, the densitometry values for HuR protein bands were divided by the respective densitometry value for the loading control (alpha tubulin). These values are then provided as a percentage of the HuR/alpha tubulin value observed for each respective control lysate (e.g. HA_Cas9 Linker 2 UBE2D1 or HA_UBE2D1 Linker 2 Cas9 depending on the fusion protein orientation tested).

### Results

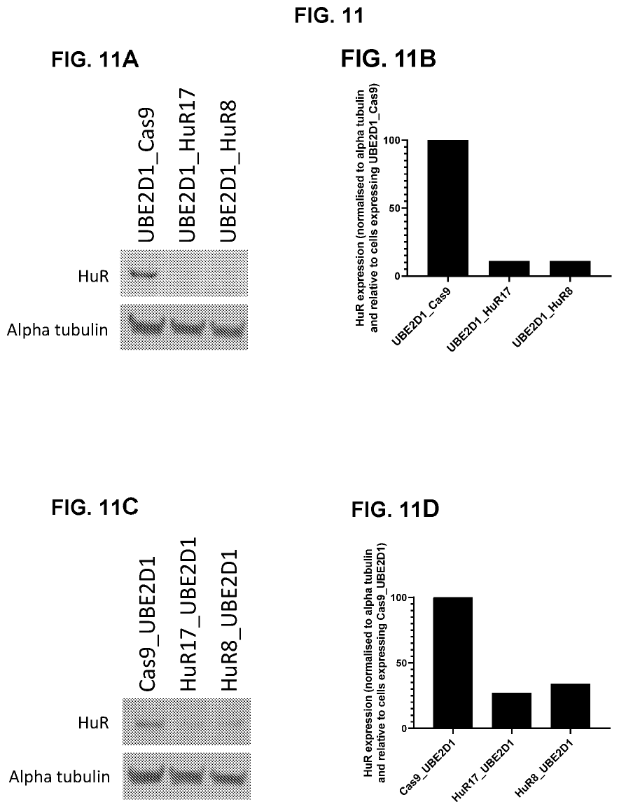

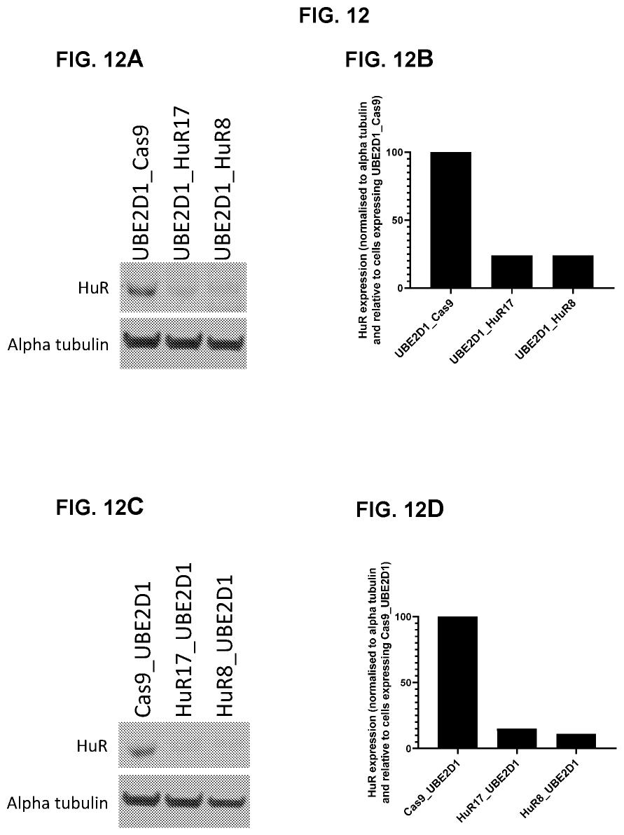

These results show that HuR protein expression was reduced, relative to control levels, in MDA-MD-231 and U20S cell lines expressing HA_UBE2D1 Linker 2 HuR17 (labelled in the figure as “UBE2D1_HuR17”), HA_UBE2D1 Linker 2 HuR8 (labelled in the figure as “UBE2D1_HuR8”), HA_HuR17 Linker 2 UBE2D1 (labelled in the figure as “HuR17_UBE2D1”) and HA_HuR8 Linker 2 UBE2D1 (labelled in the figure as “HuR8_UBE2D1”) fusion proteins (FIGS. 11 and 12). In certain examples, the HuR levels were reduced by as much as 90% compared to control levels observed for cells expressing HA_UBE2D1 Linker 2 Cas9 (labelled in the figure as “UBE2D1_Cas9”) and HA_Cas9 Linker 2 UBE2D1 (labelled in the figure as “Cas9_UBE2D1”) (FIGS. 11B and 12D respectively).

### Conclusions

These data show that UBE2D1 fusion constructs containing V_{HH} single domain antibody (nanobody) binding domains can successfully degrade target HuR (a predominantly nuclear target). The quantification of target HuR degradation shown in FIGS. 11B, 11D, 12B, and 12D suggest 65-90% of HuR protein was degraded. This implies that nuclear HuR would be included in the degraded fraction.

### Materials and Methods of Examples 1-7

### Generation of Lentiviral Particles

HEK293FT cells were seeded into T25 flasks at 5×10⁵ cells per flask or into 6 well plates at 1×10⁵ cells per well in complete media comprising: Dulbecco's modified Eagle's medium (Invitrogen) supplemented with 10% v/v heat inactivated and gamma-irradiated foetal bovine serum (FBS; SAFC), 1% v/v sodium pyruvate (×100; Sigma), 1% v/v non-essential amino acids (×100; Invitrogen), 1% v/v Glutamax-1 (×100; Invitrogen) and Geneticin (G418) (final concentration 0.35 mg/mL; Invitrogen). Cells were incubated at 37° C. and 5% CO2 for 3 days to allow adherence and 80% confluence. Following this incubation period, media was removed and replaced with Complete media in the absence of Geneticin. For each lentivirus generation the following was prepared:

TABLE 1
Reagent volumes used in lentivirus generation

| | | | | | pPACKH1 | Gene of | |
|---|---|---|---|---|---|---|---|
| | | | Dilution | | DNA | interest | |
| | | Plating | media | | @ 0.5 μg/ | plasmid | Lipofectamine |
| Vessel | Area | media | (OptiMEM) | | mL | DNA | 2000 |
| T25 | 25 cm² | 8 mL | 2 × 0.5 | mL | 7.5 μl | 0.75 μg | 25 μl |
| | | | | | (3.75 μg) | | |
| 6-well | 9 cm² | 3 mL | 2 × 180 | μl | 2.7 μl | 0.27 μg | 9 μl |
| plate | | | | | (1.35 μg) | | |

Depending on the scale of lentivirus production, different reagent volumes were used. Refer to table above for more details. One volume of diluting media (OptiMEM; Invitrogen) was combined with pPACKH1 DNA (Cambridge Bioscience) and gene of interest plasmid DNA (in pCDH_puro lentiviral plasmid vector). For each transfection, a second volume of OptiMEM was mixed with Lipofectamine 2000 (Invitrogen) for 5 minutes at room temperature. The diluted plasmid mix was then combined with the diluted Lipofectamine 2000 mix and incubated at room temperature for 20 minutes prior to addition the HEK293FT cells and incubation at 37° C. and 5% CO₂ for 48 hours. Following this incubation period, the supernatants from each sample of cells was collected and the presence of lentiviral particles confirmed using a Lenti-X™ GoStix™ Plus according to manufacturer's instructions (Takara Bio). Supernatants containing lentiviral particles were then filtered through 0.22 μm pore filters in sterile Steriflip (Millipore) tubes prior to use.

Transduction of Cells with Lentivirus

Ad293, MDA-MB-231, U20S, HCT116, HeLa and HPAC cells are grown in 6-well plates in 2 mL of appropriate media to achieve approximately 60-80% confluence. Prior to lentiviral transduction all media was removed and replaced with 2 mL of RPMI (Invitrogen) with 10% FBS, containing 16 μg/mL polybrene (final concentration 8 μg/mL; Sigma-Aldrich). 2 mL of supernatant containing lentiviral particles generated as described above was added to each well. Cells were then incubated at 37° C. and 5% CO₂ for 24 hours prior to replacing the media with fresh complete media for each cell type. Cells were then incubated at 37° C. and 5% CO₂ for a further 24 hours prior to the addition of selection antibiotic (puromycin; Thermo-Fisher Scientific). Puromycin was added at 2 μg/mL for Ad293, MDA-MB-231,U20S and HPAC cells; 4 μg/mL for HCT116 cells and 10 μg/mL HeLa cells). The cells were maintained in the relevant media containing antibiotics, at 37° C., 5% CO₂ until sufficient cells could be harvested for western blot analysis. The cell samples comprised of a pool of transduced cells.

### Western Blot Analysis and Quantification

Media was removed from the transduced cells, prior to washing with PBS. Cells were harvested using accutase (Sigma) and incubating at 37° C. for 3 minutes. The accutase was then neutralised with the addition of complete media. The cell suspension was then collected and centrifuged at 1200 rpm (300×g) for 5 minutes to pellet cells. The cell pellets were washed in PBS and transferred to 1.5 mL Eppendorf tubes. These tubes were then centrifuged at 1200 rpm (300×g) for 5 minutes and the supernatant discarded. The cell pellets are lysed in RIPA Lysis buffer (Thermo Fisher Scientific) containing a 1:100 dilution of protease and phosphatase inhibitor cocktail (Cell Signalling Technology). Lysates were incubated on ice for 30 minutes prior to clarifying, by centrifuging at 10,000 rpm (17,000×g) for 10 minutes at 4° C. The supernatant was collected in a fresh 1.5 mL Eppendorf tube and stored at −80° C. The protein concentration of each lysate was determined by BCA assay (Pierce/Thermo Fisher Scientific according to manufacturer's instructions). 40 μg lysate from each cell line then loaded per well on 4-12% BOLT gels (Thermo Fisher Scientific) and run at 200V for 25 minutes prior to transferring to membrane using the iBlot according to manufacturer's instructions (Thermo Fisher Scientific). Membranes were then blocked in Odyssey blocking buffer (Li-cor) and western blot analysis performed using appropriate antibodies (see Table 2 below):

TABLE 2
Antibody suppliers and dilutions

| | | Catalogue | |
|---|---|---|---|
| Antibody | Supplier | number | Dilution |
| Anti-KRas | LS Bioscience | # LS-C175665 | 1:2,000 |
| Anti-SHP2 | CST | # 3397 | 1:1,000 |
| Anti-SHP2 | Abcam | # ab76285 | 1:1,000 |
| Anti-alpha-tubulin | Li-cor | # 926-42213 | 1:10,000 |
| Anti-GAPDH (D16H11) | CST | #5174S | 1:4,000 |
| Anti-HA tag | Abcam | # ab137838 | 1:1,000 |
| Anti-HuR/ELAVL1 | CST | #12582 | 1:1,000 |
| Goat anti-mouse | Li-cor | # 926-68070 | 1:15,000 |
| IRDye680RD | | | |
| Goat anti-Rabbit | Li-cor | # 925-32211 | 1:15,000 |
| IRDye800 | | | |

Blots were then visualised on the Odyssey system according to manufacturer's instructions (Li-cor) and densitometry of western blot bands measured using Image Studio software according to manufacturer's instructions (Li-cor).

TABLE 3
E2 ubiquitinating enzymes, substrates, alternative
names and UniProt accession numbers

| E2 ubiquitin- | | | UniProt |
|---|---|---|---|
| conjugating | Substrate (Ubiquitin or | Alternative | accession |
| enzyme | Ubiquitin-like) | name | number |
| UBE2A | Ubiquitin | hHR6A | P49459 |
| UBE2B | Ubiquitin | hHR6B | P63146 |
| UBE2C | Ubiquitin | UbcH10 | O00762 |
| UBE2D1 | Ubiquitin | UbcH5A | P51668 |
| UBE2D2 | Ubiquitin | UbcH5B | P62837 |
| UBE2D3 | Ubiquitin | UbcH5C | P61077 |
| UBE2D4 | Ubiquitin | HBUCE1 | Q9Y2X8 |
| UBE2E1 | shared Ubiquitin and ISG15 | UbcH6 | P51965 |
| UBE2E2 | shared Ubiquitin and ISG15 | UBCH8 | Q96LR5 |
| UBE2E3 | Ubiquitin | UbcH9 | Q969T4 |
| UBE2F | NEDD8 | NCE2 | Q969M7 |
| UBE2G1 | Ubiquitin | UBE2G | P62253 |
| UBE2G2 | Ubiquitin | UBC7 | P60604 |
| UBE2H | Ubiquitin | UBCH | P62256 |
| UBE2I | SUMO | Ubc9 | P63279 |
| UBE2J1 | Ubiquitin | NCUBE1 | Q9Y385 |
| UBE2J2 | Ubiquitin | NCUBE2 | Q8N2K1 |
| UBE2K | Ubiquitin | HIP2 | P61086 |
| UBE2L3 | Ubiquitin | UbcH7 | P68036 |
| UBE2L6 | shared Ubiquitin and ISG15 | UbcH8 | O14933 |
| UBE2M | NEDD8 | Ubc12 | P61081 |
| UBE2N | Ubiquitin | Ubc13 | P61088 |
| UBE2NL | polymorphic pseudogene | | Q5JXB2 |
| | protein product is 91% | | |
| | identical to ubiquitin- | | |
| | conjugating enzyme E2N | | |
| UBE2O | Ubiquitin | E2-230K | Q9C0C9 |
| UBE2Q1 | Ubiquitin | NICE-5 | Q7Z7E8 |
| UBE2Q2 | Ubiquitin | UBE2Q2 | Q8WVN8 |
| UBE2QL | Ubiquitin | UBE2QL1 | A1L167 |
| UBE2R1 | Ubiquitin | CDC34 | P49427 |
| UBE2R2 | Ubiquitin | CDC34B | Q712K3 |
| UBE2S | Ubiquitin | E2-EPF | Q16763 |
| UBE2T | Ubiquitin | HSPC150 | Q9NPD8 |
| UBE2U | no E2 activity described yet | UBE2U | Q5VVX9 |
| UBE2V1 | Ubiquitin | UEV-1A | Q13404 |
| UBE2V2 | Ubiquitin | MMS2 | Q15819 |
| UBE2W | Ubiquitin | UBE2W | Q96B02 |
| UBE2Z | Shared Ubiquitin and FAT10 | Use1 | Q9H832 |
| UEVLD | | UEV3 | Q8IX04 |
| BIRC6 | Ubiquitin | BIRC6 | Q9NR09 |
| | | (apollon) | |
| FTS | | AKTIP | Q9H8T0 |
| TSG101 | | | Q99816 |
| UFC1 | | | Q9Y3C8 |

TABLE 4
E2 ubiquitinating enzymes, functions, and associated E1 and E3 enzymes

| E2 ubiquitin- | | | |
|---|---|---|---|
| conjugating | | | |
| enzyme | Function | Associated E1s | Associated E3s |
| UBE2A | E2 in DNA damage repair | Uba1, Uba6 | RING, HECT, RBR |
| UBE2B | E2 in DNA damage repair | Uba1, Uba6 | RING, HECT, RBR |
| UBE2C | chain-initiating E2 of APC/C | Uba1, Uba6 | RING, HECT, RBR |
| UBE2D1 | promiscuous lysine- and | Uba1, Uba6 | RING, HECT, RBR |
| | cysteine-reactive E2 | | |
| UBE2D2 | promiscuous lysine- and | Uba1, Uba6 | RING, HECT, RBR |
| | cysteine-reactive E2 | | |
| UBE2D3 | promiscuous lysine- and | Uba1, Uba6 | RING, HECT, RBR |
| | cysteine-reactive E2 | | |
| UBE2D4 | promiscuous lysine- and | Uba1, Uba6 | RING, HECT, RBR |
| | cysteine-reactive E2 | | |
| UBE2E1 | monoubiquitylating E2s by | Uba1, Uba6 | RING, HECT, RBR |
| | virtue of N-terminus | | |
| UBE2E2 | monoubiquitylating E2s by | Uba1, Uba6 | RING, HECT, RBR |
| | virtue of N-terminus | | |
| UBE2E3 | monoubiquitylating E2s by | Uba1, Uba6 | RING, HECT, RBR |
| | virtue of N-terminus | | |
| UBE2F | | APPBO1/Uba3 | Rbx1 and Rbx2 |
| UBE2G1 | K48 chain-builder in absence | Uba1, Uba6 | RING, HECT |
| | of E3 | | |
| UBE2G2 | K48 chain-builder in dependence | Uba1, Uba6 | RING, HECT, RBR |
| | of E3 (?); does ERAD | | |
| UBE2H | | Uba1, Uba6 | RING, HECT, RBR |
| UBE2I | SUMO E2 | Aos/Uba2 | RanBP2, Pc2, |
| | | | PIAS-proteins, Topors |
| UBE2J1 | E2 involved in the ERAD pathway | Uba1, Uba6 | RING, HECT, RBR |
| UBE2J2 | E2 with hydroxyl specificity | Uba1, Uba6 | RING, HECT, RBR |
| | (Ser/Thr) | | |
| UBE2K | K48 chain-builder in absence | Uba1, Uba6 | RING, HECT, RBR |
| | of E3 | | |
| UBE2L3 | Cysteine-only reactive E2 | Uba1, Uba6 | HECT, RBR, |
| | | | not with RING |
| UBE2L6 | | Uba1, Uba6 | RING, HECT, RBR |
| UBE2M | | APPBO1/Uba3 | Rbx1 and Rbx2 |
| UBE2N | K63 chain builder | Uba1, Uba6 | RING, HECT, RBR |
| UBE2NL | | | |
| UBE2O | Multi-domain E2/E3 hybrid | Uba1, Uba6 | its own N-terminus, |
| | | | RING, RBR |
| UBE2Q1 | | Uba1, Uba6 | RING |
| UBE2Q2 | | Uba1, Uba6 | RING, HECT |
| UBE2QL | | | |
| UBE2R1 | K48 chain-builder; works | Uba1, Uba6 | RING, HECT, RBR |
| | with SCF E3 ligases | | |
| UBE2R2 | K48 chain-builder; works | Uba1, Uba6 | RING, HECT, RBR |
| | with SCF E3 ligases | | |
| UBE2S | K11 chain-builder | Uba1, Uba6 | RING, HECT, RBR |
| UBE2T | | Uba1, Uba6 | RING, HECT, RBR |
| UBE2U | | | |
| UBE2V1 | Catalytically inactive | Uba1, Uba6 | RING, HECT (in |
| | | | combination with |
| | | | Ube2N, for K63 |
| | | | chain formation) |
| UBE2V2 | Catalytically inactive | Uba1, Uba6 | RING, HECT (in |
| | | | combination with |
| | | | Ube2N, for K63 |
| | | | chain formation) |
| UBE2W | Specificity for N-terminal amine | Uba1, Uba6 | RING, HECT, RBR |
| UBE2Z | Ubl E2 enzyme | Uba1, Uba6 | NA |
| UEVLD | | | |
| BIRC6 | Giant multi-domain E2/E3 hybrid | Uba1, Uba6 | its own N-terminus, |
| | | | HECT (isolated UBC) |
| FTS | | | |
| TSG101 | | | |
| UFC1 | | | |

TABLE 5
Subcellular localisation of E2 ubiquitinating
enzymes (Data source: UniProt).

| E2 ubiquitin- | Subcellular localisation | | | | |
|---|---|---|---|---|---|
| conjugating | | | | Endoplasmic | |
| enzyme | Cytosol | Nucleus | Endosome | reticulum | Membrane |
| UBE2A | Y | Y | | | |
| UBE2B | | Y | | | Y |
| UBE2C | Y | Y | | | Y |
| UBE2D1 | Y | | | | |
| UBE2D2 | Y | Y | | | |
| UBE2D3 | | | Y | | Y |
| UBE2D4 | | | | | |
| UBE2E1 | | Y | | | |
| UBE2E2 | | | | | |
| UBE2E3 | Y | Y | | | |
| UBE2F | Y | | | | |
| UBE2G1 | Y | | | | |
| UBE2G2 | Y | | | Y | |
| UBE2H | Y | | | | |
| UBE2I | Y | Y | | | |
| UBE2J1 | | | | Y | |
| UBE2J2 | | | | Y | |
| UBE2K | Y | | | | |
| UBE2L3 | Y | Y | | | |
| UBE2L6 | Y | Y | | | |
| UBE2M | Y | Y | | | |
| UBE2N | Y | Y | | | |
| UBE2NL | | | | | |
| UBE2O | Y | Y | | | |
| UBE2Q1 | Y | Y | | | |
| UBE2Q2 | Y | | | | |
| UBE2QL | | | | | |
| UBE2R1 | Y | Y | | | |
| UBE2R2 | Y | | | | |
| UBE2S | Y | Y | | | |
| UBE2T | | Y | | | |
| UBE2U | | Y | | | |
| UBE2V1 | | Y | | | |
| UBE2V2 | Y | Y | | | |
| UBE2W | | Y | | | |
| UBE2Z | Y | Y | | | |
| UEVLD | | | | | |
| BIRC6 | Y | | Y | | |
| FTS | | | | | |
| TSG101 | | | | | |
| UFC1 | | | | | |

TABLE 6
E2 ubiquitinating enzymes, number of amino acids, number of amino
acids of the UBC, and location of the catalytic cysteine residue.

| E2 ubiquitin- | Length | | |
|---|---|---|---|
| conjugating | (amino | UBC length | |
| enzyme | acids) | (amino acids) | Active site |
| UBE2A | 152 | 151 | C88 |
| UBE2B | 152 | 151 | C88 |
| UBE2C | 179 | 178 | C114 |
| UBE2D1 | 147 | 146 | C85 |
| UBE2D2 | 147 | 146 | C85 |
| UBE2D3 | 147 | 146 | C85 |
| UBE2D4 | 147 | 146 | C85 |
| UBE2E1 | 193 | 192 | C131 |
| UBE2E2 | 201 | 189 | C139 |
| UBE2E3 | 207 | 149 | C145 |
| UBE2F | 185 | 160 | C116 |
| UBE2G1 | 170 | 153 | C90 |
| UBE2G2 | 165 | 164 | C89 |
| UBE2H | 183 | 159 | C87 |
| UBE2I | 158 | 157 | C93 |
| UBE2J1 | 318 | 284 | C91 |
| UBE2J2 | 259 | 184 | C94 |
| UBE2K | 200 | 199 | C92 |
| UBE2L3 | 154 | 153 | C381 |
| UBE2L6 | 153 | 152 | C86 |
| UBE2M | 183 | 157 | C111 |
| UBE2N | 152 | 151 | C87 |
| UBE2NL | 153 | 152 | No catalytic |
| | | | cysteine |
| UBE2O | 1292 | 138 | C1040 |
| UBE2Q1 | 422 | 168 | C351 |
| UBE2Q2 | 375 | 167 | C304 |
| UBE2QL | 161 | 160 | C88 |
| UBE2R1 | 236 | 178 | C93 |
| UBE2R2 | 238 | 178 | C93 |
| UBE2S | 222 | 155 | C95 |
| UBE2T | 197 | 166 | C86 |
| UBE2U | 321 | 149 | C89 |
| UBE2V1 | 147 | 140 | Inactive - Needs |
| | | | UBE2N |
| UBE2V2 | 145 | 144 | Inactive - Needs |
| | | | UBE2N |
| UBE2W | 151 | 117 | C91 |
| UBE2Z | 354 | 145 | C188 |
| UEVLD | 471 | 182 | No catalytic |
| | | | cysteine |
| BIRC6 | 4857 | 203 | C4625 |
| FTS | 292 | 202 | No catalytic |
| | | | cysteine |
| TSG101 | 390 | 144 | Inactive |
| | | | Homologue |
| UFC1 | 167 | 166 | C116 |

TABLE 7
Full amino acid sequences of E2 ubiquitin-conjugating enzymes and
corresponding SEQ ID NOS.

| E2 ubiquitin- | | |
|---|---|---|
| conjugating | | |
| enzyme | Full amino acid sequence | SEQ ID NO: |
| UBE2A | MSTPARRRLMRDFKRLQEDPPAGVSGAPSE | SEQ ID NO: 1 |
| | NNIMVWNAVIFGPEGTPFEDGTFKLTIEFTEE | |
| | YPNKPPTVRFVSKMFHPNVYADGSICLDILQ | |
| | NRWSPTYDVSSILTSIQSLLDEPNPNSPANS | |
| | QAAQLYQENKREYEKRVSAIVEQSWRDC | |
| UBE2B | MSTPARRRLMRDFKRLQEDPPVGVSGAPSE | SEQ ID NO: 2 |
| | NNIMQWNAVIFGPEGTPFEDGTFKLVIEFSEE | |
| | YPNKPPTVRFLSKMFHPNVYADGSICLDILQN | |
| | RWSPTYDVSSILTSIQSLLDEPNPNSPANSQ | |
| | AAQLYQENKREYEKRVSAIVEQSWNDS | |
| UBE2C | MASQNRDPAATSVAAARKGAEPSGGAARG | SEQ ID NO: 3 |
| | PVGKRLQQELMTLMMSGDKGISAFPESDNL | |
| | FKWVGTIHGAAGTVYEDLRYKLSLEFPSGYP | |
| | YNAPTVKFLTPCYHPNVDTQGNICLDILKEK | |
| | WSALYDVRTILLSIQSLLGEPNIDSPLNTHAAE | |
| | LWKNPTAFKKYLQETYSKQVTSQEP | |
| UBE2D1 | MALKRIQKELSDLQRDPPAHCSAGPVGDDLF | SEQ ID NO: 4 |
| | HWQATIMGPPDSAYQGGVFFLTVHFPTDYP | |
| | FKPPKIAFTTKIYHPNINSNGSICLDILRSQWS | |
| | PALTVSKVLLSICSLLCDPNPDDPLVPDIAQIY | |
| | KSDKEKYNRHAREWTQKYAM | |
| UBE2D2 | MALKRIHKELNDLARDPPAQCSAGPVGDDM | SEQ ID NO: 5 |
| | FHWQATIMGPNDSPYQGGVFFLTIHFPTDYP | |
| | FKPPKVAFTTRIYHPNINSNGSICLDILRSQW | |
| | SPALTISKVLLSICSLLCDPNPDDPLVPEIARIY | |
| | KTDREKYNRIAREWTQKYAM | |
| UBE2D3 | MALKRINKELSDLARDPPAQCSAGPVGDDM | SEQ ID NO: 6 |
| | FHWQATIMGPNDSPYQGGVFFLTIHFPTDYP | |
| | FKPPKVAFTTRIYHPNINSNGSICLDILRSQW | |
| | SPALTISKVLLSICSLLCDPNPDDPLVPEIARIY | |
| | KTDRDKYNRISREWTQKYAM | |
| UBE2D4 | MALKRIQKELTDLQRDPPAQCSAGPVGDDLF | SEQ ID NO: 7 |
| | HWQATIMGPNDSPYQGGVFFLTIHFPTDYPF | |
| | KPPKVAFTTKIYHPNINSNGSICLDILRSQWS | |
| | PALTVSKVLLSICSLLCDPNPDDPLVPEIAHTY | |
| | KADREKYNRLAREWTQKYAM | |
| UBE2E1 | MSDDDSRASTSSSSSSSSNQQTEKETNTPK | SEQ ID NO: 8 |
| | KKESKVSMSKNSKLLSTSAKRIQKELADITLD | |
| | PPPNCSAGPKGDNIYEWRSTILGPPGSVYEG | |
| | GVFFLDITFTPEYPFKPPKVTFRTRIYHCNINS | |
| | QGVICLDILKDNWSPALTISKVLLSICSLLTDC | |
| | NPADPLVGSIATQYMTNRAEHDRMARQWTK | |
| | RYAT | |
| UBE2E2 | MSTEAQRVDDSPSTSGGSSDGDQRESVQQ | SEQ ID NO: 9 |
| | EPEREQVQPKKKEGKISSKTAAKLSTSAKRIQ | |
| | KELAEITLDPPPNCSAGPKGDNIYEWRSTILG | |
| | PPGSVYEGGVFFLDITFSPDYPFKPPKVTFRT | |
| | RIYHCNINSQGVICLDILKDNWSPALTISKVLL | |
| | SICSLLTDCNPADPLVGSIATQYMTNRAEHD | |
| | RMARQWTKRYAT | |
| UBE2E3 | MSSDRQRSDDESPSTSSGSSDADQRDPAA | SEQ ID NO: |
| | PEPEEQEERKPSATQQKKNTKLSSKTTAKLS | 10 |
| | TSAKRIQKELAEITLDPPPNCSAGPKGDNIYE | |
| | WRSTILGPPGSVYEGGVFFLDITFSSDYPFKP | |
| | PKVTFRTRIYHCNINSQGVICLDILKDNWSPA | |
| | LTISKVLLSICSLLTDCNPADPLVGSIATQYLT | |
| | NRAEHDRIARQWTKRYAT | |
| UBE2F | MLTLASKLKRDDGLKGSRTAATASDSTRRVS | SEQ ID NO: |
| | VRDKLLVKEVAELEANLPCTCKVHFPDPNKL | 11 |
| | HCFQLTVTPDEGYYQGGKFQFETEVPDAYN | |
| | MVPPKVKCLTKIWHPNITETGEICLSLLREHSI | |
| | DGTGWAPTRTLKDVVWGLNSLFTDLLNFDD | |
| | PLNIEAAEHHLRDKEDFRNKVDDYIKRYAR | |
| UBE2G1 | MTELQSALLLRRQLAELNKNPVEGFSAGLID | SEQ ID NO: |
| | DNDLYRWEVLIIGPPDTLYEGGVFKAHLTFPK | 12 |
| | DYPLRPPKMKFITEIWHPNVDKNGDVCISILH | |
| | EPGEDKYGYEKPEERWLPIHTVETIMISVISM | |
| | LADPNGDSPANVDAAKEWREDRNGEFKRKV | |
| | ARCVRKSQETAFE | |
| UBE2G2 | MAGTALKRLMAEYKQLTLNPPEGIVAGPMNE | SEQ ID NO: |
| | ENFFEWEALIMGPEDTCFEFGVFPAILSFPLD | 13 |
| | YPLSPPKMRFTCEMFHPNIYPDGRVCISILHA | |
| | PGDDPMGYESSAERWSPVQSVEKILLSVVS | |
| | MLAEPNDESGANVDASKMWRDDREQFYKIA | |
| | KQIVQKSLGL | |
| UBE2H | MSSPSPGKRRMDTDVVKLIESKHEVTILGGL | SEQ ID NO: |
| | NEFVVKFYGPQGTPYEGGVWKVRVDLPDKY | 14 |
| | PFKSPSIGFMNKIFHPNIDEASGTVCLDVINQ | |
| | TWTALYDLTNIFESFLPQLLAYPNPIDPLNGD | |
| | AAAMYLHRPEEYKQKIKEYIQKYATEEALKEQ | |
| | EEGTGDSSSESSMSDFSEDEAQDMEL | |
| UBE21 | MSGIALSRLAQERKAWRKDHPFGFVAVPTK | SEQ ID NO: |
| | NPDGTMNLMNWECAIPGKKGTPWEGGLFKL | 15 |
| | RMLFKDDYPSSPPKCKFEPPLFHPNVYPSGT | |
| | VCLSILEEDKDWRPAITIKQILLGIQELLNEPNI | |
| | QDPAQAEAYTIYCQNRVEYEKRVRAQAKKF | |
| | APS | |
| UBE2J1 | METRYNLKSPAVKRLMKEAAELKDPTDHYH | SEQ ID NO: |
| | AQPLEDNLFEWHFTVRGPPDSDFDGGVYHG | 16 |
| | RIVLPPEYPMKPPSIILLTANGRFEVGKKICLSI | |
| | SGHHPETWQPSWSIRTALLAIIGFMPTKGEG | |
| | AIGSLDYTPEERRALAKKSQDFCCEGCGSA | |
| | MKDVLLPLKSGSDSSQADQEAKELARQISFK | |
| | AEVNSSGKTISESDLNHSFSLTDLQDDIPTTF | |
| | QGATASTSYGLQNSSAASFHQPTQPVAKNT | |
| | SMSPRQRRAQQQSQRRLSTSPDVIQGHQP | |
| | RDNHTDHGGSAVLIVILTLALAALIFRRIYLAN | |
| | EYIFDFEL | |
| UBE2J2 | MSSTSSKRAPTTATQRLKQDYLRIKKDPVPYI | SEQ ID NO: |
| | CAEPLPSNILEWHYVVRGPEMTPYEGGYYH | 17 |
| | GKLIFPREFPFKPPSIYMITPNGRFKCNTRLC | |
| | LSITDFHPDTWNPAWSVSTILTGLLSFMVEK | |
| | GPTLGSIETSDFTKRQLAVQSLAFNLKDKVFC | |
| | ELFPEVVEEIKQKQKAQDELSSRPQTLPLPD | |
| | VVPDGETHLVQNGIQLLNGHAPGAVPNLAGL | |
| | QQANRHHGLLGGALANLFVIVGFAAFAYTVK | |
| | YVLRSIAQE | |
| UBE2K | MANIAVQRIKREFKEVLKSEETSKNQIKVDLV | SEQ ID NO: |
| | DENFTELRGEIAGPPDTPYEGGRYQLEIKIPE | 18 |
| | TYPFNPPKVRFITKIWHPNISSVTGAICLDILK | |
| | DQWAAAMTLRTVLLSLQALLAAAEPDDPQD | |
| | AVVANQYKQNPEMFKQTARLWAHVYAGAPV | |
| | SSPEYTKKIENLCAMGFDRNAVIVALSSKSW | |
| | DVETATELLLSN | |
| UBE2L3 | MAASRRLMKELEEIRKCGMKNFRNIQVDEAN | SEQ ID NO: |
| | LLTWQGLIVPDNPPYDKGAFRIEINFPAEYPF | 19 |
| | KPPKITFKTKIYHPNIDEKGQVCLPVISAENW | |
| | KPATKTDQVIQSLIALVNDPQPEHPLRADLAE | |
| | EYSKDRKKFCKNAEEFTKKYGEKRPVD | |
| UBE2L6 | MMASMRVVKELEDLQKKPPPYLRNLSSDDA | SEQ ID NO: |
| | NVLVWHALLLPDQPPYHLKAFNLRISFPPEYP | 20 |
| | FKPPMIKFTTKIYHPNVDENGQICLPIISSENW | |
| | KPCTKTCQVLEALNVLVNRPNIREPLRMDLA | |
| | DLLTQNPELFRKNAEEFTLRFGVDRPS | |
| UBE2M | MIKLFSLKQQKKEEESAGGTKGSSKKASAAQ | SEQ ID NO: |
| | LRIQKDINELNLPKTCDISFSDPDDLLNFKLVI | 21 |
| | CPDEGFYKSGKFVFSFKVGQGYPHDPPKVK | |
| | CETMVYHPNIDLEGNVCLNILREDWKPVLTIN | |
| | SIIYGLQYLFLEPNPEDPLNKEAAEVLQNNRR | |
| | LFEQNVQRSMRGGYIGSTYFERCLK | |
| UBE2N | MAGLPRRIIKETQRLLAEPVPGIKAEPDESNA | SEQ ID NO: |
| | RYFHVVIAGPQDSPFEGGTFKLELFLPEEYP | 22 |
| | MAAPKVRFMTKIYHPNVDKLGRICLDILKDKW | |
| | SPALQIRTVLLSIQALLSAPNPDDPLANDVAE | |
| | QWKTNEAQAIETARAWTRLYAMNNI | |
| UBE2NL | MAELPHRIIKETQRLLAEPVPGIKAEPDESNA | SEQ ID NO: |
| | RYFHVVIAGESKDSPFEGGTFKRELLLAEEY | 23 |
| | PMAAPKVRFMTKIYHPNVDKLERISLDILKDK | |
| | WSPALQIRTVLLSIQALLNAPNPDDPLANDVV | |
| | EQWKTNEAQAIETARAWTRLYAMNSI | |
| UBE2O | MADPAAPTPAAPAPAQAPAPAPEAVPAPAA | SEQ ID NO: |
| | APVPAPAPASDSASGPSSDSGPEAGSQRLL | 24 |
| | FSHDLVSGRYRGSVHFGLVRLIHGEDSDSEG | |
| | EEEGRGSSGCSEAGGAGHEEGRASPLRRG | |
| | YVRVQWYPEGVKQHVKETKLKLEDRSVVPR | |
| | DVVRHMRSTDSQCGTVIDVNIDCAVKLIGTN | |
| | CIIYPVNSKDLQHIWPFMYGDYIAYDCWLGK | |
| | VYDLKNQIILKLSNGARCSMNTEDGAKLYDV | |
| | CPHVSDSGLFFDDSYGFYPGQVLIGPAKIFS | |
| | SVQWLSGVKPVLSTKSKFRVVVEEVQVVELK | |
| | VTWITKSFCPGGTDSVSPPPSVITQENLGRV | |
| | KRLGCFDHAQRQLGERCLYVFPAKVEPAKIA | |
| | WECPEKNCAQGEGSMAKKVKRLLKKQVVRI | |
| | MSCSPDTQCSRDHSMEDPDKKGESKTKSEA | |
| | ESASPEETPDGSASPVEMQDEGAEEPHEAG | |
| | EQLPPFLLKEGRDDRLHSAEQDADDEAADD | |
| | TDDTSSVTSSASSTTSSQSGSGTSRKKSIPL | |
| | SIKNLKRKHKRKKNKITRDFKPGDRVAVEVVT | |
| | TMTSADVMWQDGSVECNIRSNDLFPVHHLD | |
| | NNEFCPGDFVVDKRVQSCPDPAVYGVVQSG | |
| | DHIGRTCMVKWFKLRPSGDDVELIGEEEDVS | |
| | VYDIADHPDFRFRTTDIVIRIGNTEDGAPHKE | |
| | DEPSVGQVARVDVSSKVEVVWADNSKTIILP | |
| | QHLYNIESEIEESDYDSVEGSTSGASSDEWE | |
| | DDSDSWETDNGLVEDEHPKIEEPPIPPLEQP | |
| | VAPEDKGVVISEEAATAAVQGAVAMAAPMA | |
| | GLMEKAGKDGPPKSFRELKEAIKILESLKNMT | |
| | VEQLLTGSPTSPTVEPEKPTREKKFLDDIKKL | |
| | QENLKKTLDNVAIVEEEKMEAVPDVERKEDK | |
| | PEGQSPVKAEWPSETPVLCQQCGGKPGVTF | |
| | TSAKGEVFSVLEFAPSNHSFKKIEFQPPEAKK | |
| | FFSTVRKEMALLATSLPEGIMVKTFEDRMDL | |
| | FSALIKGPTRTPYEDGLYLFDIQLPNIYPAVPP | |
| | HFCYLSQCSGRLNPNLYDNGKVCVSLLGTWI | |
| | GKGTERWTSKSSLLQVLISIQGLILVNEPYYN | |
| | EAGFDSDRGLQEGYENSRCYNEMALIRVVQ | |
| | SMTQLVRRPPEVFEQEIRQHFSTGGWRLVN | |
| | RIESWLETHALLEKAQALPNGVPKASSSPEP | |
| | PAVAELSDSGQQEPEDGGPAPGEASQGSD | |
| | SEGGAQGLASASRDHTDQTSETAPDASVPP | |
| | SVKPKKRRKSYRSFLPEKSGYPDIGFPLFPLS | |
| | KGFIKSIRGVLTQFRAALLEAGMPECTEDK | |
| UBE2Q1 | MQQPQPQGQQQPGPGQQLGGQGAAPGAG | SEQ ID NO: |
| | GGPGGGPGPGPCLRRELKLLESIFHRGHER | 25 |
| | FRIASACLDELSCEFLLAGAGGAGAGAAPGP | |
| | HLPPRGSVPGDPVRIHCNITESYPAVPPIWSV | |
| | ESDDPNLAAVLERLVDIKKGNTLLLQHLKRIIS | |
| | DLCKLYNLPQHPDVEMLDQPLPAEQCTQED | |
| | VSSEDEDEEMPEDTEDLDHYEMKEEEPAEG | |
| | KKSEDDGIGKENLAILEKIKKNQRQDYLNGAV | |
| | SGSVQATDRLMKELRDIYRSQSFKGGNYAV | |
| | ELVNDSLYDWNVKLLKVDQDSALHNDLQILK | |
| | EKEGADFILLNFSFKDNFPFDPPFVRVVSPVL | |
| | SGGYVLGGGAICMELLTKQGWSSAYSIESVI | |
| | MQISATLVKGKARVQFGANKSQYSLTRAQQ | |
| | SYKSLVQIHEKNGWYTPPKEDG | |
| UBE2Q2 | MSVSGLKAELKFLASIFDKNHERFRIVSWKLD | SEQ ID NO: |
| | ELHCQFLVPQQGSPHSLPPPLTLHCNITESY | 26 |
| | PSSSPIWFVDSEDPNLTSVLERLEDTKNNNL | |
| | LRQQLKWLICELCSLYNLPKHLDVEMLDQPL | |
| | PTGQNGTTEEVTSEEEEEEEEMAEDIEDLDH | |
| | YEMKEEEPISGKKSEDEGIEKENLAILEKIRKT | |
| | QRQDHLNGAVSGSVQASDRLMKELRDIYRS | |
| | QSYKTGIYSVELINDSLYDWHVKLQKVDPDS | |
| | PLHSDLQILKEKEGIEYILLNFSFKDNFPFDPP | |
| | FVRVVLPVLSGGYVLGGGALCMELLTKQGW | |
| | SSAYSIESVIMQINATLVKGKARVQFGANKNQ | |
| | YNLARAQQSYNSIVQIHEKNGWYTPPKEDG | |
| UBE2QL | MKELQDIARLSDRFISVELVDESLFDWNVKLH | SEQ ID NO: |
| | QVDKDSVLWQDMKETNTEFILLNLTFPDNFP | 27 |
| | FSPPFMRVLSPRLENGYVLDGGAICMELLTP | |
| | RGWSSAYTVEAVMRQFAASLVKGQGRICRK | |
| | AGKSKKSFSRKEAEATFKSLVKTHEKYGWVT | |
| | PPVSDG | |
| UBE2R1 | MARPLVPSSQKALLLELKGLQEEPVEGFRVT | SEQ ID NO: |
| | LVDEGDLYNWEVAIFGPPNTYYEGGYFKARL | 28 |
| | KFPIDYPYSPPAFRFLTKMWHPNIYETGDVCI | |
| | SILHPPVDDPQSGELPSERWNPTQNVRTILL | |
| | SVISLLNEPNTFSPANVDASVMYRKWKESKG | |
| | KDREYTDIIRKQVLGTKVDAERDGVKVPTTLA | |
| | EYCVKTKAPAPDEGSDLFYDDYYEDGEVEE | |
| | EADSCFGDDEDDSGTEES | |
| UBE2R2 | MAQQQMTSSQKALMLELKSLQEEPVEGFRIT | SEQ ID NO: |
| | LVDESDLYNWEVAIFGPPNTLYEGGYFKAHI | 29 |
| | KFPIDYPYSPPTFRFLTKMWHPNIYENGDVCI | |
| | SILHPPVDDPQSGELPSERWNPTQNVRTILL | |
| | SVISLLNEPNTFSPANVDASVMFRKWRDSKG | |
| | KDKEYAEIIRKQVSATKAEAEKDGVKVPTTLA | |
| | EYCIKTKVPSNDNSSDLLYDDLYDDDIDDEDE | |
| | EEEDADCYDDDDSGNEES | |
| UBE2S | MNSNVENLPPHIIRLVYKEVTTLTADPPDGIK | SEQ ID NO: |
| | VFPNEEDLTDLQVTIEGPEGTPYAGGLFRMK | 30 |
| | LLLGKDFPASPPKGYFLTKIFHPNVGANGEIC | |
| | VNVLKRDWTAELGIRHVLLTIKCLLIHPNPESA | |
| | LNEEAGRLLLENYEEYAARARLLTEIHGGAG | |
| | GPSGRAEAGRALASGTEASSTDPGAPGGPG | |
| | GAEGPMAKKHAGERDKKLAAKKKTDKKRAL | |
| | RRL | |
| UBE2T | MQRASRLKRELHMLATEPPPGITCWQDKDQ | SEQ ID NO: |
| | MDDLRAQILGGANTPYEKGVFKLEVIIPERYP | 31 |
| | FEPPQIRFLTPIYHPNIDSAGRICLDVLKLPPK | |
| | GAWRPSLNIATVLTSIQLLMSEPNPDDPLMA | |
| | DISSEFKYNKPAFLKNARQWTEKHARQKQK | |
| | ADEEEMLDNLPEAGDSRVHNSTQKRKASQL | |
| | VGIEKKFHPDV | |
| UBE2U | MHGRAYLLLHRDFCDLKENNYKGITAKPVSE | SEQ ID NO: |
| | DMMEWEVEIEGLQNSVWQGLVFQLTIHFTS | 32 |
| | EYNYAPPVVKFITIPFHPNVDPHTGQPCIDFL | |
| | DNPEKWNTNYTLSSILLALQVMLSNPVLENP | |
| | VNLEAARILVKDESLYRTILRLFNRPLQMKDD | |
| | SQELPKDPRKCIRPIKTTSFSDYYQTWSRIAT | |
| | SKATEYYRTPLLKVPNFIGQYYKWKKMDLQH | |
| | QKEWNLKYSVIKCWLARKRMPHEVTHSMEE | |
| | IKLCPTLIPTTDEIFLESPTAINSITDIYETEEEG | |
| | WKSDTSLYENDTDEPREEEVEDLISWTNTLN | |
| | TNTSED | |
| UBE2V1 | MAATTGSGVKVPRNFRLLEELEEGQKGVGD | SEQ ID NO: |
| | GTVSWGLEDDEDMTLTRWTGMIIGPPRTIYE | 33 |
| | NRIYSLKIECGPKYPEAPPFVRFVTKINMNGV | |
| | NSSNGVVDPRAISVLAKWQNSYSIKVVLQEL | |
| | RRLMMSKENMKLPQPPEGQCYSN | |
| UBE2V2 | MAVSTGVKVPRNFRLLEELEEGQKGVGDGT | SEQ ID NO: |
| | VSWGLEDDEDMTLTRWTGMIIGPPRTNYEN | 34 |
| | RIYSLKVECGPKYPEAPPSVRFVTKINMNGIN | |
| | NSSGMVDARSIPVLAKWQNSYSIKVVLQELR | |
| | RLMMSKENMKLPQPPEGQTYNN | |
| UBE2W | MASMQKRLQKELLALQNDPPPGMTLNEKSV | SEQ ID NO: |
| | QNSITQWIVDMEGAPGTLYEGEKFQLLFKFS | 35 |
| | SRYPFDSPQVMFTGENIPVHPHVYSNGHICL | |
| | SILTEDWSPALSVQSVCLSIISMLSSCKEKRR | |
| | PPDNSFYVRTCNKNPKKTKWWYHDDTC | |
| UBE2Z | MAESPTEEAATAGAGAAGPGASSVAGVVGV | SEQ ID NO: |
| | SGSGGGFGPPFLPDVWAAAAAAGGAGGPG | 36 |
| | SGLAPLPGLPPSAAAHGAALLSHWDPTLSSD | |
| | WDGERTAPQCLLRIKRDIMSIYKEPPPGMFV | |
| | VPDTVDMTKIHALITGPFDTPYEGGFFLFVFR | |
| | CPPDYPIHPPRVKLMTTGNNTVRFNPNFYRN | |
| | GKVCLSILGTWTGPAWSPAQSISSVLISIQSL | |
| | MTENPYHNEPGFEQERHPGDSKNYNECIRH | |
| | ETIRVAVCDMMEGKCPCPEPLRGVMEKSFL | |
| | EYYDFYEVACKDRLHLQGQTMQDPFGEKRG | |
| | HFDYQSLLMRLGLIRQKVLERLHNENAEMDS | |
| | DSSSSGTETDLHGSLRV | |
| UEVLD | MEFDCEGLRRLLGKYKFRDLTVEELRNVNVF | SEQ ID NO: |
| | FPHFKYSMDTYVFKDSSQKDLLNFTGTIPVM | 37 |
| | YQGNTYNIPIRFWILDSHPFAPPICFLKPTAN | |
| | MGILVGKHVDAQGRIYLPYLQNWSHPKSVIV | |
| | GLIKEMIAKFQEELPMYSLSSSDEARQVDLLA | |
| | YIAKITEGVSDTNSKSWANHENKTVNKITVVG | |
| | GGELGIACTLAISAKGIADRLVLLDLSEGTKG | |
| | ATMDLEIFNLPNVEISKDLSASAHSKVVIFTVN | |
| | SLGSSQSYLDVVQSNVDMFRALVPALGHYS | |
| | QHSVLLVASQPVEIMTYVTWKLSTFPANRVI | |
| | GIGCNLDSQRLQYIITNVLKAQTSGKEVWVIG | |
| | EQGEDKVLTWSGQEEVVSHTSQVQLSNRA | |
| | MELLRVKGQRSWSVGLSVADMVDSIVNNKK | |
| | KVHSVSALAKGYYDINSEVFLSLPCILGTNGV | |
| | SEVIKTTLKEDTVTEKLQSSASSIHSLQQQLK | |
| | L | |
| BIRC6 | MVTGGGAAPPGTVTEPLPSVIVLSAGRKMAA | SEQ ID NO: |
| | AAAAASGPGCSSAAGAGAAGVSEWLVLRDG | 38 |
| | CMHCDADGLHSLSYHPALNAILAVTSRGTIKV | |
| | IDGTSGATLQASALSAKPGGQVKCQYISAVD | |
| | KVIFVDDYAVGCRKDLNGILLLDTALQTPVSK | |
| | QDDVVQLELPVTEAQQLLSACLEKVDISSTE | |
| | GYDLFITQLKDGLKNTSHETAANHKVAKWAT | |
| | VTFHLPHHVLKSIASAIVNELKKINQNVAALPV | |
| | ASSVMDRLSYLLPSARPELGVGPGRSVDRS | |
| | LMYSEANRRETFTSWPHVGYRWAQPDPMA | |
| | QAGFYHQPASSGDDRAMCFTCSVCLVCWE | |
| | PTDEPWSEHERHSPNCPFVKGEHTQNVPLS | |
| | VTLATSPAQFPCTDGTDRISCFGSGSCPHFL | |
| | AAATKRGKICIWDVSKLMKVHLKFEINAYDPA | |
| | IVQQLILSGDPSSGVDSRRPTLAWLEDSSSC | |
| | SDIPKLEGDSDDLLEDSDSEEHSRSDSVTGH | |
| | TSQKEAMEVSLDITALSILQQPEKLQWEIVAN | |
| | VLEDTVKDLEELGANPCLTNSKSEKTKEKHQ | |
| | EQHNIPFPCLLAGGLLTYKSPATSPISSNSHR | |
| | SLDGLSRTQGESISEQGSTDNESCTNSELNS | |
| | PLVRRTLPVLLLYSIKESDEKAGKIFSQMNNI | |
| | MSKSLHDDGFTVPQIIEMELDSQEQLLLQDP | |
| | PVTYIQQFADAAANLTSPDSEKWNSVFPKPG | |
| | TLVQCLRLPKFAEEENLCIDSITPCADGIHLLV | |
| | GLRTCPVESLSAINQVEALNNLNKLNSALON | |
| | RRKGELESNLAVVNGANISVIQHESPADVQT | |
| | PLIIQPEQRNVSGGYLVLYKMNYATRIVTLEE | |
| | EPIKIQHIKDPQDTITSLILLPPDILDNREDDCE | |
| | EPIEDMQLTSKNGFEREKTSDISTLGHLVITT | |
| | QGGYVKILDLSNFEILAKVEPPKKEGTEEQDT | |
| | FVSVIYCSGTDRLCACTKGGELHFLQIGGTC | |
| | DDIDEADILVDGSLSKGIEPSSEGSKPLSNPS | |
| | SPGISGVDLLVDQPFTLEILTSLVELTRFETLT | |
| | PRFSATVPPCWVEVQQEQQQRRHPQHLHQ | |
| | QHHGDAAQHTRTWKLQTDSNSWDEHVFEL | |
| | VLPKACMVGHVDFKFVLNSNITNIPQIQVTLL | |
| | KNKAPGLGKVNALNIEVEQNGKPSLVDLNEE | |
| | MQHMDVEESQCLRLCPFLEDHKEDILCGPV | |
| | WLASGLDLSGHAGMLTLTSPKLVKGMAGGK | |
| | YRSFLIHVKAVNERGTEEICNGGMRPVVRLP | |
| | SLKHQSNKGYSLASLLAKVAAGKEKSSNVKN | |
| | ENTSGTRKSENLRGCDLLQEVSVTIRRFKKT | |
| | SISKERVQRCAMLQFSEFHEKLVNTLCRKTD | |
| | DGQITEHAQSLVLDTLCWLAGVHSNGPGSS | |
| | KEGNENLLSKTRKFLSDIVRVCFFEAGRSIAH | |
| | KCARFLALCISNGKCDPCQPAFGPVLLKALL | |
| | DNMSFLPAATTGGSVYWYFVLLNYVKDEDL | |
| | AGCSTACASLLTAVSRQLQDRLTPMEALLQT | |
| | RYGLYSSPFDPVLFDLEMSGSSCKNVYNSSI | |
| | GVQSDEIDLSDVLSGNGKVSSCTAAEGSFTS | |
| | LTGLLEVEPLHFTCVSTSDGTRIERDDAMSS | |
| | FGVTPAVGGLSSGTVGEASTALSSAAQVALQ | |
| | SLSHAMASAEQQLQVLQEKQQQLLKLQQQK | |
| | AKLEAKLHQTTAAAAAAASAVGPVHNSVPSN | |
| | PVAAPGFFIHPSDVIPPTPKTTPLFMTPPLTP | |
| | PNEAVSVVINAELAQLFPGSVIDPPAVNLAAH | |
| | NKNSNKSRMNPLGSGLALAISHASHFLQPPP | |
| | HQSIIIERMHSGARRFVTLDFGRPILLTDVLIP | |
| | TCGDLASLSIDIWTLGEEVDGRRLVVATDIST | |
| | HSLILHDLIPPPVCRFMKITVIGRYGSTNARAK | |
| | IPLGFYYGHTYILPWESELKLMHDPLKGEGE | |
| | SANQPEIDQHLAMMVALQEDIQCRYNLACHR | |
| | LETLLQSIDLPPLNSANNAQYFLRKPDKAVEE | |
| | DSRVFSAYQDCIQLQLQLNLAHNAVQRLKVA | |
| | LGASRKMLSETSNPEDLIQTSSTEQLRTIIRYL | |
| | LDTLLSLLHASNGHSVPAVLQSTFHAQACEE | |
| | LFKHLCISGTPKIRLHTGLLLVQLCGGERWW | |
| | GQFLSNVLQELYNSEQLLIFPQDRVFMLLSCI | |
| | GQRSLSNSGVLESLLNLLDNLLSPLQPQLPM | |
| | HRRTEGVLDIPMISWVVMLVSRLLDYVATVE | |
| | DEAAAAKKPLNGNQWSFINNNLHTQSLNRS | |
| | SKGSSSLDRLYSRKIRKQLVHHKQQLNLLKA | |
| | KQKALVEQMEKEKIQSNKGSSYKLLVEQAKL | |
| | KQATSKHFKDLIRLRRTAEWSRSNLDTEVTT | |
| | AKESPEIEPLPFTLAHERCISVVQKLVLFLLSM | |
| | DFTCHADLLLFVCKVLARIANATRPTIHLCEIV | |
| | NEPQLERLLLLLVGTDFNRGDISWGGAWAQ | |
| | YSLTCMLQDILAGELLAPVAAEAMEEGTVGD | |
| | DVGATAGDSDDSLQQSSVQLLETIDEPLTHDI | |
| | TGAPPLSSLEKDKEIDLELLQDLMEVDIDPLDI | |
| | DLEKDPLAAKVFKPISSTWYDYWGADYGTY | |
| | NYNPYIGGLGIPVAKPPANTEKNGSQTVSVS | |
| | VSQALDARLEVGLEQQAELMLKMMSTLEAD | |
| | SILQALTNTSPTLSQSPTGTDDSLLGGLQAAN | |
| | QTSQLIIQLSSVPMLNVCFNKLFSMLQVHHV | |
| | QLESLLQLWLTLSLNSSSTGNKENGADIFLYN | |
| | ANRIPVISLNQASITSFLTVLAWYPNTLLRTW | |
| | CLVLHSLTLMTNMQLNSGSSSAIGTQESTAH | |
| | LLVSDPNLIHVLVKFLSGTSPHGTNQHSPQV | |
| | GPTATQAMQEFLTRLQVHLSSTCPQIFSEFLL | |
| | KLIHILSTERGAFQTGQGPLDAQVKLLEFTLE | |
| | QNFEVVSVSTISAVIESVTFLVHHYITCSDKV | |
| | MSRSGSDSSVGARACFGGLFANLIRPGDAK | |
| | AVCGEMTRDQLMFDLLKLVNILVQLPLSGNR | |
| | EYSARVSVTTNTTDSVSDEEKVSGGKDGNG | |
| | SSTSVQGSPAYVADLVLANQQIMSQILSALGL | |
| | CNSSAMAMIIGASGLHLTKHENFHGGLDAISV | |
| | GDGLFTILTTLSKKASTVHMMLQPILTYMACG | |
| | YMGRQGSLATCQLSEPLLWFILRVLDTSDAL | |
| | KAFHDMGGVQLICNNMVTSTRAIVNTARSMV | |
| | STIMKFLDSGPNKAVDSTLKTRILASEPDNAE | |
| | GIHNFAPLGTITSSSPTAQPAEVLLQATPPHR | |
| | RARSAAWSYIFLPEEAWCDLTIHLPAAVLLKE | |
| | IHIQPHLASLATCPSSVSVEVSADGVNMLPLS | |
| | TPVVTSGLTYIKIQLVKAEVASAVCLRLHRPR | |
| | DASTLGLSQIKLLGLTAFGTTSSATVNNPFLP | |
| | SEDQVSKTSIGWLRLLHHCLTHISDLEGMMA | |
| | SAAAPTANLLQTCAALLMSPYCGMHSPNIEV | |
| | VLVKIGLQSTRIGLKLIDILLRNCAASGSDPTD | |
| | LNSPLLFGRLNGLSSDSTIDILYQLGTTQDPG | |
| | TKDRIQALLKWVSDSARVAAMKRSGRMNYM | |
| | CPNSSTVEYGLLMPSPSHLHCVAAILWHSYE | |
| | LLVEYDLPALLDQELFELLFNWSMSLPCNMV | |
| | LKKAVDSLLCSMCHVHPNYFSLLMGWMGIT | |
| | PPPVQCHHRLSMTDDSKKQDLSSSLTDDSK | |
| | NAQAPLALTESHLATLASSSQSPEAIKQLLDS | |
| | GLPSLLVRSLASFCFSHISSSESIAQSIDISQD | |
| | KLRRHHVPQQCNKMPITADLVAPILRFLTEVG | |
| | NSHIMKDWLGGSEVNPLWTALLFLLCHSGST | |
| | SGSHNLGAQQTSARSASLSSAATTGLTTQQ | |
| | RTAIENATVAFFLQCISCHPNNQKLMAQVLC | |
| | ELFQTSPQRGNLPTSGNISGFIRRLFLQLMLE | |
| | DEKVTMFLQSPCPLYKGRINATSHVIQHPMY | |
| | GAGHKFRTLHLPVSTTLSDVLDRVSDTPSITA | |
| | KLISEQKDDKEKKNHEEKEKVKAENGFQDNY | |
| | SVVVASGLKSQSKRAVSATPPRPPSRRGRTI | |
| | PDKIGSTSGAEAANKIITVPVFHLFHKLLAGQ | |
| | PLPAEMTLAQLLTLLYDRKLPQGYRSIDLTVK | |
| | LGSRVITDPSLSKTDSYKRLHPEKDHGDLLA | |
| | SCPEDEALTPGDECMDGILDESLLETCPIQSP | |
| | LQVFAGMGGLALIAERLPMLYPEVIQQVSAP | |
| | VVTSTTQEKPKDSDQFEWVTIEQSGELVYEA | |
| | PETVAAEPPPIKSAVQTMSPIPAHSLAAFGLF | |
| | LRLPGYAEVLLKERKHAQCLLRLVLGVTDDG | |
| | EGSHILQSPSANVLPTLPFHVLRSLFSTTPLT | |
| | TDDGVLLRRMALEIGALHLILVCLSALSHHSP | |
| | RVPNSSVNQTEPQVSSSHNPTSTEEQQLYW | |
| | AKGTGFGTGSTASGWDVEQALTKQRLEEEH | |
| | VTCLLQVLASYINPVSSAVNGEAQSSHETRG | |
| | QNSNALPSVLLELLSQSCLIPAMSSYLRNDSV | |
| | LDMARHVPLYRALLELLRAIASCAAMVPLLLP | |
| | LSTENGEEEEEQSECQTSVGTLLAKMKTCV | |
| | DTYTNRLRSKRENVKTGVKPDASDQEPEGL | |
| | TLLVPDIQKTAEIVYAATTSLRQANQEKKLGE | |
| | YSKKAAMKPKPLSVLKSLEEKYVAVMKKLQF | |
| | DTFEMVSEDEDGKLGFKVNYHYMSQVKNAN | |
| | DANSAARARRLAQEAVTLSTSLPLSSSSSVF | |
| | VRCDEERLDIMKVLITGPADTPYANGCFEFD | |
| | VYFPQDYPSSPPLVNLETTGGHSVRFNPNLY | |
| | NDGKVCLSILNTWHGRPEEKWNPQTSSFLQ | |
| | VLVSVQSLILVAEPYFNEPGYERSRGTPSGT | |
| | QSSREYDGNIRQATVKWAMLEQIRNPSPCF | |
| | KEVIHKHFYLKRVEIMAQCEEWIADIQQYSSD | |
| | KRVGRTMSHHAAALKRHTAQLREELLKLPCP | |
| | EGLDPDTDDAPEVCRATTGAEETLMHDQVK | |
| | PSSSKELPSDFQL | |
| FTS | MNPFWSMSTSSVRKRSEGEEKTLTGDVKTS | SEQ ID NO: |
| | PPRTAPKKQLPSIPKNALPITKPTSPAPAAQS | 39 |
| | TNGTHASYGPFYLEYSLLAEFTLVVKQKLPG | |
| | VYVQPSYRSALMWFGVIFIRHGLYQDGVFKF | |
| | TVYIPDNYPDGDCPRLVFDIPVFHPLVDPTSG | |
| | ELDVKRAFAKWRRNHNHIWQVLMYARRVFY | |
| | KIDTASPLNPEAAVLYEKDIQLFKSKVVDSVK | |
| | VCTARLFDQPKIEDPYAISFSPWNPSVHDEA | |
| | REKMLTQKKPEEQHNKSVHVAGLSWVKPGS | |
| | VQPFSKEEKTVAT | |
| TSG101 | MAVSESQLKKMVSKYKYRDLTVRETVNVITL | SEQ ID NO: |
| | YKDLKPVLDSYVFNDGSSRELMNLTGTIPVP | 40 |
| | YRGNTYNIPICLWLLDTYPYNPPICFVKPTSS | |
| | MTIKTGKHVDANGKIYLPYLHEWKHPQSDLL | |
| | GLIQVMIVVFGDEPPVFSRPISASYPPYQATG | |
| | PPNTSYMPGMPGGISPYPSGYPPNPSGYPG | |
| | CPYPPGGPYPATTSSQYPSQPPVTTVGPSR | |
| | DGTISEDTIRASLISAVSDKLRWRMKEEMDR | |
| | AQAELNALKRTEEDLKKGHQKLEEMVTRLD | |
| | QEVAEVDKNIELLKKKDEELSSALEKMENQS | |
| | ENNDIDEVIIPTAPLYKQILNLYAEENAIEDTIF | |
| | YLGEALRRGVIDLDVFLKHVRLLSRKQFQLR | |
| | ALMQKARKTAGLSDLY | |
| UFC1 | MADEATRRVVSEIPVLKTNAGPRDRELWVQ | SEQ ID NO: |
| | RLKEEYQSLIRYVENNKNADNDWFRLESNKE | 41 |
| | GTRWFGKCWYIHDLLKYEFDIEFDIPITYPTT | |
| | APEIAVPELDGKTAKMYRGGKICLTDHFKPL | |
| | WARNVPKFGLAHLMALGLGPWLAVEIPDLIQ | |
| | KGVIQHKEKCNQ | |

TABLE 8
UBC amino acid sequences of E2 ubiquitin-conjugating enzymes
and corresponding SEQ ID NOS.

| E2 ubiquitin- | | |
|---|---|---|
| conjugating | | |
| enzyme | UBC amino acid sequence | SEQ ID NO: |
| UBE2A | STPARRRLMRDFKRLQEDPPAGVSGAPSEN | SEQ ID NO: |
| | NIMVWNAVIFGPEGTPFEDGTFKLTIEFTEEY | 42 |
| | PNKPPTVRFVSKMFHPNVYADGSICLDILQN | |
| | RWSPTYDVSSILTSIQSLLDEPNPNSPANSQ | |
| | AAQLYQENKREYEKRVSAIVEQSWRDC | |
| UBE2B | STPARRRLMRDFKRLQEDPPVGVSGAPSEN | SEQ ID NO: |
| | NIMQWNAVIFGPEGTPFEDGTFKLVIEFSEEY | 43 |
| | PNKPPTVRFLSKMFHPNVYADGSICLDILQN | |
| | RWSPTYDVSSILTSIQSLLDEPNPNSPANSQ | |
| | AAQLYQENKREYEKRVSAIVEQSWNDS | |
| UBE2C | ASQNRDPAATSVAAARKGAEPSGGAARGPV | SEQ ID NO: |
| | GKRLQQELMTLMMSGDKGISAFPESDNLFK | 44 |
| | WVGTIHGAAGTVYEDLRYKLSLEFPSGYPYN | |
| | APTVKFLTPCYHPNVDTQGNICLDILKEKWSA | |
| | LYDVRTILLSIQSLLGEPNIDSPLNTHAAELWK | |
| | NPTAFKKYLQETYSKQVTSQEP | |
| UBE2D1 | ALKRIQKELSDLQRDPPAHCSAGPVGDDLFH | SEQ ID NO: |
| | WQATIMGPPDSAYQGGVFFLTVHFPTDYPF | 45 |
| | KPPKIAFTTKIYHPNINSNGSICLDILRSQWSP | |
| | ALTVSKVLLSICSLLCDPNPDDPLVPDIAQIYK | |
| | SDKEKYNRHAREWTQKYAM | |
| UBE2D2 | ALKRIHKELNDLARDPPAQCSAGPVGDDMF | SEQ ID NO: |
| | HWQATIMGPNDSPYQGGVFFLTIHFPTDYPF | 46 |
| | KPPKVAFTTRIYHPNINSNGSICLDILRSQWS | |
| | PALTISKVLLSICSLLCDPNPDDPLVPEIARIYK | |
| | TDREKYNRIAREWTQKYAM | |
| UBE2D3 | ALKRINKELSDLARDPPAQCSAGPVGDDMFH | SEQ ID NO: |
| | WQATIMGPNDSPYQGGVFFLTIHFPTDYPFK | 47 |
| | PPKVAFTTRIYHPNINSNGSICLDILRSQWSP | |
| | ALTISKVLLSICSLLCDPNPDDPLVPEIARIYKT | |
| | DRDKYNRISREWTQKYAM | |
| UBE2D4 | ALKRIQKELTDLQRDPPAQCSAGPVGDDLFH | SEQ ID NO: |
| | WQATIMGPNDSPYQGGVFFLTIHFPTDYPFK | 48 |
| | PPKVAFTTKIYHPNINSNGSICLDILRSQWSP | |
| | ALTVSKVLLSICSLLCDPNPDDPLVPEIAHTYK | |
| | ADREKYNRLAREWTQKYAM | |
| UBE2E1 | SDDDSRASTSSSSSSSSNQQTEKETNTPKK | SEQ ID NO: |
| | KESKVSMSKNSKLLSTSAKRIQKELADITLDP | 49 |
| | PPNCSAGPKGDNIYEWRSTILGPPGSVYEG | |
| | GVFFLDITFTPEYPFKPPKVTFRTRIYHCNINS | |
| | QGVICLDILKDNWSPALTISKVLLSICSLLTDC | |
| | NPADPLVGSIATQYMTNRAEHDRMARQWTK | |
| | RYAT | |
| UBE2E2 | STSGGSSDGDQRESVQQEPEREQVQPKKK | SEQ ID NO: |
| | EGKISSKTAAKLSTSAKRIQKELAEITLDPPPN | 50 |
| | CSAGPKGDNIYEWRSTILGPPGSVYEGGVFF | |
| | LDITFSPDYPFKPPKVTFRTRIYHCNINSQGVI | |
| | CLDILKDNWSPALTISKVLLSICSLLTDQNPAD | |
| | PLVGSIATQYMTNRAEHDRMARQWTKRYAT | |
| UBE2E3 | LSTSAKRIQKELAEITLDPPPNCSAGPKGDNI | SEQ ID NO: |
| | YEWRSTILGPPGSVYEGGVFFLDITFSSDYPF | 51 |
| | KPPKVTFRTRIYHCNINSQGVICLDILKDNWS | |
| | PALTISKVLLSICSLLTDCNPADPLVGSIATQY | |
| | LTNRAEHDRIARQWTKRYAT | |
| UBE2F | STRRVSVRDKLLVKEVAELEANLPCTCKVHF | SEQ ID NO: |
| | PDPNKLHCFQLTVTPDEGYYQGGKFQFETE | 52 |
| | VPDAYNMVPPKVKCLTKIWHPNITETGEICLS | |
| | LLREHSIDGTGWAPTRTLKDVVWGLNSLFTD | |
| | LLNFDDPLNIEAAEHHLRDKEDFRNKVDDYIK | |
| | RYAR | |
| UBE2G1 | LLLRRQLAELNKNPVEGFSAGLIDDNDLYRW | SEQ ID NO: |
| | EVLIIGPPDTLYEGGVFKAHLTFPKDYPLRPP | 53 |
| | KMKFITEIWHPNVDKNGDVCISILHEPGEDKY | |
| | GYEKPEERWLPIHTVETIMISVISMLADPNGD | |
| | SPANVDAAKEWREDRNGEFKRKVARC | |
| UBE2G2 | AGTALKRLMAEYKQLTLNPPEGIVAGPMNEE | SEQ ID NO: |
| | NFFEWEALIMGPEDTCFEFGVFPAILSFPLDY | 54 |
| | PLSPPKMRFTCEMFHPNIYPDGRVCISILHAP | |
| | GDDPMGYESSAERWSPVQSVEKILLSVVSM | |
| | LAEPNDESGANVDASKMWRDDREQFYKIAK | |
| | QIVQKSLGL | |
| UBE2H | SSPSPGKRRMDTDVVKLIESKHEVTILGGLN | SEQ ID NO: |
| | EFVVKFYGPQGTPYEGGVWKVRVDLPDKYP | 55 |
| | FKSPSIGFMNKIFHPNIDEASGTVCLDVINQT | |
| | WTALYDLTNIFESFLPQLLAYPNPIDPLNGDA | |
| | AAMYLHRPEEYKQKIKEYIQKYATEEALKEQE | |
| | EG | |
| UBE21 | SGIALSRLAQERKAWRKDHPFGFVAVPTKNP | SEQ ID NO: |
| | DGTMNLMNWECAIPGKKGTPWEGGLFKLR | 56 |
| | MLFKDDYPSSPPKCKFEPPLFHPNVYPSGTV | |
| | CLSILEEDKDWRPAITIKQILLGIQELLNEPNIQ | |
| | DPAQAEAYTIYCQNRVEYEKRVRAQAKKFAP | |
| | S | |
| UBE2J1 | ETRYNLKSPAVKRLMKEAAELKDPTDHYHAQ | SEQ ID NO: |
| | PLEDNLFEWHFTVRGPPDSDFDGGVYHGRI | 57 |
| | VLPPEYPMKPPSIILLTANGRFEVGKKICLSIS | |
| | GHHPETWQPSWSIRTALLAIIGFMPTKGEGAI | |
| | GSLDYTPEERRALAKKSQDFCCEGCGSAMK | |
| | DVLLPLKSGSDSSQADQEAKELARQISFKAE | |
| | VNSSGKTISESDLNHSFSLTDLQDDIPTTFQG | |
| | ATASTSYGLQNSSAASFHQPTQPVAKNTSM | |
| | SPRQRRAQQQSQRRLSTSPDVIQGHQPRDN | |
| | HTDHG | |
| UBE2J2 | SSTSSKRAPTTATQRLKQDYLRIKKDPVPYIC | SEQ ID NO: |
| | AEPLPSNILEWHYVVRGPEMTPYEGGYYHG | 58 |
| | KLIFPREFPFKPPSIYMITPNGRFKCNTRLCLS | |
| | ITDFHPDTWNPAWSVSTILTGLLSFMVEKGP | |
| | TLGSIETSDFTKRQLAVQSLAFNLKDKVFCEL | |
| | FPEVVEEIKQKQKAQDELSSRPQTLP | |
| UBE2K | ANIAVQRIKREFKEVLKSEETSKNQIKVDLVD | SEQ ID NO: |
| | ENFTELRGEIAGPPDTPYEGGRYQLEIKIPET | 59 |
| | YPFNPPKVRFITKIWHPNISSVTGAICLDILKD | |
| | QWAAAMTLRTVLLSLQALLAAAEPDDPQDAV | |
| | VANQYKQNPEMFKQTARLWAHVYAGAPVSS | |
| | PEYTKKIENLCAMGFDRNAVIVALSSKSWDV | |
| | ETATELLLSN | |
| UBE2L3 | AASRRLMKELEEIRKCGMKNFRNIQVDEANL | SEQ ID NO: |
| | LTWQGLIVPDNPPYDKGAFRIEINFPAEYPFK | 60 |
| | PPKITFKTKIYHPNIDEKGQVCLPVISAENWK | |
| | PATKTDQVIQSLIALVNDPQPEHPLRADLAEE | |
| | YSKDRKKFCKNAEEFTKKYGEKRPVD | |
| UBE2L6 | MASMRVVKELEDLQKKPPPYLRNLSSDDAN | SEQ ID NO: |
| | VLVWHALLLPDQPPYHLKAFNLRISFPPEYPF | 61 |
| | KPPMIKFTTKIYHPNVDENGQICLPIISSENWK | |
| | PCTKTCQVLEALNVLVNRPNIREPLRMDLAD | |
| | LLTQNPELFRKNAEEFTLRFGVDRPS | |
| UBE2M | ASAAQLRIQKDINELNLPKTCDISFSDPDDLL | SEQ ID NO: |
| | NFKLVICPDEGFYKSGKFVFSFKVGQGYPHD | 62 |
| | PPKVKCETMVYHPNIDLEGNVCLNILREDWK | |
| | PVLTINSIIYGLQYLFLEPNPEDPLNKEAAEVL | |
| | QNNRRLFEQNVQRSMRGGYIGSTYFERCLK | |
| UBE2N | AGLPRRIIKETQRLLAEPVPGIKAEPDESNAR | SEQ ID NO: |
| | YFHVVIAGPQDSPFEGGTFKLELFLPEEYPM | 63 |
| | AAPKVRFMTKIYHPNVDKLGRICLDILKDKWS | |
| | PALQIRTVLLSIQALLSAPNPDDPLANDVAEQ | |
| | WKTNEAQAIETARAWTRLYAMNNI | |
| UBE2NL | AELPHRIIKETQRLLAEPVPGIKAEPDESNAR | SEQ ID NO: |
| | YFHVVIAGESKDSPFEGGTFKRELLLAEEYP | 64 |
| | MAAPKVRFMTKIYHPNVDKLERISLDILKDKW | |
| | SPALQIRTVLLSIQALLNAPNPDDPLANDVVE | |
| | QWKTNEAQAIETARAWTRLYAMNSI | |
| UBE20 | SNHSFKKIEFQPPEAKKFFSTVRKEMALLATS | SEQ ID NO: |
| | LPEGIMVKTFEDRMDLFSALIKGPTRTPYEDG | 65 |
| | LYLFDIQLPNIYPAVPPHFCYLSQCSGRLNPN | |
| | LYDNGKVCVSLLGTWIGKGTERWTSKSSLLQ | |
| | VLISIQGLILV | |
| UBE2Q1 | SGSVQATDRLMKELRDIYRSQSFKGGNYAV | SEQ ID NO: |
| | ELVNDSLYDWNVKLLKVDQDSALHNDLQILK | 66 |
| | EKEGADFILLNFSFKDNFPFDPPFVRVVSPVL | |
| | SGGYVLGGGAICMELLTKQGWSSAYSIESVI | |
| | MQISATLVKGKARVQFGANKSQYSLTRAQQ | |
| | SYKSLVQIHEKNGW | |
| UBE2Q2 | GAVSGSVQASDRLMKELRDIYRSQSYKTGIY | SEQ ID NO: |
| | SVELINDSLYDWHVKLQKVDPDSPLHSDLQIL | 67 |
| | KEKEGIEYILLNFSFKDNFPFDPPFVRVVLPVL | |
| | SGGYVLGGGALCMELLTKQGWSSAYSIESVI | |
| | MQINATLVKGKARVQFGANKNQYNLARAQQ | |
| | SYNSIVQIHE | |
| UBE2QL | KELQDIARLSDRFISVELVDESLFDWNVKLHQ | SEQ ID NO: |
| | VDKDSVLWQDMKETNTEFILLNLTFPDNFPF | 68 |
| | SPPFMRVLSPRLENGYVLDGGAICMELLTPR | |
| | GWSSAYTVEAVMRQFAASLVKGQGRICRKA | |
| | GKSKKSFSRKEAEATFKSLVKTHEKYGWVTP | |
| | PVSDG | |
| UBE2R1 | PSSQKALLLELKGLQEEPVEGFRVTLVDEGD | SEQ ID NO: |
| | LYNWEVAIFGPPNTYYEGGYFKARLKFPIDY | 69 |
| | PYSPPAFRFLTKMWHPNIYETGDVCISILHPP | |
| | VDDPQSGELPSERWNPTQNVRTILLSVISLLN | |
| | EPNTFSPANVDASVMYRKWKESKGKDREYT | |
| | DIIRKQVLGTKVDAERDGVKVP | |
| UBE2R2 | TSSQKALMLELKSLQEEPVEGFRITLVDESDL | SEQ ID NO: |
| | YNWEVAIFGPPNTLYEGGYFKAHIKFPIDYPY | 70 |
| | SPPTFRFLTKMWHPNIYENGDVCISILHPPVD | |
| | DPQSGELPSERWNPTQNVRTILLSVISLLNEP | |
| | NTFSPANVDASVMFRKWRDSKGKDKEYAEII | |
| | RKQVSATKAEAEKDGVKVP | |
| UBE2S | NSNVENLPPHIIRLVYKEVTTLTADPPDGIKVF | SEQ ID NO: |
| | PNEEDLTDLQVTIEGPEGTPYAGGLFRMKLL | 71 |
| | LGKDFPASPPKGYFLTKIFHPNVGANGEICVN | |
| | VLKRDWTAELGIRHVLLTIKCLLIHPNPESALN | |
| | EEAGRLLLENYEEYAARARLLTEIHG | |
| UBE2T | QRASRLKRELHMLATEPPPGITCWQDKDQM | SEQ ID NO: |
| | DDLRAQILGGANTPYEKGVFKLEVIIPERYPF | 72 |
| | EPPQIRFLTPIYHPNIDSAGRICLDVLKLPPKG | |
| | AWRPSLNIATVLTSIQLLMSEPNPDDPLMADI | |
| | SSEFKYNKPAFLKNARQWTEKHARQKQKAD | |
| | EEEMLDNLP | |
| UBE2U | HGRAYLLLHRDFCDLKENNYKGITAKPVSED | SEQ ID NO: |
| | MMEWEVEIEGLQNSVWQGLVFQLTIHFTSEY | 73 |
| | NYAPPVVKFITIPFHPNVDPHTGQPCIDFLDN | |
| | PEKWNTNYTLSSILLALQVMLSNPVLENPVNL | |
| | EAARILVKDESLYRTILRLFNRP | |
| UBE2V1 | GVKVPRNFRLLEELEEGQKGVGDGTVSWGL | SEQ ID NO: |
| | EDDEDMTLTRWTGMIIGPPRTIYENRIYSLKIE | 74 |
| | CGPKYPEAPPFVRFVTKINMNGVNSSNGVV | |
| | DPRAISVLAKWQNSYSIKVVLQELRRLMMSK | |
| | ENMKLPQPPEGQCYSN | |
| UBE2V2 | AVSTGVKVPRNFRLLEELEEGQKGVGDGTV | SEQ ID NO: |
| | SWGLEDDEDMTLTRWTGMIIGPPRTNYENRI | 75 |
| | YSLKVECGPKYPEAPPSVRFVTKINMNGINN | |
| | SSGMVDARSIPVLAKWQNSYSIKVVLQELRR | |
| | LMMSKENMKLPQPPEGQTYNN | |
| UBE2W | MASMQKRLQKELLALQNDPPPGMTLNEKSV | SEQ ID NO: |
| | QNSITQWIVDMEGAPGTLYEGEKFQLLFKFS | 76 |
| | SRYPFDSPQVMFTGENIPVHPHVYSNGHICL | |
| | SILTEDWSPALSVQSVCLSIISMLS | |
| UBE2Z | MSIYKEPPPGMFVVPDTVDMTKIHALITGPFD | SEQ ID NO: |
| | TPYEGGFFLFVFRCPPDYPIHPPRVKLMTTG | 77 |
| | NNTVRFNPNFYRNGKVCLSILGTWTGPAWS | |
| | PAQSISSVLISIQSLMTENPYHNEPGFEQERH | |
| | PGDSKNYNECIRHETIRVAV | |
| UEVLD | MEFDCEGLRRLLGKYKFRDLTVEELRNVNVF | SEQ ID NO: |
| | FPHFKYSMDTYVFKDSSQKDLLNFTGTIPVM | 78 |
| | YQGNTYNIPIRFWILDSHPFAPPICFLKPTAN | |
| | MGILVGKHVDAQGRIYLPYLQNWSHPKSVIV | |
| | GLIKEMIAKFQEELPMYSLSSSDEARQVDLLA | |
| | YIAKITEGVSDTNSKSWANHENKTV | |
| BIRC6 | ANQEKKLGEYSKKAAMKPKPLSVLKSLEEKY | SEQ ID NO: |
| | VAVMKKLQFDTFEMVSEDEDGKLGFKVNYH | 79 |
| | YMSQVKNANDANSAARARRLAQEAVTLSTS | |
| | LPLSSSSSVFVRCDEERLDIMKVLITGPADTP | |
| | YANGCFEFDVYFPQDYPSSPPLVNLETTGG | |
| | HSVRFNPNLYNDGKVCLSILNTWHGRPEEK | |
| | WNPQTSSFLQVLVSVQSLIL | |
| FTS | PPRTAPKKQLPSIPKNALPITKPTSPAPAAQS | SEQ ID NO: |
| | TNGTHASYGPFYLEYSLLAEFTLVVKQKLPG | 80 |
| | VYVQPSYRSALMWFGVIFIRHGLYQDGVFKF | |
| | TVYIPDNYPDGDCPRLVFDIPVFHPLVDPTSG | |
| | ELDVKRAFAKWRRNHNHIWQVLMYARRVFY | |
| | KIDTASPLNPEAAVLYEKDIQLFKSKVVDSVK | |
| | VCTARLFDQPKIED | |
| TSG101 | AVSESQLKKMVSKYKYRDLTVRETVNVITLY | SEQ ID NO: |
| | KDLKPVLDSYVFNDGSSRELMNLTGTIPVPY | 81 |
| | RGNTYNIPICLWLLDTYPYNPPICFVKPTSSM | |
| | TIKTGKHVDANGKIYLPYLHEWKHPQSDLLG | |
| | LIQVMIVVFGDEPPVFSRP | |
| UFC1 | ADEATRRVVSEIPVLKTNAGPRDRELWVQRL | SEQ ID NO: |
| | KEEYQSLIRYVENNKNADNDWFRLESNKEG | 82 |
| | TRWFGKCWYIHDLLKYEFDIEFDIPITYPTTAP | |
| | EIAVPELDGKTAKMYRGGKICLTDHFKPLWA | |
| | RNVPKFGLAHLMALGLGPWLAVEIPDLIQKG | |
| | VIQHKEKCNQ | |

TABLE 9
UBC nucleic acid sequences of E2 ubiquitin-conjugating enzymes and
corresponding SEQ ID NOS.

| E2 ubiquitin- | | |
|---|---|---|
| conjugating | | |
| enzyme | UBC nucleic acid sequence | SEQ ID NO: |
| UBE2A | TCCACCCCGGCTCGGCGGCGCCTCATGCG | SEQ ID NO: |
| | GGACTTCAAGAGGTTGCAGGAGGATCCTCC | 83 |
| | AGCCGGAGTCAGCGGGGCTCCGTCCGAGA | |
| | ACAACATAATGGTGTGGAACGCGGTCATTTT | |
| | CGGGCCTGAAGGGACCCCGTTTGAGGATGG | |
| | AACATTTAAACTTACAATAGAATTCACTGAAG | |
| | AATATCCAAATAAACCACCTACAGTTAGATTT | |
| | GTCTCTAAGATGTTCCATCCAAATGTCTATG | |
| | CAGATGGTAGTATATGTCTGGACATACTTCA | |
| | GAACCGTTGGAGTCCAACCTATGATGTGTCT | |
| | TCCATTCTAACATCCATACAGTCTCTGTTGG | |
| | ATGAACCCAATCCCAATAGTCCAGCAAACAG | |
| | CCAGGCTGCTCAGCTGTACCAGGAGAACAA | |
| | ACGGGAATATGAAAAGCGTGTTTCTGCAATA | |
| | GTAGAACAAAGCTGGCGTGATTGT | |
| UBE2B | AGCACACCAGCTCGGCGGAGACTGATGAGA | SEQ ID NO: |
| | GACTTCAAGCGGCTGCAAGAGGACCCTCCT | 84 |
| | GTGGGAGTTTCTGGCGCCCCTAGCGAGAAC | |
| | AACATCATGCAGTGGAACGCCGTGATCTTC | |
| | GGCCCTGAGGGCACCCCTTTTGAGGACGGC | |
| | ACCTTCAAGCTGGTCATCGAGTTCAGCGAG | |
| | GAATACCCCAACAAGCCTCCTACCGTGCGG | |
| | TTCCTGAGCAAGATGTTTCACCCCAACGTGT | |
| | ACGCCGACGGCAGCATCTGTCTGGACATCC | |
| | TGCAGAACAGATGGTCCCCAACCTACGATG | |
| | TGTCCAGCATCCTGACCAGCATCCAGAGCC | |
| | TGCTGGACGAGCCCAATCCTAACAGCCCTG | |
| | CCAATTCTCAGGCCGCTCAGCTGTACCAAG | |
| | AGAACAAGCGCGAGTACGAGAAGCGGGTGT | |
| | CCGCCATCGTTGAGCAGAGCTGGAATGACA | |
| | GC | |
| UBE2C | GCCAGCCAGAACAGAGATCCTGCCGCCACA | SEQ ID NO: |
| | TCTGTGGCCGCTGCTAGAAAAGGCGCTGAG | 85 |
| | CCTTCTGGCGGAGCTGCCAGAGGACCTGTG | |
| | GGAAAGAGACTGCAGCAAGAGCTGATGACC | |
| | CTGATGATGAGCGGCGACAAGGGCATCTCC | |
| | GCCTTTCCAGAGAGCGACAACCTGTTCAAAT | |
| | GGGTCGGAACCATCCACGGCGCTGCCGGC | |
| | ACAGTGTATGAGGACCTGAGATACAAGCTG | |
| | AGCCTGGAATTTCCCAGCGGCTACCCCTAC | |
| | AATGCCCCTACCGTGAAGTTTCTGACCCCTT | |
| | GCTATCACCCCAACGTGGACACCCAGGGCA | |
| | ACATCTGCCTGGACATCCTGAAAGAGAAGT | |
| | GGAGCGCCCTGTACGATGTGCGGACAATCC | |
| | TGCTGAGCATCCAGTCTCTGCTGGGCGAGC | |
| | CCAACATCGACAGCCCTCTGAATACTCACGC | |
| | CGCCGAGCTGTGGAAGAACCCCACCGCCTT | |
| | TAAGAAGTACCTGCAAGAGACATACAGCAAG | |
| | CAAGTGACCAGCCAAGAGCCT | |
| UBE2D1 | GCCCTGAAGAGAATCCAGAAAGAGCTGAGC | SEQ ID NO: |
| | GACCTGCAGAGAGATCCTCCTGCTCACTGTT | 86 |
| | CTGCTGGCCCTGTGGGAGATGACCTGTTCC | |
| | ACTGGCAAGCCACCATCATGGGCCCTCCAG | |
| | ATAGCGCCTATCAAGGCGGCGTGTTCTTCCT | |
| | GACCGTGCACTTCCCCACAGACTACCCCTT | |
| | CAAGCCTCCTAAGATCGCCTTCACCACCAAG | |
| | ATCTATCACCCCAACATCAACAGCAACGGCA | |
| | GCATCTGCCTGGACATCCTGAGAAGCCAAT | |
| | GGTCCCCTGCTCTGACCGTGTCCAAGGTGC | |
| | TGCTGAGCATCTGCAGCCTGCTGTGCGACC | |
| | CCAATCCTGACGATCCTCTGGTGCCTGATAT | |
| | CGCCCAGATCTACAAGAGCGACAAAGAGAA | |
| | GTACAACCGGCACGCCAGAGAGTGGACCCA | |
| | GAAATACGCTATG | |
| UBE2D2 | GCTCTGAAGAGAATCCACAAAGAGCTGAAC | SEQ ID NO: |
| | GACCTGGCCAGAGATCCTCCTGCTCAGTGT | 87 |
| | TCTGCTGGCCCTGTGGGCGACGACATGTTT | |
| | CACTGGCAAGCCACCATCATGGGCCCCAAC | |
| | GACAGCCCTTATCAAGGCGGCGTGTTCTTC | |
| | CTGACCATTCACTTCCCCACAGACTACCCCT | |
| | TCAAGCCTCCTAAGGTGGCCTTCACCACCA | |
| | GAATCTATCACCCCAACATCAACAGCAACGG | |
| | CAGCATCTGCCTGGACATCCTGAGAAGCCA | |
| | ATGGTCCCCTGCTCTGACCATCAGCAAGGT | |
| | GCTGCTGAGCATCTGCAGCCTGCTGTGCGA | |
| | CCCCAATCCTGACGATCCTCTGGTGCCTGA | |
| | GATCGCCCGGATCTACAAGACCGACAGAGA | |
| | GAAGTACAACCGGATCGCCAGAGAGTGGAC | |
| | CCAGAAATACGCCATG | |
| UBE2D3 | GCTCTGAAGCGGATCAACAAAGAGCTGAGC | SEQ ID NO: |
| | GACCTGGCCAGAGATCCTCCTGCTCAGTGT | 88 |
| | TCTGCTGGCCCTGTGGGCGACGACATGTTT | |
| | CACTGGCAAGCCACCATCATGGGCCCCAAC | |
| | GACAGCCCTTATCAAGGCGGCGTGTTCTTC | |
| | CTGACCATTCACTTCCCCACAGACTACCCCT | |
| | TCAAGCCTCCTAAGGTGGCCTTCACCACCA | |
| | GAATCTATCACCCCAACATCAACAGCAACGG | |
| | CAGCATCTGCCTGGACATCCTGAGAAGCCA | |
| | ATGGTCCCCTGCTCTGACCATCAGCAAGGT | |
| | GCTGCTGAGCATCTGCAGCCTGCTGTGCGA | |
| | CCCCAATCCTGACGATCCTCTGGTGCCTGA | |
| | GATCGCCCGGATCTACAAGACCGACCGGGA | |
| | CAAGTACAACCGGATCAGCAGAGAGTGGAC | |
| | CCAGAAATACGCCATG | |
| UBE2D4 | GCTCTGAAGAGAATCCAGAAAGAGCTGACC | SEQ ID NO: |
| | GACCTGCAGCGGGACCCTCCTGCTCAATGT | 89 |
| | TCTGCTGGACCCGTGGGCGACGACCTGTTT | |
| | CATTGGCAAGCCACCATCATGGGCCCCAAC | |
| | GACAGCCCTTATCAAGGCGGCGTGTTCTTC | |
| | CTGACCATTCACTTCCCCACAGACTACCCCT | |
| | TCAAGCCTCCTAAGGTGGCCTTCACCACCAA | |
| | GATCTATCACCCCAACATCAACAGCAACGGC | |
| | AGCATCTGCCTGGACATCCTGAGAAGCCAA | |
| | TGGTCCCCTGCTCTGACCGTGTCCAAGGTG | |
| | CTGCTGAGCATCTGCAGCCTGCTGTGCGAC | |
| | CCCAATCCTGACGATCCTCTGGTGCCTGAG | |
| | ATCGCCCACACCTACAAGGCCGACAGAGAG | |
| | AAGTACAACCGGCTGGCCAGAGAGTGGACC | |
| | CAGAAATACGCTATG | |
| UBE2E1 | AGCGACGATGACAGCAGAGCCAGCACCTCT | SEQ ID NO: |
| | AGCAGCAGCAGCTCCAGCAGCAATCAGCAG | 90 |
| | ACCGAGAAAGAGACAAACACGCCCAAGAAA | |
| | AAAGAAAGCAAGGTGTCCATGAGCAAGAAC | |
| | AGCAAGCTGCTGAGCACCAGCGCCAAGAGA | |
| | ATCCAGAAAGAGCTGGCCGACATCACACTG | |
| | GACCCTCCACCTAATTGCAGCGCCGGACCT | |
| | AAGGGCGACAACATCTATGAGTGGCGGAGC | |
| | ACCATCCTGGGACCACCTGGCTCTGTTTATG | |
| | AAGGCGGCGTGTTCTTCCTGGACATCACCTT | |
| | CACACCTGAGTACCCCTTCAAGCCTCCTAAA | |
| | GTGACCTTCCGGACCAGAATCTACCACTGC | |
| | AACATCAACAGCCAGGGCGTGATCTGCCTG | |
| | GATATCCTGAAGGACAATTGGAGCCCCGCT | |
| | CTGACCATCAGCAAGGTGCTGCTGTCCATCT | |
| | GCAGCCTGCTGACCGACTGCAATCCTGCCG | |
| | ATCCTCTCGTGGGCTCTATCGCCACACAGTA | |
| | CATGACCAACAGAGCCGAGCACGACCGGAT | |
| | GGCCAGACAGTGGACAAAGAGATACGCCAC | |
| | A | |
| UBE2E2 | AGCACATCTGGCGGAAGCTCCGATGGCGAT | SEQ ID NO: |
| | CAGAGGGAATCTGTGCAGCAAGAGCCCGAG | 91 |
| | CGAGAACAGGTGCAGCCCAAGAAGAAAGAG | |
| | GGCAAGATCAGCAGCAAGACCGCCGCCAAG | |
| | CTGAGCACAAGCGCCAAGAGAATCCAGAAA | |
| | GAGCTGGCCGAGATCACACTGGACCCTCCA | |
| | CCTAATTGCAGCGCCGGACCTAAGGGCGAC | |
| | AACATCTATGAGTGGCGGAGCACCATCCTG | |
| | GGACCACCTGGCTCTGTTTATGAAGGCGGC | |
| | GTGTTCTTCCTGGACATCACATTCAGCCCTG | |
| | ACTACCCCTTCAAGCCTCCTAAAGTGACCTT | |
| | CCGGACCAGAATCTACCACTGCAACATCAAC | |
| | AGCCAGGGCGTGATCTGCCTGGATATCCTG | |
| | AAGGACAATTGGAGCCCCGCTCTGACCATC | |
| | AGCAAGGTGCTGCTGAGCATCTGCAGCCTG | |
| | CTGACCGACTGCAATCCTGCCGATCCTCTC | |
| | GTGGGCTCTATCGCCACACAGTACATGACC | |
| | AACAGAGCCGAGCACGACCGGATGGCCAGA | |
| | CAGTGGACAAAGAGATACGCCACA | |
| UBE2E3 | TTATCCACTAGTGCTAAAAGAATTCAGAAGG | SEQ ID NO: |
| | AGCTAGCTGAAATAACCCTTGATCCTCCTCC | 92 |
| | TAATTGCAGTGCTGGGCCTAAAGGAGATAAC | |
| | ATTTATGAATGGAGATCAACTATACTTGGTC | |
| | CACCGGGTTCTGTATATGAAGGTGGTGTGTT | |
| | TTTTCTGGATATCACATTTTCATCAGATTATC | |
| | CATTTAAGCCACCAAAGGTTACTTTCCGCAC | |
| | CAGAATCTATCACTGCAACATCAACAGTCAG | |
| | GGAGTCATCTGTCTGGACATCCTTAAAGACA | |
| | ACTGGAGTCCCGCTTTGACTATTTCAAAGGT | |
| | TTTGCTGTCTATTTGTTCCCTTTTGACAGACT | |
| | GCAACCCTGCGGATCCTCTGGTTGGAAGCA | |
| | TAGCCACTCAGTATTTGACCAACAGAGCAGA | |
| | ACACGACAGGATAGCCAGACAGTGGACCAA | |
| | GAGATACGCAACA | |
| UBE2F | AGCACCAGACGGGTGTCAGTGCGGGATAAG | SEQ ID NO: |
| | CTGCTGGTCAAAGAGGTGGCCGAGCTGGAA | 93 |
| | GCCAACCTGCCTTGTACATGCAAGGTGCAC | |
| | TTCCCCGATCCTAACAAGCTGCACTGCTTTC | |
| | AGCTGACCGTGACACCCGACGAGGGCTACT | |
| | ATCAAGGCGGCAAGTTCCAGTTCGAGACAG | |
| | AGGTGCCCGACGCCTACAACATGGTGCCTC | |
| | CAAAAGTGAAGTGCCTGACCAAGATCTGGC | |
| | ACCCCAACATCACCGAGACAGGCGAGATCT | |
| | GTCTGAGCCTGCTGAGAGAGCACAGCATCG | |
| | ACGGAACAGGATGGGCCCCTACCAGAACAC | |
| | TGAAGGATGTTGTGTGGGGCCTGAACTCCC | |
| | TGTTCACCGACCTGCTGAACTTCGACGACC | |
| | CTCTGAATATCGAGGCCGCCGAGCACCACC | |
| | TGAGAGACAAAGAGGACTTCCGGAACAAGG | |
| | TGGACGACTACATCAAGAGATACGCCCGG | |
| UBE2G1 | CTGCTGCTGAGAAGGCAGCTGGCCGAGCTG | SEQ ID NO: |
| | AACAAGAACCCCGTGGAAGGCTTTTCTGCC | 94 |
| | GGCCTGATCGACGACAACGACCTGTACAGA | |
| | TGGGAAGTGCTGATCATCGGCCCTCCAGAC | |
| | ACACTGTATGAAGGCGGCGTGTTCAAGGCC | |
| | CACCTGACATTCCCCAAGGACTACCCTCTGC | |
| | GGCCTCCTAAGATGAAGTTCATCACCGAGAT | |
| | CTGGCACCCCAACGTGGACAAGAATGGCGA | |
| | CGTGTGCATCAGCATCCTGCACGAGCCTGG | |
| | CGAGGATAAGTACGGCTACGAGAAGCCCGA | |
| | GGAACGGTGGCTGCCTATCCACACCGTGGA | |
| | AACCATCATGATCAGCGTGATCTCCATGCTG | |
| | GCCGATCCTAACGGCGACAGCCCTGCCAAT | |
| | GTGGATGCCGCCAAAGAGTGGCGCGAGGA | |
| | CAGAAACGGCGAGTTCAAGAGAAAGGTGGC | |
| | CCGGTGT | |
| UBE2G2 | GCCGGAACAGCCCTGAAAAGACTGATGGCC | SEQ ID NO: |
| | GAGTACAAGCAGCTGACCCTGAATCCTCCT | 95 |
| | GAGGGCATTGTGGCTGGCCCTATGAACGAA | |
| | GAGAACTTCTTCGAGTGGGAAGCCCTGATC | |
| | ATGGGCCCCGAGGATACCTGCTTTGAGTTC | |
| | GGAGTGTTCCCCGCTATCCTGAGCTTCCCT | |
| | CTGGACTACCCTCTGAGCCCTCCTAAGATG | |
| | CGGTTTACCTGCGAGATGTTTCACCCCAACA | |
| | TCTACCCCGACGGCAGAGTGTGCATCAGCA | |
| | TTCTGCATGCCCCTGGCGACGACCCTATGG | |
| | GCTATGAATCTTCTGCCGAGCGGTGGTCCC | |
| | CTGTGCAGAGCGTGGAAAAGATCCTGCTGA | |
| | GCGTGGTGTCCATGCTGGCCGAGCCTAATG | |
| | ATGAGAGCGGCGCCAATGTGGACGCCAGCA | |
| | AAATGTGGCGGGACGACAGAGAGCAGTTCT | |
| | ACAAGATCGCCAAGCAGATCGTGCAGAAGT | |
| | CCCTGGGATTG | |
| UBE2H | AGCTCTCCATCTCCTGGCAAGCGGAGAATG | SEQ ID NO: |
| | GACACCGACGTGGTCAAGCTGATCGAGAGC | 96 |
| | AAGCACGAAGTGACCATCCTCGGCGGCCTG | |
| | AACGAGTTCGTCGTGAAGTTCTATGGACCCC | |
| | AGGGCACCCCTTATGAAGGCGGCGTTTGGA | |
| | AAGTGCGCGTGGACCTGCCTGACAAGTACC | |
| | CCTTTAAGAGCCCCAGCATCGGCTTCATGAA | |
| | CAAGATCTTTCACCCCAACATCGACGAGGC | |
| | CAGCGGCACCGTTTGTCTGGACGTGATCAA | |
| | CCAGACCTGGACAGCCCTGTACGACCTGAC | |
| | CAATATCTTCGAGAGCTTCCTGCCTCAGCTG | |
| | CTGGCTTACCCCAATCCTATCGACCCTCTGA | |
| | ATGGCGACGCCGCTGCCATGTATCTGCACA | |
| | GACCCGAAGAGTACAAGCAGAAGATCAAAG | |
| | AGTACATCCAGAAGTACGCCACCGAGGAAG | |
| | CCCTGAAAGAGCAAGAGGAAGGC | |
| UBE21 | AGCGGAATTGCCCTGAGCAGACTGGCCCAA | SEQ ID NO: |
| | GAGAGAAAGGCCTGGCGGAAGGATCACCCC | 97 |
| | TTCGGCTTTGTGGCCGTGCCTACCAAGAATC | |
| | CCGACGGCACCATGAACCTGATGAACTGGG | |
| | AGTGCGCTATCCCCGGCAAGAAGGGCACAC | |
| | CTTGGGAAGGCGGACTGTTCAAGCTGCGGA | |
| | TGCTGTTCAAGGACGACTACCCTAGCAGCC | |
| | CTCCTAAGTGCAAGTTCGAGCCTCCTCTGTT | |
| | TCACCCCAACGTGTACCCTAGCGGCACCGT | |
| | GTGTCTGAGCATCCTGGAAGAGGACAAGGA | |
| | TTGGAGGCCCGCCATCACCATCAAGCAGAT | |
| | CCTGCTGGGCATCCAAGAGCTGCTGAACGA | |
| | GCCCAACATTCAGGACCCTGCTCAGGCCGA | |
| | GGCCTACACCATCTACTGCCAGAACAGAGT | |
| | GGAATACGAGAAGAGAGTCAGAGCCCAGGC | |
| | CAAGAAGTTCGCCCCATCT | |
| UBE2J1 | GAGACCCGCTACAACCTGAAGAGTCCGGCT | SEQ ID NO: |
| | GTTAAACGTTTAATGAAAGAAGCGGCAGAAT | 98 |
| | TGAAAGATCCAACAGATCATTACCATGCGCA | |
| | GCCTTTAGAGGATAACCTTTTTGAATGGCAC | |
| | TTCACGGTTAGAGGGCCCCCAGACTCCGAT | |
| | TTTGATGGAGGAGTTTATCACGGGCGGATA | |
| | GTACTGCCACCAGAGTATCCCATGAAACCAC | |
| | CAAGCATTATTCTCCTAACGGCTAATGGTCG | |
| | ATTTGAAGTGGGCAAGAAAATCTGTTTGAGC | |
| | ATCTCAGGCCATCATCCTGAAACTTGGCAGC | |
| | CTTCGTGGAGTATAAGGACAGCATTATTAGC | |
| | CATCATTGGGTTTATGCCAACAAAAGGAGAG | |
| | GGAGCCATAGGTTCTCTAGATTACACTCCTG | |
| | AGGAAAGAAGAGCACTTGCCAAAAAATCACA | |
| | AGATTTCTGTTGTGAAGGATGTGGCTCTGCC | |
| | ATGAAGGATGTCCTGTTGCCTTTAAAATCTG | |
| | GAAGCGATTCAAGCCAAGCTGACCAAGAAG | |
| | CCAAAGAACTGGCTAGGCAAATAAGCTTTAA | |
| | GGCAGAAGTCAATTCATCTGGAAAGACTATC | |
| | TCTGAGTCAGACTTAAACCACTCTTTTTCACT | |
| | AACTGATTTACAAGATGATATACCTACAACAT | |
| | TCCAGGGTGCTACGGCC | |
| UBE2J2 | AGCAGCACCAGCTCTAAGAGAGCCCCTACA | SEQ ID NO: |
| | ACCGCCACACAGCGGCTGAAGCAGGACTAC | 99 |
| | CTGCGGATCAAGAAAGACCCCGTGCCTTAC | |
| | ATCTGCGCCGAGCCTCTGCCTAGCAACATC | |
| | CTGGAATGGCACTACGTCGTGCGGGGACCT | |
| | GAGATGACACCTTACGAAGGCGGCTACTAC | |
| | CACGGCAAGCTGATCTTCCCCAGAGAGTTC | |
| | CCATTCAAGCCACCTAGCATCTACATGATCA | |
| | CCCCTAACGGCCGGTTCAAGTGCAACACCA | |
| | GACTGTGCCTGAGCATCACCGACTTTCACC | |
| | CCGACACCTGGAATCCCGCTTGGAGCGTGT | |
| | CCACAATCCTGACAGGCCTGCTGAGCTTCAT | |
| | GGTGGAAAAGGGCCCTACACTGGGCAGCAT | |
| | CGAGACAAGCGACTTCACCAAGAGACAGCT | |
| | GGCCGTGCAGAGCCTGGCCTTCAACCTGAA | |
| | GGACAAGGTGTTCTGCGAGCTGTTCCCCGA | |
| | GGTGGTGGAAGAGATCAAGCAGAAGCAGAA | |
| | GGCCCAGGACGAGCTGAGCAGCAGACCTCA | |
| | AACACTGCCT | |
| UBE2K | GCCAATATCGCCGTGCAGAGAATCAAGCGC | SEQ ID NO: |
| | GAGTTCAAAGAGGTGCTGAAGTCCGAGGAA | 100 |
| | ACCAGCAAGAACCAGATCAAGGTGGACCTG | |
| | GTGGACGAGAACTTCACCGAGCTGAGAGGC | |
| | GAGATTGCCGGACCTCCTGACACACCTTAT | |
| | GAAGGCGGCAGATACCAGCTGGAAATCAAG | |
| | ATCCCCGAGACATACCCCTTCAATCCTCCAA | |
| | AAGTGCGGTTCATCACCAAGATCTGGCACC | |
| | CCAACATCAGCAGCGTGACCGGCGCCATCT | |
| | GTCTGGACATCCTGAAGGATCAGTGGGCCG | |
| | CTGCCATGACACTGAGAACAGTTCTGCTGA | |
| | GTCTGCAGGCCCTGCTGGCTGCTGCTGAAC | |
| | CTGATGATCCACAGGATGCCGTGGTGGCCA | |
| | ACCAGTACAAGCAGAACCCCGAGATGTTCA | |
| | AGCAGACCGCCAGACTGTGGGCCCATGTGT | |
| | ATGCTGGTGCTCCCGTTAGCAGCCCCGAGT | |
| | ACACCAAGAAAATCGAGAACCTGTGCGCCA | |
| | TGGGCTTCGACAGAAACGCCGTGATTGTGG | |
| | CCCTGAGCAGCAAGAGCTGGGATGTCGAAA | |
| | CAGCCACCGAACTGCTGCTGTCCAAC | |
| UBE2L3 | GCCGCTAGTCGGAGACTGATGAAGGAACTG | SEQ ID NO: |
| | GAAGAGATCCGGAAGTGCGGCATGAAGAAC | 101 |
| | TTCCGGAACATCCAGGTGGACGAGGCCAAC | |
| | CTGCTGACATGGCAGGGACTGATCGTGCCC | |
| | GACAATCCTCCTTACGACAAGGGCGCCTTC | |
| | AGAATCGAGATCAACTTCCCCGCCGAGTATC | |
| | CCTTCAAGCCTCCTAAGATCACCTTCAAGAC | |
| | CAAGATCTATCACCCCAACATCGACGAGAAG | |
| | GGCCAAGTGTGCCTGCCTGTGATCAGCGCC | |
| | GAGAATTGGAAGCCTGCCACCAAGACCGAC | |
| | CAAGTGATCCAGTCTCTGATCGCCCTGGTCA | |
| | ACGACCCTCAGCCTGAACATCCTCTGAGAG | |
| | CCGATCTGGCCGAAGAGTACAGCAAGGACC | |
| | GGAAGAAGTTCTGCAAGAACGCTGAAGAGT | |
| | TCACCAAGAAGTACGGCGAGAAGCGGCCCG | |
| | TGGAT | |
| UBE2L6 | ATGGCCAGCATGCGGGTCGTGAAAGAGCTG | SEQ ID NO: |
| | GAAGATCTGCAGAAGAAGCCTCCTCCTTACC | 102 |
| | TGCGGAACCTGAGCAGCGACGATGCCAATG | |
| | TGCTTGTGTGGCATGCCCTGCTGCTGCCCG | |
| | ATCAGCCTCCTTATCACCTGAAGGCCTTTAA | |
| | CCTGCGGATCAGCTTCCCACCTGAGTACCC | |
| | CTTCAAGCCTCCTATGATCAAGTTCACCACC | |
| | AAGATCTATCACCCCAACGTGGACGAGAAC | |
| | GGCCAGATCTGCCTGCCTATCATCAGCAGC | |
| | GAGAACTGGAAGCCCTGCACCAAGACCTGC | |
| | CAGGTGCTGGAAGCTCTGAACGTGCTGGTC | |
| | AACAGACCCAACATCCGCGAGCCTCTGAGA | |
| | ATGGACCTGGCCGATCTGCTGACACAGAAC | |
| | CCCGAGCTGTTCCGGAAGAACGCCGAAGAG | |
| | TTCACCCTGAGATTCGGCGTGGACAGACCT | |
| | AGC | |
| UBE2M | GCCTCTGCTGCCCAGCTGAGAATCCAGAAG | SEQ ID NO: |
| | GACATCAACGAGCTGAACCTGCCTAAGACC | 103 |
| | TGCGACATCAGCTTCAGCGACCCCGACGAC | |
| | CTGCTGAACTTCAAGCTGGTCATCTGCCCC | |
| | GACGAGGGCTTCTACAAGAGCGGCAAGTTC | |
| | GTGTTCAGCTTCAAAGTCGGCCAGGGCTAC | |
| | CCTCACGACCCTCCAAAAGTGAAGTGCGAG | |
| | ACAATGGTGTATCACCCCAACATCGACCTGG | |
| | AAGGCAACGTGTGCCTGAACATCCTGCGCG | |
| | AGGATTGGAAGCCCGTGCTGACCATCAACA | |
| | GCATCATCTACGGCCTGCAGTACCTGTTCCT | |
| | GGAACCTAATCCTGAGGACCCTCTGAACAAA | |
| | GAGGCCGCCGAAGTCCTGCAGAACAACAGA | |
| | AGGCTGTTCGAGCAGAACGTGCAGCGGTCT | |
| | ATGAGAGGCGGCTACATTGGCAGCACCTAC | |
| | TTCGAGAGATGCCTGAAG | |
| UBE2N | GCCGGGCTGCCCCGCAGGATCATCAAGGAA | SEQ ID NO: |
| | ACCCAGCGTTTGCTGGCAGAACCAGTTCCT | 104 |
| | GGCATCAAAGCCGAACCAGATGAGAGCAAC | |
| | GCCCGTTATTTTCATGTGGTCATTGCTGGCC | |
| | CTCAGGATTCCCCCTTTGAGGGAGGGACTT | |
| | TTAAACTTGAACTATTCCTTCCAGAAGAATAC | |
| | CCAATGGCAGCCCCTAAAGTACGTTTCATGA | |
| | CCAAAATTTATCATCCTAATGTAGACAAGTTG | |
| | GGAAGAATATGTTTAGATATTTTGAAAGATAA | |
| | GTGGTCCCCAGCACTGCAGATCCGCACAGT | |
| | TCTGCTATCGATCCAGGCCTTGTTAAGTGCT | |
| | CCCAATCCAGATGATCCATTAGCAAATGATG | |
| | TAGCGGAGCAGTGGAAGACCAACGAAGCCC | |
| | AAGCCATAGAAACAGCTAGAGCATGGACTA | |
| | GGCTATATGCCATGAATAATATT | |
| UBE2NL | GCCGAGCTGCCCCACAGGATCATCAAGGAA | SEQ ID NO: |
| | ACCCAGCGTTTGCTGGCAGAGCCAGTTCCT | 105 |
| | GGCATCAAAGCAGAACCAGATGAAAGCAAC | |
| | GCCCGTTATTTTCATGTGGTCATTGCTGGGG | |
| | AATCAAAGGATTCCCCCTTTGAGGGAGGGA | |
| | CTTTTAAACGTGAACTATTACTTGCAGAAGA | |
| | ATACCCAATGGCAGCCCCTAAAGTACGTTTC | |
| | ATGACCAAAATTTATCATCCAAATGTAGACAA | |
| | GTTGGAAAGAATAAGTTTAGATATTTTGAAA | |
| | GATAAGTGGTCCCCAGCCCTGCAGATCCGC | |
| | ACAGTTCTGCTATCGATCCAGGCCTTGTTAA | |
| | ATGCTCCCAATCCAGATGATCCATTAGCAAA | |
| | TGATGTAGTGGAGCAGTGGAAGACCAACGA | |
| | AGCCCAAGCCATTGAAACAGCTAGAGCATG | |
| | GACTAGGCTATATGCCATGAATAGTATT | |
| UBE20 | AGCAACCACAGCTTCAAGAAGATCGAGTTCC | SEQ ID NO: |
| | AGCCACCTGAGGCCAAGAAATTCTTCAGCA | 106 |
| | CCGTGCGGAAAGAGATGGCCCTGCTGGCTA | |
| | CATCTCTGCCCGAGGGCATCATGGTCAAGA | |
| | CCTTCGAGGACCGGATGGACCTGTTCAGCG | |
| | CCCTGATCAAGGGCCCCACCAGAACACCTT | |
| | ATGAGGACGGCCTGTACCTGTTCGACATCC | |
| | AGCTGCCTAACATCTACCCCGCCGTGCCTC | |
| | CACACTTCTGCTACCTGTCTCAGTGCAGCG | |
| | GCAGACTGAACCCCAACCTGTACGACAACG | |
| | GCAAAGTGTGCGTGTCCCTGCTCGGCACAT | |
| | GGATCGGAAAGGGCACCGAGAGATGGACCA | |
| | GCAAGTCTAGCCTGCTGCAGGTCCTGATCA | |
| | GCATCCAGGGACTGATCCTGGTG | |
| UBE2Q1 | AGCGGATCTGTGCAGGCCACCGACCGGCTT | SEQ ID NO: |
| | ATGAAGGAACTGCGGGACATCTACAGAAGC | 107 |
| | CAGAGCTTCAAAGGCGGCAACTACGCCGTG | |
| | GAACTGGTCAACGACAGCCTGTACGACTGG | |
| | AACGTGAAGCTGCTGAAGGTGGACCAGGAT | |
| | AGCGCCCTGCACAACGACCTGCAGATCCTG | |
| | AAAGAGAAAGAGGGCGCCGACTTCATCCTG | |
| | CTGAACTTCAGCTTCAAGGACAACTTCCCCT | |
| | TCGATCCTCCATTCGTGCGCGTGGTGTCTC | |
| | CTGTTCTGTCTGGCGGATATGTGCTTGGCG | |
| | GCGGAGCCATCTGTATGGAACTGCTGACAA | |
| | AGCAAGGCTGGTCCAGCGCCTACAGCATCG | |
| | AGAGCGTGATCATGCAGATCAGCGCCACAC | |
| | TGGTCAAGGGCAAAGCCAGAGTGCAGTTCG | |
| | GCGCCAACAAGAGCCAGTACAGCCTGACAA | |
| | GAGCCCAGCAGAGCTACAAGTCCCTGGTGC | |
| | AGATCCACGAGAAGAACGGCTGG | |
| UBE2Q2 | GGCGCTGTGTCTGGATCTGTGCAGGCCTCT | SEQ ID NO: |
| | GACCGGCTGATGAAGGAACTGCGGGACATC | 108 |
| | TACAGAAGCCAGAGCTACAAGACCGGCATC | |
| | TACAGCGTGGAACTGATCAACGACAGCCTG | |
| | TACGACTGGCACGTGAAGCTGCAGAAGGTG | |
| | GACCCTGATAGCCCTCTGCACAGCGACCTG | |
| | CAGATCCTGAAAGAGAAAGAGGGCATCGAG | |
| | TACATCCTGCTGAACTTCAGCTTCAAGGACA | |
| | ACTTCCCCTTCGATCCTCCATTCGTGCGCGT | |
| | GGTGCTGCCTGTTCTGTCTGGCGGATATGT | |
| | GCTTGGAGGCGGAGCCCTGTGTATGGAACT | |
| | GCTGACAAAGCAAGGCTGGTCCAGCGCCTA | |
| | CAGCATCGAGAGCGTGATCATGCAGATCAA | |
| | CGCCACACTGGTCAAGGGCAAAGCCAGAGT | |
| | GCAGTTCGGCGCCAACAAGAACCAGTACAA | |
| | CCTGGCTAGAGCCCAGCAGTCCTACAACAG | |
| | CATCGTGCAGATCCACGAA | |
| UBE2QL | AAGGAGCTGCAGGACATCGCGCGCCTTAGC | SEQ ID NO: |
| | GACCGCTTCATCTCCGTGGAGCTGGTGGAC | 109 |
| | GAGAGCCTGTTCGACTGGAACGTGAAGCTG | |
| | CACCAGGTGGACAAGGACTCGGTGCTGTGG | |
| | CAGGACATGAAGGAGACCAACACCGAGTTC | |
| | ATCCTGCTCAACCTCACCTTCCCCGACAACT | |
| | TCCCCTTCTCGCCGCCCTTCATGCGGGTGC | |
| | TCAGCCCGCGCCTGGAGAACGGCTACGTGC | |
| | TGGACGGCGGCGCCATCTGCATGGAGCTGC | |
| | TCACGCCGCGCGGCTGGTCCAGCGCCTACA | |
| | CCGTGGAGGCCGTCATGCGCCAGTTCGCAG | |
| | CCAGCCTGGTCAAGGGCCAGGGACGGATCT | |
| | GTAGAAAAGCTGGCAAATCAAAAAAGTCCTT | |
| | CAGTCGCAAGGAAGCTGAAGCTACCTTTAA | |
| | GAGTTTGGTGAAGACGCATGAAAAATATGGT | |
| | TGGGTCACCCCGCCCGTGTCCGACGGC | |
| UBE2R1 | CCTAGCTCTCAGAAGGCCCTGCTGCTGGAA | SEQ ID NO: |
| | CTGAAGGGCCTGCAAGAGGAACCCGTGGAA | 110 |
| | GGCTTCAGAGTGACCCTGGTGGATGAGGGC | |
| | GACCTGTACAATTGGGAAGTCGCCATCTTCG | |
| | GCCCTCCAAACACCTACTACGAAGGCGGCT | |
| | ACTTCAAGGCCCGGCTGAAGTTCCCCATCG | |
| | ACTACCCTTATAGCCCTCCTGCCTTCCGGTT | |
| | CCTGACCAAGATGTGGCACCCCAACATCTA | |
| | CGAGACAGGCGACGTGTGCATCAGCATTCT | |
| | GCACCCTCCAGTGGACGATCCTCAGTCTGG | |
| | CGAGCTGCCAAGCGAGAGATGGAACCCCAC | |
| | ACAGAACGTGCGGACCATCCTGCTGAGCGT | |
| | GATCAGCCTGCTGAACGAGCCCAACACATT | |
| | CAGCCCCGCCAATGTGGATGCCAGCGTGAT | |
| | GTACCGGAAGTGGAAAGAGTCCAAGGGCAA | |
| | AGACAGAGAGTACACCGACATCATCCGGAA | |
| | ACAGGTGCTGGGCACAAAGGTGGACGCCGA | |
| | GCGAGATGGCGTGAAAGTTCCT | |
| UBE2R2 | ACCAGCTCGCAGAAGGCCCTGATGCTCGAG | SEQ ID NO: |
| | CTGAAATCCCTGCAGGAGGAACCGGTGGAG | 111 |
| | GGCTTCCGGATCACCCTGGTGGACGAGTCC | |
| | GACCTCTACAACTGGGAGGTGGCCATCTTC | |
| | GGACCCCCCAACACCCTCTACGAAGGCGGC | |
| | TACTTCAAGGCGCATATTAAATTTCCTATTGA | |
| | CTACCCCTATTCACCACCTACCTTCAGATTC | |
| | TTGACCAAAATGTGGCACCCCAACATTTATG | |
| | AGAATGGAGATGTATGCATTTCGATTCTTCA | |
| | TCCGCCTGTAGATGACCCACAGAGTGGAGA | |
| | ACTGCCTTCTGAAAGGTGGAATCCTACTCAG | |
| | AATGTGAGGACTATCCTATTAAGTGTAATCT | |
| | CACTGCTTAATGAGCCCAACACCTTCTCCCC | |
| | AGCCAATGTCGATGCTTCAGTTATGTTCAGG | |
| | AAATGGAGAGACAGTAAAGGAAAAGACAAA | |
| | GAATATGCTGAAATTATTAGGAAACAAGTTT | |
| | CAGCCACTAAGGCCGAAGCAGAAAAGGATG | |
| | GAGTGAAGGTCCCCACAACCCTGGCGGAAT | |
| | ACTGCATCAAAACTAAAGTGCCT | |
| UBE2S | AACAGCAACGTGGAAAACCTGCCTCCTCAC | SEQ ID NO: |
| | ATCATCCGGCTGGTGTACAAAGAAGTGACC | 112 |
| | ACACTGACCGCCGATCCTCCAGACGGCATC | |
| | AAGGTGTTCCCCAACGAAGAGGACCTGACC | |
| | GACCTGCAAGTGACCATCGAAGGCCCTGAG | |
| | GGCACACCTTATGCTGGCGGCCTGTTCAGA | |
| | ATGAAGCTGCTGCTGGGCAAAGACTTCCCC | |
| | GCATCTCCTCCAAAGGGCTACTTCCTGACCA | |
| | AGATCTTTCACCCCAACGTGGGCGCCAACG | |
| | GCGAGATCTGTGTGAACGTGCTGAAGAGAG | |
| | ACTGGACCGCCGAGCTGGGAATCAGACACG | |
| | TGCTGCTGACCATCAAGTGCCTGCTGATTCA | |
| | CCCTAATCCTGAGAGCGCCCTGAACGAGGA | |
| | AGCTGGCAGACTGCTGCTCGAAAACTACGA | |
| | GGAATACGCCGCCAGAGCCAGGCTGCTGAC | |
| | AGAGATTCATGGA | |
| UBE2T | CAGAGAGCCAGCAGACTGAAGCGCGAGCTG | SEQ ID NO: |
| | CATATGCTGGCCACAGAACCTCCACCTGGC | 113 |
| | ATCACCTGTTGGCAGGACAAGGACCAGATG | |
| | GACGACCTGAGAGCCCAGATTCTCGGCGGA | |
| | GCCAACACACCTTATGAGAAGGGCGTGTTC | |
| | AAGCTGGAAGTGATCATCCCCGAGAGATAC | |
| | CCCTTCGAGCCTCCTCAGATCCGGTTTCTGA | |
| | CCCCTATCTATCACCCCAACATCGACAGCGC | |
| | CGGCAGAATCTGTCTGGACGTGCTGAAGCT | |
| | GCCTCCTAAAGGCGCTTGGAGGCCCAGCCT | |
| | GAATATCGCCACAGTGCTGACCAGCATCCA | |
| | GCTGCTGATGAGCGAGCCCAATCCTGACGA | |
| | TCCCCTGATGGCCGATATCAGCAGCGAGTT | |
| | CAAGTACAACAAGCCCGCCTTCCTGAAGAA | |
| | CGCCAGACAGTGGACAGAGAAGCACGCCC | |
| | GGCAGAAGCAGAAGGCCGACGAGGAAGAG | |
| | ATGCTGGACAACCTGCCT | |
| UBE2U | CACGGCAGAGCCTATCTGCTGCTGCACAGA | SEQ ID NO: |
| | GATTTCTGCGACCTGAAAGAGAACAACTACA | 114 |
| | AGGGCATCACCGCCAAGCCTGTGTCCGAGG | |
| | ACATGATGGAATGGGAAGTCGAGATCGAGG | |
| | GCCTGCAGAACTCTGTGTGGCAGGGCCTTG | |
| | TGTTCCAGCTGACCATCCACTTCACCAGCGA | |
| | GTACAACTACGCCCCTCCTGTGGTCAAGTTC | |
| | ATCACAATCCCTTTTCACCCCAACGTGGACC | |
| | CTCACACCGGCCAGCCTTGCATCGACTTTCT | |
| | GGACAACCCCGAGAAGTGGAACACCAACTA | |
| | CACCCTGAGCAGCATCCTGCTGGCCCTGCA | |
| | AGTGATGCTGAGCAACCCCGTGCTGGAAAA | |
| | CCCCGTGAATCTGGAAGCCGCCAGAATCCT | |
| | GGTCAAGGACGAGAGCCTGTACCGGACCAT | |
| | CCTGCGGCTGTTCAACAGACCT | |
| UBE2V1 | GGAGTAAAAGTCCCTCGCAATTTCCGACTGT | SEQ ID NO: |
| | TGGAAGAACTCGAAGAAGGCCAGAAAGGAG | 115 |
| | TAGGAGATGGCACAGTTAGCTGGGGTCTAG | |
| | AAGATGACGAAGACATGACACTTACAAGATG | |
| | GACAGGGATGATAATTGGGCCTCCAAGAAC | |
| | AATTTATGAAAACCGAATATACAGCCTTAAAA | |
| | TAGAATGTGGACCTAAATACCCAGAAGCACC | |
| | CCCCTTTGTAAGATTTGTAACAAAAATTAATA | |
| | TGAATGGAGTAAATAGTTCTAATGGAGTGGT | |
| | GGACCCAAGAGCCATATCAGTGCTAGCAAA | |
| | ATGGCAGAATTCATATAGCATCAAAGTTGTC | |
| | CTGCAAGAGCTTCGGCGCCTAATGATGTCTA | |
| | AAGAAAATATGAAACTCCCTCAGCCGCCCGA | |
| | AGGACAGTGTTACAGCAAT | |
| UBE2V2 | GCCGTGTCTACAGGCGTGAAGGTGCCCAGA | SEQ ID NO: |
| | AACTTCCGGCTGCTGGAAGAACTGGAAGAG | 116 |
| | GGCCAGAAAGGCGTCGGAGATGGCACAGT | |
| | GTCTTGGGGCCTCGAAGATGACGAGGACAT | |
| | GACCCTGACCAGATGGACCGGCATGATCAT | |
| | CGGCCCTCCACGGACCAACTACGAGAACCG | |
| | GATCTACTCCCTGAAGGTGGAATGCGGCCC | |
| | TAAGTACCCTGAGGCTCCTCCTAGCGTCAG | |
| | ATTCGTGACCAAGATCAACATGAACGGCATC | |
| | AACAACAGCAGCGGCATGGTGGACGCCAGA | |
| | TCTATTCCTGTGCTGGCCAAGTGGCAGAACA | |
| | GCTACAGCATCAAGGTGGTGCTGCAAGAGC | |
| | TGCGGCGGCTGATGATGAGCAAAGAAAACA | |
| | TGAAGCTGCCCCAGCCACCTGAGGGACAGA | |
| | CCTACAACAAT | |
| UBE2W | GCCAGCATGCAGAAGCGGCTGCAGAAAGAA | SEQ ID NO: |
| | CTGCTGGCCCTGCAGAACGATCCTCCTCCT | 117 |
| | GGCATGACCCTGAACGAGAAGTCCGTGCAG | |
| | AACAGCATCACCCAGTGGATCGTGGACATG | |
| | GAAGGCGCCCCTGGCACACTGTATGAGGGC | |
| | GAGAAATTCCAGCTGCTGTTCAAGTTCAGCA | |
| | GCAGATACCCCTTCGACAGCCCTCAAGTGA | |
| | TGTTCACCGGCGAGAACATCCCTGTGCACC | |
| | CTCACGTGTACAGCAACGGCCACATCTGCC | |
| | TGAGCATCCTGACCGAGGATTGGAGCCCTG | |
| | CTCTGAGCGTGCAGAGCGTGTGTCTGAGCA | |
| | TCATCTCCATGCTGAGC | |
| UBE2Z | ATGTCCATTTATAAGGAGCCTCCTCCAGGAA | SEQ ID NO: |
| | TGTTCGTTGTACCTGATACTGTTGACATGAC | 118 |
| | TAAGATTCATGCATTGATCACAGGCCCATTT | |
| | GACACTCCTTATGAAGGGGGTTTCTTCCTGT | |
| | TCGTGTTTCGGTGTCCGCCCGACTATCCCAT | |
| | CCACCCACCTCGGGTCAAACTGATGACAAC | |
| | GGGCAATAACACAGTGAGGTTTAACCCCAA | |
| | CTTCTACCGCAATGGGAAAGTCTGCTTGAGT | |
| | ATTCTAGGTACATGGACTGGACCTGCCTGG | |
| | AGCCCAGCCCAGAGCATCTCCTCAGTGCTC | |
| | ATCTCTATCCAGTCCCTGATGACTGAGAACC | |
| | CCTATCACAATGAGCCCGGCTTTGAACAGG | |
| | AGAGACATCCAGGAGACAGCAAAAACTATAA | |
| | TGAATGTATCCGGCACGAGACCATCAGAGTT | |
| | GCAGTC | |
| UEVLD | GAGTTCGACTGCGAGGGCCTGAGACGGCTG | SEQ ID NO: |
| | CTTGGCAAGTACAAGTTCAGGGACCTAACTG | 119 |
| | TGGAAGAACTAAGGAATGTAAATGTATTTTT | |
| | CCCACATTTCAAATATTCCATGGACACCTAT | |
| | GTTTTTAAAGATAGTTCTCAGAAAGACCTGC | |
| | TGAATTTTACTGGCACAATTCCTGTGATGTAT | |
| | CAGGGTAATACATATAACATACCAATTCGTTT | |
| | CTGGATTTTGGATTCTCACCCTTTCGCTCCC | |
| | CCTATTTGCTTCTTGAAGCCAACTGCAAATA | |
| | TGGGAATCTTAGTCGGAAAACATGTGGATGC | |
| | TCAAGGCAGAATATATTTGCCCTATCTCCAA | |
| | AACTGGAGCCATCCTAAATCTGTCATTGTTG | |
| | GATTAATTAAAGAAATGATTGCCAAGTTTCAA | |
| | GAGGAACTTCCCATGTATTCTCTATCATCAT | |
| | CTGATGAGGCACGGCAGGTAGACTTGCTAG | |
| | CCTATATTGCAAAAATCACTGAAGGTGTTTC | |
| | AGATACAAATTCAAAGAGCTGGGCAAATCAT | |
| | GAGAATAAAACAGTCAATAAAATTACTGTGG | |
| | TTGGAGGTGGAGAACTCGGTATTGCCTGCA | |
| | CATTAGCAATTTCAGCAAAGGGTATTGCAGA | |
| | CAGGCTTGTCCTCTTAGACCTCTCAGAAGG | |
| | GACTAAAGGAGCCACGATGGACCTTGAAAT | |
| | CTTCAACCTTCCTAATGTGGAGATCAGCAAA | |
| | GATTTGTCTGCCTCTGCTCATTCCAAGGTGG | |
| | TGATCTTCACAGTCAACTCTTTGGGTAGTTC | |
| | TCAGTCGTACCTTGATGTGGTACAGAGCAAT | |
| | GTGGATATGTTCAGAGCCCTTGTCCCAGCTC | |
| | TGGGACATTATAGTCAACACAGTGTCCTGCT | |
| | CGTTGCATCTCAACCAGTGGAAATCATGACC | |
| | TATGTAACATGGAAACTGAGTACATTTCCTG | |
| | CAAATCGAGTGATCGGAATTGGATGTAATCT | |
| | GGATTCACAGAGATTACAGTATATTATTACAA | |
| | ATGTTTTGAAGGCACAGACTTCAGGCAAAGA | |
| | AGTATGGGTTATTGGCGAGCAAGGAGAAGA | |
| | CAAAGTG | |
| BIRC6 | GCCAATCAAGAGAAGAAGCTGGGCGAGTAC | SEQ ID NO: |
| | AGCAAGAAAGCCGCCATGAAGCCCAAGCCT | 120 |
| | CTGAGCGTGCTGAAGTCCCTGGAAGAGAAA | |
| | TACGTGGCCGTGATGAAGAAGCTCCAGTTC | |
| | GACACCTTCGAGATGGTGTCCGAGGACGAG | |
| | GATGGCAAGCTGGGCTTCAAAGTGAACTAC | |
| | CACTACATGAGCCAAGTGAAGAACGCCAAC | |
| | GACGCCAACTCTGCCGCCAGAGCTAGAAGG | |
| | CTGGCTCAAGAAGCCGTGACTCTGAGCACA | |
| | AGCCTGCCACTGAGCAGCAGCAGCTCCGTG | |
| | TTCGTCAGATGTGACGAGGAACGGCTGGAC | |
| | ATCATGAAGGTGCTGATCACAGGCCCTGCC | |
| | GACACACCTTACGCCAATGGCTGCTTCGAG | |
| | TTCGACGTGTACTTCCCTCAAGACTACCCTA | |
| | GCAGCCCTCCTCTGGTCAACCTGGAAACAA | |
| | CAGGCGGACACAGCGTGCGGTTCAACCCCA | |
| | ACCTGTACAACGACGGCAAAGTGTGCCTGA | |
| | GCATCCTGAACACCTGGCACGGCAGACCCG | |
| | AGGAAAAGTGGAATCCTCAGACCAGCAGCT | |
| | TCCTGCAGGTCCTGGTGTCTGTGCAGAGCC | |
| | TGATTCTG | |
| FTS | CCTCCACGAACTGCACCAAAGAAACAGCTG | SEQ ID NO: |
| | CCTTCTATTCCCAAAAATGCTTTGCCCATAA | 121 |
| | CTAAGCCTACATCTCCTGCCCCAGCAGCAC | |
| | AGTCAACAAATGGCACGCATGCGTCCTATG | |
| | GACCCTTCTACCTGGAATACTCTCTTCTTGC | |
| | AGAATTTACCTTGGTTGTGAAGCAGAAGCTA | |
| | CCAGGCGTCTATGTGCAGCCATCTTATCGCT | |
| | CTGCATTAATGTGGTTTGGAGTAATATTCATA | |
| | CGGCATGGACTTTACCAAGATGGCGTATTTA | |
| | AGTTTACAGTTTACATCCCTGATAACTATCCA | |
| | GATGGTGACTGTCCACGCTTGGTGTTCGATA | |
| | TTCCTGTCTTTCACCCGCTAGTTGATCCCAC | |
| | CTCAGGTGAGCTGGATGTGAAGAGAGCATT | |
| | TGCAAAATGGAGGCGGAACCATAATCATATT | |
| | TGGCAGGTATTAATGTATGCAAGGAGAGTTT | |
| | TCTACAAGATTGATACAGCAAGCCCCCTGAA | |
| | CCCAGAGGCTGCAGTACTGTATGAAAAAGAT | |
| | ATTCAGCTTTTTAAAAGTAAAGTTGTTGACAG | |
| | TGTTAAGGTGTGCACTGCTCGTTTGTTTGAC | |
| | CAACCTAAAATAGAAGAC | |
| TSG101 | GCCGTGTCTGAGAGCCAGCTGAAGAAAATG | SEQ ID NO: |
| | GTGTCCAAGTACAAGTACCGGGACCTGACC | 122 |
| | GTGCGGGAAACCGTGAATGTGATCACCCTG | |
| | TACAAGGACCTGAAGCCTGTGCTGGACAGC | |
| | TACGTGTTCAACGATGGCAGCAGCCGCGAG | |
| | CTGATGAACCTGACAGGCACAATCCCCGTG | |
| | CCTTACCGGGGCAACACCTACAACATCCCC | |
| | ATCTGCCTGTGGCTGCTGGACACATACCCC | |
| | TACAATCCTCCAATCTGCTTCGTGAAGCCCA | |
| | CCAGCAGCATGACCATCAAGACCGGCAAAC | |
| | ACGTGGACGCCAACGGCAAGATCTACCTGC | |
| | CTTACCTGCACGAGTGGAAGCACCCTCAGT | |
| | CTGATCTGCTGGGCCTGATCCAAGTGATGAT | |
| | CGTGGTGTTCGGCGACGAGCCTCCTGTGTT | |
| | CAGTAGACCT | |
| UFC1 | GCCGATGAGGCCACACGAAGAGTGGTGTCT | SEQ ID NO: |
| | GAGATCCCCGTGCTGAAAACAAACGCTGGC | 123 |
| | CCCAGAGACAGAGAACTGTGGGTGCAGCGG | |
| | CTGAAAGAGGAATACCAGAGCCTGATCCGC | |
| | TACGTCGAGAACAACAAGAACGCCGACAAC | |
| | GACTGGTTCCGGCTGGAAAGCAACAAAGAA | |
| | GGCACCCGTTGGTTCGGCAAGTGCTGGTAC | |
| | ATCCACGACCTGCTGAAGTACGAGTTCGAC | |
| | ATCGAGTTCGATATCCCCATCACATACCCCA | |
| | CCACCGCTCCAGAGATTGCCGTGCCTGAGC | |
| | TGGATGGCAAGACCGCCAAGATGTACAGAG | |
| | GCGGCAAGATCTGCCTGACCGACCACTTCA | |
| | AACCCCTGTGGGCCAGAAACGTGCCCAAGT | |
| | TTGGACTGGCCCATCTGATGGCCCTCGGAC | |
| | TTGGACCTTGGCTGGCCGTGGAAATCCCCG | |
| | ACCTGATTCAGAAAGGCGTGATCCAGCACA | |
| | AAGAGAAGTGCAACCAG | |

TABLE 10
Expression tags, targeting domains, linkers, regulation domains and
corresponding SEQ ID NOS.
Fusion polypeptide domains

| Expression tag | Amino acid sequence | SEQ ID NO: |
|---|---|---|
| HA tag | YPYDVPDYA | SEQ ID NO: |
| | | 124 |
| Glu-Glu | CEEEEYMPME | SEQ ID NO: |
| | | 125 |
| Targeting domains | Amino acid sequence | SEQ ID NO: |
| aCS3 | VSSVPTKLEVVAATPTSLLISWDAPAV | SEQ ID NO: |
| | TVDYYVITYGETGHWPWVWQEFEVP | 126 |
| | GSKSTATISGLKPGVDYTITVYAGSYS | |
| | SYYYYGSPISINYRT | |
| aCS3_full | EFAMVSSVPTKLEVVAATPTSLLISWD | SEQ ID NO: |
| | APAVTVDYYVITYGETGHWPWVWQE | 127 |
| | FEVPGSKSTATISGLKPGVDYTITVYA | |
| | GSYSSYYYYGSPISINYRT | |
| aCS3(V33R) | VSSVPTKLEVVAATPTSLLISWDAPAV | SEQ ID NO: |
| | TVDYYRITYGETGHWPWVWQEFEVP | 128 |
| | GSKSTATISGLKPGVDYTITVYAGSYS | |
| | SYYYYGSPISINYRT | |
| aCS3(K7Q) | VSSVPTQLEVVAATPTSLLISWDAPAV | SEQ ID NO: |
| | TVDYYVITYGETGHWPWVWQEFEVP | 129 |
| | GSKSTATISGLKPGVDYTITVYAGSYS | |
| | SYYYYGSPISINYRT | |
| aCS3(K55Y) | VSSVPTKLEVVAATPTSLLISWDAPAV | SEQ ID NO: |
| | TVDYYVITYGETGHWPWVWQEFEVP | 130 |
| | GSYSTATISGLKPGVDYTITVYAGSYS | |
| | SYYYYGSPISINYRT | |
| aCS3(K64H) | VSSVPTKLEVVAATPTSLLISWDAPAV | SEQ ID NO: |
| | TVDYYVITYGETGHWPWVWQEFEVP | 131 |
| | GSYSTATISGLHPGVDYTITVYAGSYS | |
| | SYYYYGSPISINYRT | |
| aCS3(K7Q/K55Y) | VSSVPTQLEVVAATPTSLLISWDAPAV | SEQ ID NO: |
| | TVDYYVITYGETGHWPWVWQEFEVP | 132 |
| | GSYSTATISGLKPGVDYTITVYAGSYS | |
| | SYYYYGSPISINYRT | |
| aCS3(K7Q/K64H) | VSSVPTQLEVVAATPTSLLISWDAPAV | SEQ ID NO: |
| | TVDYYVITYGETGHWPWVWQEFEVP | 133 |
| | GSYSTATISGLHPGVDYTITVYAGSYS | |
| | SYYYYGSPISINYRT | |
| aCS3(K55Y/K64H) | VSSVPTQLEVVAATPTSLLISWDAPAV | SEQ ID NO: |
| | TVDYYVITYGETGHWPWVWQEFEVP | 134 |
| | GSYSTATISGLHPGVDYTITVYAGSYS | |
| | SYYYYGSPISINYRT | |
| aCS3 | VSSVPTQLEVVAATPTSLLISWDAPAV | SEQ ID NO: |
| (K7Q/K55Y/K64H) | TVDYYVITYGETGHWPWVWQEFEVP | 135 |
| | GSYSTATISGLHPGVDYTITVYAGSYS | |
| | SYYYYGSPISINYRT | |
| aCS3 | VSSVPTQLEVVAATPTSLLISWDAPAV | SEQ ID NO: |
| (K7Q/K55Y/K64H/V33R) | TVDYYRITYGETGHWPWVWQEFEVP | 257 |
| | GSYSTATISGLHPGVDYTITVYAGSYS | |
| | SYYYYGSPISINYRT | |
| K19 | DLGKKLLEAARAGQDDEVRILMANGA | SEQ ID NO: |
| | DVNASDRWGWTPLHLAAWWGHLEIV | 138 |
| | EVLLKRGADVSAADLHGQSPLHLAAM | |
| | VGHLEIVEVLLKYGADVNAKDTMGAT | |
| | PLHLAARSGHLEIVEELLKNGADMNA | |
| | QDKFGKTTFDISTDNGNEDLAEILQKL | |
| E3_5 | DLGKKLLEAARAGQDDEVRILMANGA | SEQ ID NO: |
| | DVNATDNDGYTPLHLAASNGHLEIVE | 139 |
| | VLLKNGADVNASDLTGITPLHLAAATG | |
| | HLEIVEVLLKHGADVNAYDNDGHTPL | |
| | HLAAKYGHLEIVEVLLKHGADVNAQD | |
| | KFGKTAFDISIDNGNEDLAEILQ | |
| Linkers | Amino acid sequence | SEQ ID NO: |
| Linker 1 (Short) | LEGGGGSSR | SEQ ID NO: |
| | | 141 |
| Linker 2 (Long) | LEGGGGSGGGGSGGGGSSR | SEQ ID NO: |
| | | 142 |
| Linker 3 | AAAGGGGSGGGGSGGGGSGT | SEQ ID NO: |
| | | 143 |
| Linker 4 | GGGGG | SEQ ID NO: |
| | | 144 |
| Linker 5 | GGGGSGGGGSGGGGS | SEQ ID NO: |
| | | 145 |
| Linker 6 | GGGGS | SEQ ID NO: |
| | | 146 |
| Linker 7 | LEGGSR | SEQ ID NO: |
| | | 211 |
| Linker 8 | LEGGGSGGSSR | SEQ ID NO: |
| | | 212 |
| Linker 9 | LEGGGGSGGGSSR | SEQ ID NO: |
| | | 213 |
| Linker 10 | LEGGGSGGGSGGGSSR | SEQ ID NO: |
| | | 214 |
| Linker 11 | LEGGGGSGPSGGGGPSGSR | SEQ ID NO: |
| | | 215 |
| Linker 12 | LESNGGGGSPAPAPGGGGSGSSR | SEQ ID NO: |
| | | 216 |
| Linker 13 | LEGGGGSYPYDVPDYASGGGGSSR | SEQ ID NO: |
| | | 217 |
| Linker 14 | TGGSAGGSGGSAGGSGGSAGGSGG | SEQ ID NO: |
| | SA | 218 |
| Linker 15 | AGSGGSTGSGGSPTPSTSGGSTGSG | SEQ ID NO: |
| | GAS | 219 |
| Linker 16 | AGSGGSGGSGGSGNSSTSGGSGGS | SEQ ID NO: |
| | GGAS | 220 |
| Linker 17 | GGSPVPSTPGGGSGGGSGGSPVPST | SEQ ID NO: |
| | PGS | 221 |
| Linker 18 | SPGTGSPGTGSPGTGSPGTGSPGTG | SEQ ID NO: |
| | SPG | 222 |
| Regulation domains | Amino acid sequence | SEQ ID NO: |
| VHL | AMPRRAENWDEAEVGAEEAGVEEYG | SEQ ID NO: |
| | PEEDGGEESGAEESGPEESGPEELG | 147 |
| | AEEEMEVGRPRPVLRSVNSREPSQVI | |
| | FCNRSPRVVLPVWLNFDGEPQPYPTL | |
| | PPGTGRRIHSYRGHLWLFRDAGTHD | |
| | GLLVNQTELFVPSLNVDGQPIFANITL | |
| | PVYTLKERCLQVVRSLVKPENYRRLDI | |
| | VRSLYEDLEDHPNVQKDLERLTQERI | |
| | AHQRMGD | |
| UBE2B | STPARRRLMRDFKRLQEDPPVGVSG | SEQ ID NO: |
| | APSENNIMQWNAVIFGPEGTPFEDGT | 43 |
| | FKLVIEFSEEYPNKPPTVRFLSKMFHP | |
| | NVYADGSICLDILQNRWSPTYDVSSIL | |
| | TSIQSLLDEPNPNSPANSQAAQLYQE | |
| | NKREYEKRVSAIVEQSWNDS | |
| UBE2B (C88A) | STPARRRLMRDFKRLQEDPPVGVSG | SEQ ID NO: |
| | APSENNIMQWNAVIFGPEGTPFEDGT | 280 |
| | FKLVIEFSEEYPNKPPTVRFLSKMFHP | |
| | NVYADGSIALDILQNRWSPTYDVSSIL | |
| | TSIQSLLDEPNPNSPANSQAAQLYQE | |
| | NKREYEKRVSAIVEQSWNDS | |
| UBE2C | ASQNRDPAATSVAAARKGAEPSGGA | SEQ ID NO: |
| | ARGPVGKRLQQELMTLMMSGDKGIS | 44 |
| | AFPESDNLFKWVGTIHGAAGTVYEDL | |
| | RYKLSLEFPSGYPYNAPTVKFLTPCY | |
| | HPNVDTQGNICLDILKEKWSALYDVR | |
| | TILLSIQSLLGEPNIDSPLNTHAAELWK | |
| | NPTAFKKYLQETYSKQVTSQEP | |
| UBE2D1 | ALKRIQKELSDLQRDPPAHCSAGPVG | SEQ ID NO: |
| | DDLFHWQATIMGPPDSAYQGGVFFLT | 45 |
| | VHFPTDYPFKPPKIAFTTKIYHPNINSN | |
| | GSICLDILRSQWSPALTVSKVLLSICSL | |
| | LCDPNPDDPLVPDIAQIYKSDKEKYNR | |
| | HAREWTQKYAM | |
| UBE2D1(C85A) | ALKRIQKELSDLQRDPPAHCSAGPVG | SEQ ID NO: |
| | DDLFHWQATIMGPPDSAYQGGVFFLT | 148 |
| | VHFPTDYPFKPPKIAFTTKIYHPNINSN | |
| | GSIALDILRSQWSPALTVSKVLLSICSL | |
| | LCDPNPDDPLVPDIAQIYKSDKEKYNR | |
| | HAREWTQKYAM | |
| UBE2D1 (F62A) | ALKRIQKELSDLQRDPPAHCSAGPVG | SEQ ID NO: |
| | DDLFHWQATIMGPPDSAYQGGVFFLT | 281 |
| | VHFPTDYPAKPPKIAFTTKIYHPNINSN | |
| | GSICLDILRSQWSPALTVSKVLLSICSL | |
| | LCDPNPDDPLVPDIAQIYKSDKEKYNR | |
| | HAREWTQKY | |
| UBE2D2 | ALKRIHKELNDLARDPPAQCSAGPVG | SEQ ID NO: |
| | DDMFHWQATIMGPNDSPYQGGVFFL | 46 |
| | TIHFPTDYPFKPPKVAFTTRIYHPNINS | |
| | NGSICLDILRSQWSPALTISKVLLSICS | |
| | LLCDPNPDDPLVPEIARIYKTDREKYN | |
| | RIAREWTQKYAM | |
| UBE2D3 | ALKRINKELSDLARDPPAQCSAGPVG | SEQ ID NO: |
| | DDMFHWQATIMGPNDSPYQGGVFFL | 47 |
| | TIHFPTDYPFKPPKVAFTTRIYHPNINS | |
| | NGSICLDILRSQWSPALTISKVLLSICS | |
| | LLCDPNPDDPLVPEIARIYKTDRDKYN | |
| | RISREWTQKYAM | |
| UBE2E1 | SDDDSRASTSSSSSSSSNQQTEKET | SEQ ID NO: |
| | NTPKKKESKVSMSKNSKLLSTSAKRI | 49 |
| | QKELADITLDPPPNCSAGPKGDNIYE | |
| | WRSTILGPPGSVYEGGVFFLDITFTPE | |
| | YPFKPPKVTFRTRIYHCNINSQGVICL | |
| | DILKDNWSPALTISKVLLSICSLLTDCN | |
| | PADPLVGSIATQYMTNRAEHDRMAR | |
| | QWTKRYAT | |
| UBE2F | STRRVSVRDKLLVKEVAELEANLPCT | SEQ ID NO: |
| | CKVHFPDPNKLHCFQLTVTPDEGYYQ | 52 |
| | GGKFQFETEVPDAYNMVPPKVKCLTK | |
| | IWHPNITETGEICLSLLREHSIDGTGW | |
| | APTRTLKDVVWGLNSLFTDLLNFDDP | |
| | LNIEAAEHHLRDKEDFRNKVDDYIKRY | |
| | AR | |
| UBE2G1 | LLLRRQLAELNKNPVEGFSAGLIDDND | SEQ ID NO: |
| | LYRWEVLIIGPPDTLYEGGVFKAHLTF | 53 |
| | PKDYPLRPPKMKFITEIWHPNVDKNG | |
| | DVCISILHEPGEDKYGYEKPEERWLPI | |
| | HTVETIMISVISMLADPNGDSPANVDA | |
| | AKEWREDRNGEFKRKVARC | |
| UBE2G2 | AGTALKRLMAEYKQLTLNPPEGIVAG | SEQ ID NO: |
| | PMNEENFFEWEALIMGPEDTCFEFGV | 54 |
| | FPAILSFPLDYPLSPPKMRFTCEMFHP | |
| | NIYPDGRVCISILHAPGDDPMGYESSA | |
| | ERWSPVQSVEKILLSVVSMLAEPNDE | |
| | SGANVDASKMWRDDREQFYKIAKQIV | |
| | QKSLGL | |
| UBE2H | SSPSPGKRRMDTDVVKLIESKHEVTIL | SEQ ID NO: |
| | GGLNEFVVKFYGPQGTPYEGGVWKV | 55 |
| | RVDLPDKYPFKSPSIGFMNKIFHPNID | |
| | EASGTVCLDVINQTWTALYDLTNIFES | |
| | FLPQLLAYPNPIDPLNGDAAAMYLHR | |
| | PEEYKQKIKEYIQKYATEEALKEQEEG | |
| UBE21 | SGIALSRLAQERKAWRKDHPFGFVAV | SEQ ID NO: |
| | PTKNPDGTMNLMNWECAIPGKKGTP | 56 |
| | WEGGLFKLRMLFKDDYPSSPPKCKF | |
| | EPPLFHPNVYPSGTVCLSILEEDKDW | |
| | RPAITIKQILLGIQELLNEPNIQDPAQAE | |
| | AYTIYCQNRVEYEKRVRAQAKKFAPS | |
| UBE2J2 | SSTSSKRAPTTATQRLKQDYLRIKKDP | SEQ ID NO: |
| | VPYICAEPLPSNILEWHYVVRGPEMT | 58 |
| | PYEGGYYHGKLIFPREFPFKPPSIYMI | |
| | TPNGRFKCNTRLCLSITDFHPDTWNP | |
| | AWSVSTILTGLLSFMVEKGPTLGSIET | |
| | SDFTKRQLAVQSLAFNLKDKVFCELF | |
| | PEVVEEIKQKQKAQDELSSRPQTLP | |
| UBE2K | ANIAVQRIKREFKEVLKSEETSKNQIK | SEQ ID NO: |
| | VDLVDENFTELRGEIAGPPDTPYEGG | 59 |
| | RYQLEIKIPETYPFNPPKVRFITKIWHP | |
| | NISSVTGAICLDILKDQWAAAMTLRTV | |
| | LLSLQALLAAAEPDDPQDAVVANQYK | |
| | QNPEMFKQTARLWAHVYAGAPVSSP | |
| | EYTKKIENLCAMGFDRNAVIVALSSKS | |
| | WDVETATELLLSN | |
| UBE2L3 | AASRRLMKELEEIRKCGMKNFRNIQV | SEQ ID NO: |
| | DEANLLTWQGLIVPDNPPYDKGAFRI | 60 |
| | EINFPAEYPFKPPKITFKTKIYHPNIDE | |
| | KGQVCLPVISAENWKPATKTDQVIQS | |
| | LIALVNDPQPEHPLRADLAEEYSKDRK | |
| | KFCKNAEEFTKKYGEKRPVD | |
| UBE2L6 | MASMRVVKELEDLQKKPPPYLRNLSS | SEQ ID NO: |
| | DDANVLVWHALLLPDQPPYHLKAFNL | 61 |
| | RISFPPEYPFKPPMIKFTTKIYHPNVDE | |
| | NGQICLPIISSENWKPCTKTCQVLEAL | |
| | NVLVNRPNIREPLRMDLADLLTQNPEL | |
| | FRKNAEEFTLRFGVDRPS | |
| UBE2M | ASAAQLRIQKDINELNLPKTCDISFSDP | SEQ ID NO: |
| | DDLLNFKLVICPDEGFYKSGKFVFSFK | 62 |
| | VGQGYPHDPPKVKCETMVYHPNIDLE | |
| | GNVCLNILREDWKPVLTINSIIYGLQYL | |
| | FLEPNPEDPLNKEAAEVLQNNRRLFE | |
| | QNVQRSMRGGYIGSTYFERCLK | |
| UBE20 | SNHSFKKIEFQPPEAKKFFSTVRKEM | SEQ ID NO: |
| | ALLATSLPEGIMVKTFEDRMDLFSALI | 65 |
| | KGPTRTPYEDGLYLFDIQLPNIYPAVP | |
| | PHFCYLSQCSGRLNPNLYDNGKVCV | |
| | SLLGTWIGKGTERWTSKSSLLQVLISI | |
| | QGLILV | |
| UBE2Q1 | SGSVQATDRLMKELRDIYRSQSFKGG | SEQ ID NO: |
| | NYAVELVNDSLYDWNVKLLKVDQDSA | 66 |
| | LHNDLQILKEKEGADFILLNFSFKDNF | |
| | PFDPPFVRVVSPVLSGGYVLGGGAIC | |
| | MELLTKQGWSSAYSIESVIMQISATLV | |
| | KGKARVQFGANKSQYSLTRAQQSYK | |
| | SLVQIHEKNGW | |
| UBE2Q2 | GAVSGSVQASDRLMKELRDIYRSQSY | SEQ ID NO: |
| | KTGIYSVELINDSLYDWHVKLQKVDPD | 67 |
| | SPLHSDLQILKEKEGIEYILLNFSFKDN | |
| | FPFDPPFVRVVLPVLSGGYVLGGGAL | |
| | CMELLTKQGWSSAYSIESVIMQINATL | |
| | VKGKARVQFGANKNQYNLARAQQSY | |
| | NSIVQIHE | |
| UBE2R1 | PSSQKALLLELKGLQEEPVEGFRVTL | SEQ ID NO: |
| | VDEGDLYNWEVAIFGPPNTYYEGGYF | 69 |
| | KARLKFPIDYPYSPPAFRFLTKMWHP | |
| | NIYETGDVCISILHPPVDDPQSGELPS | |
| | ERWNPTQNVRTILLSVISLLNEPNTFS | |
| | PANVDASVMYRKWKESKGKDREYTD | |
| | IIRKQVLGTKVDAERDGVKVP | |
| UBE2S | NSNVENLPPHIIRLVYKEVTTLTADPP | SEQ ID NO: |
| | DGIKVFPNEEDLTDLQVTIEGPEGTPY | 71 |
| | AGGLFRMKLLLGKDFPASPPKGYFLT | |
| | KIFHPNVGANGEICVNVLKRDWTAEL | |
| | GIRHVLLTIKCLLIHPNPESALNEEAGR | |
| | LLLENYEEYAARARLLTEIHG | |
| UBE2T | QRASRLKRELHMLATEPPPGITCWQD | SEQ ID NO: |
| | KDQMDDLRAQILGGANTPYEKGVFKL | 72 |
| | EVIIPERYPFEPPQIRFLTPIYHPNIDSA | |
| | GRICLDVLKLPPKGAWRPSLNIATVLT | |
| | SIQLLMSEPNPDDPLMADISSEFKYNK | |
| | PAFLKNARQWTEKHARQKQKADEEE | |
| | MLDNLP | |
| UBE2U | HGRAYLLLHRDFCDLKENNYKGITAK | SEQ ID NO: |
| | PVSEDMMEWEVEIEGLQNSVWQGLV | 73 |
| | FQLTIHFTSEYNYAPPVVKFITIPFHPN | |
| | VDPHTGQPCIDFLDNPEKWNTNYTLS | |
| | SILLALQVMLSNPVLENPVNLEAARILV | |
| | KDESLYRTILRLFNRP | |
| UBE2W | MASMQKRLQKELLALQNDPPPGMTL | SEQ ID NO: |
| | NEKSVQNSITQWIVDMEGAPGTLYEG | 76 |
| | EKFQLLFKFSSRYPFDSPQVMFTGEN | |
| | IPVHPHVYSNGHICLSILTEDWSPALS | |
| | VQSVCLSIISMLS | |
| BIRC6 | ANQEKKLGEYSKKAAMKPKPLSVLKS | SEQ ID NO: |
| | LEEKYVAVMKKLQFDTFEMVSEDEDG | 79 |
| | KLGFKVNYHYMSQVKNANDANSAAR | |
| | ARRLAQEAVTLSTSLPLSSSSSVFVR | |
| | CDEERLDIMKVLITGPADTPYANGCFE | |
| | FDVYFPQDYPSSPPLVNLETTGGHSV | |
| | RFNPNLYNDGKVCLSILNTWHGRPEE | |
| | KWNPQTSSFLQVLVSVQSLIL | |
| UFC1 | ADEATRRVVSEIPVLKTNAGPRDREL | SEQ ID NO: |
| | WVQRLKEEYQSLIRYVENNKNADND | 82 |
| | WFRLESNKEGTRWFGKCWYIHDLLK | |
| | YEFDIEFDIPITYPTTAPEIAVPELDGKT | |
| | AKMYRGGKICLTDHFKPLWARNVPKF | |
| | GLAHLMALGLGPWLAVEIPDLIQKGVI | |
| | QHKEKCNQ | |

TABLE 11
HA-tagged regulation and targeting domains used as experimental
controls, and corresponding SEQ ID NOS.

| Expression tag | | |
|---|---|---|
| Regulation domain | Amino acid sequence | SEQ ID NO: |
| HA_VHL | YPYDVPDYAAMPRRAENWDEAEV | SEQ ID NO: |
| | GAEEAGVEEYGPEEDGGEESGAE | 168 |
| | ESGPEESGPEELGAEEEMEVGRP | |
| | RPVLRSVNSREPSQVIFCNRSPRV | |
| | VLPVWLNFDGEPQPYPTLPPGTG | |
| | RRIHSYRGHLWLFRDAGTHDGLL | |
| | VNQTELFVPSLNVDGQPIFANITLP | |
| | VYTLKERCLQVVRSLVKPENYRRL | |
| | DIVRSLYEDLEDHPNVQKDLERLT | |
| | QERIAHQRMGD | |
| HA_UBE2D1 | YPYDVPDYAALKRIQKELSDLQRD | SEQ ID NO: |
| | PPAHCSAGPVGDDLFHWQATIMG | 169 |
| | PPDSAYQGGVFFLTVHFPTDYPFK | |
| | PPKIAFTTKIYHPNINSNGSICLDIL | |
| | RSQWSPALTVSKVLLSICSLLCDP | |
| | NPDDPLVPDIAQIYKSDKEKYNRH | |
| | AREWTQKYAM | |
| Expression tag_ | | |
| Targeting domain | Amino acid sequence | SEQ ID NO: |
| HA_aCS3 | YPYDVPDYAEFAMVSSVPTKLEVV | SEQ ID NO: |
| | AATPTSLLISWDAPAVTVDYYVITY | 149 |
| | GETGHWPWVWQEFEVPGSKSTA | |
| | TISGLKPGVDYTITVYAGSYSSYYY | |
| | YGSPISINYRT | |
| HA_K19 | YPYDVPDYADLGKKLLEAARAGQ | SEQ ID NO: |
| | DDEVRILMANGADVNASDRWGWT | 150 |
| | PLHLAAWWGHLEIVEVLLKRGAD | |
| | VSAADLHGQSPLHLAAMVGHLEIV | |
| | EVLLKYGADVNAKDTMGATPLHL | |
| | AARSGHLEIVEELLKNGADMNAQ | |
| | DKFGKTTFDISTDNGNEDLAEILQK | |
| | L | |
| HA_E3_5 | YPYDVPDYADLGKKLLEAARAGQ | SEQ ID NO: |
| | DDEVRILMANGADVNATDNDGYT | 151 |
| | PLHLAASNGHLEIVEVLLKNGADV | |
| | NASDLTGITPLHLAAATGHLEIVEV | |
| | LLKHGADVNAYDNDGHTPLHLAA | |
| | KYGHLEIVEVLLKHGADVNAQDKF | |
| | GKTAFDISIDNGNEDLAEILQ | |

TABLE 12A
Core E2 fusion polypeptide sequences of the disclosure and corresponding
SEQ ID NOS.
Fusion polypeptides

| Expression tag_ | | |
|---|---|---|
| Regulation domain_ | | |
| Linker_ | | |
| Targeting domain | Amino acid sequence | SEQ ID NO: |
| HA_UBE2B_Linker | YPYDVPDYASTPARRRLMRDFKR | SEQ ID NO: |
| 2_aCS3 | LQEDPPVGVSGAPSENNIMQWNA | 156 |
| | VIFGPEGTPFEDGTFKLVIEFSEEY | |
| | PNKPPTVRFLSKMFHPNVYADGSI | |
| | CLDILQNRWSPTYDVSSILTSIQSL | |
| | LDEPNPNSPANSQAAQLYQENKR | |
| | EYEKRVSAIVEQSWNDSLEGGGG | |
| | SGGGGSGGGGSSRVSSVPTKLEV | |
| | VAATPTSLLISWDAPAVTVDYYVIT | |
| | YGETGHWPWVWQEFEVPGSKST | |
| | ATISGLKPGVDYTITVYAGSYSSYY | |
| | YYGSPISINYRT | |
| HA_UBE2C_Linker | YPYDVPDYAASQNRDPAATSVAA | SEQ ID NO: |
| 2_aCS3 | ARKGAEPSGGAARGPVGKRLQQ | 157 |
| | ELMTLMMSGDKGISAFPESDNLFK | |
| | WVGTIHGAAGTVYEDLRYKLSLEF | |
| | PSGYPYNAPTVKFLTPCYHPNVDT | |
| | QGNICLDILKEKWSALYDVRTILLSI | |
| | QSLLGEPNIDSPLNTHAAELWKNP | |
| | TAFKKYLQETYSKQVTSQEPLEGG | |
| | GGSGGGGSGGGGSSRVSSVPTKL | |
| | EVVAATPTSLLISWDAPAVTVDYYV | |
| | ITYGETGHWPWVWQEFEVPGSKS | |
| | TATISGLKPGVDYTITVYAGSYSSY | |
| | YYYGSPISINYRT | |
| HA_UBE2D1_Linker | YPYDVPDYAALKRIQKELSDLQRD | SEQ ID NO: |
| 1_aCS3 | PPAHCSAGPVGDDLFHWQATIMG | 158 |
| | PPDSAYQGGVFFLTVHFPTDYPFK | |
| | PPKIAFTTKIYHPNINSNGSICLDIL | |
| | RSQWSPALTVSKVLLSICSLLCDP | |
| | NPDDPLVPDIAQIYKSDKEKYNRH | |
| | AREWTQKYAMLEGGGGSSRVSS | |
| | VPTKLEVVAATPTSLLISWDAPAVT | |
| | VDYYVITYGETGHWPWVWQEFEV | |
| | PGSKSTATISGLKPGVDYTITVYAG | |
| | SYSSYYYYGSPISINYRT | |
| HA_UBE2D1_Linker | YPYDVPDYAALKRIQKELSDLQRD | SEQ ID NO: |
| 2_aCS3 | PPAHCSAGPVGDDLFHWQATIMG | 159 |
| | PPDSAYQGGVFFLTVHFPTDYPFK | |
| | PPKIAFTTKIYHPNINSNGSICLDIL | |
| | RSQWSPALTVSKVLLSICSLLCDP | |
| | NPDDPLVPDIAQIYKSDKEKYNRH | |
| | AREWTQKYAMLEGGGGSGGGGS | |
| | GGGGSSRVSSVPTKLEVVAATPTS | |
| | LLISWDAPAVTVDYYVITYGETGH | |
| | WPWVWQEFEVPGSKSTATISGLK | |
| | PGVDYTITVYAGSYSSYYYYGSPIS | |
| | INYRT | |
| HA_UBE2D1_Linker | YPYDVPDYAALKRIQKELSDLQRD | SEQ ID NO: |
| 2_aCS3 (V33R) | PPAHCSAGPVGDDLFHWQATIMG | 160 |
| | PPDSAYQGGVFFLTVHFPTDYPFK | |
| | PPKIAFTTKIYHPNINSNGSICLDIL | |
| | RSQWSPALTVSKVLLSICSLLCDP | |
| | NPDDPLVPDIAQIYKSDKEKYNRH | |
| | AREWTQKYAMLEGGGGSGGGGS | |
| | GGGGSSRVSSVPTKLEVVAATPTS | |
| | LLISWDAPAVTVDYYRITYGETGH | |
| | WPWVWQEFEVPGSKSTATISGLK | |
| | PGVDYTITVYAGSYSSYYYYGSPIS | |
| | INYRT | |
| HA_UBE2D1_Linker | YPYDVPDYAALKRIQKELSDLQRD | SEQ ID NO: |
| 2_aCS3 (K7Q) | PPAHCSAGPVGDDLFHWQATIMG | 161 |
| | PPDSAYQGGVFFLTVHFPTDYPFK | |
| | PPKIAFTTKIYHPNINSNGSICLDIL | |
| | RSQWSPALTVSKVLLSICSLLCDP | |
| | NPDDPLVPDIAQIYKSDKEKYNRH | |
| | AREWTQKYAMLEGGGGSGGGGS | |
| | GGGGSSRVSSVPTQLEVVAATPTS | |
| | LLISWDAPAVTVDYYVITYGETGH | |
| | WPWVWQEFEVPGSKSTATISGLK | |
| | PGVDYTITVYAGSYSSYYYYGSPIS | |
| | INYRT | |
| HA_UBE2D1_Linker | YPYDVPDYAALKRIQKELSDLQRD | SEQ ID NO: |
| 2_aCS3 (K55Y) | PPAHCSAGPVGDDLFHWQATIMG | 162 |
| | PPDSAYQGGVFFLTVHFPTDYPFK | |
| | PPKIAFTTKIYHPNINSNGSICLDIL | |
| | RSQWSPALTVSKVLLSICSLLCDP | |
| | NPDDPLVPDIAQIYKSDKEKYNRH | |
| | AREWTQKYAMLEGGGGSGGGGS | |
| | GGGGSSRVSSVPTKLEVVAATPTS | |
| | LLISWDAPAVTVDYYVITYGETGH | |
| | WPWVWQEFEVPGSYSTATISGLK | |
| | PGVDYTITVYAGSYSSYYYYGSPIS | |
| | INYRT | |
| HA_UBE2D1_Linker | YPYDVPDYAALKRIQKELSDLQRD | SEQ ID NO: |
| 2_aCS3 (K64H) | PPAHCSAGPVGDDLFHWQATIMG | 163 |
| | PPDSAYQGGVFFLTVHFPTDYPFK | |
| | PPKIAFTTKIYHPNINSNGSICLDIL | |
| | RSQWSPALTVSKVLLSICSLLCDP | |
| | NPDDPLVPDIAQIYKSDKEKYNRH | |
| | AREWTQKYAMLEGGGGSGGGGS | |
| | GGGGSSRVSSVPTKLEVVAATPTS | |
| | LLISWDAPAVTVDYYVITYGETGH | |
| | WPWVWQEFEVPGSKSTATISGLH | |
| | PGVDYTITVYAGSYSSYYYYGSPIS | |
| | INYRT | |
| HA_UBE2D1_Linker | YPYDVPDYAALKRIQKELSDLQRD | SEQ ID NO: |
| 2_aCS3 (K7Q, K55Y) | PPAHCSAGPVGDDLFHWQATIMG | 164 |
| | PPDSAYQGGVFFLTVHFPTDYPFK | |
| | PPKIAFTTKIYHPNINSNGSICLDIL | |
| | RSQWSPALTVSKVLLSICSLLCDP | |
| | NPDDPLVPDIAQIYKSDKEKYNRH | |
| | AREWTQKYAMLEGGGGSGGGGS | |
| | GGGGSSRVSSVPTQLEVVAATPTS | |
| | LLISWDAPAVTVDYYVITYGETGH | |
| | WPWVWQEFEVPGSYSTATISGLK | |
| | PGVDYTITVYAGSYSSYYYYGSPIS | |
| | INYRT | |
| HA_UBE2D1_Linker | YPYDVPDYAALKRIQKELSDLQRD | SEQ ID NO: |
| 2_aCS3 (K7Q, K64H) | PPAHCSAGPVGDDLFHWQATIMG | 165 |
| | PPDSAYQGGVFFLTVHFPTDYPFK | |
| | PPKIAFTTKIYHPNINSNGSICLDIL | |
| | RSQWSPALTVSKVLLSICSLLCDP | |
| | NPDDPLVPDIAQIYKSDKEKYNRH | |
| | AREWTQKYAMLEGGGGSGGGGS | |
| | GGGGSSRVSSVPTQLEVVAATPTS | |
| | LLISWDAPAVTVDYYVITYGETGH | |
| | WPWVWQEFEVPGSKSTATISGLH | |
| | PGVDYTITVYAGSYSSYYYYGSPIS | |
| | INYRT | |
| HA_UBE2D1_Linker | YPYDVPDYAALKRIQKELSDLQRD | SEQ ID NO: |
| 2_aCS3 (K55Y, K64H) | PPAHCSAGPVGDDLFHWQATIMG | 166 |
| | PPDSAYQGGVFFLTVHFPTDYPFK | |
| | PPKIAFTTKIYHPNINSNGSICLDIL | |
| | RSQWSPALTVSKVLLSICSLLCDP | |
| | NPDDPLVPDIAQIYKSDKEKYNRH | |
| | AREWTQKYAMLEGGGGSGGGGS | |
| | GGGGSSRVSSVPTKLEVVAATPTS | |
| | LLISWDAPAVTVDYYVITYGETGH | |
| | WPWVWQEFEVPGSYSTATISGLH | |
| | PGVDYTITVYAGSYSSYYYYGSPIS | |
| | INYRT | |
| HA_UBE2D1_Linker | YPYDVPDYAALKRIQKELSDLQRD | SEQ ID NO: |
| 2_aCS3 (K7Q, K55Y, | PPAHCSAGPVGDDLFHWQATIMG | 167 |
| K64H) | PPDSAYQGGVFFLTVHFPTDYPFK | |
| | PPKIAFTTKIYHPNINSNGSICLDIL | |
| | RSQWSPALTVSKVLLSICSLLCDP | |
| | NPDDPLVPDIAQIYKSDKEKYNRH | |
| | AREWTQKYAMLEGGGGSGGGGS | |
| | GGGGSSRVSSVPTQLEVVAATPTS | |
| | LLISWDAPAVTVDYYVITYGETGH | |
| | WPWVWQEFEVPGSYSTATISGLH | |
| | PGVDYTITVYAGSYSSYYYYGSPIS | |
| | INYRT | |
| HA_UBE2D1(C85A)_ | YPYDVPDYAALKRIQKELSDLQRD | SEQ ID NO: |
| Linker 2_aCS3 | PPAHCSAGPVGDDLFHWQATIMG | 170 |
| | PPDSAYQGGVFFLTVHFPTDYPFK | |
| | PPKIAFTTKIYHPNINSNGSIALDIL | |
| | RSQWSPALTVSKVLLSICSLLCDP | |
| | NPDDPLVPDIAQIYKSDKEKYNRH | |
| | AREWTQKYAMLEGGGGSGGGGS | |
| | GGGGSSRVSSVPTKLEVVAATPTS | |
| | LLISWDAPAVTVDYYVITYGETGH | |
| | WPWVWQEFEVPGSKSTATISGLK | |
| | PGVDYTITVYAGSYSSYYYYGSPIS | |
| | INYRT | |
| HA_UBE2D2_Linker | YPYDVPDYAALKRIHKELNDLARD | SEQ ID NO: |
| 2_aCS3 | PPAQCSAGPVGDDMFHWQATIMG | 17″ |
| | PNDSPYQGGVFFLTIHFPTDYPFK | |
| | PPKVAFTTRIYHPNINSNGSICLDIL | |
| | RSQWSPALTISKVLLSICSLLCDPN | |
| | PDDPLVPEIARIYKTDREKYNRIAR | |
| | EWTQKYAMLEGGGGSGGGGSGG | |
| | GGSSRVSSVPTKLEVVAATPTSLLI | |
| | SWDAPAVTVDYYVITYGETGHWP | |
| | WVWQEFEVPGSKSTATISGLKPGV | |
| | DYTITVYAGSYSSYYYYGSPISINY | |
| | RT | |
| HA_UBE2D3_Linker | YPYDVPDYAALKRINKELSDLARD | SEQ ID NO: |
| 2_aCS3 | PPAQCSAGPVGDDMFHWQATIMG | 172 |
| | PNDSPYQGGVFFLTIHFPTDYPFK | |
| | PPKVAFTTRIYHPNINSNGSICLDIL | |
| | RSQWSPALTISKVLLSICSLLCDPN | |
| | PDDPLVPEIARIYKTDRDKYNRISR | |
| | EWTQKYAMLEGGGGSGGGGSGG | |
| | GGSSRVSSVPTKLEVVAATPTSLLI | |
| | SWDAPAVTVDYYVITYGETGHWP | |
| | WVWQEFEVPGSKSTATISGLKPGV | |
| | DYTITVYAGSYSSYYYYGSPISINY | |
| | RT | |
| HA_UBE2E1_Linker | YPYDVPDYASDDDSRASTSSSSSS | SEQ ID NO: |
| 2_aCS3 | SSNQQTEKETNTPKKKESKVSMS | 173 |
| | KNSKLLSTSAKRIQKELADITLDPP | |
| | PNCSAGPKGDNIYEWRSTILGPPG | |
| | SVYEGGVFFLDITFTPEYPFKPPKV | |
| | TFRTRIYHCNINSQGVICLDILKDN | |
| | WSPALTISKVLLSICSLLTDCNPAD | |
| | PLVGSIATQYMTNRAEHDRMARQ | |
| | WTKRYATLEGGGGSGGGGSGGG | |
| | GSSRVSSVPTKLEVVAATPTSLLIS | |
| | WDAPAVTVDYYVITYGETGHWPW | |
| | VWQEFEVPGSKSTATISGLKPGVD | |
| | YTITVYAGSYSSYYYYGSPISINYRT | |
| HA_UBE2F_Linker | YPYDVPDYASTRRVSVRDKLLVKE | SEQ ID NO: |
| 2_aCS3 | VAELEANLPCTCKVHFPDPNKLHC | 174 |
| | FQLTVTPDEGYYQGGKFQFETEVP | |
| | DAYNMVPPKVKCLTKIWHPNITET | |
| | GEICLSLLREHSIDGTGWAPTRTL | |
| | KDVVWGLNSLFTDLLNFDDPLNIE | |
| | AAEHHLRDKEDFRNKVDDYIKRYA | |
| | RLEGGGGSGGGGSGGGGSSRVS | |
| | SVPTKLEVVAATPTSLLISWDAPAV | |
| | TVDYYVITYGETGHWPWVWQEFE | |
| | VPGSKSTATISGLKPGVDYTITVYA | |
| | GSYSSYYYYGSPISINYRT | |
| HA_UBE2G1_Linker | YPYDVPDYALLLRRQLAELNKNPV | SEQ ID NO: |
| 2_aCS3 | EGFSAGLIDDNDLYRWEVLIIGPPD | 175 |
| | TLYEGGVFKAHLTFPKDYPLRPPK | |
| | MKFITEIWHPNVDKNGDVCISILHE | |
| | PGEDKYGYEKPEERWLPIHTVETI | |
| | MISVISMLADPNGDSPANVDAAKE | |
| | WREDRNGEFKRKVARCLEGGGG | |
| | SGGGGSGGGGSSRVSSVPTKLEV | |
| | VAATPTSLLISWDAPAVTVDYYVIT | |
| | YGETGHWPWVWQEFEVPGSKST | |
| | ATISGLKPGVDYTITVYAGSYSSYY | |
| | YYGSPISINYRT | |
| HA_UBE2G2_Linker | YPYDVPDYAAGTALKRLMAEYKQ | SEQ ID NO: |
| 2_aCS3 | LTLNPPEGIVAGPMNEENFFEWEA | 176 |
| | LIMGPEDTCFEFGVFPAILSFPLDY | |
| | PLSPPKMRFTCEMFHPNIYPDGRV | |
| | CISILHAPGDDPMGYESSAERWSP | |
| | VQSVEKILLSVVSMLAEPNDESGA | |
| | NVDASKMWRDDREQFYKIAKQIV | |
| | QKSLGLLEGGGGSGGGGSGGGG | |
| | SSRVSSVPTKLEVVAATPTSLLISW | |
| | DAPAVTVDYYVITYGETGHWPWV | |
| | WQEFEVPGSKSTATISGLKPGVDY | |
| | TITVYAGSYSSYYYYGSPISINYRT | |
| HA_UBE2H_Linker | YPYDVPDYASSPSPGKRRMDTDV | SEQ ID NO: |
| 2_aCS3 | VKLIESKHEVTILGGLNEFVVKFYG | 177 |
| | PQGTPYEGGVWKVRVDLPDKYPF | |
| | KSPSIGFMNKIFHPNIDEASGTVCL | |
| | DVINQTWTALYDLTNIFESFLPQLL | |
| | AYPNPIDPLNGDAAAMYLHRPEEY | |
| | KQKIKEYIQKYATEEALKEQEEGL | |
| | EGGGGSGGGGSGGGGSSRVSSV | |
| | PTKLEVVAATPTSLLISWDAPAVTV | |
| | DYYVITYGETGHWPWVWQEFEVP | |
| | GSKSTATISGLKPGVDYTITVYAGS | |
| | YSSYYYYGSPISINYRT | |
| HA_UBE21_Linker | YPYDVPDYASGIALSRLAQERKA | SEQ ID NO: |
| 2_aCS3 | WRKDHPFGFVAVPTKNPDGTMNL | 178 |
| | MNWECAIPGKKGTPWEGGLFKLR | |
| | MLFKDDYPSSPPKCKFEPPLFHPN | |
| | VYPSGTVCLSILEEDKDWRPAITIK | |
| | QILLGIQELLNEPNIQDPAQAEAYTI | |
| | YCQNRVEYEKRVRAQAKKFAPSL | |
| | EGGGGSGGGGSGGGGSSRVSSV | |
| | PTKLEVVAATPTSLLISWDAPAVTV | |
| | DYYVITYGETGHWPWVWQEFEVP | |
| | GSKSTATISGLKPGVDYTITVYAGS | |
| | YSSYYYYGSPISINYRT | |
| HA_UBE2J2_Linker | YPYDVPDYASSTSSKRAPTTATQR | SEQ ID NO: |
| 2_aCS3 | LKQDYLRIKKDPVPYICAEPLPSNI | 179 |
| | LEWHYVVRGPEMTPYEGGYYHGK | |
| | LIFPREFPFKPPSIYMITPNGRFKC | |
| | NTRLCLSITDFHPDTWNPAWSVST | |
| | ILTGLLSFMVEKGPTLGSIETSDFT | |
| | KRQLAVQSLAFNLKDKVFCELFPE | |
| | VVEEIKQKQKAQDELSSRPQTLPL | |
| | EGGGGSGGGGSGGGGSSRVSSV | |
| | PTKLEVVAATPTSLLISWDAPAVTV | |
| | DYYVITYGETGHWPWVWQEFEVP | |
| | GSKSTATISGLKPGVDYTITVYAGS | |
| | YSSYYYYGSPISINYRT | |
| HA_UBE2K_Linker | YPYDVPDYAANIAVQRIKREFKEVL | SEQ ID NO: |
| 2_aCS3 | KSEETSKNQIKVDLVDENFTELRG | 180 |
| | EIAGPPDTPYEGGRYQLEIKIPETY | |
| | PFNPPKVRFITKIWHPNISSVTGAIC | |
| | LDILKDQWAAAMTLRTVLLSLQAL | |
| | LAAAEPDDPQDAVVANQYKQNPE | |
| | MFKQTARLWAHVYAGAPVSSPEY | |
| | TKKIENLCAMGFDRNAVIVALSSK | |
| | SWDVETATELLLSNLEGGGGSGG | |
| | GGSGGGGSSRVSSVPTKLEVVAA | |
| | TPTSLLISWDAPAVTVDYYVITYGE | |
| | TGHWPWVWQEFEVPGSKSTATIS | |
| | GLKPGVDYTITVYAGSYSSYYYYG | |
| | SPISINYRT | |
| HA_UBE2L3_Linker | YPYDVPDYAAASRRLMKELEEIRK | SEQ ID NO: |
| 2_aCS3 | CGMKNFRNIQVDEANLLTWQGLIV | 181 |
| | PDNPPYDKGAFRIEINFPAEYPFKP | |
| | PKITFKTKIYHPNIDEKGQVCLPVIS | |
| | AENWKPATKTDQVIQSLIALVNDP | |
| | QPEHPLRADLAEEYSKDRKKFCK | |
| | NAEEFTKKYGEKRPVDLEGGGGS | |
| | GGGGSGGGGSSRVSSVPTKLEVV | |
| | AATPTSLLISWDAPAVTVDYYVITY | |
| | GETGHWPWVWQEFEVPGSKSTA | |
| | TISGLKPGVDYTITVYAGSYSSYYY | |
| | YGSPISINYRT | |
| HA_UBE2L6_Linker | YPYDVPDYAMASMRVVKELEDLQ | SEQ ID NO: |
| 2_aCS3 | KKPPPYLRNLSSDDANVLVWHAL | 182 |
| | LLPDQPPYHLKAFNLRISFPPEYPF | |
| | KPPMIKFTTKIYHPNVDENGQICLPI | |
| | ISSENWKPCTKTCQVLEALNVLVN | |
| | RPNIREPLRMDLADLLTQNPELFR | |
| | KNAEEFTLRFGVDRPSLEGGGGS | |
| | GGGGSGGGGSSRVSSVPTKLEVV | |
| | AATPTSLLISWDAPAVTVDYYVITY | |
| | GETGHWPWVWQEFEVPGSKSTA | |
| | TISGLKPGVDYTITVYAGSYSSYYY | |
| | YGSPISINYRT | |
| HA_UBE2M_Linker | YPYDVPDYAASAAQLRIQKDINEL | SEQ ID NO: |
| 2_aCS3 | NLPKTCDISFSDPDDLLNFKLVICP | 183 |
| | DEGFYKSGKFVFSFKVGQGYPHD | |
| | PPKVKCETMVYHPNIDLEGNVCLN | |
| | ILREDWKPVLTINSIIYGLQYLFLEP | |
| | NPEDPLNKEAAEVLQNNRRLFEQ | |
| | NVQRSMRGGYIGSTYFERCLKLEG | |
| | GGGSGGGGSGGGGSSRVSSVPT | |
| | KLEVVAATPTSLLISWDAPAVTVDY | |
| | YVITYGETGHWPWVWQEFEVPGS | |
| | KSTATISGLKPGVDYTITVYAGSYS | |
| | SYYYYGSPISINYRT | |
| HA_UBE20_Linker | YPYDVPDYASNHSFKKIEFQPPEA | SEQ ID NO: |
| 2_aCS3 | KKFFSTVRKEMALLATSLPEGIMV | 184 |
| | KTFEDRMDLFSALIKGPTRTPYED | |
| | GLYLFDIQLPNIYPAVPPHFCYLSQ | |
| | CSGRLNPNLYDNGKVCVSLLGTWI | |
| | GKGTERWTSKSSLLQVLISIQGLIL | |
| | VLEGGGGSGGGGSGGGGSSRVS | |
| | SVPTKLEVVAATPTSLLISWDAPAV | |
| | TVDYYVITYGETGHWPWVWQEFE | |
| | VPGSKSTATISGLKPGVDYTITVYA | |
| | GSYSSYYYYGSPISINYRT | |
| HA_UBE2Q1_Linker | YPYDVPDYASGSVQATDRLMKEL | SEQ ID NO: |
| 2_aCS3 | RDIYRSQSFKGGNYAVELVNDSLY | 185 |
| | DWNVKLLKVDQDSALHNDLQILK | |
| | EKEGADFILLNFSFKDNFPFDPPFV | |
| | RVVSPVLSGGYVLGGGAICMELLT | |
| | KQGWSSAYSIESVIMQISATLVKG | |
| | KARVQFGANKSQYSLTRAQQSYK | |
| | SLVQIHEKNGWLEGGGGSGGGGS | |
| | GGGGSSRVSSVPTKLEVVAATPTS | |
| | LLISWDAPAVTVDYYVITYGETGH | |
| | WPWVWQEFEVPGSKSTATISGLK | |
| | PGVDYTITVYAGSYSSYYYYGSPIS | |
| | INYRT | |
| HA_UBE2Q2_Linker | YPYDVPDYAGAVSGSVQASDRLM | SEQ ID NO: |
| 2_aCS3 | KELRDIYRSQSYKTGIYSVELINDS | 186 |
| | LYDWHVKLQKVDPDSPLHSDLQIL | |
| | KEKEGIEYILLNFSFKDNFPFDPPF | |
| | VRVVLPVLSGGYVLGGGALCMEL | |
| | LTKQGWSSAYSIESVIMQINATLVK | |
| | GKARVQFGANKNQYNLARAQQS | |
| | YNSIVQIHELEGGGGSGGGGSGG | |
| | GGSSRVSSVPTKLEVVAATPTSLLI | |
| | SWDAPAVTVDYYVITYGETGHWP | |
| | WVWQEFEVPGSKSTATISGLKPGV | |
| | DYTITVYAGSYSSYYYYGSPISINY | |
| | RT | |
| HA_UBE2R1_Linker | YPYDVPDYAPSSQKALLLELKGLQ | SEQ ID NO: |
| 2_aCS3 | EEPVEGFRVTLVDEGDLYNWEVAI | 187 |
| | FGPPNTYYEGGYFKARLKFPIDYP | |
| | YSPPAFRFLTKMWHPNIYETGDVC | |
| | ISILHPPVDDPQSGELPSERWNPT | |
| | QNVRTILLSVISLLNEPNTFSPANV | |
| | DASVMYRKWKESKGKDREYTDIIR | |
| | KQVLGTKVDAERDGVKVPLEGGG | |
| | GSGGGGSGGGGSSRVSSVPTKLE | |
| | VVAATPTSLLISWDAPAVTVDYYVI | |
| | TYGETGHWPWVWQEFEVPGSKS | |
| | TATISGLKPGVDYTITVYAGSYSSY | |
| | YYYGSPISINYRT | |
| HA_UBE2S_Linker | YPYDVPDYANSNVENLPPHIIRLVY | SEQ ID NO: |
| 2_aCS3 | KEVTTLTADPPDGIKVFPNEEDLT | 188 |
| | DLQVTIEGPEGTPYAGGLFRMKLL | |
| | LGKDFPASPPKGYFLTKIFHPNVG | |
| | ANGEICVNVLKRDWTAELGIRHVL | |
| | LTIKCLLIHPNPESALNEEAGRLLL | |
| | ENYEEYAARARLLTEIHGLEGGGG | |
| | SGGGGSGGGGSSRVSSVPTKLEV | |
| | VAATPTSLLISWDAPAVTVDYYVIT | |
| | YGETGHWPWVWQEFEVPGSKST | |
| | ATISGLKPGVDYTITVYAGSYSSYY | |
| | YYGSPISINYRT | |
| HA_UBE2T_Linker | YPYDVPDYAQRASRLKRELHMLA | SEQ ID NO: |
| 2_aCS3 | TEPPPGITCWQDKDQMDDLRAQIL | 189 |
| | GGANTPYEKGVFKLEVIIPERYPFE | |
| | PPQIRFLTPIYHPNIDSAGRICLDVL | |
| | KLPPKGAWRPSLNIATVLTSIQLL | |
| | MSEPNPDDPLMADISSEFKYNKPA | |
| | FLKNARQWTEKHARQKQKADEE | |
| | EMLDNLPLEGGGGSGGGGSGGG | |
| | GSSRVSSVPTKLEVVAATPTSLLIS | |
| | WDAPAVTVDYYVITYGETGHWPW | |
| | VWQEFEVPGSKSTATISGLKPGVD | |
| | YTITVYAGSYSSYYYYGSPISINYRT | |
| HA_UBE2U_Linker | YPYDVPDYAHGRAYLLLHRDFCDL | SEQ ID NO: |
| 2_aCS3 | KENNYKGITAKPVSEDMMEWEVEI | 190 |
| | EGLQNSVWQGLVFQLTIHFTSEYN | |
| | YAPPVVKFITIPFHPNVDPHTGQPC | |
| | IDFLDNPEKWNTNYTLSSILLALQV | |
| | MLSNPVLENPVNLEAARILVKDES | |
| | LYRTILRLFNRPLEGGGGSGGGGS | |
| | GGGGSSRVSSVPTKLEVVAATPTS | |
| | LLISWDAPAVTVDYYVITYGETGH | |
| | WPWVWQEFEVPGSKSTATISGLK | |
| | PGVDYTITVYAGSYSSYYYYGSPIS | |
| | INYRT | |
| HA_UBE2W_Linker | YPYDVPDYAMASMQKRLQKELLA | SEQ ID NO: |
| 2_aCS3 | LQNDPPPGMTLNEKSVQNSITQWI | 191 |
| | VDMEGAPGTLYEGEKFQLLFKFS | |
| | SRYPFDSPQVMFTGENIPVHPHVY | |
| | SNGHICLSILTEDWSPALSVQSVC | |
| | LSIISMLSLEGGGGSGGGGSGGG | |
| | GSSRVSSVPTKLEVVAATPTSLLIS | |
| | WDAPAVTVDYYVITYGETGHWPW | |
| | VWQEFEVPGSKSTATISGLKPGVD | |
| | YTITVYAGSYSSYYYYGSPISINYRT | |
| HA_BIRC6_Linker | YPYDVPDYAANQEKKLGEYSKKA | SEQ ID NO: |
| 2_aCS3 | AMKPKPLSVLKSLEEKYVAVMKK | 192 |
| | LQFDTFEMVSEDEDGKLGFKVNY | |
| | HYMSQVKNANDANSAARARRLA | |
| | QEAVTLSTSLPLSSSSSVFVRCDE | |
| | ERLDIMKVLITGPADTPYANGCFEF | |
| | DVYFPQDYPSSPPLVNLETTGGHS | |
| | VRFNPNLYNDGKVCLSILNTWHGR | |
| | PEEKWNPQTSSFLQVLVSVQSLIL | |
| | LEGGGGSGGGGSGGGGSSRVSS | |
| | VPTKLEVVAATPTSLLISWDAPAVT | |
| | VDYYVITYGETGHWPWVWQEFEV | |
| | PGSKSTATISGLKPGVDYTITVYAG | |
| | SYSSYYYYGSPISINYRT | |
| HA_UFC1_Linker | YPYDVPDYAADEATRRVVSEIPVL | SEQ ID NO: |
| 2_aCS3 | KTNAGPRDRELWVQRLKEEYQSL | 193 |
| | IRYVENNKNADNDWFRLESNKEG | |
| | TRWFGKCWYIHDLLKYEFDIEFDIP | |
| | ITYPTTAPEIAVPELDGKTAKMYRG | |
| | GKICLTDHFKPLWARNVPKFGLAH | |
| | LMALGLGPWLAVEIPDLIQKGVIQ | |
| | HKEKCNQLEGGGGSGGGGSGGG | |
| | GSSRVSSVPTKLEVVAATPTSLLIS | |
| | WDAPAVTVDYYVITYGETGHWPW | |
| | VWQEFEVPGSKSTATISGLKPGVD | |
| | YTITVYAGSYSSYYYYGSPISINYRT | |
| HA_UBE2D1_Linker | YPYDVPDYAALKRIQKELSDLQRD | SEQ ID NO: |
| 4_aCS3 | PPAHCSAGPVGDDLFHWQATIMG | 194 |
| | PPDSAYQGGVFFLTVHFPTDYPFK | |
| | PPKIAFTTKIYHPNINSNGSICLDIL | |
| | RSQWSPALTVSKVLLSICSLLCDP | |
| | NPDDPLVPDIAQIYKSDKEKYNRH | |
| | AREWTQKYAMGGGGGVSSVPTK | |
| | LEVVAATPTSLLISWDAPAVTVDYY | |
| | VITYGETGHWPWVWQEFEVPGSK | |
| | STATISGLKPGVDYTITVYAGSYSS | |
| | YYYYGSPISINYRT | |
| HA_UBE2D1_Linker | YPYDVPDYAALKRIQKELSDLQRD | SEQ ID NO: |
| 2_K19 | PPAHCSAGPVGDDLFHWQATIMG | 254 |
| | PPDSAYQGGVFFLTVHFPTDYPFK | |
| | PPKIAFTTKIYHPNINSNGSICLDIL | |
| | RSQWSPALTVSKVLLSICSLLCDP | |
| | NPDDPLVPDIAQIYKSDKEKYNRH | |
| | AREWTQKYAMLEGGGGSGGGGS | |
| | GGGGSSRDLGKKLLEAARAGQDD | |
| | EVRILMANGADVNASDRWGWTPL | |
| | HLAAWWGHLEIVEVLLKRGADVSA | |
| | ADLHGQSPLHLAAMVGHLEIVEVLL | |
| | KYGADVNAKDTMGATPLHLAARS | |
| | GHLEIVEELLKNGADMNAQDKFGK | |
| | TTFDISTDNGNEDLAEILQKL | |
| HA_UBE2B_Linker | YPYDVPDYASTPARRRLMRDFKR | SEQ ID NO: |
| 2_K19 | LQEDPPVGVSGAPSENNIMQWNA | 256 |
| | VIFGPEGTPFEDGTFKLVIEFSEEY | |
| | PNKPPTVRFLSKMFHPNVYADGSI | |
| | CLDILQNRWSPTYDVSSILTSIQSL | |
| | LDEPNPNSPANSQAAQLYQENKR | |
| | EYEKRVSAIVEQSWNDSLEGGGG | |
| | SGGGGSGGGGSSRDLGKKLLEAA | |
| | RAGQDDEVRILMANGADVNASDR | |
| | WGWTPLHLAAWWGHLEIVEVLLK | |
| | RGADVSAADLHGQSPLHLAAMVG | |
| | HLEIVEVLLKYGADVNAKDTMGAT | |
| | PLHLAARSGHLEIVEELLKNGADM | |
| | NAQDKFGKTTFDISTDNGNEDLAEI | |
| | LQKL | |
| Expression tag_ | | |
| Targeting domain | | |
| Linker | | |
| Regulation domain | Amino acid sequence | SEQ ID NO: |
| HA_aCS3_Linker | YPYDVPDYAEFAMVSSVPTKLEVV | SEQ ID NO: |
| 1_UBE2D1 | AATPTSLLISWDAPAVTVDYYVITY | 202 |
| | GETGHWPWVWQEFEVPGSKSTA | |
| | TISGLKPGVDYTITVYAGSYSSYYY | |
| | YGSPISINYRTLEGGGGSSRALKRI | |
| | QKELSDLQRDPPAHCSAGPVGDD | |
| | LFHWQATIMGPPDSAYQGGVFFLT | |
| | VHFPTDYPFKPPKIAFTTKIYHPNIN | |
| | SNGSICLDILRSQWSPALTVSKVLL | |
| | SICSLLCDPNPDDPLVPDIAQIYKSD | |
| | KEKYNRHAREWTQKYAM | |
| HA_aCS3_Linker | YPYDVPDYAEFAMVSSVPTKLEVV | SEQ ID NO: |
| 2_UBE2D1 | AATPTSLLISWDAPAVTVDYYVITY | 203 |
| | GETGHWPWVWQEFEVPGSKSTA | |
| | TISGLKPGVDYTITVYAGSYSSYYY | |
| | YGSPISINYRTLEGGGGSGGGGSG | |
| | GGGSSRALKRIQKELSDLQRDPPA | |
| | HCSAGPVGDDLFHWQATIMGPPD | |
| | SAYQGGVFFLTVHFPTDYPFKPPKI | |
| | AFTTKIYHPNINSNGSICLDILRSQW | |
| | SPALTVSKVLLSICSLLCDPNPDDP | |
| | LVPDIAQIYKSDKEKYNRHAREWT | |
| | QKYAM | |
| HA_K19_linker | YPYDVPDYADLGKKLLEAARAGQ | SEQ ID NO: |
| 3_UBE2D1 | DDEVRILMANGADVNASDRWGWT | 204 |
| | PLHLAAWWGHLEIVEVLLKRGAD | |
| | VSAADLHGQSPLHLAAMVGHLEIV | |
| | EVLLKYGADVNAKDTMGATPLHL | |
| | AARSGHLEIVEELLKNGADMNAQ | |
| | DKFGKTTFDISTDNGNEDLAEILQK | |
| | LAAAGGGGSGGGGSGGGGSGTA | |
| | LKRIQKELSDLQRDPPAHCSAGPV | |
| | GDDLFHWQATIMGPPDSAYQGGV | |
| | FFLTVHFPTDYPFKPPKIAFTTKIYH | |
| | PNINSNGSICLDILRSQWSPALTVS | |
| | KVLLSICSLLCDPNPDDPLVPDIAQI | |
| | YKSDKEKYNRHAREWTQKYAM | |
| HA_E3_5_linker | YPYDVPDYADLGKKLLEAARAGQ | SEQ ID NO: |
| 3_UBE2D1 | DDEVRILMANGADVNATDNDGYT | 205 |
| | PLHLAASNGHLEIVEVLLKNGADV | |
| | NASDLTGITPLHLAAATGHLEIVEV | |
| | LLKHGADVNAYDNDGHTPLHLAA | |
| | KYGHLEIVEVLLKHGADVNAQDKF | |
| | GKTAFDISIDNGNEDLAEILQAAAG | |
| | GGGSGGGGSGGGGSGTALKRIQK | |
| | ELSDLQRDPPAHCSAGPVGDDLFH | |
| | WQATIMGPPDSAYQGGVFFLTVHF | |
| | PTDYPFKPPKIAFTTKIYHPNINSNG | |
| | SICLDILRSQWSPALTVSKVLLSICS | |
| | LLCDPNPDDPLVPDIAQIYKSDKEK | |
| | YNRHAREWTQKYAM | |
| HA_aCS3(V33R)_ | YPYDVPDYAEFAMVSSVPTKLEVV | SEQ ID NO: |
| Linker 2_UBE2D1 | AATPTSLLISWDAPAVTVDYYRITY | 195 |
| | GETGHWPWVWQEFEVPGSKSTA | |
| | TISGLKPGVDYTITVYAGSYSSYYY | |
| | YGSPISINYRTLEGGGGSGGGGSG | |
| | GGGSSRALKRIQKELSDLQRDPPA | |
| | HCSAGPVGDDLFHWQATIMGPPD | |
| | SAYQGGVFFLTVHFPTDYPFKPPKI | |
| | AFTTKIYHPNINSNGSICLDILRSQW | |
| | SPALTVSKVLLSICSLLCDPNPDDP | |
| | LVPDIAQIYKSDKEKYNRHAREWT | |
| | QKYAM | |
| HA_K19_Linker | YPYDVPDYADLGKKLLEAARAGQ | SEQ ID NO: |
| 2_UBE2D1 | DDEVRILMANGADVNASDRWGWT | 253 |
| | PLHLAAWWGHLEIVEVLLKRGAD | |
| | VSAADLHGQSPLHLAAMVGHLEIV | |
| | EVLLKYGADVNAKDTMGATPLHL | |
| | AARSGHLEIVEELLKNGADMNAQ | |
| | DKFGKTTFDISTDNGNEDLAEILQK | |
| | LLEGGGGSGGGGSGGGGSSRAL | |
| | KRIQKELSDLQRDPPAHCSAGPVG | |
| | DDLFHWQATIMGPPDSAYQGGVF | |
| | FLTVHFPTDYPFKPPKIAFTTKIYHP | |
| | NINSNGSICLDILRSQWSPALTVSK | |
| | VLLSICSLLCDPNPDDPLVPDIAQIY | |
| | KSDKEKYNRHAREWTQKYAM | |
| HA_K19_Linker | YPYDVPDYADLGKKLLEAARAGQ | SEQ ID NO: |
| 2_UBE2B | DDEVRILMANGADVNASDRWGWT | 255 |
| | PLHLAAWWGHLEIVEVLLKRGAD | |
| | VSAADLHGQSPLHLAAMVGHLEIV | |
| | EVLLKYGADVNAKDTMGATPLHL | |
| | AARSGHLEIVEELLKNGADMNAQ | |
| | DKFGKTTFDISTDNGNEDLAEILQK | |
| | LLEGGGGSGGGGSGGGGSSRST | |
| | PARRRLMRDFKRLQEDPPVGVSG | |
| | APSENNIMQWNAVIFGPEGTPFED | |
| | GTFKLVIEFSEEYPNKPPTVRFLSK | |
| | MFHPNVYADGSICLDILQNRWSPT | |
| | YDVSSILTSIQSLLDEPNPNSPANS | |
| | QAAQLYQENKREYEKRVSAIVEQS | |
| | WNDS | |
| HA_E3_5_Linker | YPYDVPDYADLGKKLLEAARAGQ | SEQ ID NO: |
| 2_UBE2B | DDEVRILMANGADVNATDNDGYT | 274 |
| | PLHLAASNGHLEIVEVLLKNGADV | |
| | NASDLTGITPLHLAAATGHLEIVEV | |
| | LLKHGADVNAYDNDGHTPLHLAA | |
| | KYGHLEIVEVLLKHGADVNAQDKF | |
| | GKTAFDISIDNGNEDLAEILQLEGG | |
| | GGSGGGGSGGGGSSRSTPARRR | |
| | LMRDFKRLQEDPPVGVSGAPSEN | |
| | NIMQWNAVIFGPEGTPFEDGTFKL | |
| | VIEFSEEYPNKPPTVRFLSKMFHPN | |
| | VYADGSICLDILQNRWSPTYDVSSI | |
| | LTSIQSLLDEPNPNSPANSQAAQLY | |
| | QENKREYEKRVSAIVEQSWNDS | |
| HA_E3_5_Linker | YPYDVPDYAAMPRRAENWDEAEV | SEQ ID NO: |
| 2_UBE2D1 | GAEEAGVEEYGPEEDGGEESGAE | 275 |
| | ESGPEESGPEELGAEEEMEVGRP | |
| | RPVLRSVNSREPSQVIFCNRSPRV | |
| | VLPVWLNFDGEPQPYPTLPPGTG | |
| | RRIHSYRGHLWLFRDAGTHDGLL | |
| | VNQTELFVPSLNVDGQPIFANITLP | |
| | VYTLKERCLQVVRSLVKPENYRRL | |
| | DIVRSLYEDLEDHPNVQKDLERLT | |
| | QERIAHQRMGDLEGGGGSGGGG | |
| | SGGGGSSRDLGKKLLEAARAGQD | |
| | DEVRILMANGADVNATDNDGYTPL | |
| | HLAASNGHLEIVEVLLKNGADVNAS | |
| | DLTGITPLHLAAATGHLEIVEVLLKH | |
| | GADVNAYDNDGHTPLHLAAKYGHL | |
| | EIVEVLLKHGADVNAQDKFGKTAF | |
| | DISIDNGNEDLAEILQ | |
| Regulation domain | | |
| Linker | | |
| Targeting domain | | |
| Expression tag | Amino acid sequence | SEQ ID NO: |
| UBE2D1_Linker | ALKRIQKELSDLQRDPPAHCSAGP | SEQ ID NO: |
| 7_aCS3(K7Q, K55Y, | VGDDLFHWQATIMGPPDSAYQGG | 236 |
| K64H)_HA | VFFLTVHFPTDYPFKPPKIAFTTKIY | |
| | HPNINSNGSICLDILRSQWSPALTV | |
| | SKVLLSICSLLCDPNPDDPLVPDIA | |
| | QIYKSDKEKYNRHAREWTQKYAML | |
| | EGGSRVSSVPTQLEVVAATPTSLL | |
| | ISWDAPAVTVDYYVITYGETGHWP | |
| | WVWQEFEVPGSYSTATISGLHPG | |
| | VDYTITVYAGSYSSYYYYGSPISIN | |
| | YRTGGYPYDVPDYA | |
| UBE2D1_Linker | ALKRIQKELSDLQRDPPAHCSAGP | SEQ ID NO: |
| 8_aCS3 | VGDDLFHWQATIMGPPDSAYQGG | 237 |
| (K7Q, K55Y, K64H)_HA | VFFLTVHFPTDYPFKPPKIAFTTKIY | |
| | HPNINSNGSICLDILRSQWSPALTV | |
| | SKVLLSICSLLCDPNPDDPLVPDIA | |
| | QIYKSDKEKYNRHAREWTQKYAML | |
| | EGGGSGGSSRVSSVPTQLEVVAA | |
| | TPTSLLISWDAPAVTVDYYVITYGE | |
| | TGHWPWVWQEFEVPGSYSTATIS | |
| | GLHPGVDYTITVYAGSYSSYYYYG | |
| | SPISINYRTGGYPYDVPDYA | |
| UBE2D1_Linker | ALKRIQKELSDLQRDPPAHCSAGP | SEQ ID NO: |
| 9_aCS3 | VGDDLFHWQATIMGPPDSAYQGG | 238 |
| (K7Q, K55Y, K64H)_HA | VFFLTVHFPTDYPFKPPKIAFTTKIY | |
| | HPNINSNGSICLDILRSQWSPALTV | |
| | SKVLLSICSLLCDPNPDDPLVPDIA | |
| | QIYKSDKEKYNRHAREWTQKYAML | |
| | EGGGGSGGGSSRVSSVPTQLEVV | |
| | AATPTSLLISWDAPAVTVDYYVITY | |
| | GETGHWPWVWQEFEVPGSYSTA | |
| | TISGLHPGVDYTITVYAGSYSSYYY | |
| | YGSPISINYRTGGYPYDVPDYA | |
| UBE2D1_Linker | ALKRIQKELSDLQRDPPAHCSAGP | SEQ ID NO: |
| 10_aCS3(K7Q, K55Y, | VGDDLFHWQATIMGPPDSAYQGG | 239 |
| K64H)_HA | VFFLTVHFPTDYPFKPPKIAFTTKIY | |
| | HPNINSNGSICLDILRSQWSPALTV | |
| | SKVLLSICSLLCDPNPDDPLVPDIA | |
| | QIYKSDKEKYNRHAREWTQKYAML | |
| | EGGGSGGGSGGGSSRVSSVPTQL | |
| | EVVAATPTSLLISWDAPAVTVDYY | |
| | VITYGETGHWPWVWQEFEVPGSY | |
| | STATISGLHPGVDYTITVYAGSYSS | |
| | YYYYGSPISINYRTGGYPYDVPDYA | |
| UBE2D1_Linker | ALKRIQKELSDLQRDPPAHCSAGP | SEQ ID NO: |
| 2_aCS3(K7Q, K55Y, | VGDDLFHWQATIMGPPDSAYQGG | 240 |
| K64H)_HA | VFFLTVHFPTDYPFKPPKIAFTTKIY | |
| | HPNINSNGSICLDILRSQWSPALTV | |
| | SKVLLSICSLLCDPNPDDPLVPDIA | |
| | QIYKSDKEKYNRHAREWTQKYAML | |
| | EGGGGSGGGGSGGGGSSRVSSV | |
| | PTQLEVVAATPTSLLISWDAPAVT | |
| | VDYYVITYGETGHWPWVWQEFEV | |
| | PGSYSTATISGLHPGVDYTITVYAG | |
| | SYSSYYYYGSPISINYRTGGYPYDV | |
| | PDYA | |
| UBE2D1_Linker | ALKRIQKELSDLQRDPPAHCSAGP | SEQ ID NO: |
| 11_aCS3(K7Q, K55Y, | VGDDLFHWQATIMGPPDSAYQGG | 241 |
| K64H)_HA | VFFLTVHFPTDYPFKPPKIAFTTKIY | |
| | HPNINSNGSICLDILRSQWSPALTV | |
| | SKVLLSICSLLCDPNPDDPLVPDIA | |
| | QIYKSDKEKYNRHAREWTQKYAML | |
| | EGGGGSGPSGGGGPSGSRVSSV | |
| | PTQLEVVAATPTSLLISWDAPAVT | |
| | VDYYVITYGETGHWPWVWQEFEV | |
| | PGSYSTATISGLHPGVDYTITVYAG | |
| | SYSSYYYYGSPISINYRTGGYPYDV | |
| | PDYA | |
| UBE2D1_Linker | ALKRIQKELSDLQRDPPAHCSAGP | SEQ ID NO: |
| 12_aCS3(K7Q, K55Y, | VGDDLFHWQATIMGPPDSAYQGG | 242 |
| K64H)_HA | VFFLTVHFPTDYPFKPPKIAFTTKIY | |
| | HPNINSNGSICLDILRSQWSPALTV | |
| | SKVLLSICSLLCDPNPDDPLVPDIA | |
| | QIYKSDKEKYNRHAREWTQKYAML | |
| | ESNGGGGSPAPAPGGGGSGSSR | |
| | VSSVPTQLEVVAATPTSLLISWDA | |
| | PAVTVDYYVITYGETGHWPWVWQ | |
| | EFEVPGSYSTATISGLHPGVDYTIT | |
| | VYAGSYSSYYYYGSPISINYRTGG | |
| | YPYDVPDYA | |
| UBE2D1_Linker | ALKRIQKELSDLQRDPPAHCSAGP | SEQ ID NO: |
| 13_aCS3(K7Q, K55Y, | VGDDLFHWQATIMGPPDSAYQGG | 243 |
| K64H) | VFFLTVHFPTDYPFKPPKIAFTTKIY | |
| (HA tag is contained | HPNINSNGSICLDILRSQWSPALTV | |
| within the linker) | SKVLLSICSLLCDPNPDDPLVPDIA | |
| | QIYKSDKEKYNRHAREWTQKYAML | |
| | EGGGGSYPYDVPDYASGGGGSS | |
| | RVSSVPTQLEVVAATPTSLLISWD | |
| | APAVTVDYYVITYGETGHWPWVW | |
| | QEFEVPGSYSTATISGLHPGVDYTI | |
| | TVYAGSYSSYYYYGSPISINYRT | |
| UBE2D1_Linker | ALKRIQKELSDLQRDPPAHCSAGP | SEQ ID NO: |
| 14_aCS3(K7Q, K55Y, | VGDDLFHWQATIMGPPDSAYQGG | 244 |
| K64H)_HA | VFFLTVHFPTDYPFKPPKIAFTTKIY | |
| | HPNINSNGSICLDILRSQWSPALTV | |
| | SKVLLSICSLLCDPNPDDPLVPDIA | |
| | QIYKSDKEKYNRHAREWTQKYAM | |
| | TGGSAGGSGGSAGGSGGSAGGS | |
| | GGSAVSSVPTQLEVVAATPTSLLIS | |
| | WDAPAVTVDYYVITYGETGHWPW | |
| | VWQEFEVPGSYSTATISGLHPGVD | |
| | YTITVYAGSYSSYYYYGSPISINYRT | |
| | GGYPYDVPDYA | |
| UBE2D1_Linker | ALKRIQKELSDLQRDPPAHCSAGP | SEQ ID NO: |
| 15_aCS3(K7Q, K55Y, | VGDDLFHWQATIMGPPDSAYQGG | 245 |
| K64H)_HA | VFFLTVHFPTDYPFKPPKIAFTTKIY | |
| | HPNINSNGSICLDILRSQWSPALTV | |
| | SKVLLSICSLLCDPNPDDPLVPDIA | |
| | QIYKSDKEKYNRHAREWTQKYAM | |
| | AGSGGSTGSGGSPTPSTSGGSTG | |
| | SGGASVSSVPTQLEVVAATPTSLLI | |
| | SWDAPAVTVDYYVITYGETGHWP | |
| | WVWQEFEVPGSYSTATISGLHPG | |
| | VDYTITVYAGSYSSYYYYGSPISIN | |
| | YRTGGYPYDVPDYA | |
| UBE2D1_Linker | ALKRIQKELSDLQRDPPAHCSAGP | SEQ ID NO: |
| 16_aCS3(K7Q, K55Y, | VGDDLFHWQATIMGPPDSAYQGG | 246 |
| K64H)_HA | VFFLTVHFPTDYPFKPPKIAFTTKIY | |
| | HPNINSNGSICLDILRSQWSPALTV | |
| | SKVLLSICSLLCDPNPDDPLVPDIA | |
| | QIYKSDKEKYNRHAREWTQKYAM | |
| | AGSGGSGGSGGSGNSSTSGGSG | |
| | GSGGASVSSVPTQLEVVAATPTSL | |
| | LISWDAPAVTVDYYVITYGETGHW | |
| | PWVWQEFEVPGSYSTATISGLHP | |
| | GVDYTITVYAGSYSSYYYYGSPISI | |
| | NYRTGGYPYDVPDYA | |
| UBE2D1_Linker | ALKRIQKELSDLQRDPPAHCSAGP | SEQ ID NO: |
| 17_aCS3(K7Q, K55Y, | VGDDLFHWQATIMGPPDSAYQGG | 247 |
| K64H)_HA | VFFLTVHFPTDYPFKPPKIAFTTKIY | |
| | HPNINSNGSICLDILRSQWSPALTV | |
| | SKVLLSICSLLCDPNPDDPLVPDIA | |
| | QIYKSDKEKYNRHAREWTQKYAM | |
| | GGSPVPSTPGGGSGGGSGGSPVP | |
| | STPGSVSSVPTQLEVVAATPTSLLI | |
| | SWDAPAVTVDYYVITYGETGHWP | |
| | WVWQEFEVPGSYSTATISGLHPG | |
| | VDYTITVYAGSYSSYYYYGSPISIN | |
| | YRTGGYPYDVPDYA | |
| UBE2D1_Linker | ALKRIQKELSDLQRDPPAHCSAGP | SEQ ID NO: |
| 18_aCS3(K7Q, K55Y, | VGDDLFHWQATIMGPPDSAYQGG | 248 |
| K64H)_HA | VFFLTVHFPTDYPFKPPKIAFTTKIY | |
| | HPNINSNGSICLDILRSQWSPALTV | |
| | SKVLLSICSLLCDPNPDDPLVPDIA | |
| | QIYKSDKEKYNRHAREWTQKYAM | |
| | SPGTGSPGTGSPGTGSPGTGSPG | |
| | TGSPGVSSVPTQLEVVAATPTSLLI | |
| | SWDAPAVTVDYYVITYGETGHWP | |
| | WVWQEFEVPGSYSTATISGLHPG | |
| | VDYTITVYAGSYSSYYYYGSPISIN | |
| | YRTGGYPYDVPDYA | |
| UBE2D1(C85A)_ | ALKRIQKELSDLQRDPPAHCSAGP | SEQ ID NO: |
| Linker2_aCS3(K7Q, | VGDDLFHWQATIMGPPDSAYQGG | 266 |
| K55Y, K64H)_HA | VFFLTVHFPTDYPFKPPKIAFTTKIY | |
| | HPNINSNGSIALDILRSQWSPALTV | |
| | SKVLLSICSLLCDPNPDDPLVPDIA | |
| | QIYKSDKEKYNRHAREWTQKYAML | |
| | EGGGGSGGGGSGGGGSSRVSSV | |
| | PTQLEVVAATPTSLLISWDAPAVT | |
| | VDYYVITYGETGHWPWVWQEFEV | |
| | PGSYSTATISGLHPGVDYTITVYAG | |
| | SYSSYYYYGSPISINYRTGGYPYDV | |
| | PDYA | |
| UBE2D1_Linker2_aCS3 | ALKRIQKELSDLQRDPPAHCSAGP | SEQ ID NO: |
| (K7Q, K55Y, K64H, | VGDDLFHWQATIMGPPDSAYQGG | 267 |
| V33R)_HA | VFFLTVHFPTDYPFKPPKIAFTTKIY | |
| | HPNINSNGSICLDILRSQWSPALTV | |
| | SKVLLSICSLLCDPNPDDPLVPDIA | |
| | QIYKSDKEKYNRHAREWTQKYAML | |
| | EGGGGSGGGGSGGGGSSRVSSV | |
| | PTQLEVVAATPTSLLISWDAPAVT | |
| | VDYYRITYGETGHWPWVWQEFEV | |
| | PGSYSTATISGLHPGVDYTITVYAG | |
| | SYSSYYYYGSPISINYRTGGYPYDV | |
| | PDYA | |
| UBE2D1(C85A)_ | ALKRIQKELSDLQRDPPAHCSAGP | SEQ ID NO: |
| Linker2_aCS3(K7Q, | VGDDLFHWQATIMGPPDSAYQGG | 268 |
| K55Y, K64H, V33R)_HA | VFFLTVHFPTDYPFKPPKIAFTTKIY | |
| | HPNINSNGSIALDILRSQWSPALTV | |
| | SKVLLSICSLLCDPNPDDPLVPDIA | |
| | QIYKSDKEKYNRHAREWTQKYAML | |
| | EGGGGSGGGGSGGGGSSRVSSV | |
| | PTQLEVVAATPTSLLISWDAPAVT | |
| | VDYYRITYGETGHWPWVWQEFEV | |
| | PGSYSTATISGLHPGVDYTITVYAG | |
| | SYSSYYYYGSPISINYRTGGYPYDV | |
| | PDYA | |
| UBE2D1(F62A) | ALKRIQKELSDLQRDPPAHCSAGP | SEQ ID NO: |
| Linker2_aCS3(K7Q, | VGDDLFHWQATIMGPPDSAYQGG | 269 |
| K55Y, K64H)_HA | VFFLTVHFPTDYPAKPPKIAFTTKIY | |
| | HPNINSNGSICLDILRSQWSPALTV | |
| | SKVLLSICSLLCDPNPDDPLVPDIA | |
| | QIYKSDKEKYNRHAREWTQKYAML | |
| | EGGGGSGGGGSGGGGSSRVSSV | |
| | PTQLEVVAATPTSLLISWDAPAVT | |
| | VDYYVITYGETGHWPWVWQEFEV | |
| | PGSYSTATISGLHPGVDYTITVYAG | |
| | SYSSYYYYGSPISINYRTGGYPYDV | |
| | PDYA | |
| UBE2B_ | STPARRRLMRDFKRLQEDPPVGV | SEQ ID NO: |
| Linker2_aCS3(K7Q, | SGAPSENNIMQWNAVIFGPEGTPF | 270 |
| K55Y, K64H)_HA | EDGTFKLVIEFSEEYPNKPPTVRFL | |
| | SKMFHPNVYADGSICLDILQNRWS | |
| | PTYDVSSILTSIQSLLDEPNPNSPA | |
| | NSQAAQLYQENKREYEKRVSAIVE | |
| | QSWNDSLEGGGGSGGGGGGGG | |
| | SSRVSSVPTQLEVVAATPTSLLIS | |
| | WDAPAVTVDYYVITYGETGHWPW | |
| | VWQEFEVPGSYSTATISGLHPGVD | |
| | YTITVYAGSYSSYYYYGSPISINYRT | |
| | GGYPYDVPDYA | |
| UBE2B(C88A)_Linker2_ | STPARRRLMRDFKRLQEDPPVGV | SEQ ID NO: |
| aCS3(K7Q, K55Y, | SGAPSENNIMQWNAVIFGPEGTPF | 271 |
| K64H)_HA | EDGTFKLVIEFSEEYPNKPPTVRFL | |
| | SKMFHPNVYADGSIALDILQNRWS | |
| | PTYDVSSILTSIQSLLDEPNPNSPA | |
| | NSQAAQLYQENKREYEKRVSAIVE | |
| | QSWNDSLEGGGGSGGGGSGGGG | |
| | SSRVSSVPTQLEVVAATPTSLLIS | |
| | WDAPAVTVDYYVITYGETGHWPW | |
| | VWQEFEVPGSYSTATISGLHPGVD | |
| | YTITVYAGSYSSYYYYGSPISINYRT | |
| | GGYPYDVPDYA | |
| UBE2B_Linker2_aCS3 | STPARRRLMRDFKRLQEDPPVGV | SEQ ID NO: |
| (K7Q, K55Y, K64H, | SGAPSENNIMQWNAVIFGPEGTPF | 272 |
| V33R)_HA | EDGTFKLVIEFSEEYPNKPPTVRFL | |
| | SKMFHPNVYADGSICLDILQNRWS | |
| | PTYDVSSILTSIQSLLDEPNPNSPA | |
| | NSQAAQLYQENKREYEKRVSAIVE | |
| | QSWNDSLEGGGGSGGGGSGGGG | |
| | SSRVSSVPTQLEVVAATPTSLLIS | |
| | WDAPAVTVDYYRITYGETGHWPW | |
| | VWQEFEVPGSYSTATISGLHPGVD | |
| | YTITVYAGSYSSYYYYGSPISINYRT | |
| | GGYPYDVPDYA | |
| UBE2B(C88A)_Linker2_ | STPARRRLMRDFKRLQEDPPVGV | SEQ ID NO: |
| aCS3(K7Q, K55Y, K64H, | SGAPSENNIMQWNAVIFGPEGTPF | 273 |
| V33R)_HA | EDGTFKLVIEFSEEYPNKPPTVRFL | |
| | SKMFHPNVYADGSIALDILQNRWS | |
| | PTYDVSSILTSIQSLLDEPNPNSPA | |
| | NSQAAQLYQENKREYEKRVSAIVE | |
| | QSWNDSLEGGGGSGGGGSGGGG | |
| | SSRVSSVPTQLEVVAATPTSLLIS | |
| | WDAPAVTVDYYRITYGETGHWPW | |
| | VWQEFEVPGSYSTATISGLHPGVD | |
| | YTITVYAGSYSSYYYYGSPISINYRT | |
| | GGYPYDVPDYA | |

TABLE 12B
Fusion polypeptide containing E3 and corresponding SEQ ID NOS.
Fusion polypeptides

| Expression tag_ | | |
|---|---|---|
| Regulation domain_ | | |
| Linker_ | | |
| Targeting domain | Amino acid sequence | SEQ ID NO: |
| HA_VHL_Linker 1_aCS3 | YPYDVPDYAAMPRRAENWDEAEV | SEQ ID NO: |
| | GAEEAGVEEYGPEEDGGEESGAE | 152 |
| | ESGPEESGPEELGAEEEMEVGRP | |
| | RPVLRSVNSREPSQVIFCNRSPRV | |
| | VLPVWLNFDGEPQPYPTLPPGTG | |
| | RRIHSYRGHLWLFRDAGTHDGLL | |
| | VNQTELFVPSLNVDGQPIFANITLP | |
| | VYTLKERCLQVVRSLVKPENYRRL | |
| | DIVRSLYEDLEDHPNVQKDLERLT | |
| | QERIAHQRMGDLEGGGGSSRVSS | |
| | VPTKLEVVAATPTSLLISWDAPAVT | |
| | VDYYVITYGETGHWPWVWQEFEV | |
| | PGSKSTATISGLKPGVDYTITVYAG | |
| | SYSSYYYYGSPISINYRT | |
| HA_VHL_Linker 2_aCS3 | YPYDVPDYAAMPRRAENWDEAEV | SEQ ID NO: |
| | GAEEAGVEEYGPEEDGGEESGAE | 153 |
| | ESGPEESGPEELGAEEEMEVGRP | |
| | RPVLRSVNSREPSQVIFCNRSPRV | |
| | VLPVWLNFDGEPQPYPTLPPGTG | |
| | RRIHSYRGHLWLFRDAGTHDGLL | |
| | VNQTELFVPSLNVDGQPIFANITLP | |
| | VYTLKERCLQVVRSLVKPENYRRL | |
| | DIVRSLYEDLEDHPNVQKDLERLT | |
| | QERIAHQRMGDLEGGGGSGGGG | |
| | SGGGGSSRVSSVPTKLEVVAATPT | |
| | SLLISWDAPAVTVDYYVITYGETGH | |
| | WPWVWQEFEVPGSKSTATISGLK | |
| | PGVDYTITVYAGSYSSYYYYGSPIS | |
| | INYRT | |
| HA_VHL_Linker 4_aCS3 | YPYDVPDYAAMPRRAENWDEAEV | SEQ ID NO: |
| | GAEEAGVEEYGPEEDGGEESGAE | 154 |
| | ESGPEESGPEELGAEEEMEVGRP | |
| | RPVLRSVNSREPSQVIFCNRSPRV | |
| | VLPVWLNFDGEPQPYPTLPPGTG | |
| | RRIHSYRGHLWLFRDAGTHDGLL | |
| | VNQTELFVPSLNVDGQPIFANITLP | |
| | VYTLKERCLQVVRSLVKPENYRRL | |
| | DIVRSLYEDLEDHPNVQKDLERLT | |
| | QERIAHQRMGDGGGGGEFAMVSS | |
| | VPTKLEVVAATPTSLLISWDAPAVT | |
| | VDYYVITYGETGHWPWVWQEFEV | |
| | PGSKSTATISGLKPGVDYTITVYAG | |
| | SYSSYYYYGSPISINYRT | |
| HA_VHL_Linker | YPYDVPDYAAMPRRAENWDEAEV | SEQ ID NO: |
| 2_aCS3(V33R) | GAEEAGVEEYGPEEDGGEESGAE | 155 |
| | ESGPEESGPEELGAEEEMEVGRP | |
| | RPVLRSVNSREPSQVIFCNRSPRV | |
| | VLPVWLNFDGEPQPYPTLPPGTG | |
| | RRIHSYRGHLWLFRDAGTHDGLL | |
| | VNQTELFVPSLNVDGQPIFANITLP | |
| | VYTLKERCLQVVRSLVKPENYRRL | |
| | DIVRSLYEDLEDHPNVQKDLERLT | |
| | QERIAHQRMGDLEGGGGSGGGG | |
| | SGGGGSSRVSSVPTKLEVVAATPT | |
| | SLLISWDAPAVTVDYYRITYGETGH | |
| | WPWVWQEFEVPGSKSTATISGLK | |
| | PGVDYTITVYAGSYSSYYYYGSPIS | |
| | INYRT | |
| HA_VHL_Linker2_E3_5 | YPYDVPDYAAMPRRAENWDEAEV | SEQ ID NO: |
| | GAEEAGVEEYGPEEDGGEESGAE | 276 |
| | ESGPEESGPEELGAEEEMEVGRP | |
| | RPVLRSVNSREPSQVIFCNRSPRV | |
| | VLPVWLNFDGEPQPYPTLPPGTG | |
| | RRIHSYRGHLWLFRDAGTHDGLL | |
| | VNQTELFVPSLNVDGQPIFANITLP | |
| | VYTLKERCLQVVRSLVKPENYRRL | |
| | DIVRSLYEDLEDHPNVQKDLERLT | |
| | QERIAHQRMGDLEGGGGSGGGG | |
| | SGGGGSSRDLGKKLLEAARAGQD | |
| | DEVRILMANGADVNATDNDGYTPL | |
| | HLAASNGHLEIVEVLLKNGADVNAS | |
| | DLTGITPLHLAAATGHLEIVEVLLKH | |
| | GADVNAYDNDGHTPLHLAAKYGHL | |
| | EIVEVLLKHGADVNAQDKFGKTAF | |
| | DISIDNGNEDLAEILQ | |
| HA_VHL_Linker2_K19 | YPYDVPDYAAMPRRAENWDEAEV | SEQ ID NO: |
| | GAEEAGVEEYGPEEDGGEESGAE | 277 |
| | ESGPEESGPEELGAEEEMEVGRP | |
| | RPVLRSVNSREPSQVIFCNRSPRV | |
| | VLPVWLNFDGEPQPYPTLPPGTG | |
| | RRIHSYRGHLWLFRDAGTHDGLL | |
| | VNQTELFVPSLNVDGQPIFANITLP | |
| | VYTLKERCLQVVRSLVKPENYRRL | |
| | DIVRSLYEDLEDHPNVQKDLERLT | |
| | QERIAHQRMGDLEGGGGSGGGG | |
| | SGGGGSSRDLGKKLLEAARAGQD | |
| | DEVRILMANGADVNASDRWGWTP | |
| | LHLAAWWGHLEIVEVLLKRGADVS | |
| | AADLHGQSPLHLAAMVGHLEIVEV | |
| | LLKYGADVNAKDTMGATPLHLAAR | |
| | SGHLEIVEELLKNGADMNAQDKFG | |
| | KTTFDISTDNGNEDLAEILQKL | |
| Expression tag_ | | |
| Targeting domain_ | | |
| Linker_ | | |
| Regulation domain | Amino acid sequence | SEQ ID NO: |
| HA_aCS3_Linker 1_VHL | YPYDVPDYAEFAMVSSVPTKLEVV | SEQ ID NO: |
| | AATPTSLLISWDAPAVTVDYYVITY | 196 |
| | GETGHWPWVWQEFEVPGSKSTA | |
| | TISGLKPGVDYTITVYAGSYSSYYY | |
| | YGSPISINYRTLEGGGGSSR | |
| | AMPRRAENWDEAEVGAEEAGVEE | |
| | YGPEEDGGEESGAEESGPEESGP | |
| | EELGAEEEMEAGRPRPVLRSVNS | |
| | REPSQVIFCNRSPRVVLPVWLNFD | |
| | GEPQPYPTLPPGTGRRIHSYRGHL | |
| | WLFRDAGTHDGLLVNQTELFVPSL | |
| | NVDGQPIFANITLPVYTLKERCLQV | |
| | VRSLVKPENYRRLDIVRSLYEDLED | |
| | HPNVQKDLERLTQERIAHQRMGD | |
| HA_aCS3_Linker | YPYDVPDYAEFAMVSSVPTKLEVV | SEQ ID NO: |
| 2_VHL | AATPTSLLISWDAPAVTVDYYVITY | 197 |
| | GETGHWPWVWQEFEVPGSKSTA | |
| | TISGLKPGVDYTITVYAGSYSSYYY | |
| | YGSPISINYRTLEGGGGSGGGGSG | |
| | GGGSSRAMPRRAENWDEAEVGA | |
| | EEAGVEEYGPEEDGGEESGAEES | |
| | GPEESGPEELGAEEEMEAGRPRP | |
| | VLRSVNSREPSQVIFCNRSPRVVL | |
| | PVWLNFDGEPQPYPTLPPGTGRRI | |
| | HSYRGHLWLFRDAGTHDGLLVNQ | |
| | TELFVPSLNVDGQPIFANITLPVYTL | |
| | KERCLQVVRSLVKPENYRRLDIVR | |
| | SLYEDLEDHPNVQKDLERLTQERIA | |
| | HQRMGD | |
| HA_K19_linker 3_VHL | YPYDVPDYADLGKKLLEAARAGQ | SEQ ID NO: |
| | DDEVRILMANGADVNASDRWGWT | 198 |
| | PLHLAAWWGHLEIVEVLLKRGAD | |
| | VSAADLHGQSPLHLAAMVGHLEIV | |
| | EVLLKYGADVNAKDTMGATPLHL | |
| | AARSGHLEIVEELLKNGADMNAQ | |
| | DKFGKTTFDISTDNGNEDLAEILQK | |
| | LAAAGGGGSGGGGSGGGGSGTP | |
| | RRAENWDEAEVGAEEAGVEEYGP | |
| | EEDGGEESGAEESGPEESGPEEL | |
| | GAEEEMEAGRPRPVLRSVNSREP | |
| | SQVIFCNRSPRVVLPVWLNFDGEP | |
| | QPYPTLPPGTGRRIHSYRGHLWLF | |
| | RDAGTHDGLLVNQTELFVPSLNVD | |
| | GQPIFANITLPVYTLKERCLQVVRS | |
| | LVKPENYRRLDIVRSLYEDLEDHPN | |
| | VQKDLERLTQERIAHQRMGD | |
| HA_E3_5_linker 3_VHL | YPYDVPDYADLGKKLLEAARAGQ | SEQ ID NO: |
| | DDEVRILMANGADVNATDNDGYT | 199 |
| | PLHLAASNGHLEIVEVLLKNGADV | |
| | NASDLTGITPLHLAAATGHLEIVEV | |
| | LLKHGADVNAYDNDGHTPLHLAA | |
| | KYGHLEIVEVLLKHGADVNAQDKF | |
| | GKTAFDISIDNGNEDLAEILQAAAG | |
| | GGGSGGGGSGGGGSGTPRRAEN | |
| | WDEAEVGAEEAGVEEYGPEEDGG | |
| | EESGAEESGPEESGPEELGAEEE | |
| | MEAGRPRPVLRSVNSREPSQVIFC | |
| | NRSPRVVLPVWLNFDGEPQPYPTL | |
| | PPGTGRRIHSYRGHLWLFRDAGTH | |
| | DGLLVNQTELFVPSLNVDGQPIFAN | |
| | ITLPVYTLKERCLQVVRSLVKPENY | |
| | RRLDIVRSLYEDLEDHPNVQKDLE | |
| | RLTQERIAHQRMGD | |
| HA_aCS3_Linker 4_VHL | YPYDVPDYAEFAMVSSVPTKLEVV | SEQ ID NO: |
| | AATPTSLLISWDAPAVTVDYYVITY | 200 |
| | GETGHWPWVWQEFEVPGSKSTA | |
| | TISGLKPGVDYTITVYAGSYSSYYY | |
| | YGSPISINYRTGGGGGAMPRRAEN | |
| | WDEAEVGAEEAGVEEYGPEEDGG | |
| | EESGAEESGPEESGPEELGAEEE | |
| | MEVGRPRPVLRSVNSREPSQVIFC | |
| | NRSPRVVLPVWLNFDGEPQPYPTL | |
| | PPGTGRRIHSYRGHLWLFRDAGTH | |
| | DGLLVNQTELFVPSLNVDGQPIFAN | |
| | ITLPVYTLKERCLQVVRSLVKPENY | |
| | RRLDIVRSLYEDLEDHPNVQKDLE | |
| | RLTQERIAHQRMGD | |
| HA_aCS3(V33R)_ | YPYDVPDYAEFAMVSSVPTKLEVV | SEQ ID NO: |
| Linker 2_VHL | AATPTSLLISWDAPAVTVDYYRITY | 201 |
| | GETGHWPWVWQEFEVPGSKSTA | |
| | TISGLKPGVDYTITVYAGSYSSYYY | |
| | YGSPISINYRTLEGGGGSGGGGSG | |
| | GGGSSRAMPRRAENWDEAEVGA | |
| | EEAGVEEYGPEEDGGEESGAEES | |
| | GPEESGPEELGAEEEMEAGRPRP | |
| | VLRSVNSREPSQVIFCNRSPRVVL | |
| | PVWLNFDGEPQPYPTLPPGTGRRI | |
| | HSYRGHLWLFRDAGTHDGLLVNQ | |
| | TELFVPSLNVDGQPIFANITLPVYTL | |
| | KERCLQVVRSLVKPENYRRLDIVR | |
| | SLYEDLEDHPNVQKDLERLTQERIA | |
| | HQRMGD | |
| HA_E3_5_Linker 2_VHL | YPYDVPDYADLGKKLLEAARAGQ | SEQ ID NO: |
| | DDEVRILMANGADVNATDNDGYT | 278 |
| | PLHLAASNGHLEIVEVLLKNGADV | |
| | NASDLTGITPLHLAAATGHLEIVEV | |
| | LLKHGADVNAYDNDGHTPLHLAA | |
| | KYGHLEIVEVLLKHGADVNAQDKF | |
| | GKTAFDISIDNGNEDLAEILQLEGG | |
| | GGSGGGGSGGGGSSRAMPRRAE | |
| | NWDEAEVGAEEAGVEEYGPEEDG | |
| | GEESGAEESGPEESGPEELGAEE | |
| | EMEVGRPRPVLRSVNSREPSQVIF | |
| | CNRSPRVVLPVWLNFDGEPQPYP | |
| | TLPPGTGRRIHSYRGHLWLFRDAG | |
| | THDGLLVNQTELFVPSLNVDGQPIF | |
| | ANITLPVYTLKERCLQVVRSLVKPE | |
| | NYRRLDIVRSLYEDLEDHPNVQKD | |
| | LERLTQERIAHQRMGD | |
| HA_K19_Linker2_VHL | YPYDVPDYADLGKKLLEAARAGQ | SEQ ID NO: |
| | DDEVRILMANGADVNASDRWGWT | 279 |
| | PLHLAAWWGHLEIVEVLLKRGAD | |
| | VSAADLHGQSPLHLAAMVGHLEIV | |
| | EVLLKYGADVNAKDTMGATPLHL | |
| | AARSGHLEIVEELLKNGADMNAQ | |
| | DKFGKTTFDISTDNGNEDLAEILQK | |
| | LLEGGGGSGGGGSGGGGSSRAM | |
| | PRRAENWDEAEVGAEEAGVEEYG | |
| | PEEDGGEESGAEESGPEESGPEE | |
| | LGAEEEMEVGRPRPVLRSVNSRE | |
| | PSQVIFCNRSPRVVLPVWLNFDGE | |
| | PQPYPTLPPGTGRRIHSYRGHLWL | |
| | FRDAGTHDGLLVNQTELFVPSLNV | |
| | DGQPIFANITLPVYTLKERCLQVVR | |
| | SLVKPENYRRLDIVRSLYEDLEDHP | |
| | NVQKDLERLTQERIAHQRMGD | |

TABLE 12C
Polynucleotide sequences encoding fusion polypeptides of the disclosure

| | DNA sequence | SEQ ID NO |
|---|---|---|
| UBE2D1_Linker | CCTTGAAGCGGATTCAGAAAGAGCTGAGCG | 223 |
| 7_aCS3(K7Q, K55Y, | ACCTGCAGAGGGACCCTCCTGCTCACTGTT | |
| K64H)_HA | CTGCTGGACCCGTGGGAGATGACCTGTTCC | |
| | ACTGGCAAGCCACCATCATGGGCCCTCCAG | |
| | ATAGCGCCTATCAAGGCGGCGTGTTCTTCCT | |
| | GACCGTGCACTTCCCCACAGACTACCCCTT | |
| | CAAGCCTCCTAAGATCGCCTTCACCACCAAG | |
| | ATCTATCACCCCAACATCAACAGCAACGGCA | |
| | GCATCTGCCTGGACATCCTGAGAAGCCAAT | |
| | GGTCCCCTGCTCTGACCGTGTCCAAGGTGC | |
| | TGCTGAGCATCTGCAGCCTGCTGTGCGACC | |
| | CCAATCCTGACGATCCTCTGGTGCCTGATAT | |
| | CGCCCAGATCTACAAGAGCGACAAAGAGAA | |
| | GTACAACCGGCACGCCAGAGAGTGGACCCA | |
| | GAAATACGCTATGCTGGAAGGCGGCTCTAG | |
| | AGTGTCCAGCGTGCCCACACAGCTGGAAGT | |
| | GGTTGCCGCCACACCTACAAGCCTGCTGAT | |
| | CTCTTGGGATGCCCCTGCCGTGACAGTGGA | |
| | CTACTACGTGATCACCTACGGCGAGACAGG | |
| | CCACTGGCCTTGGGTCTGGCAAGAGTTTGA | |
| | AGTGCCCGGCAGCTACAGCACCGCCACAAT | |
| | TTCTGGACTGCACCCCGGCGTGGACTACAC | |
| | CATCACAGTGTACGCCGGCTCCTACAGCAG | |
| | CTACTACTACTATGGCAGCCCCATCAGCATC | |
| | AACTACCGGACAGGCGGCTACCCCTACGAC | |
| | GTGCCAGATTATGCTTGA | |
| UBE2D1_Linker | GCCTTGAAGCGGATTCAGAAAGAGCTGAGC | 224 |
| 8_aCS3 | GACCTGCAGAGGGACCCTCCTGCTCACTGT | |
| (K7Q, K55Y, K64H)_ | TCTGCTGGACCCGTGGGAGATGACCTGTTC | |
| HA | CACTGGCAAGCCACCATCATGGGCCCTCCA | |
| | GATAGCGCCTATCAAGGCGGCGTGTTCTTC | |
| | CTGACCGTGCACTTCCCCACAGACTACCCC | |
| | TTCAAGCCTCCTAAGATCGCCTTCACCACCA | |
| | AGATCTATCACCCCAACATCAACAGCAACGG | |
| | CAGCATCTGCCTGGACATCCTGAGAAGCCA | |
| | ATGGTCCCCTGCTCTGACCGTGTCCAAGGT | |
| | GCTGCTGAGCATCTGCAGCCTGCTGTGCGA | |
| | CCCCAATCCTGACGATCCTCTGGTGCCTGAT | |
| | ATCGCCCAGATCTACAAGAGCGACAAAGAG | |
| | AAGTACAACCGGCACGCCAGAGAGTGGACC | |
| | CAGAAATACGCTATGCTGGAAGGCGGCGGA | |
| | AGCGGCGGATCTTCTAGAGTGTCCAGCGTG | |
| | CCCACACAGCTGGAAGTGGTTGCCGCCACA | |
| | CCTACAAGCCTGCTGATCTCTTGGGATGCC | |
| | CCTGCCGTGACAGTGGACTACTACGTGATC | |
| | ACCTACGGCGAGACAGGCCACTGGCCTTGG | |
| | GTCTGGCAAGAGTTTGAAGTGCCCGGCAGC | |
| | TACAGCACCGCCACAATTTCTGGACTGCACC | |
| | CCGGCGTGGACTACACCATCACAGTGTACG | |
| | CCGGCTCCTACAGCAGCTACTACTACTATGG | |
| | CAGCCCCATCAGCATCAACTACCGGACAGG | |
| | CGGCTACCCCTACGACGTGCCAGATTATGC | |
| | TTGA | |
| UBE2D1_Linker | GCCTTGAAGCGGATTCAGAAAGAGCTGAGC | 225 |
| 9_aCS3 | GACCTGCAGAGGGACCCTCCTGCTCACTGT | |
| (K7Q, K55Y, K64H)_ | TCTGCTGGACCCGTGGGAGATGACCTGTTC | |
| HA | CACTGGCAAGCCACCATCATGGGCCCTCCA | |
| | GATAGCGCCTATCAAGGCGGCGTGTTCTTC | |
| | CTGACCGTGCACTTCCCCACAGACTACCCC | |
| | TTCAAGCCTCCTAAGATCGCCTTCACCACCA | |
| | AGATCTATCACCCCAACATCAACAGCAACGG | |
| | CAGCATCTGCCTGGACATCCTGAGAAGCCA | |
| | ATGGTCCCCTGCTCTGACCGTGTCCAAGGT | |
| | GCTGCTGAGCATCTGCAGCCTGCTGTGCGA | |
| | CCCCAATCCTGACGATCCTCTGGTGCCTGAT | |
| | ATCGCCCAGATCTACAAGAGCGACAAAGAG | |
| | AAGTACAACCGGCACGCCAGAGAGTGGACC | |
| | CAGAAATACGCTATGCTGGAAGGCGGCGGA | |
| | GGATCTGGTGGTGGATCTTCTAGAGTGTCC | |
| | AGCGTGCCCACACAGCTGGAAGTGGTTGCC | |
| | GCCACACCTACAAGCCTGCTGATCTCTTGG | |
| | GATGCCCCTGCCGTGACAGTGGACTACTAC | |
| | GTGATCACCTACGGCGAGACAGGCCACTGG | |
| | CCTTGGGTCTGGCAAGAGTTTGAAGTGCCC | |
| | GGCAGCTACAGCACCGCCACAATTTCTGGA | |
| | CTGCACCCCGGCGTGGACTACACCATCACA | |
| | GTGTACGCCGGCTCCTACAGCAGCTACTAC | |
| | TACTATGGCAGCCCCATCAGCATCAACTACC | |
| | GGACAGGCGGCTACCCCTACGACGTGCCAG | |
| | ATTATGCTTGA | |
| UBE2D1_Linker | GCCTTGAAGCGGATTCAGAAAGAGCTGAGC | 226 |
| 10_aCS3(K7Q, K55Y, | GACCTGCAGAGGGACCCTCCTGCTCACTGT | |
| K64H)_HA | TCTGCTGGACCCGTGGGAGATGACCTGTTC | |
| | CACTGGCAAGCCACCATCATGGGCCCTCCA | |
| | GATAGCGCCTATCAAGGCGGCGTGTTCTTC | |
| | CTGACCGTGCACTTCCCCACAGACTACCCC | |
| | TTCAAGCCTCCTAAGATCGCCTTCACCACCA | |
| | AGATCTATCACCCCAACATCAACAGCAACGG | |
| | CAGCATCTGCCTGGACATCCTGAGAAGCCA | |
| | ATGGTCCCCTGCTCTGACCGTGTCCAAGGT | |
| | GCTGCTGAGCATCTGCAGCCTGCTGTGCGA | |
| | CCCCAATCCTGACGATCCTCTGGTGCCTGAT | |
| | ATCGCCCAGATCTACAAGAGCGACAAAGAG | |
| | AAGTACAACCGGCACGCCAGAGAGTGGACC | |
| | CAGAAATACGCTATGCTGGAAGGCGGCGGA | |
| | AGCGGCGGAGGGAGCGGTGGTGGATCTTC | |
| | TAGAGTGTCCAGCGTGCCCACACAGCTGGA | |
| | AGTGGTTGCCGCCACACCTACAAGCCTGCT | |
| | GATCTCTTGGGATGCCCCTGCCGTGACAGT | |
| | GGACTACTACGTGATCACCTACGGCGAGAC | |
| | AGGCCACTGGCCTTGGGTCTGGCAAGAGTT | |
| | TGAAGTGCCCGGCAGCTACAGCACCGCCAC | |
| | AATTTCTGGACTGCACCCCGGCGTGGACTA | |
| | CACCATCACAGTGTACGCCGGCTCCTACAG | |
| | CAGCTACTACTACTATGGCAGCCCCATCAGC | |
| | ATCAACTACCGGACAGGCGGCTACCCCTAC | |
| | GACGTGCCAGATTATGCTTGA | |
| UBE2D1_Linker | GCCTTGAAGCGGATTCAGAAAGAGCTGAGC | 227 |
| 2_aCS3(K7Q, K55Y, | GACCTGCAGAGGGACCCTCCTGCTCACTGT | |
| K64H)_HA | TCTGCTGGACCCGTGGGAGATGACCTGTTC | |
| | CACTGGCAAGCCACCATCATGGGCCCTCCA | |
| | GATAGCGCCTATCAAGGCGGCGTGTTCTTC | |
| | CTGACCGTGCACTTCCCCACAGACTACCCC | |
| | TTCAAGCCTCCTAAGATCGCCTTCACCACCA | |
| | AGATCTATCACCCCAACATCAACAGCAACGG | |
| | CAGCATCTGCCTGGACATCCTGAGAAGCCA | |
| | ATGGTCCCCTGCTCTGACCGTGTCCAAGGT | |
| | GCTGCTGAGCATCTGCAGCCTGCTGTGCGA | |
| | CCCCAATCCTGACGATCCTCTGGTGCCTGAT | |
| | ATCGCCCAGATCTACAAGAGCGACAAAGAG | |
| | AAGTACAACCGGCACGCCAGAGAGTGGACC | |
| | CAGAAATACGCTATGCTGGAAGGCGGCGGA | |
| | GGATCTGGCGGAGGCGGTAGCGGTGGTGG | |
| | TGGATCTTCTAGAGTGTCCAGCGTGCCCAC | |
| | ACAGCTGGAAGTGGTTGCCGCCACACCTAC | |
| | AAGCCTGCTGATCTCTTGGGATGCCCCTGC | |
| | CGTGACAGTGGACTACTACGTGATCACCTAC | |
| | GGCGAGACAGGCCACTGGCCTTGGGTCTG | |
| | GCAAGAGTTTGAAGTGCCCGGCAGCTACAG | |
| | CACCGCCACAATTTCTGGACTGCACCCCGG | |
| | CGTGGACTACACCATCACAGTGTACGCCGG | |
| | CTCCTACAGCAGCTACTACTACTATGGCAGC | |
| | CCCATCAGCATCAACTACCGGACAGGCGGC | |
| | TACCCCTACGACGTGCCAGATTATGCTTGA | |
| UBE2D1_Linker | GCCTTGAAGCGGATTCAGAAAGAGCTGAGC | 228 |
| 11_aCS3(K7Q, K55Y, | GACCTGCAGAGGGACCCTCCTGCTCACTGT | |
| K64H)_HA | TCTGCTGGACCCGTGGGAGATGACCTGTTC | |
| | CACTGGCAAGCCACCATCATGGGCCCTCCA | |
| | GATAGCGCCTATCAAGGCGGCGTGTTCTTC | |
| | CTGACCGTGCACTTCCCCACAGACTACCCC | |
| | TTCAAGCCTCCTAAGATCGCCTTCACCACCA | |
| | AGATCTATCACCCCAACATCAACAGCAACGG | |
| | CAGCATCTGCCTGGACATCCTGAGAAGCCA | |
| | ATGGTCCCCTGCTCTGACCGTGTCCAAGGT | |
| | GCTGCTGAGCATCTGCAGCCTGCTGTGCGA | |
| | CCCCAATCCTGACGATCCTCTGGTGCCTGAT | |
| | ATCGCCCAGATCTACAAGAGCGACAAAGAG | |
| | AAGTACAACCGGCACGCCAGAGAGTGGACC | |
| | CAGAAATACGCTATGCTGGAAGGCGGCGGA | |
| | GGCTCTGGACCTTCTGGTGGCGGAGGACCA | |
| | TCTGGCTCTAGAGTGTCTAGCGTGCCCACA | |
| | CAGCTGGAAGTGGTGGCCGCTACACCTACA | |
| | AGCCTGCTGATCTCTTGGGATGCCCCTGCC | |
| | GTGACAGTGGACTACTACGTGATCACCTAC | |
| | GGCGAGACAGGCCACTGGCCTTGGGTCTG | |
| | GCAAGAGTTTGAAGTGCCCGGCAGCTACAG | |
| | CACCGCCACAATTTCTGGACTGCACCCCGG | |
| | CGTGGACTACACCATCACAGTGTACGCCGG | |
| | CTCCTACAGCAGCTACTACTACTATGGCAGC | |
| | CCCATCAGCATCAACTACCGGACAGGCGGC | |
| | TACCCCTACGACGTGCCAGATTATGCTTGA | |
| UBE2D1_Linker | GCCCTGAAGAGAATCCAGAAAGAGCTGAGC | 229 |
| 12_aCS3(K7Q, K55Y, | GACCTGCAGCGGGATCCTCCTGCTCACTGT | |
| K64H)_HA | TCTGCTGGACCCGTGGGAGATGACCTGTTC | |
| | CACTGGCAAGCCACCATCATGGGCCCTCCA | |
| | GATAGCGCCTATCAAGGCGGCGTGTTCTTC | |
| | CTGACCGTGCACTTCCCCACAGACTACCCC | |
| | TTCAAGCCTCCTAAGATCGCCTTCACCACCA | |
| | AGATCTATCACCCCAACATCAACAGCAACGG | |
| | CAGCATCTGCCTGGACATCCTGAGAAGCCA | |
| | ATGGTCCCCTGCTCTGACCGTGTCCAAGGT | |
| | GCTGCTGAGCATCTGCAGCCTGCTGTGCGA | |
| | CCCCAATCCTGACGATCCTCTGGTGCCTGAT | |
| | ATCGCCCAGATCTACAAGAGCGACAAAGAG | |
| | AAGTACAACCGGCACGCCAGAGAGTGGACC | |
| | CAGAAATACGCCATGCTGGAAAGCAATGGC | |
| | GGCGGAGGATCTCCAGCTCCTGCTCCTGGC | |
| | GGAGGCGGATCTGGATCTTCTAGAGTGTCC | |
| | AGCGTGCCCACACAGCTGGAAGTGGTTGCC | |
| | GCCACACCTACAAGCCTGCTGATCTCTTGG | |
| | GATGCCCCTGCCGTGACAGTGGACTACTAC | |
| | GTGATCACCTACGGCGAGACAGGCCACTGG | |
| | CCTTGGGTCTGGCAAGAGTTTGAAGTGCCC | |
| | GGCAGCTACAGCACCGCCACAATTTCTGGA | |
| | CTGCACCCCGGCGTGGACTACACCATCACA | |
| | GTGTACGCCGGCTCCTACAGCAGCTACTAC | |
| | TACTATGGCAGCCCCATCAGCATCAACTACC | |
| | GGACAGGCGGCTACCCCTACGACGTGCCAG | |
| | ATTATGCTTGA | |
| UBE2D1_Linker | GCCTTGAAGCGGATTCAGAAAGAGCTGAGC | 230 |
| 13_aCS3(K7Q, K55Y, | GACCTGCAGAGGGACCCTCCTGCTCACTGT | |
| K64H) | TCTGCTGGACCCGTGGGAGATGACCTGTTC | |
| (HA tag is contained | CACTGGCAAGCCACCATCATGGGCCCTCCA | |
| within the linker) | GATAGCGCCTATCAAGGCGGCGTGTTCTTC | |
| | CTGACCGTGCACTTCCCCACAGACTACCCC | |
| | TTCAAGCCTCCTAAGATCGCCTTCACCACCA | |
| | AGATCTATCACCCCAACATCAACAGCAACGG | |
| | CAGCATCTGCCTGGACATCCTGAGAAGCCA | |
| | ATGGTCCCCTGCTCTGACCGTGTCCAAGGT | |
| | GCTGCTGAGCATCTGCAGCCTGCTGTGCGA | |
| | CCCCAATCCTGACGATCCTCTGGTGCCTGAT | |
| | ATCGCCCAGATCTACAAGAGCGACAAAGAG | |
| | AAGTACAACCGGCACGCCAGAGAGTGGACC | |
| | CAGAAATACGCCATGTCTCCCGGAACAGGC | |
| | TCTCCTGGCACAGGCAGTCCTGGAACTGGA | |
| | TCACCAGGCACAGGTTCTCCAGGCACTGGA | |
| | AGCCCTGGTGTCAGCTCTGTGCCTACACAG | |
| | CTGGAAGTGGTGGCCGCCACACCTACAAGC | |
| | CTGCTGATCTCTTGGGATGCCCCTGCCGTG | |
| | ACAGTGGACTACTACGTGATCACCTACGGC | |
| | GAGACAGGCCACTGGCCTTGGGTCTGGCAA | |
| | GAGTTTGAAGTGCCCGGCAGCTACAGCACC | |
| | GCCACAATTTCTGGACTGCACCCCGGCGTG | |
| | GACTACACCATCACAGTGTACGCCGGCTCC | |
| | TACAGCAGCTACTACTACTATGGCAGCCCCA | |
| | TCAGCATCAACTACCGGACAGGCGGCTACC | |
| | CCTACGACGTGCCAGATTATGCTTGA | |
| UBE2D1_Linker | GCCTTGAAGCGGATTCAGAAAGAGCTGAGC | 231 |
| 14_aCS3(K7Q, K55Y, | GACCTGCAGAGGGACCCTCCTGCTCACTGT | |
| K64H)_HA | TCTGCTGGACCCGTGGGAGATGACCTGTTC | |
| | CACTGGCAAGCCACCATCATGGGCCCTCCA | |
| | GATAGCGCCTATCAAGGCGGCGTGTTCTTC | |
| | CTGACCGTGCACTTCCCCACAGACTACCCC | |
| | TTCAAGCCTCCTAAGATCGCCTTCACCACCA | |
| | AGATCTATCACCCCAACATCAACAGCAACGG | |
| | CAGCATCTGCCTGGACATCCTGAGAAGCCA | |
| | ATGGTCCCCTGCTCTGACCGTGTCCAAGGT | |
| | GCTGCTGAGCATCTGCAGCCTGCTGTGCGA | |
| | CCCCAATCCTGACGATCCTCTGGTGCCTGAT | |
| | ATCGCCCAGATCTACAAGAGCGACAAAGAG | |
| | AAGTACAACCGGCACGCCAGAGAGTGGACC | |
| | CAGAAATACGCTATGACAGGCGGATCTGCC | |
| | GGCGGAAGCGGAGGTAGTGCTGGTGGATCT | |
| | GGTGGTTCAGCTGGTGGCAGCGGAGGATCT | |
| | GCTGTCAGCTCTGTGCCTACACAGCTGGAA | |
| | GTGGTGGCCGCCACACCTACAAGCCTGCTG | |
| | ATCTCTTGGGATGCCCCTGCCGTGACAGTG | |
| | GACTACTACGTGATCACCTACGGCGAGACA | |
| | GGCCACTGGCCTTGGGTCTGGCAAGAGTTT | |
| | GAAGTGCCCGGCAGCTACAGCACCGCCACA | |
| | ATTTCTGGACTGCACCCCGGCGTGGACTAC | |
| | ACCATCACAGTGTACGCCGGCTCCTACAGC | |
| | AGCTACTACTACTATGGCAGCCCCATCAGCA | |
| | TCAACTACCGGACAGGCGGCTACCCCTACG | |
| | ACGTGCCAGATTATGCTTGA | |
| UBE2D1_Linker | TATTCACTCGAGGCCGCCACCATGGCCTTG | 232 |
| 15_aCS3(K7Q, K55Y, | AAGCGGATTCAGAAAGAGCTGAGCGACCTG | |
| K64H)_HA | CAGAGGGACCCTCCTGCTCACTGTTCTGCT | |
| | GGACCCGTGGGAGATGACCTGTTCCACTGG | |
| | CAAGCCACCATCATGGGCCCTCCAGATAGC | |
| | GCCTATCAAGGCGGCGTGTTCTTCCTGACC | |
| | GTGCACTTCCCCACAGACTACCCCTTCAAGC | |
| | CTCCTAAGATCGCCTTCACCACCAAGATCTA | |
| | TCACCCCAACATCAACAGCAACGGCAGCAT | |
| | CTGCCTGGACATCCTGAGAAGCCAATGGTC | |
| | CCCTGCTCTGACCGTGTCCAAGGTGCTGCT | |
| | GAGCATCTGCAGCCTGCTGTGCGACCCCAA | |
| | TCCTGACGATCCTCTGGTGCCTGATATCGCC | |
| | CAGATCTACAAGAGCGACAAAGAGAAGTAC | |
| | AACCGGCACGCCAGAGAGTGGACCCAGAAA | |
| | TACGCTATGGCCGGCTCTGGCGGCTCTACA | |
| | GGATCTGGTGGAAGCCCTACACCTAGCACA | |
| | TCTGGCGGAAGCACAGGTTCTGGCGGAGCC | |
| | TCTGTTAGCAGCGTGCCAACACAGCTGGAA | |
| | GTGGTGGCCGCCACACCTACAAGCCTGCTG | |
| | ATCTCTTGGGATGCCCCTGCCGTGACAGTG | |
| | GACTACTACGTGATCACCTACGGCGAGACA | |
| | GGCCACTGGCCTTGGGTCTGGCAAGAGTTT | |
| | GAAGTGCCCGGCAGCTACAGCACCGCCACA | |
| | ATTTCTGGACTGCACCCCGGCGTGGACTAC | |
| | ACCATCACAGTGTACGCCGGCTCCTACAGC | |
| | AGCTACTACTACTATGGCAGCCCCATCAGCA | |
| | TCAACTACCGGACAGGCGGCTACCCCTACG | |
| | ACGTGCCAGATTATGCTTGACTAGTGCATCA | |
| | CA | |
| UBE2D1_Linker | GCCTTGAAGCGGATTCAGAAAGAGCTGAGC | 233 |
| 16_aCS3(K7Q, K55Y, | GACCTGCAGAGGGACCCTCCTGCTCACTGT | |
| K64H)_HA | TCTGCTGGACCCGTGGGAGATGACCTGTTC | |
| | CACTGGCAAGCCACCATCATGGGCCCTCCA | |
| | GATAGCGCCTATCAAGGCGGCGTGTTCTTC | |
| | CTGACCGTGCACTTCCCCACAGACTACCCC | |
| | TTCAAGCCTCCTAAGATCGCCTTCACCACCA | |
| | AGATCTATCACCCCAACATCAACAGCAACGG | |
| | CAGCATCTGCCTGGACATCCTGAGAAGCCA | |
| | ATGGTCCCCTGCTCTGACCGTGTCCAAGGT | |
| | GCTGCTGAGCATCTGCAGCCTGCTGTGCGA | |
| | CCCCAATCCTGACGATCCTCTGGTGCCTGAT | |
| | ATCGCCCAGATCTACAAGAGCGACAAAGAG | |
| | AAGTACAACCGGCACGCCAGAGAGTGGACC | |
| | CAGAAATACGCTATGGCCGGCTCTGGTGGC | |
| | AGCGGAGGTTCTGGTGGATCTGGCAATAGC | |
| | AGCACAAGCGGCGGATCTGGCGGAAGTGG | |
| | CGGAGCCTCTGTTAGCTCTGTGCCCACACA | |
| | GCTGGAAGTGGTGGCCGCCACACCTACAAG | |
| | CCTGCTGATCTCTTGGGATGCCCCTGCCGT | |
| | GACAGTGGACTACTACGTGATCACCTACGG | |
| | CGAGACAGGCCACTGGCCTTGGGTCTGGCA | |
| | AGAGTTTGAAGTGCCCGGCAGCTACAGCAC | |
| | CGCCACAATTTCTGGACTGCACCCCGGCGT | |
| | GGACTACACCATCACAGTGTACGCCGGCTC | |
| | CTACAGCAGCTACTACTACTATGGCAGCCCC | |
| | ATCAGCATCAACTACCGGACAGGCGGCTAC | |
| | CCCTACGACGTGCCAGATTATGCTTGA | |
| UBE2D1_Linker | GCCTTGAAGCGGATTCAGAAAGAGCTGAGC | 234 |
| 17_aCS3(K7Q, K55Y, | GACCTGCAGAGGGACCCTCCTGCTCACTGT | |
| K64H)_HA | TCTGCTGGACCCGTGGGAGATGACCTGTTC | |
| | CACTGGCAAGCCACCATCATGGGCCCTCCA | |
| | GATAGCGCCTATCAAGGCGGCGTGTTCTTC | |
| | CTGACCGTGCACTTCCCCACAGACTACCCC | |
| | TTCAAGCCTCCTAAGATCGCCTTCACCACCA | |
| | AGATCTATCACCCCAACATCAACAGCAACGG | |
| | CAGCATCTGCCTGGACATCCTGAGAAGCCA | |
| | ATGGTCCCCTGCTCTGACCGTGTCCAAGGT | |
| | GCTGCTGAGCATCTGCAGCCTGCTGTGCGA | |
| | CCCCAATCCTGACGATCCTCTGGTGCCTGAT | |
| | ATCGCCCAGATCTACAAGAGCGACAAAGAG | |
| | AAGTACAACCGGCACGCCAGAGAGTGGACC | |
| | CAGAAATACGCTATGGGCGGCAGCCCTGTG | |
| | CCTTCTACACCTGGCGGAGGAAGCGGCGGA | |
| | GGATCTGGTGGATCTCCAGTGCCTAGTACA | |
| | CCAGGCAGCGTGTCCTCTGTGCCCACACAG | |
| | CTGGAAGTGGTGGCCGCCACACCTACAAGC | |
| | CTGCTGATCTCTTGGGATGCCCCTGCCGTG | |
| | ACAGTGGACTACTACGTGATCACCTACGGC | |
| | GAGACAGGCCACTGGCCTTGGGTCTGGCAA | |
| | GAGTTTGAAGTGCCCGGCAGCTACAGCACC | |
| | GCCACAATTTCTGGACTGCACCCCGGCGTG | |
| | GACTACACCATCACAGTGTACGCCGGCTCC | |
| | TACAGCAGCTACTACTACTATGGCAGCCCCA | |
| | TCAGCATCAACTACCGGACAGGCGGCTACC | |
| | CCTACGACGTGCCAGATTATGCTTGA | |
| UBE2D1_Linker | GCCTTGAAGCGGATTCAGAAAGAGCTGAGC | 235 |
| 18_aCS3(K7Q, K55Y, | GACCTGCAGAGGGACCCTCCTGCTCACTGT | |
| K64H)_HA | TCTGCTGGACCCGTGGGAGATGACCTGTTC | |
| | CACTGGCAAGCCACCATCATGGGCCCTCCA | |
| | GATAGCGCCTATCAAGGCGGCGTGTTCTTC | |
| | CTGACCGTGCACTTCCCCACAGACTACCCC | |
| | TTCAAGCCTCCTAAGATCGCCTTCACCACCA | |
| | AGATCTATCACCCCAACATCAACAGCAACGG | |
| | CAGCATCTGCCTGGACATCCTGAGAAGCCA | |
| | ATGGTCCCCTGCTCTGACCGTGTCCAAGGT | |
| | GCTGCTGAGCATCTGCAGCCTGCTGTGCGA | |
| | CCCCAATCCTGACGATCCTCTGGTGCCTGAT | |
| | ATCGCCCAGATCTACAAGAGCGACAAAGAG | |
| | AAGTACAACCGGCACGCCAGAGAGTGGACC | |
| | CAGAAATACGCCATGTCTCCCGGAACAGGC | |
| | TCTCCTGGCACAGGCAGTCCTGGAACTGGA | |
| | TCACCAGGCACAGGTTCTCCAGGCACTGGA | |
| | AGCCCTGGTGTCAGCTCTGTGCCTACACAG | |
| | CTGGAAGTGGTGGCCGCCACACCTACAAGC | |
| | CTGCTGATCTCTTGGGATGCCCCTGCCGTG | |
| | ACAGTGGACTACTACGTGATCACCTACGGC | |
| | GAGACAGGCCACTGGCCTTGGGTCTGGCAA | |
| | GAGTTTGAAGTGCCCGGCAGCTACAGCACC | |
| | GCCACAATTTCTGGACTGCACCCCGGCGTG | |
| | GACTACACCATCACAGTGTACGCCGGCTCC | |
| | TACAGCAGCTACTACTACTATGGCAGCCCCA | |
| | TCAGCATCAACTACCGGACAGGCGGCTACC | |
| | CCTACGACGTGCCAGATTATGCTTGA | |
| HA_K19_Linker | TATCCCTACGACGTGCCCGATTATGCCGAC | 249 |
| 2_UBE2D1 | CTGGGCAAGAAGCTGCTGGAAGCTGCTAGA | |
| | GCCGGCCAGGACGATGAAGTGCGGATTCTG | |
| | ATGGCCAACGGCGCCGATGTGAACGCCTCT | |
| | GATAGATGGGGATGGACCCCTCTGCATCTG | |
| | GCTGCTTGGTGGGGACACCTGGAAATCGTG | |
| | GAAGTGCTGCTGAAGAGAGGGGCCGACGTT | |
| | TCAGCCGCTGATCTGCATGGACAGAGCCCA | |
| | CTGCATCTCGCCGCCATGGTTGGACACCTT | |
| | GAGATTGTCGAGGTCCTGCTGAAGTATGGC | |
| | GCTGACGTGAACGCTAAGGACACCATGGGA | |
| | GCCACACCACTGCACCTGGCAGCTAGATCT | |
| | GGCCATCTGGAAATTGTCGAAGAACTGCTCA | |
| | AGAATGGGGCAGACATGAACGCCCAGGACA | |
| | AGTTCGGCAAGACCACCTTCGACATCAGCA | |
| | CCGACAACGGCAACGAGGACCTGGCCGAG | |
| | ATCCTGCAGAAACTGCTCGAGGGAGGCGGA | |
| | GGATCTGGCGGAGGTGGAAGTGGCGGAGG | |
| | CGGCTCTTCTAGAGCCCTGAAGAGAATCCA | |
| | GAAAGAGCTGAGCGACCTGCAGAGAGATCC | |
| | TCCTGCTCACTGTTCTGCTGGCCCTGTGGG | |
| | AGATGACCTGTTCCACTGGCAAGCCACCAT | |
| | CATGGGCCCTCCAGATAGCGCCTATCAAGG | |
| | CGGCGTGTTCTTCCTGACCGTGCACTTCCC | |
| | CACAGACTACCCCTTCAAGCCTCCTAAGATC | |
| | GCCTTCACCACCAAGATCTATCACCCCAACA | |
| | TCAACAGCAACGGCAGCATCTGCCTGGACA | |
| | TCCTGAGAAGCCAATGGTCCCCTGCTCTGA | |
| | CCGTGTCCAAGGTGCTGCTGAGCATCTGCA | |
| | GCCTGCTGTGCGACCCCAATCCTGACGATC | |
| | CTCTGGTGCCTGATATCGCCCAGATCTACAA | |
| | GAGCGACAAAGAGAAGTACAACCGGCACGC | |
| | CAGAGAGTGGACCCAGAAATACGCTATGTG | |
| | A | |
| HA_UBE2D1_Linker | TATCCCTACGACGTGCCCGATTATGCCGCC | 250 |
| 2_K19 | CTGAAGAGAATCCAGAAAGAGCTGAGCGAC | |
| | CTGCAGAGAGATCCTCCTGCTCACTGTTCTG | |
| | CTGGCCCTGTGGGAGATGACCTGTTCCACT | |
| | GGCAAGCCACCATCATGGGCCCTCCAGATA | |
| | GCGCCTATCAAGGCGGCGTGTTCTTCCTGA | |
| | CCGTGCACTTCCCCACAGACTACCCCTTCAA | |
| | GCCTCCTAAGATCGCCTTCACCACCAAGATC | |
| | TATCACCCCAACATCAACAGCAACGGCAGC | |
| | ATCTGCCTGGACATCCTGAGAAGCCAATGG | |
| | TCCCCTGCTCTGACCGTGTCCAAGGTGCTG | |
| | CTGAGCATCTGCAGCCTGCTGTGCGACCCC | |
| | AATCCTGACGATCCTCTGGTGCCTGATATCG | |
| | CCCAGATCTACAAGAGCGACAAAGAGAAGT | |
| | ACAACCGGCACGCCAGAGAGTGGACCCAGA | |
| | AATACGCTATGCTCGAGGGAGGCGGAGGAT | |
| | CTGGCGGAGGTGGAAGTGGCGGAGGCGGC | |
| | TCTTCTAGAGACCTGGGCAAGAAGCTGCTG | |
| | GAAGCTGCTAGAGCCGGCCAGGACGATGAA | |
| | GTGCGGATTCTGATGGCCAACGGCGCCGAT | |
| | GTGAACGCCTCTGATAGATGGGGATGGACC | |
| | CCTCTGCATCTGGCTGCTTGGTGGGGACAC | |
| | CTGGAAATCGTGGAAGTGCTGCTGAAGAGA | |
| | GGGGCCGACGTTTCAGCCGCTGATCTGCAT | |
| | GGACAGAGCCCACTGCATCTCGCCGCCATG | |
| | GTTGGACACCTTGAGATTGTCGAGGTCCTG | |
| | CTGAAGTATGGCGCTGACGTGAACGCTAAG | |
| | GACACCATGGGAGCCACACCACTGCACCTG | |
| | GCAGCTAGATCTGGCCATCTGGAAATTGTC | |
| | GAAGAACTGCTCAAGAATGGGGCAGACATG | |
| | AACGCCCAGGACAAGTTCGGCAAGACCACC | |
| | TTCGACATCAGCACCGACAACGGCAACGAG | |
| | GACCTGGCCGAGATCCTGCAGAAACTGTGA | |
| HA_K19_Linker | TATCCCTACGACGTGCCCGATTATGCCGAC | 251 |
| 2_UBE2B | CTGGGCAAGAAGCTGCTGGAAGCCGCTAGA | |
| | GCCGGCCAGGATGATGAAGTGCGGATCCTG | |
| | ATGGCCAACGGCGCCGATGTGAATGCCTCT | |
| | GATAGATGGGGCTGGACCCCTCTGCATCTG | |
| | GCTGCTTGGTGGGGACACCTGGAAATCGTG | |
| | GAAGTGCTGCTGAAGAGAGGGGCCGACGTT | |
| | TCAGCCGCTGATCTGCATGGACAGAGCCCA | |
| | CTGCATCTCGCCGCCATGGTTGGACACCTT | |
| | GAGATTGTCGAGGTCCTGCTGAAGTATGGC | |
| | GCTGACGTGAACGCCAAGGACACCATGGGA | |
| | GCCACACCACTTCATCTGGCCGCTAGATCT | |
| | GGCCATCTGGAAATTGTCGAAGAACTGCTCA | |
| | AGAATGGGGCCGATATGAACGCCCAGGATA | |
| | AGTTCGGCAAGACCACCTTCGACATCAGCA | |
| | CCGACAACGGCAACGAGGACCTGGCCGAG | |
| | ATCCTGCAGAAACTGCTTGAAGGCGGCGGA | |
| | GGATCTGGCGGAGGTGGAAGCGGAGGCGG | |
| | TGGAAGCTCTAGATCTACCCCTGCTCGGCG | |
| | GAGACTGATGCGGGACTTCAAGAGACTGCA | |
| | AGAGGACCCTCCTGTGGGAGTGTCTGGCGC | |
| | CCCTAGCGAGAACAACATCATGCAGTGGAA | |
| | CGCCGTGATCTTCGGCCCTGAGGGCACCCC | |
| | TTTTGAGGACGGCACCTTCAAGCTGGTCATC | |
| | GAGTTCAGCGAGGAATACCCCAACAAGCCT | |
| | CCTACCGTGCGGTTCCTGAGCAAGATGTTTC | |
| | ACCCCAACGTGTACGCCGACGGCAGCATCT | |
| | GTCTGGACATCCTCCAGAACAGATGGTCCC | |
| | CAACCTACGATGTGTCCAGCATCCTGACCA | |
| | GCATCCAGAGCCTGCTGGACGAGCCTAATC | |
| | CTAACAGCCCCGCCAATTCTCAGGCCGCTC | |
| | AGCTGTACCAAGAGAACAAGCGCGAGTACG | |
| | AGAAGCGGGTGTCCGCCATCGTTGAGCAGA | |
| | GCTGGAACGACAGCTGA | |
| HA_UBE2B_Linker | TATCCCTACGACGTGCCCGATTATGCCAGCA | 252 |
| 2_K19 | CACCAGCTCGTCGGAGACTGATGCGGGACT | |
| | TCAAGCGGCTGCAAGAGGATCCACCTGTGG | |
| | GAGTTTCTGGCGCCCCTAGCGAGAACAACA | |
| | TCATGCAGTGGAACGCCGTGATCTTCGGCC | |
| | CTGAGGGCACCCCTTTTGAGGACGGCACCT | |
| | TCAAGCTGGTCATCGAGTTCAGCGAGGAAT | |
| | ACCCCAACAAGCCTCCTACCGTGCGGTTCC | |
| | TGAGCAAGATGTTTCACCCCAACGTGTACGC | |
| | CGACGGCAGCATCTGTCTGGACATCCTGCA | |
| | GAACAGATGGTCCCCAACCTACGATGTGTC | |
| | CAGCATCCTGACCAGCATCCAGAGCCTGCT | |
| | GGACGAGCCCAATCCTAACAGCCCTGCCAA | |
| | TTCTCAGGCCGCTCAGCTGTACCAAGAGAA | |
| | CAAGCGCGAGTACGAGAAGCGGGTGTCCG | |
| | CCATCGTTGAGCAGAGCTGGAACGATAGCC | |
| | TTGAAGGCGGCGGAGGATCTGGCGGAGGT | |
| | GGAAGCGGAGGCGGTGGATCTTCTAGAGAT | |
| | CTGGGCAAGAAGCTGCTGGAAGCCGCTAGA | |
| | GCCGGCCAGGATGATGAAGTGCGGATCCTG | |
| | ATGGCCAACGGCGCCGATGTGAATGCCTCT | |
| | GATAGATGGGGCTGGACCCCTCTGCATCTG | |
| | GCTGCTTGGTGGGGACACCTGGAAATCGTG | |
| | GAAGTGCTGCTGAAGAGAGGGGCCGACGTT | |
| | TCAGCCGCTGATCTGCATGGACAGAGCCCT | |
| | CTGCACCTTGCCGCCATGGTTGGACACCTT | |
| | GAGATTGTCGAGGTCCTGCTGAAGTATGGC | |
| | GCTGACGTGAACGCCAAGGACACCATGGGA | |
| | GCCACACCACTTCATCTGGCCGCCAGAAGC | |
| | GGCCATCTGGAAATTGTCGAAGAACTGCTCA | |
| | AGAATGGGGCCGATATGAACGCCCAGGATA | |
| | AGTTCGGCAAGACCACCTTCGACATCAGCA | |
| | CCGACAACGGCAATGAGGACCTGGCCGAGA | |
| | TCCTGCAAAAGCTCTGA | |
| UBE2D1(C85A)_ | GCCTTGAAGCGGATTCAGAAAGAGCTGAGC | 258 |
| Linker2_aCS3(K7Q, | GACCTGCAGAGGGACCCTCCTGCTCACTGT | |
| K55Y, K64H)_HA | TCTGCTGGACCCGTGGGAGATGACCTGTTC | |
| | CACTGGCAAGCCACCATCATGGGCCCTCCA | |
| | GATAGCGCCTATCAAGGCGGCGTGTTCTTC | |
| | CTGACCGTGCACTTCCCCACAGACTACCCC | |
| | TTCAAGCCTCCTAAGATCGCCTTCACCACCA | |
| | AGATCTATCACCCCAACATCAACAGCAACGG | |
| | CAGCATCGCCCTGGACATCCTGAGAAGCCA | |
| | ATGGTCCCCTGCTCTGACCGTGTCCAAGGT | |
| | GCTGCTGAGCATCTGCAGCCTGCTGTGCGA | |
| | CCCCAATCCTGACGATCCTCTGGTGCCTGAT | |
| | ATCGCCCAGATCTACAAGAGCGACAAAGAG | |
| | AAGTACAACCGGCACGCCAGAGAGTGGACC | |
| | CAGAAATACGCTATGCTGGAAGGCGGCGGA | |
| | GGATCTGGCGGAGGCGGTAGCGGTGGTGG | |
| | TGGATCTTCTAGAGTGTCCAGCGTGCCCAC | |
| | ACAGCTGGAAGTGGTTGCCGCCACACCTAC | |
| | AAGCCTGCTGATCTCTTGGGATGCCCCTGC | |
| | CGTGACAGTGGACTACTACGTGATCACCTAC | |
| | GGCGAGACAGGCCACTGGCCTTGGGTCTG | |
| | GCAAGAGTTTGAAGTGCCCGGCAGCTACAG | |
| | CACCGCCACAATTTCTGGACTGCACCCCGG | |
| | CGTGGACTACACCATCACAGTGTACGCCGG | |
| | CTCCTACAGCAGCTACTACTACTATGGCAGC | |
| | CCCATCAGCATCAACTACCGGACAGGCGGC | |
| | TACCCCTACGACGTGCCAGATTATGCT | |
| UBE2D1_Linker2_ | GCCTTGAAGCGGATTCAGAAAGAGCTGAGC | 259 |
| aCS3(K7Q, K55Y, | GACCTGCAGAGGGACCCTCCTGCTCACTGT | |
| K64H, V33R)_HA | TCTGCTGGACCCGTGGGAGATGACCTGTTC | |
| | CACTGGCAAGCCACCATCATGGGCCCTCCA | |
| | GATAGCGCCTATCAAGGCGGCGTGTTCTTC | |
| | CTGACCGTGCACTTCCCCACAGACTACCCC | |
| | TTCAAGCCTCCTAAGATCGCCTTCACCACCA | |
| | AGATCTATCACCCCAACATCAACAGCAACGG | |
| | CAGCATCTGCCTGGACATCCTGAGAAGCCA | |
| | ATGGTCCCCTGCTCTGACCGTGTCCAAGGT | |
| | GCTGCTGAGCATCTGCAGCCTGCTGTGCGA | |
| | CCCCAATCCTGACGATCCTCTGGTGCCTGAT | |
| | ATCGCCCAGATCTACAAGAGCGACAAAGAG | |
| | AAGTACAACCGGCACGCCAGAGAGTGGACC | |
| | CAGAAATACGCTATGCTGGAAGGCGGCGGA | |
| | GGATCTGGCGGAGGCGGTAGCGGTGGTGG | |
| | TGGATCTTCTAGAGTGTCCAGCGTGCCCAC | |
| | ACAGCTGGAAGTGGTTGCCGCCACACCTAC | |
| | AAGCCTGCTGATCTCTTGGGATGCCCCTGC | |
| | CGTGACAGTGGACTACTACCGGATCACCTA | |
| | CGGCGAGACAGGCCACTGGCCTTGGGTCTG | |
| | GCAAGAGTTTGAAGTGCCCGGCAGCTACAG | |
| | CACCGCCACAATTTCTGGACTGCACCCCGG | |
| | CGTGGACTACACCATCACAGTGTACGCCGG | |
| | CTCCTACAGCAGCTACTACTACTATGGCAGC | |
| | CCCATCAGCATCAACTACCGGACAGGCGGC | |
| | TACCCCTACGACGTGCCAGATTATGCT | |
| UBE2D1(C85A)_ | GCCTTGAAGCGGATTCAGAAAGAGCTGAGC | 260 |
| Linker2_aCS3(K7Q, | GACCTGCAGAGGGACCCTCCTGCTCACTGT | |
| K55Y, K64H, V33R)_ | TCTGCTGGACCCGTGGGAGATGACCTGTTC | |
| HA | CACTGGCAAGCCACCATCATGGGCCCTCCA | |
| | GATAGCGCCTATCAAGGCGGCGTGTTCTTC | |
| | CTGACCGTGCACTTCCCCACAGACTACCCC | |
| | TTCAAGCCTCCTAAGATCGCCTTCACCACCA | |
| | AGATCTATCACCCCAACATCAACAGCAACGG | |
| | CAGCATCGCCCTGGACATCCTGAGAAGCCA | |
| | ATGGTCCCCTGCTCTGACCGTGTCCAAGGT | |
| | GCTGCTGAGCATCTGCAGCCTGCTGTGCGA | |
| | CCCCAATCCTGACGATCCTCTGGTGCCTGAT | |
| | ATCGCCCAGATCTACAAGAGCGACAAAGAG | |
| | AAGTACAACCGGCACGCCAGAGAGTGGACC | |
| | CAGAAATACGCTATGCTGGAAGGCGGCGGA | |
| | GGATCTGGCGGAGGCGGTAGCGGTGGTGG | |
| | TGGATCTTCTAGAGTGTCCAGCGTGCCCAC | |
| | ACAGCTGGAAGTGGTTGCCGCCACACCTAC | |
| | AAGCCTGCTGATCTCTTGGGATGCCCCTGC | |
| | CGTGACAGTGGACTACTACCGGATCACCTA | |
| | CGGCGAGACAGGCCACTGGCCTTGGGTCTG | |
| | GCAAGAGTTTGAAGTGCCCGGCAGCTACAG | |
| | CACCGCCACAATTTCTGGACTGCACCCCGG | |
| | CGTGGACTACACCATCACAGTGTACGCCGG | |
| | CTCCTACAGCAGCTACTACTACTATGGCAGC | |
| | CCCATCAGCATCAACTACCGGACAGGCGGC | |
| | TACCCCTACGACGTGCCAGATTATGCT | |
| UBE2D1(F62A)_ | GCCTTGAAGCGGATTCAGAAAGAGCTGAGC | 261 |
| Linker2_aCS3(K7Q, | GACCTGCAGAGGGACCCTCCTGCTCACTGT | |
| K55Y, K64H)_HA | TCTGCTGGACCCGTGGGAGATGACCTGTTC | |
| | CACTGGCAAGCCACCATCATGGGCCCTCCA | |
| | GATAGCGCCTATCAAGGCGGCGTGTTCTTC | |
| | CTGACCGTGCACTTCCCCACAGACTACCCC | |
| | GCCAAGCCTCCTAAGATCGCCTTCACCACC | |
| | AAGATCTATCACCCCAACATCAACAGCAACG | |
| | GCAGCATCTGCCTGGACATCCTGAGAAGCC | |
| | AATGGTCCCCTGCTCTGACCGTGTCCAAGG | |
| | TGCTGCTGAGCATCTGCAGCCTGCTGTGCG | |
| | ACCCCAATCCTGACGATCCTCTGGTGCCTG | |
| | ATATCGCCCAGATCTACAAGAGCGACAAAGA | |
| | GAAGTACAACCGGCACGCCAGAGAGTGGAC | |
| | CCAGAAATACGCTATGCTGGAAGGCGGCGG | |
| | AGGATCTGGCGGAGGCGGTAGCGGTGGTG | |
| | GTGGATCTTCTAGAGTGTCCAGCGTGCCCA | |
| | CACAGCTGGAAGTGGTTGCCGCCACACCTA | |
| | CAAGCCTGCTGATCTCTTGGGATGCCCCTG | |
| | CCGTGACAGTGGACTACTACGTGATCACCTA | |
| | CGGCGAGACAGGCCACTGGCCTTGGGTCTG | |
| | GCAAGAGTTTGAAGTGCCCGGCAGCTACAG | |
| | CACCGCCACAATTTCTGGACTGCACCCCGG | |
| | CGTGGACTACACCATCACAGTGTACGCCGG | |
| | CTCCTACAGCAGCTACTACTACTATGGCAGC | |
| | CCCATCAGCATCAACTACCGGACAGGCGGC | |
| | TACCCCTACGACGTGCCAGATTATGCT | |
| UBE2B_ | AGCACACCAGCTCGGCGGAGACTGATGAGA | 262 |
| Linker2_aCS3(K7Q, | GACTTCAAGCGGCTGCAAGAGGACCCTCCT | |
| K55Y, K64H)_HA | GTGGGAGTTTCTGGCGCCCCTAGCGAGAAC | |
| | AACATCATGCAGTGGAACGCCGTGATCTTC | |
| | GGCCCTGAGGGCACCCCTTTTGAGGACGGC | |
| | ACCTTCAAGCTGGTCATCGAGTTCAGCGAG | |
| | GAATACCCCAACAAGCCTCCTACCGTGCGG | |
| | TTCCTGAGCAAGATGTTTCACCCCAACGTGT | |
| | ACGCCGACGGCAGCATCTGTCTGGACATCC | |
| | TGCAGAACAGATGGTCCCCAACCTACGATG | |
| | TGTCCAGCATCCTGACCAGCATCCAGAGCC | |
| | TGCTGGACGAGCCCAATCCTAACAGCCCTG | |
| | CCAATTCTCAGGCCGCTCAGCTGTACCAAG | |
| | AGAACAAGCGCGAGTACGAGAAGCGGGTGT | |
| | CCGCCATCGTTGAGCAGAGCTGGAATGACA | |
| | GCCTGGAAGGCGGCGGAGGATCTGGCGGA | |
| | GGCGGTAGCGGTGGTGGTGGATCTTCTAGA | |
| | GTGTCCAGCGTGCCCACACAGCTGGAAGTG | |
| | GTTGCCGCCACACCTACAAGCCTGCTGATC | |
| | TCTTGGGATGCCCCTGCCGTGACAGTGGAC | |
| | TACTACGTGATCACCTACGGCGAGACAGGC | |
| | CACTGGCCTTGGGTCTGGCAAGAGTTTGAA | |
| | GTGCCCGGCAGCTACAGCACCGCCACAATT | |
| | TCTGGACTGCACCCCGGCGTGGACTACACC | |
| | ATCACAGTGTACGCCGGCTCCTACAGCAGC | |
| | TACTACTACTATGGCAGCCCCATCAGCATCA | |
| | ACTACCGGACAGGCGGCTACCCCTACGACG | |
| | TGCCAGATTATGCT | |
| UBE2B(C88A)_ | AGCACACCAGCTCGGCGGAGACTGATGAGA | 263 |
| Linker2_aCS3(K7Q, | GACTTCAAGCGGCTGCAAGAGGACCCTCCT | |
| K55Y, K64H)_HA | GTGGGAGTTTCTGGCGCCCCTAGCGAGAAC | |
| | AACATCATGCAGTGGAACGCCGTGATCTTC | |
| | GGCCCTGAGGGCACCCCTTTTGAGGACGGC | |
| | ACCTTCAAGCTGGTCATCGAGTTCAGCGAG | |
| | GAATACCCCAACAAGCCTCCTACCGTGCGG | |
| | TTCCTGAGCAAGATGTTTCACCCCAACGTGT | |
| | ACGCCGACGGCAGCATCGCGCTGGACATCC | |
| | TGCAGAACAGATGGTCCCCAACCTACGATG | |
| | TGTCCAGCATCCTGACCAGCATCCAGAGCC | |
| | TGCTGGACGAGCCCAATCCTAACAGCCCTG | |
| | CCAATTCTCAGGCCGCTCAGCTGTACCAAG | |
| | AGAACAAGCGCGAGTACGAGAAGCGGGTGT | |
| | CCGCCATCGTTGAGCAGAGCTGGAATGACA | |
| | GCCTGGAAGGCGGCGGAGGATCTGGCGGA | |
| | GGCGGTAGCGGTGGTGGTGGATCTTCTAGA | |
| | GTGTCCAGCGTGCCCACACAGCTGGAAGTG | |
| | GTTGCCGCCACACCTACAAGCCTGCTGATC | |
| | TCTTGGGATGCCCCTGCCGTGACAGTGGAC | |
| | TACTACGTGATCACCTACGGCGAGACAGGC | |
| | CACTGGCCTTGGGTCTGGCAAGAGTTTGAA | |
| | GTGCCCGGCAGCTACAGCACCGCCACAATT | |
| | TCTGGACTGCACCCCGGCGTGGACTACACC | |
| | ATCACAGTGTACGCCGGCTCCTACAGCAGC | |
| | TACTACTACTATGGCAGCCCCATCAGCATCA | |
| | ACTACCGGACAGGCGGCTACCCCTACGACG | |
| | TGCCAGATTATGCT | |
| UBE2B_Linker2_ | AGCACACCAGCTCGGCGGAGACTGATGAGA | 264 |
| aCS3(K7Q, K55Y, | GACTTCAAGCGGCTGCAAGAGGACCCTCCT | |
| K64H, V33R)_HA | GTGGGAGTTTCTGGCGCCCCTAGCGAGAAC | |
| | AACATCATGCAGTGGAACGCCGTGATCTTC | |
| | GGCCCTGAGGGCACCCCTTTTGAGGACGGC | |
| | ACCTTCAAGCTGGTCATCGAGTTCAGCGAG | |
| | GAATACCCCAACAAGCCTCCTACCGTGCGG | |
| | TTCCTGAGCAAGATGTTTCACCCCAACGTGT | |
| | ACGCCGACGGCAGCATCTGTCTGGACATCC | |
| | TGCAGAACAGATGGTCCCCAACCTACGATG | |
| | TGTCCAGCATCCTGACCAGCATCCAGAGCC | |
| | TGCTGGACGAGCCCAATCCTAACAGCCCTG | |
| | CCAATTCTCAGGCCGCTCAGCTGTACCAAG | |
| | AGAACAAGCGCGAGTACGAGAAGCGGGTGT | |
| | CCGCCATCGTTGAGCAGAGCTGGAATGACA | |
| | GCCTGGAAGGCGGCGGAGGATCTGGCGGA | |
| | GGCGGTAGCGGTGGTGGTGGATCTTCTAGA | |
| | GTGTCCAGCGTGCCCACACAGCTGGAAGTG | |
| | GTTGCCGCCACACCTACAAGCCTGCTGATC | |
| | TCTTGGGATGCCCCTGCCGTGACAGTGGAC | |
| | TACTACCGGATCACCTACGGCGAGACAGGC | |
| | CACTGGCCTTGGGTCTGGCAAGAGTTTGAA | |
| | GTGCCCGGCAGCTACAGCACCGCCACAATT | |
| | TCTGGACTGCACCCCGGCGTGGACTACACC | |
| | ATCACAGTGTACGCCGGCTCCTACAGCAGC | |
| | TACTACTACTATGGCAGCCCCATCAGCATCA | |
| | ACTACCGGACAGGCGGCTACCCCTACGACG | |
| | TGCCAGATTATGCT | |
| UBE2B(C88A)_ | AGCACACCAGCTCGGCGGAGACTGATGAGA | 265 |
| Linker2_aCS3(K7Q, | GACTTCAAGCGGCTGCAAGAGGACCCTCCT | |
| K55Y, K64H, V33R)_HA | GTGGGAGTTTCTGGCGCCCCTAGCGAGAAC | |
| | AACATCATGCAGTGGAACGCCGTGATCTTC | |
| | GGCCCTGAGGGCACCCCTTTTGAGGACGGC | |
| | ACCTTCAAGCTGGTCATCGAGTTCAGCGAG | |
| | GAATACCCCAACAAGCCTCCTACCGTGCGG | |
| | TTCCTGAGCAAGATGTTTCACCCCAACGTGT | |
| | ACGCCGACGGCAGCATCGCGCTGGACATCC | |
| | TGCAGAACAGATGGTCCCCAACCTACGATG | |
| | TGTCCAGCATCCTGACCAGCATCCAGAGCC | |
| | TGCTGGACGAGCCCAATCCTAACAGCCCTG | |
| | CCAATTCTCAGGCCGCTCAGCTGTACCAAG | |
| | AGAACAAGCGCGAGTACGAGAAGCGGGTGT | |
| | CCGCCATCGTTGAGCAGAGCTGGAATGACA | |
| | GCCTGGAAGGCGGCGGAGGATCTGGCGGA | |
| | GGCGGTAGCGGTGGTGGTGGATCTTCTAGA | |
| | GTGTCCAGCGTGCCCACACAGCTGGAAGTG | |
| | GTTGCCGCCACACCTACAAGCCTGCTGATC | |
| | TCTTGGGATGCCCCTGCCGTGACAGTGGAC | |
| | TACTACCGGATCACCTACGGCGAGACAGGC | |
| | CACTGGCCTTGGGTCTGGCAAGAGTTTGAA | |
| | GTGCCCGGCAGCTACAGCACCGCCACAATT | |
| | TCTGGACTGCACCCCGGCGTGGACTACACC | |
| | ATCACAGTGTACGCCGGCTCCTACAGCAGC | |
| | TACTACTACTATGGCAGCCCCATCAGCATCA | |
| | ACTACCGGACAGGCGGCTACCCCTACGACG | |
| | TGCCAGATTATGCT | |

1. A molecule comprising
(a) a regulation domain comprising an E2 ubiquitin or ubiquitin-like conjugating domain which has an amino acid sequence having at least 80% sequence identity to a human E2 enzyme or a functional part thereof, and
(b) a targeting domain capable of targeting the regulation domain to a substrate
wherein the targeting domain has an amino acid sequence having at least 80% sequence identity to any one of SEQ ID NOs: 126-135, 138-139, 257 and/or the regulation domain has the amino acid sequence having at least 80% sequence identity to any one of SEQ ID NOs: 1-82.

2-24. (canceled)

25. A molecule according to claim 1, wherein the targeting domain has an amino acid sequence of any one of SEQ ID NOs: 126-135, 138-139, 257 or a variant thereof with up to 20 amino acid modifications, and/or wherein the regulation domain has an amino acid sequence of any one of SEQ ID NOs: 1-82 or a variant thereof with up to 30 amino acid modifications.

26. A molecule according to claim 1 wherein:
the targeting domain is a variant of the amino acid sequence of any one of SEQ ID NOs: 126-135, 138-139, 257 in which one or more of the lysine residues has been substituted with another amino acid and/or deleted; and/or
the regulation domain is a variant of the amino acid sequence of any one of SEQ ID NOs: 42-82 in which one or more lysine residues has been substituted with another amino acid and/or deleted.

27. A molecule according to claim 1, wherein the substrate is an intracellular polypeptide.

28. A molecule according to claim 1, wherein the substrate is localised in one or more of the plasma membrane, cytoplasm, nucleus, endosome, endoplasmic reticulum, mitochondria and Golgi apparatus.

29. A molecule according to claim 1, wherein the substrate is localised in the nucleus.

30. A molecule according to claim 1, wherein the substrate is an oncogenic protein, a signalling protein, a GPCR, a post-translationally modified protein, an adhesion protein, a receptor, a cell-cycle protein, a checkpoint protein, a viral protein, a prion protein, a bacterial protein, a parasitic protein, a fungal protein, a DNA binding protein, a structural protein, an enzyme, an immunogen, an antigen, and/or a pathogenic protein.

31. A molecule according to claim 1 wherein the substrate is selected from the group consisting of Ras, KRas, SHP2, human rhinovirus (HRV) protease 3C, muscarinic acetylcholine receptor 2 (M2R), beta-2 adrenergic receptor (β2-AR), crossover junction endonuclease MUS81 (MUS81) and human antigen R (HuR).

32. A molecule according to claim 1, wherein the regulation domain and targeting domain are joined by a linker.

33. (canceled)

34. (canceled)

35. A molecule according to claim 32, wherein the linker comprises the peptide GGGGS (SEQ ID NO: 146), GGGGSGGGGSGGGGS (SEQ ID NO: 145), LEGGGGSSR (SEQ ID NO: 141), LEGGGGSGGGGSGGGGSSR (SEQ ID NO: 142), AAAGGGGSGGGGSGGGGSGT (SEQ ID NO: 143), GGGGG (SEQ ID NO: 144), LEGGSR (SEQ ID NO: 211), LEGGGSGGSSR (SEQ ID NO: 212), LEGGGGSGGGSSR (SEQ ID NO: 213), LEGGGSGGGSGGGSSR (SEQ ID NO: 214), LEGGGGSGPSGGGGPSGSR (SEQ ID NO: 215), LESNGGGGSPAPAPGGGGSGSSR (SEQ ID NO: 216), LEGGGGSYPYDVPDYASGGGGSSR (SEQ ID NO: 217), TGGSAGGSGGSAGGSGGSAGGSGGSA (SEQ ID NO: 218), AGSGGSTGSGGSPTPSTSGGSTGSGGAS (SEQ ID NO: 219), AGSGGSGGSGGSGNSSTSGGSGGSGGAS (SEQ ID NO: 220), GGSPVPSTPGGGSGGGSGGSPVPSTPGS (SEQ ID NO: 221), or SPGTGSPGTGSPGTGSPGTGSPGTGSPG (SEQ ID NO: 222).

36. A molecule according to claim 1, wherein the molecule is a fusion polypeptide.

37. A molecule according to claim 1, wherein the regulation domain is N-terminal to the targeting domain.

38. A molecule according to claim 1, wherein the regulation domain is C-terminal to the targeting domain.

39. A molecule according to claim 1, wherein the E3 ubiquitin or ubiquitin-like ligase or functional part thereof is one that comprises one or more domains selected from the group consisting of a RING (Really Interesting New Gene) domain, a U-box domain, a HECT (homologous to E6-AP carboxyl terminus) domain, and an RBR domain.

40. A molecule according to claim 1, further comprising a detectable marker.

41. (canceled)

42. A molecule according to claim 1, wherein the molecule is a protein having the amino acid sequence of any one of SEQ ID NOs: 156-167, 171-195, 202-204, 236-248, 253-256, 267, 270 and 272.

43. A molecule according to claim 1, wherein the molecule comprises a subcellular localisation signal, such as a nuclear localisation signal, a mitochondrial localisation signal or an endosomal localisation signal.

44. A molecule according to claim 1, wherein the molecule is capable of decreasing the amount of a substrate by at least 20% compared to the amount of the substrate in the absence of the molecule,
optionally wherein the molecule decreases the amount of the substrate in a cell by at least 20% compared to the amount of the substrate in a cell that is otherwise substantially the same, but which does not contain the molecule.

45. A compound comprising (i) a molecule according to claim 1 and (ii) a targeting moiety capable of targeting the molecule to a cell.

46. A compound according to claim 45, wherein the targeting moiety is a binding partner such as an antibody.

47. A compound according to claim 45 wherein the targeting moiety is a polypeptide which is fused to the molecule.

48-72. (canceled)

73. A method of preventing or treating a disease or condition mediated by an aberrant level of a substrate or form thereof in a subject, the method comprising administering a molecule comprising
(a) a regulation domain comprising an E2 ubiquitin or ubiquitin-like conjugating domain which has an amino acid sequence having at least 80% sequence identity to a human E2 enzyme or a functional part thereof, and
(b) a targeting domain capable of targeting the regulation domain to a substrate
wherein the targeting domain has an amino acid sequence having at least 80% sequence identity to any one of SEQ ID NOs: 126-135, 138-139, 257 and/or the regulation domain has the amino acid sequence having at least 80% sequence identity to any one of SEQ ID NOs: 1-82.

74-85. (canceled)

86. A molecule comprising
(a) a regulation domain comprising a human E2 enzyme comprising an E2 ubiquitin or ubiquitin-like conjugating domain
(b) a targeting domain capable of targeting the regulation domain to a substrate;
wherein the human E2 enzyme is selected from the group consisting of UBE2D1 (UbcH5A) (SEQ ID NO: 4) and UBE2D2 (UbcH5B) (SEQ ID NO: 5).
